(19) United States
(12) Patent Application Publication
(10) Pub. No.: US 2022/0081412 A1
(43) Pub. Date: Mar. 17, 2022

(54) QUINOLINYL-PYRAZINE-CARBOXAMIDE COMPOUNDS AND USES THEREOF

(71) Applicant: The Regents of the University of Michigan, Ann Arbor, MI (US)

(72) Inventors: Nouri Neamati, Ann Arbor, MI (US); Joyeeta Roy, Ann Arbor, MI (US)

(21) Appl. No.: 17/416,876

(22) PCT Filed: Dec. 20, 2019
(86) PCT No.: PCT/US2019/067879

§ 371 Date: Jun. 21, 2021

Related U.S. Application Data
(60) Provisional application No. 62/782,852, filed on Dec. 20, 2018.

(51) Int. Cl. C07D 401/12; C07D 401/14; C07D 471/04; C07D 417/14; C07D 451/02; C07D 471/08; C07D 403/04; C07D 487/04; C07D 491/107; C07D 471/10; C07D 495/10; C07D 417/12

(52) U.S. Cl. CPC C07D 401/12; C07D 401/14; C07D 471/04; C07D 417/14; C07D 451/02; C07D 417/12; C07D 403/04; C07D 487/04; C07D 491/107; C07D 471/10; C07D 495/10; C07D 471/08

(57) ABSTRACT

This invention is in the field of medicinal chemistry. In particular, the invention relates to anew class of small-molecules having a quinolinyl-pyrazine-carboxamide (or similar) structure which function as activators of the cholesterol biosynthesis pathway within cancer cells and/or immune cells, which function as activators of the cell cycle regulation pathway within cancer cells and/or immune cells, and which function as up-regulators of HMGCS1 protein expression within cancer cells and/or immune cells, and which function as effective therapeutic agents for treating, ameliorating, and preventing various forms of cancer and other inflammatory disease.

### CROSS REFERENCE TO RELATED APPLICATION

This application claims priority to and the benefit of U.S. Provisional Application No. 62/782,852, filed Dec. 20, 2018, which is hereby incorporated by reference in its entirety.

### FIELD OF THE INVENTION

This invention is in the field of medicinal chemistry. In particular, the invention relates to a new class of small-molecules having a quinolinyl-pyrazine-carboxamide (or similar) structure which function as activators of the cholesterol biosynthesis pathway within cancer cells and/or immune cells, which function as activators of the cell cycle regulation pathway within cancer cells and/or immune cells, and which function as up-regulators of HMGCS1 protein expression within cancer cells and/or immune cells, and which function as effective therapeutic agents for treating, ameliorating, and preventing various forms of cancer and other inflammatory diseases.

### INTRODUCTION

Cancer is the second most common cause of death in the United States. As of 2015, the cancer death rate for men and women combined had fallen 26% from its peak in 1991. This decline translates to nearly 2.4 million deaths averted during this time period (Cancer Facts & Figures American Cancer Society). This improvement reflects progress in diagnosing at early stage and improvements in treatment. There is still urgent need for the development of effective anticancer drugs with low toxicity.

### SUMMARY

Experiments conducted during the course of developing embodiments for the present invention resulted in the design, synthesis, and characterization of a series of small-molecules having a quinolinyl-pyrazine-carboxamide (or similar) structure that are useful for the treatment and prevention of cancer and inflammatory diseases. The active scaffold was identified using a phenotypic screening of a library of 20,000 small molecules representing 5 million compounds. Approximately 70 novel analogs were designed, synthesized and tested in multiple cancer cell lines using MTT and colony formation assays. In the lead optimization campaign many compounds showed activity less than 1 micromolar. Few representative compounds having a novel structure and drug like properties showed in vitro cytotoxicity in a panel of 61 cancer cell lines with unique selectivity across certain cancer cells.

Such compounds were shown to function as activators of the cholesterol biosynthesis pathway within cancer cells and/or immune cells (e.g., activate gene expression of one or more of AVPI1, CCNG2, TUBA1A, H2AFX, and HIST1H3C). For example, nascent RNA sequencing and mass-spectrometry-based proteomics of cells treated with representative compounds revealed induction of cholesterol biosynthesis pathway based upon the up-regulation of representative genes such as NEU1, INSIG1, DDIT4 and DHCR7 and down-regulation of genes such as GPR135, SPDYA, ABCA1 and HRH4.

Such compounds were shown to function as activators of the cell cycle regulation pathway within cancer cells and/or immune cells (e.g., activate gene expression of one or more of AVPI1, CCNG2, TUBA1A, H2AFX, and HIST1H3C).

Such compounds were shown to function as up-regulators of HMGCS1 protein expression within cancer cells and/or immune cells.

As such, the compounds are useful for treating, ameliorating, and preventing various forms of cancer and other inflammatory diseases.

As such, the present invention provides a new class of small-molecules having a quinolinyl-pyrazine-carboxamide (or similar) structure which function as activators of the cholesterol biosynthesis pathway within cancer cells and/or immune cells, which function as activators of the cell cycle regulation pathway within cancer cells and/or immune cells, and which function as up-regulators of the Hydroxymethylglutaryl-CoA synthase, cytoplasmic (HMGCS1) protein expression within cancer cells and/or immune cells, and which function as effective therapeutic agents for treating, ameliorating, and preventing various forms of cancer and other inflammatory diseases.

Accordingly, the present invention contemplates that exposure of animals (e.g., humans) suffering from cancer (e.g., and/or cancer related disorders) to therapeutically effective amounts of drug(s) having a quinolinyl-pyrazine-carboxamide (or similar) structure that are useful in treating, ameliorating, and preventing various forms of cancer will inhibit the growth of cancer cells or supporting cells outright and/or render such cells as a population more susceptible to the cell death-inducing activity of cancer therapeutic drugs or radiation therapies. In some embodiments, the therapeutic effect occurs through, for example, activating the cholesterol biosynthesis pathway within cancer cells. In some embodiments, the therapeutic effect occurs through, for example, activating the cell cycle regulation pathway within cancer cells. In some embodiments, the therapeutic effect occurs through, for example, up-regulating expression of the HMGCS1 within cancer cells. The present invention contemplates that such compounds having a quinolinyl-pyrazine-carboxamide (or similar) structure satisfy an unmet need for the treatment of multiple cancer types, either when administered as monotherapy to induce cell growth inhibition, apoptosis and/or cell cycle arrest in cancer cells, or when administered in a temporal relationship with additional agent(s), such as other cell death-inducing or cell cycle disrupting cancer therapeutic drugs or radiation therapies (combination therapies), so as to render a greater proportion of the cancer cells or supportive cells susceptible to executing the apoptosis program compared to the corresponding proportion of cells in an animal treated only with the cancer therapeutic drug or radiation therapy alone.

In certain embodiments of the invention, combination treatment of animals with a therapeutically effective amount of a compound of the present invention and a course of an anti cancer agent produces a greater tumor response and clinical benefit in such animals compared to those treated with the compound or anticancer drugs/radiation alone. Since the doses for all approved anticancer drugs and radiation treatments are known, the present invention contemplates the various combinations of them with the present compounds.

The Applicants have found that certain compounds having a quinolinyl-pyrazine-carboxamide (or similar) structure function as activators of the cholesterol biosynthesis pathway within cancer cells and/or immune cells (e.g., activating gene expression within one or more of the genes listed in Tables III-XIX) (e.g., activating gene expression of one or more of INSIG1, DHCR7, MVK and MSMO1) (e.g., de-activating gene expression of one or more of GPR135, SPDYA, ABCA1 and HRH4), function as activators of the cell cycle regulation pathway within cancer cells and/or immune cells (e.g., activating gene expression one or more of AVPI1, CCNG2, TUBA1A, H2AFX, and HIST1H3C), function as up-regulators of HMGCS1 protein expression within cancer cells and/or immune cells, and function as effective therapeutic agents for treating, ameliorating, and preventing various forms of cancer and other inflammatory diseases. Thus, the present invention relates to certain compounds having a quinolinyl-pyrazine-carboxamide (or similar) structure useful for increasing the sensitivity of cells to inducers of apoptosis and/or cell cycle arrest.

Certain compounds having a quinolinyl-pyrazine-carboxamide (or similar) structure may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, quinolinyl-pyrazine-carboxamide (or similar) compounds encompassed within Formula IA or IB are provided:

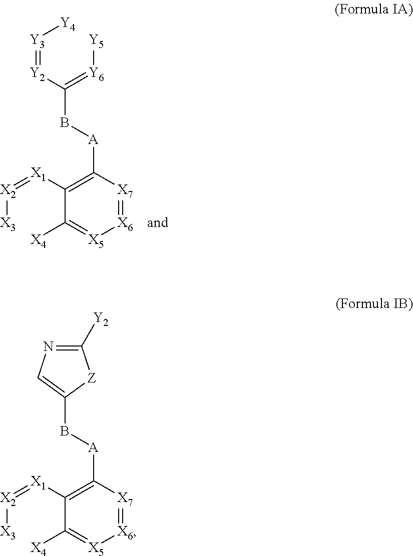

including pharmaceutically acceptable salts (e.g., 2,2,2-trifluoroacetate (TFA) salts and other salts) (e.g., physiologically tolerated acid addition salts), solvates, and/or prodrugs thereof.

Formulas IA and IB are not limited to a particular chemical moiety for A, B, X1, X2, X3, X4, X5, X6, X7, Y2, Y3, Y4, Y5, Y6 and Z. In some embodiments, the particular chemical moiety for A, B, X1, X2, X3, X4, X5, X6, X7, Y2, Y3, Y4, Y5, Y6 and Z independently include any chemical moiety that permits the resulting compound capable of activating the cholesterol biosynthesis pathway within cancer cells and/or immune cells (e.g., activating gene expression within one or more of the genes listed in Tables III-XIX) (e.g., activating gene expression of one or more of INSIG1, DHCR7, MVK and MSMO1) (e.g., de-activating gene expression of one or more of GPR135, SPDYA, ABCA1 and HRH4). In some embodiments, the particular chemical moiety for A, B, X1, X2, X3, X4, X5, X6, X7, Y2, Y3, Y4, Y5, and Y6 independently include any chemical moiety that permits the resulting compound capable of activating the cell cycle regulation pathway within cancer cells and/or immune cells (e.g., activating gene expression one or more of AVPI1, CCNG2, TUBA1A, H2AFX, and HIST1H3C). In some embodiments, the particular chemical moiety for A, B, X1, X2, X3, X4, X5, X6, X7, Y2, Y3, Y4, Y5, and Y6 independently include any chemical moiety that permits the resulting compound capable of up-regulating HMGCS1 protein expression within cancer cells and/or immune cells. In some embodiments, the particular chemical moiety for A, B, X1, X2, X3, X4, X5, X6, X7, Y2, Y3, Y4, Y5, and Y6 independently include any chemical moiety that permits the resulting compound capable of serving as an effective therapeutic agent for treating, ameliorating, and preventing various forms of cancer and other inflammatory diseases.

In some embodiments, X¹ is either CH or N.

In some embodiments, X², X³, X⁴, X⁵, X⁶ and X⁷ are independently selected from CR¹ or N, with the proviso that at least three of them must be CR¹.

In some embodiments, Y², Y³, Y⁴, Y⁵, Y⁶ are independently CH, CR² or N. In some embodiments, Y⁶ is a bond, in which case one of Y³, Y⁴, or Y⁵ is NR³, O, or S, while the other two may be CR² or N.

In some embodiments, A and B are independently selected from a group consisting of NH, C═O, C═S, CH₂, C(R³)₂, CF₂, C—NMe₂, C═N—OR⁴, C═N—N(R⁵)₂, C═N—SO₂R⁶, or C═N—CN.

In some embodiments, Z is either O, S or NH.

In some embodiments, R¹ is independently H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆thioalkoxy, C₁₋₆ thioalkoxy-C₃₋₇ cycloalkyl. C₁₋₆thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR¹⁰, SO₂R¹⁰, NO₂, COR⁷, C₁₋₆ alkyl-COR⁷, N(R¹⁰)C₂₋₆ alkyl-NR¹⁰R¹⁰, —N(R¹⁰)C₂₋₆ alkyl-R⁷, N(C₂₋₆ alkyl)₂-NR¹⁰, —O(CH₂)ₚR⁷, —S(CH₂)ₚR⁷, or —N(R¹⁰)C(═O)(CH₂)ₚR⁷, with a proviso that not more than three R¹ can be other than H.

In some embodiments, R² is independently H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆thioalkoxy-C₃₋₇ cycloalkyl, C₁₋₆thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR¹⁰, SO₂R¹⁰, NO₂, COR⁷, C₁₋₆ alkyl-COR⁷, N(R¹⁰)C₂₋₆alkyl-NR¹⁰R¹⁰,

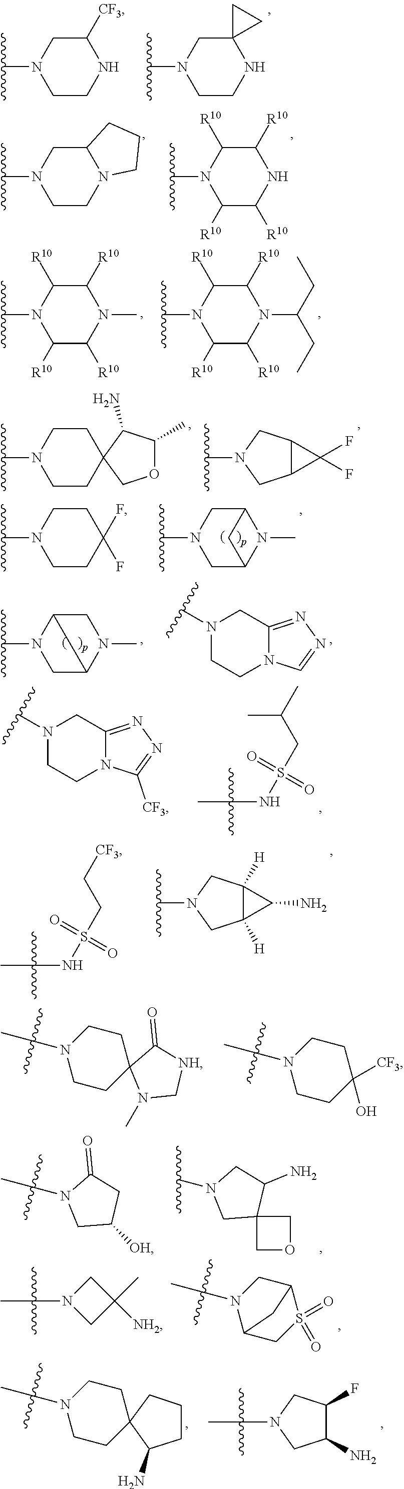

CF₃, CO₂Et, CO₂H, —N(R¹⁰)C₂₋₆ alkyl-R⁷, —O(CH₂)ₚR⁷, —S(CH₂)ₚR⁷, or —N(R¹⁰)C(═O)(CH₂)ₚR⁷, with a proviso that not more than two R² can be other than H.

In some embodiments, R³ is hydrogen, C₁₋₆ alkyl, C₃₋₆ alkenyl, C₃₋₆alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, or C₁₋₆ alkyl-C₄₋₇ heterocycloalkyl.

In some embodiments, R⁴ is H or C₁₋₆ alkyl.

In some embodiments, each R⁵ is independently H or C₁₋₆ alkyl, or the two R⁵, taken together with the N atom to which they are both attached, form a heterocycloalkyl ring of 4-7 members, containing up to one other heteroatom selected from O, S, or NR³; R⁶ is C₁₋₆ alkyl or CF₃.

In some embodiments, R⁷ is OH, NR⁸R⁹, O(CH₂)_{q}NR⁸R⁹. C₁₋₆ alkoxy, C₁₋₆ alkoxy-C₁₋₆ alkoxy, C₂₋₆ hydroxy alkoxy, cyclopropyl,

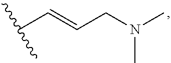 [SMILES: */C=C/CN(C)C]

oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, diazepanylamino, all of which may be optionally substituted with OH, OR¹⁰, oxo, halogen, R¹⁰, CH₂OR¹⁰, CH₂NR⁸R⁹ or CH₂CH₂CONR⁸R⁹.

In some embodiments, R⁸ and R⁹ are each independently H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁₋₆ acyl, 4-12 membered monocyclic or bicyclic heterocyclyl, 4-12 membered monocyclic or bicyclic heterocyclyl-C₁-C₆ alkyl-, C₆₋C₁₂ aryl, 5-11 membered heteroaryl; wherein R⁸ and R⁹ may be further independently substituted with up to three substituents chosen from hydroxyl, C₁₋₆alkoxy, C₁₋₆ hydroxyalkyl, C₁₋₆alkoxy-C₁₋₆ alkyl, C₁₋₆ alkoxy-C₁₋₆alkoxy, C₂₋₆ hydroxyalkoxy, oxo, thiono, cyano or halo; or alternatively, R⁸ and R⁹, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R¹⁰ is independently H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂— alkyl, NH—C₂₋₆ alkyl-NR⁸R⁹, C₁₋₆ alkoxy-C₁₋₆ alkyl or C₂₋₆ alkyl-NR⁸R⁹; alternatively, two R¹⁰ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, X1 is N, and A is NH thereby rendering a compound encompassed within Formula II

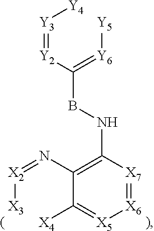

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵ Y⁶ are independently selected from CH, CR² or N; or Y⁶ is a bond, in which case one of Y³, Y⁴, or Y⁵ is NR³, O, or S, while the other two may be CR² or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;

wherein R¹, R², R³, R⁴, R⁵, R⁶, (R⁷-R¹⁰ embedded in R¹ and R²) are as described within Formula I.

In some embodiments, X1 is N, and A is C═O thereby rendering a compound encompassed within Formula III

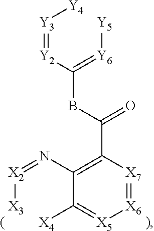

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵ Y⁶ are independently selected from CH, CR² or N;

wherein B is selected from a group consisting of NH, CH₂, C(R³)₂, C—NMe₂, C═N—OR⁴, C═N—N(R⁵)₂;

wherein R¹, R², R³, R⁴, R⁵, (R⁷-R¹⁰ embedded in R¹ and R²) are as described within Formula I.

In some embodiments, X1 is N, and A is CH₂ thereby rendering a compound encompassed within Formula IV

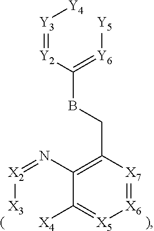

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵, Y⁶ are independently selected from CH, CR² or N; or Y⁶ is a bond, in which case one of Y³, Y⁴, or Y⁵ is NR³, O, or S, while the other two may be CR² or N;

wherein B selected from a group consisting of NH, C═O, C═S, CH₂, C(R³)₂, CF₂, C—NMe₂, C═N—OR⁴, C═N—N(R⁵)₂;

wherein R¹, R², R³, R⁴, R⁵, (R⁷-R¹⁰ embedded in R¹ and R²) are as described in Formula I.

In some embodiments, X1 is N, A is NH, and Y4 is C—R2 thereby rendering a compound encompassed within Formula V

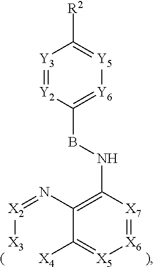

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁵, Y⁶ are independently selected from CH or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within Formula I.

In some embodiments, X1 is N, A is NH, and X2 is C—O—CH3 thereby rendering a compound encompassed within Formula VI

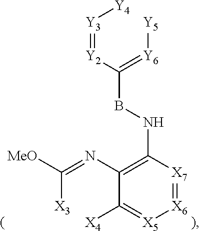

including pharmaceutically acceptable salts (e.g., 2,2,2-trifluoroacetate (TFA) salts and other salts) (e.g., physiologically tolerated acid addition salts), solvates, and/or prodrugs thereof,

wherein X³, X⁴, X⁵, X⁶ and X⁷ are independently selected from CR¹ or N;

wherein Y², Y³, Y⁴, Y⁵, Y⁶ are independently selected from CH, CR² or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within Formula I.

In some embodiments, X1 is N, A is NH, and X6 is C—F thereby rendering a compound encompassed within Formula VII

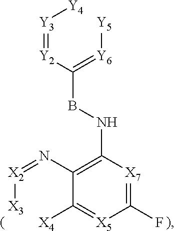

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵ and X⁷ are independently selected from CR¹ or N;

wherein Y², Y³, Y⁴, Y⁵, Y⁶ are independently selected from CH, CR² or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within Formula I.

In some embodiments, X1 is N, A is NH, B is CH, and X6 is C—CH3 thereby rendering a compound encompassed within Formula VIII

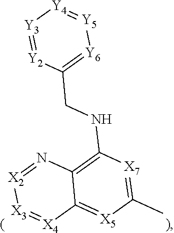

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵ and X⁷ are independently selected from CR¹ or N, with the proviso that at least two of them must be CR¹;

wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;

wherein R¹, R², R³, (R⁷-R¹⁰ embedded in R¹ and R²) are as described within Formula I.

In some embodiments, X1 is N, and A is NH, thereby rendering a compound encompassed within Formula IX

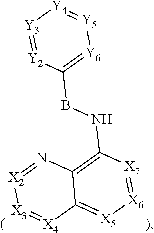

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;

wherein B is selected from a group consisting of NH, C═O, C═S, CH₂, C(R³)₂, CF₂, C—NMe₂, C═N—OR⁴, C═N—N(R⁵)₂, C═N—SO₂R⁶, C═N—CN;

wherein R¹ is N(C₂₋₆ alkyl)₂—NH;

wherein R² is selected from H or Me;

wherein R³, R⁴, R⁵, R⁶ are as described within Formula I.

In some embodiments, X1 is N, X2 is CH, X3 is CH, X4 is CH, X5 is CH, X6 is C—R1, X7 is CH, and Y4 is

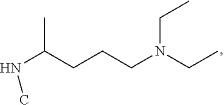

thereby rendering a compound encompassed within Formula X

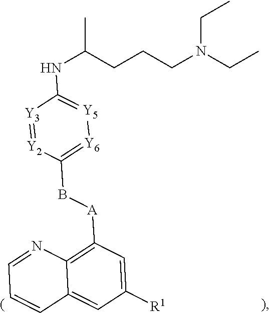

including pharmaceutically acceptable salts (e.g., 2,2,2-trifluoroacetate (TFA) salts and other salts) (e.g., physiologically tolerated acid addition salts), solvates, and/or prodrugs thereof,

wherein Y², Y³, Y⁵ Y⁶ are independently CH or N;

wherein A and B selected from a group consisting of NH, C═O, C═S, CFh, C(R³)₂, CF₂, C—NMe₂, C═N—OR⁴, C═N—N(R⁵)₂, C═N—SO₂R⁶, C═N—CN;
wherein R¹, R³, R⁴, R⁵, R⁶, (R⁷-R¹⁰ embedded in R¹) are as described within Formula I.

In some embodiments, A is NH, B is C═O, X1 is N, X2 is CH, X3 is CH, X4 is CH, X5 is CH, X6 is C—R1, X7 is CH, and Y4 is

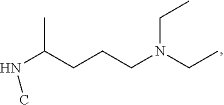

thereby rendering a compound encompassed within Formula XI

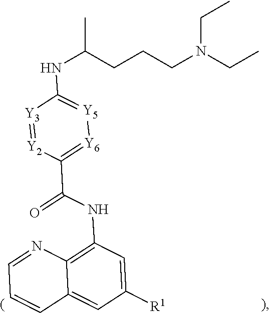

including pharmaceutically acceptable salts (e.g., 2,2,2-trifluoroacetate (TFA) salts and other salts) (e.g., physiologically tolerated acid addition salts), solvates, and/or prodrugs thereof,

wherein Y², Y³, Y⁵, Y⁶ are independently CH or N;

wherein R¹, (R⁷-R¹⁰ embedded in R¹) are as described within Formula I.

In some embodiments, A is NH, B is C═O, X1 is N, and X2 is C—R1, thereby rendering a compound encompassed within Formula XII

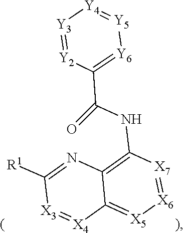

including pharmaceutically acceptable salts (e.g., 2,2,2-trifluoroacetate (TFA) salts and other salts) (e.g., physiologically tolerated acid addition salts), solvates, and/or prodrugs thereof,

wherein X³, X⁴, X⁵, X⁶ and X⁷ are independently selected from CH or N;

wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;

wherein R¹, R², R³, (R⁷-R¹⁰ embedded in R¹ and R²) are as described within Formula I.

In some embodiments, A is C═O, B is NH, X1 is N, X6 is C—R1, and Y4 is C—R2, thereby rendering a compound encompassed within Formula XIII

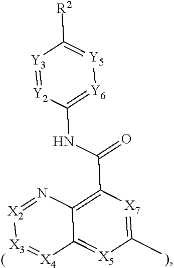

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, and X⁷ are independently selected from CR¹ or N, with the proviso that at least two of them must be CR¹;
wherein Y², Y³, Y⁵, Y⁶ are independently CH or N;
wherein R¹, R², (R⁷-R¹⁰ embedded in R¹ and R²) are as described within Formula I.

In some embodiments, A is NH, B is C═O, X1 is N, X2 is CH, X3 is CH, X4 is CH, X5 is CH, X6 is C—R1, X7 is CH, Y2 is N, Y3 is CH, Y4 is C—R2, Y5 is N, and Y6 is CH, thereby rendering a compound encompassed within Formula XIV

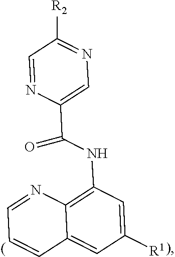

including pharmaceutically acceptable salts (e.g., 2,2,2-trifluoroacetate (TFA) salts and other salts) (e.g., physiologically tolerated acid addition salts), solvates, and/or prodrugs thereof,

wherein R¹ is independently H, Me and halogen;

wherein R², (R⁷-R¹⁰ embedded in R²) are as described within Formula I.

In some embodiments the compounds are encompassed within Formula XV:

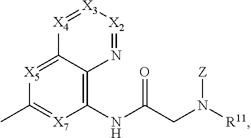

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, and X⁷ are independently selected from CR¹ or N, with the proviso that at least two of them must be CR¹;

wherein Z is independently C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-membered mono- or bicyclo-heteroaryl), C₁₋₆thioalkoxy, C₁₋₆thioalkoxy, C₁₋₆thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, C₂₋₆ alkyl-NR¹⁰R¹⁰, —C₂₋₆alkyl-R⁷;

wherein R¹¹ is H or Me;

wherein R⁷ and R¹⁰, (R⁸-R⁹ embedded in R⁷ and R¹⁰) are as described within Formula I.

In some embodiments, compounds shown in Table I are contemplated for Formula I.

TABLE I

| SL | | |
|---|---|---|
| NO | Structure | Name |
| J1 | 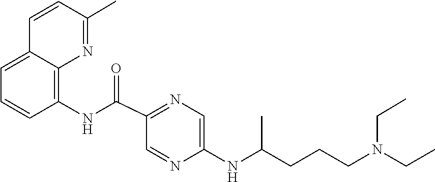 [SMILES: CCN(CC)CCCC(C)Nc1cnc(C(=O)Nc2cccc3ccc(C)nc23)cn1] | 5-((5-(diethylamino)pentan-2- yl)amino)-N-(2-methylquinolin-8- yl)pyrazine-2-carboxamide |
| J2 | 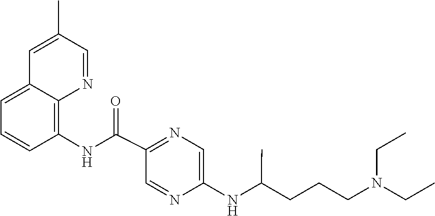 [SMILES: CCN(CC)CCCC(C)Nc1cnc(C(=O)Nc2cccc3cc(C)cnc23)cn1] | 5-((5-(diethylamino)pentan-2- yl)amino)-N-(3-methylquinolin-8- yl)pyrazine-2-carboxamide |
| J3 | 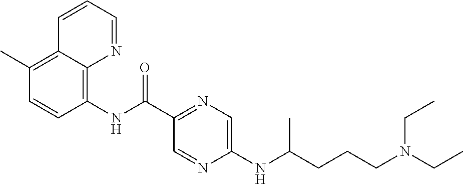 [SMILES: CCN(CC)CCCC(C)Nc1cnc(C(=O)Nc2ccc(C)c3cccnc23)cn1] | 5-((5-(diethylamino)pentan-2- yl)amino)-N-(5-methylquinolin-8- yl)pyrazine-2-carboxamide |
| J4 (JR-1- 235) | 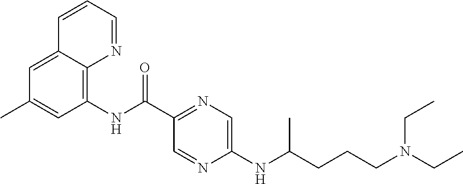 [SMILES: CCN(CC)CCCC(C)Nc1cnc(C(=O)Nc2cc(C)cc3cccnc23)cn1] | 5-((5-(diethylamino)pentan-2- yl)amino)-N-(6-methylquinolin-8- yl)pyrazine-2-carboxamide. |
| J5 | 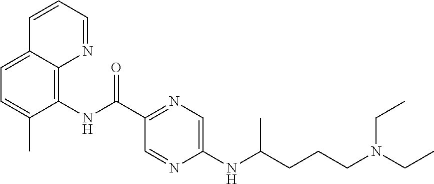 [SMILES: CCN(CC)CCCC(C)Nc1cnc(C(=O)Nc2c(C)ccc3cccnc23)cn1] | 5-((5-(diethylamino)pentan-2- yl)amino)-N-(7-methylquinolin-8- yl)pyrazine-2-carboxamide |
| J6 | 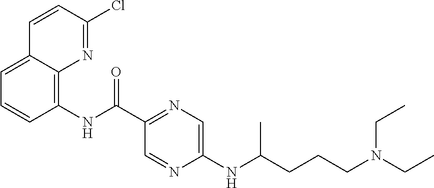 [SMILES: CCN(CC)CCCC(C)Nc1cnc(C(=O)Nc2cccc3ccc(Cl)nc23)cn1] | N-(2-chloroquinolin-8-yl)-5-((5- (diethylamino)pentan-2- yl)amino)pyrazine-2-carboxamide |
| J7 | 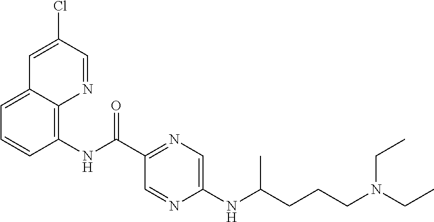 [SMILES: CCN(CC)CCCC(C)Nc1cnc(C(=O)Nc2cccc3cc(Cl)cnc23)cn1] | N-(3-chloroquinolin-8-yl)-5-((5- (diethylamino)pentan-2- yl)amino)pyrazine-2-carboxamide |
| J8 | 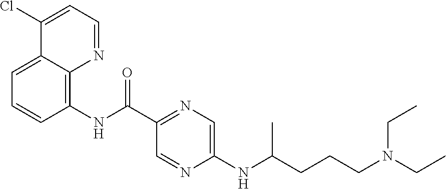 [SMILES: CCN(CC)CCCC(C)Nc1cnc(C(=O)Nc2cccc3c(Cl)ccnc23)cn1] | N-(4-chloroquinolin-8-yl)-5-((5- (diethylamino)pentan-2- yl)amino)pyrazine-2-carboxamide |
| J9 (JR-1- 157) | 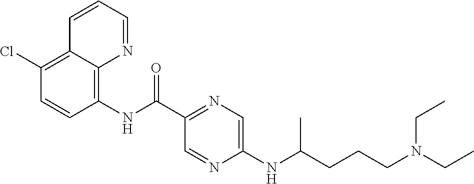 [SMILES: CCN(CC)CCCC(C)Nc1cnc(C(=O)Nc2ccc(Cl)c3cccnc23)cn1] | N-(5-chloroquinolin-8-yl)-5-((5- (diethylamino)pentan-2- yl)amino)pyrazine-2-carboxamide |
| J10 | 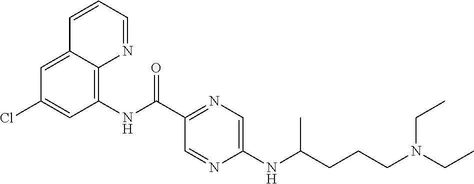 [SMILES: CCN(CC)CCCC(C)Nc1cnc(C(=O)Nc2cc(Cl)cc3cccnc23)cn1] | N-(6-chloroquinolin-8-yl)-5-((5- (diethylamino)pentan-2- yl)amino)pyrazine-2-carboxamide. |
| J11 (JR-1- 242) | 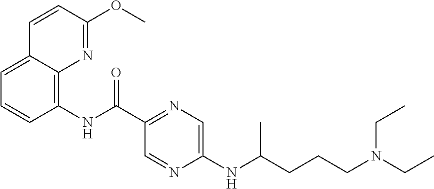 [SMILES: CCN(CC)CCCC(C)Nc1cnc(C(=O)Nc2cccc3ccc(OC)nc23)cn1] | 5-((5-(diethylamino)pentan-2- yl)amino)-N-(2-methoxyquinolin-8- yl)pyrazine-2-carboxamide. |
| J12 | 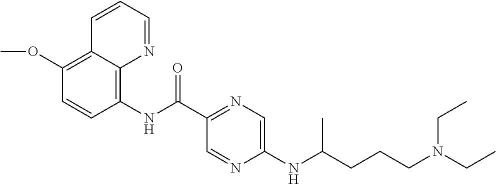 [SMILES: CCN(CC)CCCC(C)Nc1cnc(C(=O)Nc2ccc(OC)c3cccnc23)cn1] | 5-((5-(diethylamino)pentan-2- yl)amino)-N-(5-methoxyquinolin-8- yl)pyrazine-2-carboxamide. |
| J13 | 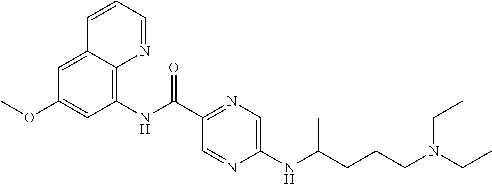 [SMILES: CCN(CC)CCCC(C)Nc1cnc(C(=O)Nc2cc(OC)cc3cccnc23)cn1] | 5-((5-(diethylamino)pentan-2- yl)amino)-N-(6-methoxyquinolin-8- yl)pyrazine-2-carboxamide. |
| J14 | 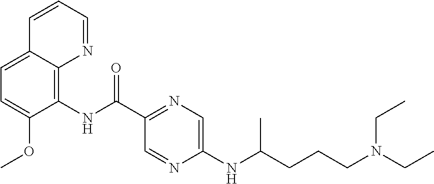 [SMILES: CCN(CC)CCCC(C)Nc1cnc(C(=O)Nc2c(OC)ccc3cccnc23)cn1] | 5-((5-(diethylamino)pentan-2- yl)amino)-N-(7-methoxyquinolin-8- yl)pyrazine-2-carboxamide |
| J15 | 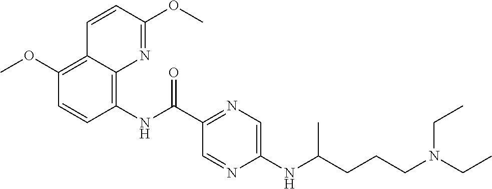 [SMILES: CCN(CC)CCCC(C)Nc1cnc(C(=O)Nc2ccc(OC)c3ccc(OC)nc23)cn1] | 5-((5-(diethylamino)pentan-2- yl)amino)-N-(2,5-dimethoxyquinolin-8- yl)pyrazine-2-carboxamide |
| J16 | 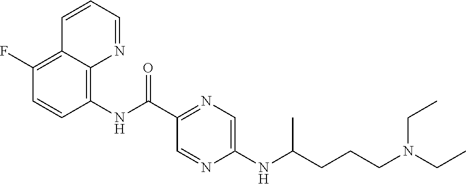 [SMILES: CCN(CC)CCCC(C)Nc1cnc(C(=O)Nc2ccc(F)c3cccnc23)cn1] | 5-((5-(diethylamino)pentan-2- yl)amino)-N-(5-fluoroquinolin-8- yl)pyrazine-2-carboxamide |
| J17 (JR-2- 298) | 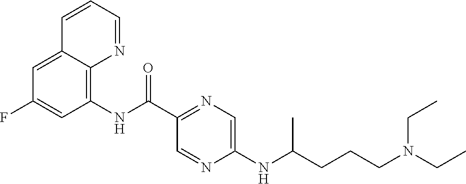 [SMILES: CCN(CC)CCCC(C)Nc1cnc(C(=O)Nc2cc(F)cc3cccnc23)cn1] | 5-((5-(diethylamino)pentan-2- yl)amino)-N-(6-fluoroquinolin-8- yl)pyrazine-2-carboxamide |
| J18 | 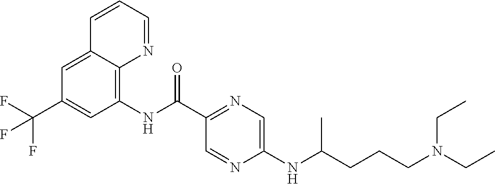 [SMILES: CCN(CC)CCCC(C)Nc1cnc(C(=O)Nc2cc(C(F)(F)F)cc3cccnc23)cn1] | 5-((5-(diethylamino)pentan-2- yl)amino)-N-(6- (trifluoromethyl)quinolin-8-yl)pyrazine- 2-carboxamide. |
| J19 | 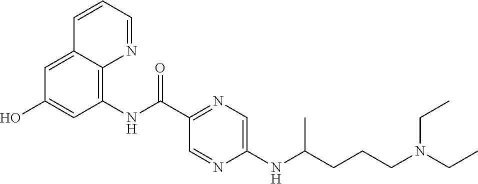 [SMILES: CCN(CC)CCCC(C)Nc1cnc(C(=O)Nc2cc(O)cc3cccnc23)cn1] | 5-((5-(diethylamino)pentan-2- yl)amino)-N-(6-hydroxyquinolin-8- yl)pyrazine-2-carboxamide. |
| J20 | 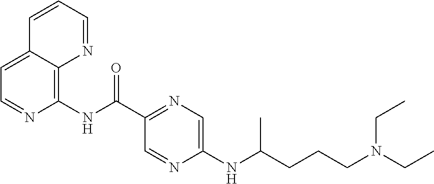 [SMILES: CCN(CC)CCCC(C)Nc1cnc(C(=O)Nc2nccc3cccnc23)cn1] | 5-((5-(diethylamino)pentan-2- yl)amino)-N-(1,7-naphthyridin-8- yl)pyrazine-2-carboxamide |
| J21 | 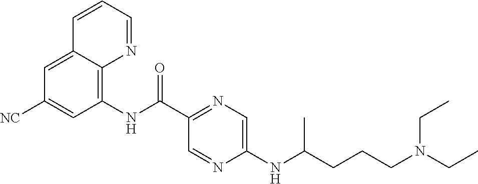 [SMILES: CCN(CC)CCCC(C)Nc1cnc(C(=O)Nc2cc(C#N)cc3cccnc23)cn1] | N-(6-cyanoquinolin-8-yl)-5-((5- (diethylamino)pentan-2- yl)amino)pyrazine-2-carboxamide |
| J22 | 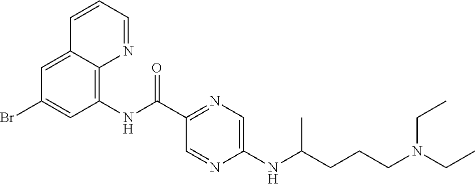 [SMILES: CCN(CC)CCCC(C)Nc1cnc(C(=O)Nc2cc(Br)cc3cccnc23)cn1] | N-(6-bromoquinolin-8-yl)-5-((5- (diethylamino)pentan-2- yl)amino)pyrazine-2-carboxamide |
| J23 | 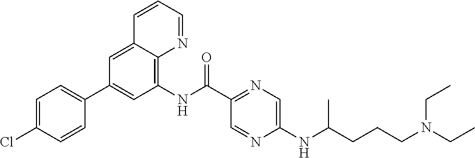 [SMILES: CCN(CC)CCCC(C)Nc1cnc(C(=O)Nc2cc(-c3ccc(Cl)cc3)cc3cccnc23)cn1] | N-(6-(4-chlorophenyl)quinolin-8-yl)-5- ((5-(diethylamino)pentan-2- yl)amino)pyrazine-2-carboxamide |
| J24 | 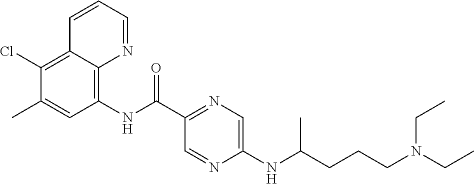 [SMILES: CCN(CC)CCCC(C)Nc1cnc(C(=O)Nc2cc(C)c(Cl)c3cccnc23)cn1] | N-(5-chloro-6-methylquinolin-8-yl)-5- ((5-(diethylamino)pentan-2- yl)amino)pyrazine-2-carboxamide |
| J25 | 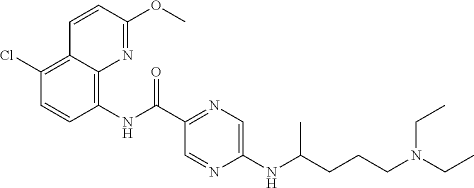 [SMILES: CCN(CC)CCCC(C)Nc1cnc(C(=O)Nc2ccc(Cl)c3ccc(OC)nc23)cn1] | N-(5-chloro-2-methoxyquinolin-8-yl)-5- ((5-(diethylamino)pentan-2- yl)amino)pyrazine-2-carboxamide |
| J26 | 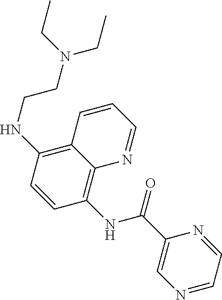 [SMILES: CCN(CC)CCNc1ccc(NC(=O)c2cnccn2)c2ncccc12] | N-(5-((2- (diethylamino)ethyl)amino)quinolin-8- yl)pyrazine-2-carboxamide |
| J27 | 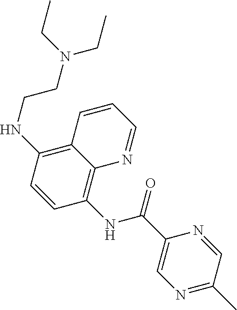 [SMILES: CCN(CC)CCNc1ccc(NC(=O)c2cnc(C)cn2)c2ncccc12] | N-(5-((2- (diethylamino)ethyl)amino)quinolin-8- yl)-5-methylpyrazine-2-carboxamide |
| J28 (JR-1- 272) | 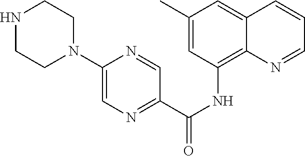 [SMILES: Cc1cc(NC(=O)c2cnc(N3CCNCC3)cn2)c2ncccc2c1] | N-(6-methylquinolin-8-yl)-5-(piperazin- 1-yl)pyrazine-2-carboxamide |
| J29 | 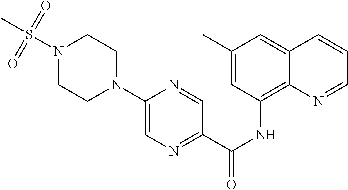 [SMILES: Cc1cc(NC(=O)c2cnc(N3CCN(S(C)(=O)=O)CC3)cn2)c2ncccc2c1] | N-(6-methylquinolin-8-yl)-5-(4- (methylsulfonyl)piperazin-1- yl)pyrazine-2-carboxamide |
| J30 | 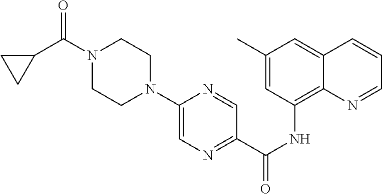 [SMILES: Cc1cc(NC(=O)c2cnc(N3CCN(C(=O)C4CC4)CC3)cn2)c2ncccc2c1] | 5-(4-(cyclopropanecarbonyl)piperazin- 1-yl)-N-(6-methylquinolin-8- yl)pyrazine-2-carboxamide |
| J31 | 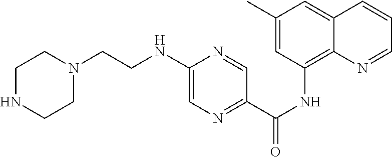 [SMILES: Cc1cc(NC(=O)c2cnc(NCCN3CCNCC3)cn2)c2ncccc2c1] | N-(6-methylquinolin-8-yl)-5-((2- (piperazin-1-yl)ethyl)amino)pyrazine-2- carboxamide |
| J32 | 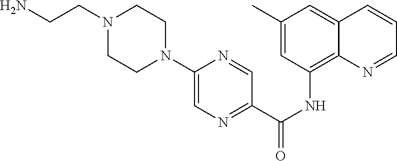 [SMILES: Cc1cc(NC(=O)c2cnc(N3CCN(CCN)CC3)cn2)c2ncccc2c1] | 5-(4-(2-aminoethyl)piperazin-1-yl)-N- (6-methylquinolin-8-yl)pyrazine-2- carboxamide |
| J33 | 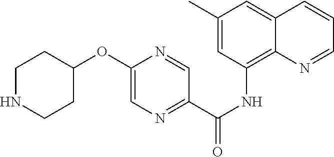 [SMILES: Cc1cc(NC(=O)c2cnc(OC3CCNCC3)cn2)c2ncccc2c1] | N-(6-methylquinolin-8-yl)-5-(piperidin- 4-yloxy)pyrazine-2-carboxamide |
| J34 | 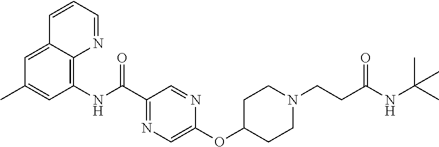 [SMILES: Cc1cc(NC(=O)c2cnc(OC3CCN(CCC(=O)NC(C)(C)C)CC3)cn2)c2ncccc2c1] | 5-((1-(3-(tert-butylamino)-3- oxopropyl)piperidin-4-yl)oxy)-N-(6- methylquinolin-8-yl)pyrazine-2- carboxamide |
| J35 | 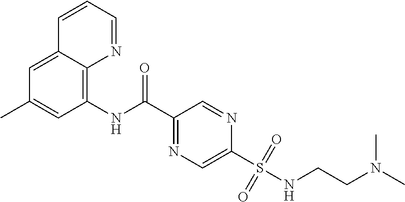 | 5-(N-(2- (dimethylamino)ethyl)sulfamoyl)-N-(6- methylquinolin-8-yl)pyrazine-2- carboxamide |
| J36 | 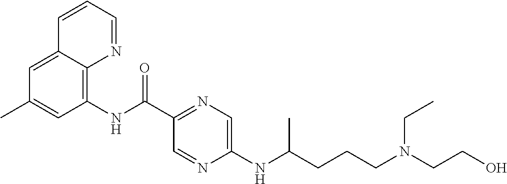 [SMILES: CCN(CCO)CCCC(C)Nc1cnc(C(=O)Nc2cc(C)cc3cccnc23)cn1] | 5-((5-(ethyl(2- hydroxyethyl)amino)pentan-2- yl)amino)-N-(6-methylquinolin-8- yl)pyrazine-2-carboxamide |
| J37 | 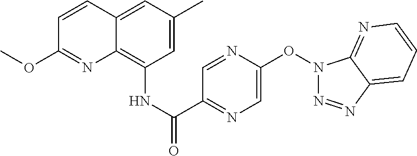 [SMILES: COc1ccc2cc(C)cc(NC(=O)c3cnc(On4nnc5cccnc54)cn3)c2n1] | 5-((3H-[1,2,3]triazolo[4,5-b]pyridin-3- yl)oxy)-N-(2-methoxy-6- methylquinolin-8-yl)pyrazine-2- carboxamide |
| J38 | 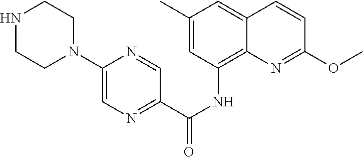 [SMILES: COc1ccc2cc(C)cc(NC(=O)c3cnc(N4CCNCC4)cn3)c2n1] | N-(2-methoxy-6-methylquinolin-8-yl)- 5-(piperazin-1-yl)pyrazine-2- carboxamide |
| J39 | 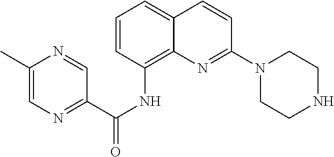 [SMILES: Cc1cnc(C(=O)Nc2cccc3ccc(N4CCNCC4)nc23)cn1] | 5-methyl-N-(2-(piperazin-1-yl)quinolin- 8-yl)pyrazine-2-carboxamide |
| J40 | 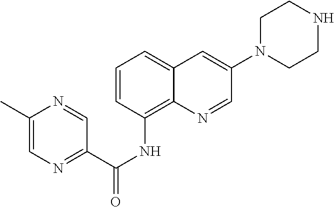 [SMILES: Cc1cnc(C(=O)Nc2cccc3cc(N4CCNCC4)cnc23)cn1] | 5-methyl-N-(3-(piperazin-1-yl)quinolin- 8-yl)pyrazine-2-carboxamide |
| J41 | 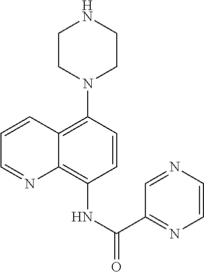 [SMILES: O=C(Nc1ccc(N2CCNCC2)c2cccnc12)c1cnccn1] | N-(5-(piperazin-1-yl)quinolin-8- yl)pyrazine-2-carboxamide |
| J42 | 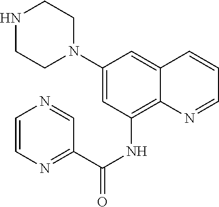 [SMILES: O=C(Nc1cc(N2CCNCC2)cc2cccnc12)c1cnccn1] | N-(6-(piperazin-1-yl)quinolin-8- yl)pyrazine-2-carboxamide |
| J43 | 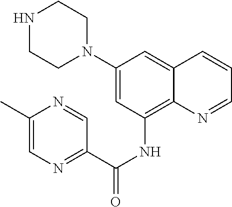 [SMILES: Cc1cnc(C(=O)Nc2cc(N3CCNCC3)cc3cccnc23)cn1] | 5-methyl-N-(6-(piperazin-1-yl)quinolin- 8-yl)pyrazine-2-carboxamide |
| J44 (JR-3- 6) | 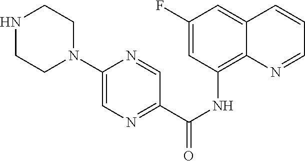 [SMILES: O=C(Nc1cc(F)cc2cccnc12)c1cnc(N2CCNCC2)cn1] | N-(6-fluoroquinolin-8-yl)-5-(piperazin- 1-yl)pyrazine-2-carboxamide |
| J45 | 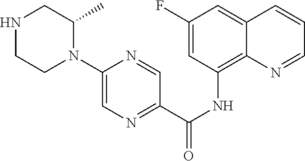 [SMILES: C[C@H]1CNCCN1c1cnc(C(=O)Nc2cc(F)cc3cccnc23)cn1] | (R)-N-(6-fluoroquinolin-8-yl)-5-(2- methylpiperazin-1-yl)pyrazine-2- carboxamide |
| J46 | 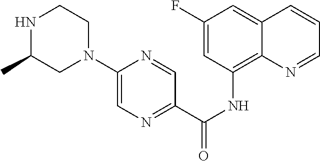 [SMILES: C[C@@H]1CN(c2cnc(C(=O)Nc3cc(F)cc4cccnc34)cn2)CCN1] | (R)-N-(6-fluoroquinolin-8-yl)-5-(3- methylpiperazin-1-yl)pyrazine-2- carboxamide |
| J47 | 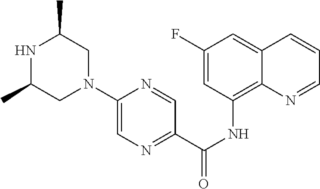 [SMILES: C[C@@H]1CN(c2cnc(C(=O)Nc3cc(F)cc4cccnc34)cn2)C[C@H](C)N1] | 5-((3S,5R)-3,5-dimethylpiperazin-1-yl)- N-(6-fluoroquinolin-8-yl)pyrazine-2- carboxamide |
| J48 | 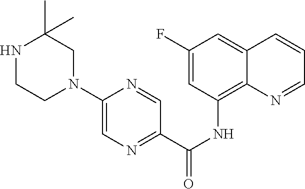 [SMILES: CC1(C)CN(c2cnc(C(=O)Nc3cc(F)cc4cccnc34)cn2)CCN1] | 5-(3,3-dimethylpiperazin-1-yl)-N-(6- fluoroquinolin-8-yl)pyrazine-2- carboxamide |
| J49 | 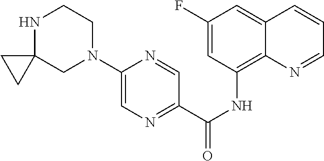 [SMILES: O=C(Nc1cc(F)cc2cccnc12)c1cnc(N2CCNC3(CC3)C2)cn1] | N-(6-fluoroquinolin-8-yl)-5-(4,7- diazaspiro[2.5]octan-7-yl)pyrazine-2- carboxamide |
| J50 | 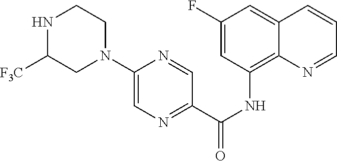 [SMILES: O=C(Nc1cc(F)cc2cccnc12)c1cnc(N2CCNC(C(F)(F)F)C2)cn1] | N-(6-fluoroquinolin-8-yl)-5-(3- (trifluoromethyl)piperazin-1- yl)pyrazine-2-carboxamide |
| J51 | 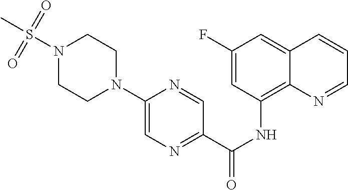 [SMILES: CS(=O)(=O)N1CCN(c2cnc(C(=O)Nc3cc(F)cc4cccnc34)cn2)CC1] | N-(6-fluoroquinolin-8-yl)-5-(4- (methylsulfonyl)piperazin-1- yl)pyrazine-2-carboxamide |
| J52 | 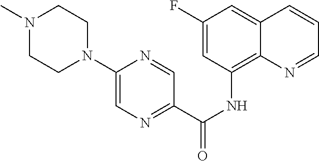 [SMILES: CN1CCN(c2cnc(C(=O)Nc3cc(F)cc4cccnc34)cn2)CC1] | N-(6-fluoroquinolin-8-yl)-5-(4- methylpiperazin-1-yl)pyrazine-2- carboxamide |
| J53 | 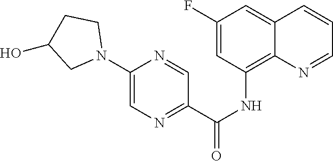 [SMILES: O=C(Nc1cc(F)cc2cccnc12)c1cnc(N2CCC(O)C2)cn1] | N-(6-fluoroquinolin-8-yl)-5-(3- hydroxypyrrolidin-1-yl)pyrazine-2- carboxamide |
| J54 | 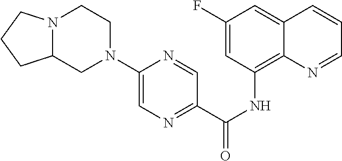 [SMILES: O=C(Nc1cc(F)cc2cccnc12)c1cnc(N2CCN3CCCC3C2)cn1] | N-(6-fluoroquinolin-8-yl)-5- (hexahydropyrrolo[1,2-a]pyrazin-2(1H)- yl)pyrazine-2-carboxamide |
| J55 | 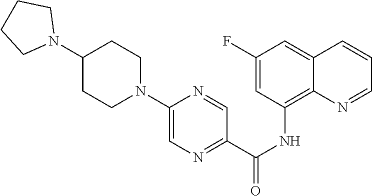 [SMILES: O=C(Nc1cc(F)cc2cccnc12)c1cnc(N2CCC(N3CCCC3)CC2)cn1] | N-(6-fluoroquinolin-8-yl)-5-(4- (pyrrolidin-1-yl)piperidin-1-yl)pyrazine- 2-carboxamide |
| J56 | 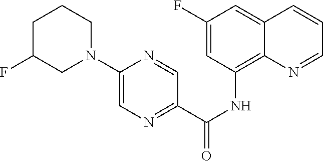 [SMILES: O=C(Nc1cc(F)cc2cccnc12)c1cnc(N2CCCC(F)C2)cn1] | 5-(3-fluoropiperidin-1-yl)-N-(6- fluoroquinolin-8-yl)pyrazine-2- carboxamide |
| J57 | 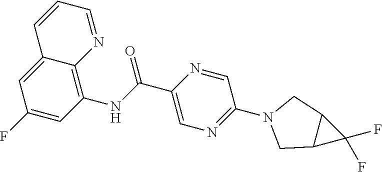 [SMILES: O=C(Nc1cc(F)cc2cccnc12)c1cnc(N2CC3C(C2)C3(F)F)cn1] | 5-(6,6-difluoro-3- azabicyclo[3.1.0]hexan-3-yl)-N-(6- fluoroquinolin-8-yl)pyrazine-2- carboxamide |
| J58 | 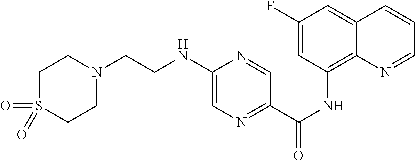 [SMILES: O=C(Nc1cc(F)cc2cccnc12)c1cnc(NCCN2CCS(=O)(=O)CC2)cn1] | 5-((2-(1,1- dioxidothiomorpholino)ethyl)amino)-N- (6-fluoroquinolin-8-yl)pyrazine-2- carboxamide |
| J59 | 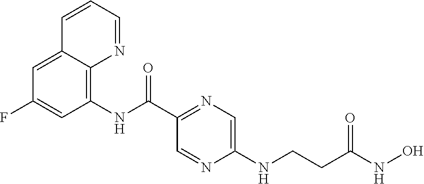 [SMILES: O=C(CCNc1cnc(C(=O)Nc2cc(F)cc3cccnc23)cn1)NO] | N-(6-fluoroquinolin-8-yl)-5-((3- (hydroxyamino)-3- oxopropyl)amino)pyrazine-2- carboxamide |
| J60 | 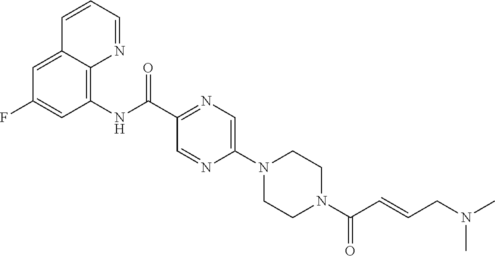 [SMILES: CN(C)C/C=C/C(=O)N1CCN(c2cnc(C(=O)Nc3cc(F)cc4cccnc34)cn2)CC1] | (E)-5-(4-(4-(dimethylamino)but-2- enoyl)piperazin-1-yl)-N-(6- fluoroquinolin-8-yl)pyrazine-2- carboxamide |
| J61 | 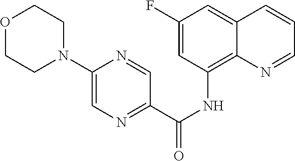 [SMILES: O=C(Nc1cc(F)cc2cccnc12)c1cnc(N2CCOCC2)cn1] | N-(6-fluoroquinolin-8-yl)-5- morpholinopyrazine-2-carboxamide |
| J62 | 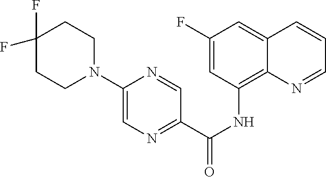 [SMILES: O=C(Nc1cc(F)cc2cccnc12)c1cnc(N2CCC(F)(F)CC2)cn1] | 5-(4,4-difluoropiperidin-1-yl)-N-(6- fluoroquinolin-8-yl)pyrazine-2- carboxamide |
| J63 | 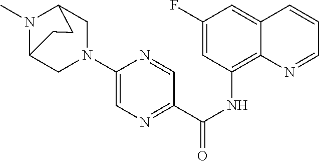 [SMILES: CN1C2CCC1CN(c1cnc(C(=O)Nc3cc(F)cc4cccnc34)cn1)C2] | N-(6-fluoroquinolin-8-yl)-5-(8-methyl- 3,8-diazabicyclo[3.2.1]octan-3- yl)pyrazine-2-carboxamide |
| J64 | 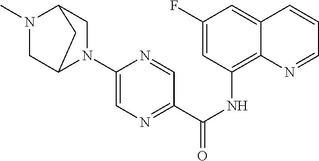 [SMILES: CN1CC2CC1CN2c1cnc(C(=O)Nc2cc(F)cc3cccnc23)cn1] | N-(6-fluoroquinolin-8-yl)-5-(5-methyl- 2,5-diazabicyclo[2.2.1]heptan-2- yl)pyrazine-2-carboxamide |
| J65 | 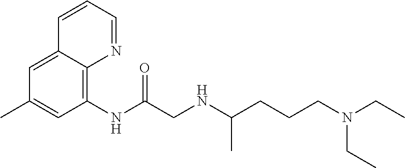 [SMILES: CCN(CC)CCCC(C)NCC(=O)Nc1cc(C)cc2cccnc12] | 2-((5-(diethylamino)pentan-2- yl)amino)-N-(6-methylquinolin-8- yl)acetamide |
| J66 | 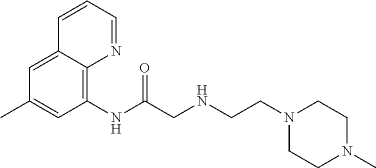 | 2-((2-(4-methylpiperazin-1- yl)ethyl)amino)-N-(6-methylquinolin-8- yl)acetamide |
| J67 | 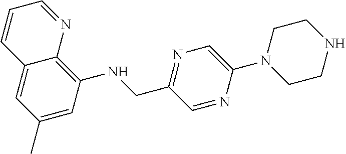 [SMILES: Cc1cc(NCc2cnc(N3CCNCC3)cn2)c2ncccc2c1] | 6-methyl-N-((5-(piperazin-1-yl)pyrazin- 2-yl)methyl)quinolin-8-amine |
| J68 | 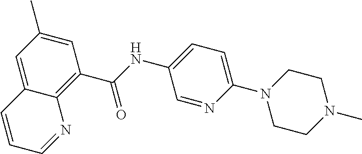 [SMILES: Cc1cc(C(=O)Nc2ccc(N3CCN(C)CC3)nc2)c2ncccc2c1] | 6-methyl-N-(6-(4-methylpiperazin-1- yl)pyridin-3-yl)quinoline-8- carboxamide |
| J69 | 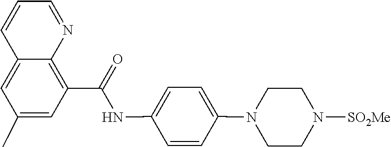 [SMILES: Cc1cc(C(=O)Nc2ccc(N3CCN(S(C)(=O)=O)CC3)cc2)c2ncccc2c1] | 6-methyl-N-(4-(4- (methylsulfonyl)piperazin-1- yl)phenyl)quinoline-8-carboxamide |
| J70 | 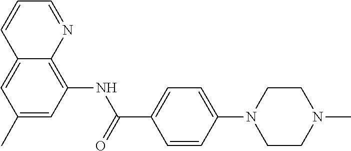 [SMILES: Cc1cc(NC(=O)c2ccc(N3CCN(C)CC3)cc2)c2ncccc2c1] | 4-(4-methylpiperazin-1-yl)-N-(6- methylquinolin-8-yl)benzamide |
| J71 | 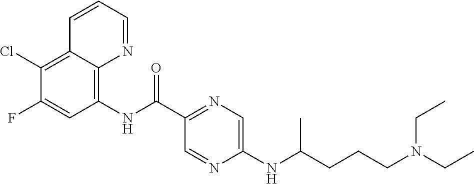 [SMILES: CCN(CC)CCCC(C)Nc1cnc(C(=O)Nc2cc(F)c(Cl)c3cccnc23)cn1] | N-(5-chloro-6-fluoroquinolin-8-yl)-5- ((5-(diethylamino)pentan-2- yl)amino)pyrazine-2-carboxamide |
| J72 | 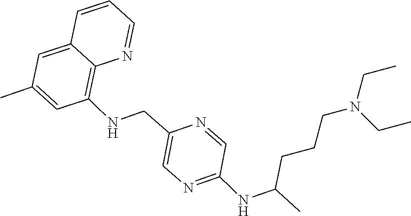 [SMILES: CCN(CC)CCCC(C)Nc1cnc(CNc2cc(C)cc3cccnc23)cn1] | N1,N1-diethyl-N4-(5-(((6- methylquinolin-8- yl)amino)methyl)pyrazin-2-yl)pentane- 1,4-diamine |
| J73 | 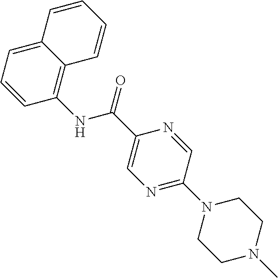 [SMILES: CN1CCN(c2cnc(C(=O)Nc3cccc4ccccc34)cn2)CC1] | 5-(4-methylpiperazin-1-yl)-N- (naphthalen-1-yl)pyrazine-2- carboxamide |
| J74 | 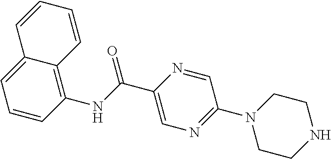 [SMILES: O=C(Nc1cccc2ccccc12)c1cnc(N2CCNCC2)cn1] | N-(naphthalen-1-yl)-5-(piperazin-1- yl)pyrazine-2-carboxamide |
| J75 | 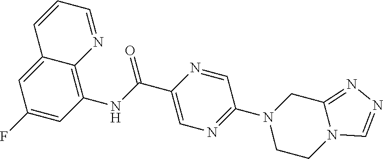 [SMILES: O=C(Nc1cc(F)cc2cccnc12)c1cnc(N2CCn3cnnc3C2)cn1] | 5-(5,6-dihydro-[1,2,4]triazolo[4,3- a]pyrazin-7(8H)-yl)-N-(6- fluoroquinolin-8-yl)pyrazine-2- carboxamide |
| J76 | 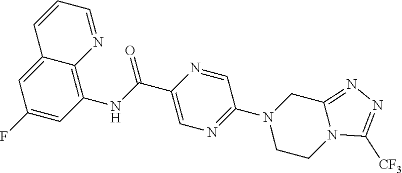 [SMILES: O=C(Nc1cc(F)cc2cccnc12)c1cnc(N2CCn3c(nnc3C(F)(F)F)C2)cn1] | N-(6-fluoroquinolin-8-yl)-5-(3- (trifluoromethyl)-5,6-dihydro- [1,2,4]triazolo[4,3-a]pyrazin-7(8H)- yl)pyrazine-2-carboxamide |
| J77 | 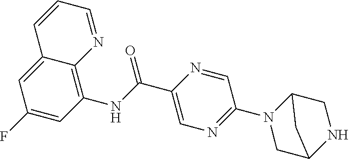 [SMILES: O=C(Nc1cc(F)cc2cccnc12)c1cnc(N2CC3CC2CN3)cn1] | 5-(2,5-diazabicyclo[2.2.1]heptan-2-yl)- N-(6-fluoroquinolin-8-yl)pyrazine-2- carboxamide |
| J78 | 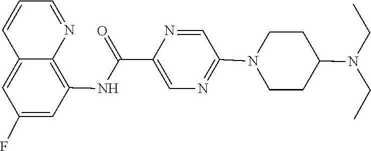 [SMILES: CCN(CC)C1CCN(c2cnc(C(=O)Nc3cc(F)cc4cccnc34)cn2)CC1] | 5-(4-(diethylamino)piperidin-1-yl)-N- (6-fluoroquinolin-8-yl)pyrazine-2- carboxamide |
| J79 | 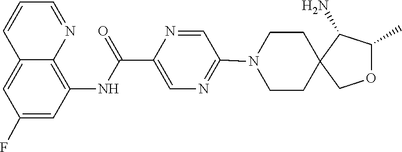 [SMILES: C[C@@H]1OCC2(CCN(c3cnc(C(=O)Nc4cc(F)cc5cccnc45)cn3)CC2)[C@@H]1N] | 5-((3S,4S)-4-amino-3-methyl-2-oxa-8- azaspiro[4.5]decan-8-yl)-N-(6- fluoroquinolin-8-yl)pyrazine-2- carboxamide |
| J80 | 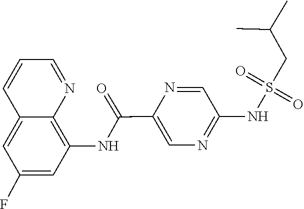 [SMILES: CC(C)CS(=O)(=O)Nc1cnc(C(=O)Nc2cc(F)cc3cccnc23)cn1] | N-(6-fluoroquinolin-8-yl)-5-((2- methylpropyl)sulfonamido)pyrazine-2- carboxamide |
| J81 | 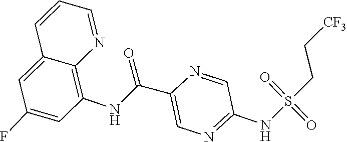 [SMILES: O=C(Nc1cc(F)cc2cccnc12)c1cnc(NS(=O)(=O)CCC(F)(F)F)cn1] | N-(6-fluoroquinolin-8-yl)-5-((3,3,3- trifluoropropyl)sulfonamido)pyrazine-2- carboxamide |
| J82 | 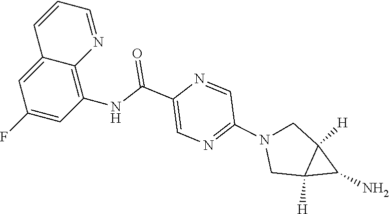 [SMILES: N[C@@H]1[C@H]2CN(c3cnc(C(=O)Nc4cc(F)cc5cccnc45)cn3)C[C@@H]12] | 5-((1R,5S,6s)-6-amino-3- azabicyclo[3.1.0]hexan-3-yl)-N-(6- fluoroquinolin-8-yl)pyrazine-2- carboxamide |
| J83 | 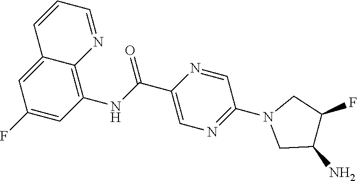 [SMILES: N[C@H]1CN(c2cnc(C(=O)Nc3cc(F)cc4cccnc34)cn2)C[C@H]1F] | 5-((3S,4R)-3-amino-4-fluoropyrrolidin- 1-yl)-N-(6-fluoroquinolin-8-yl)pyrazine- 2-carboxamide |
| J84 | 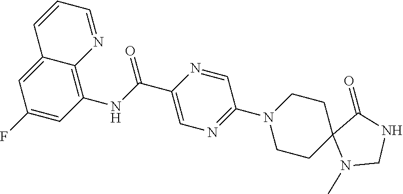 [SMILES: CN1CNC(=O)C12CCN(c1cnc(C(=O)Nc3cc(F)cc4cccnc34)cn1)CC2] | N-(6-fluoroquinolin-8-yl)-5-(1-methyl- 4-oxo-1,3,8-triazaspiro[4.5]decan-8- yl)pyrazine-2-carboxamide |
| J85 | 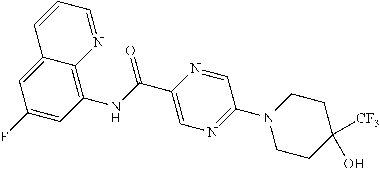 [SMILES: O=C(Nc1cc(F)cc2cccnc12)c1cnc(N2CCC(O)(C(F)(F)F)CC2)cn1] | N-(6-fluoroquinolin-8-yl)-5-(4-hydroxy- 4-(trifluoromethyl)piperidin-1- yl)pyrazine-2-carboxamide |
| J86 | 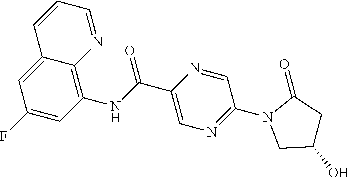 [SMILES: O=C(Nc1cc(F)cc2cccnc12)c1cnc(N2C[C@@H](O)CC2=O)cn1] | (S)-N-(6-fluoroquinolin-8-yl)-5-(4- hydroxy-2-oxopyrrolidin-1-yl)pyrazine- 2-carboxamide |
| J87 | 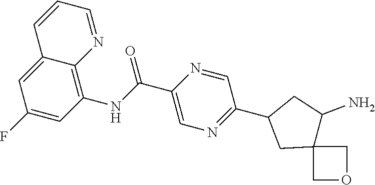 [SMILES: NC1CC(c2cnc(C(=O)Nc3cc(F)cc4cccnc34)cn2)CC12COC2] | 5-(8-amino-2-oxa-6-azaspiro[3.4]octan- 6-yl)-N-(6-fluoroquinolin-8-yl)pyrazine- 2-carboxamide |
| J88 | 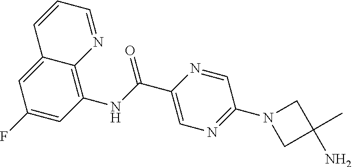 [SMILES: CC1(N)CN(c2cnc(C(=O)Nc3cc(F)cc4cccnc34)cn2)C1] | 5-(3-amino-3-methylazetidin-1-yl)-N- (6-fluoroquinolin-8-yl)pyrazine-2- carboxamide |
| J89 | 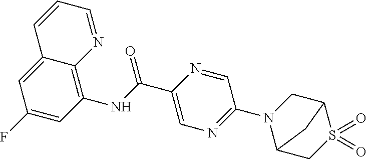 [SMILES: O=C(Nc1cc(F)cc2cccnc12)c1cnc(N2CC3CC2CS3(=O)=O)cn1] | 5-(2,2-dioxido-2-thia-5- azabicyclo[2.2.1]heptan-5-yl)-N-(6- fluoroquinolin-8-yl)pyrazine-2- carboxamide |
| J90 | 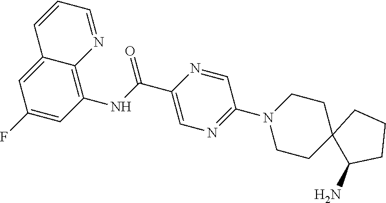 [SMILES: N[C@@H]1CCCC12CCN(c1cnc(C(=O)Nc3cc(F)cc4cccnc34)cn1)CC2] | (R)-5-(1-amino-8-azaspiro[4.5]decan-8- yl)-N-(6-fluoroquinolin-8-yl)pyrazine-2- carboxamide |
| J91 | 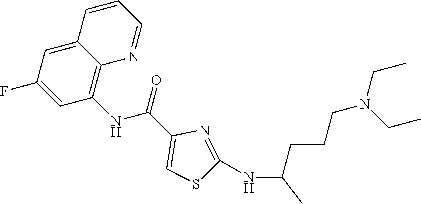 [SMILES: CCN(CC)CCCC(C)Nc1nc(C(=O)Nc2cc(F)cc3cccnc23)cs1] | 2-((5-(diethylamino)pentan-2- yl)amino)-N-(6-fluoroquinolin-8- yl)thiazole-4-carboxamide |
| J92 | 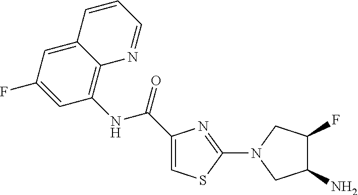 [SMILES: N[C@H]1CN(c2nc(C(=O)Nc3cc(F)cc4cccnc34)cs2)C[C@H]1F] | 2-((3S,4R)-3-amino-4-fluoropyrrolidin- 1-yl)-N-(6-fluoroquinolin-8-yl)thiazole- 4-carboxamide |
| J93 | 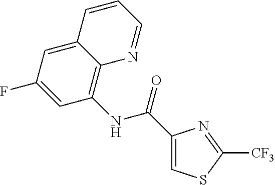 [SMILES: O=C(Nc1cc(F)cc2cccnc12)c1csc(C(F)(F)F)n1] | N-(6-fluoroquinolin-8-yl)-2- (trifluoromethyl)thiazole-4-carboxamide |
| J94 | 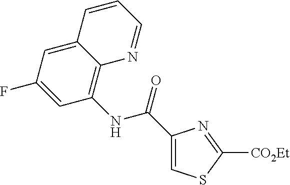 [SMILES: CCOC(=O)c1nc(C(=O)Nc2cc(F)cc3cccnc23)cs1] | ethyl 4-((6-fluoroquinolin-8- yl)carbamoyl)thiazole-2-carboxylate |
| J95 | 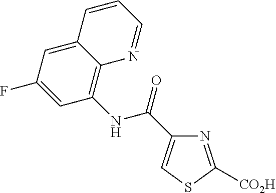 [SMILES: O=C(Nc1cc(F)cc2cccnc12)c1csc(C(=O)O)n1] | 4-((6-fluoroquinolin-8- yl)carbamoyl)thiazole-2-carboxylic acid |

The invention further provides processes for preparing any of the compounds of the present invention through following at least a portion of the techniques recited in the experimental section.

The compounds of the invention are useful for the treatment, amelioration, or prevention of hyperproliferative disorders (e.g., diabetes) (e.g., cancer) (e.g., leukemia, colon cancer, CNS cancer, Non-Small lung cancer, melanoma, ovarian cancer, renal cancer, breast cancer, prostate cancer, esophageal cancer, cervical cancer and colorectal cancer), and other inflammatory diseases (e.g., chronic auto immune disorder, or a viral infection).

The compounds of the invention are useful for the treatment, amelioration, or prevention of disorders, such as those responsive to induction of apoptotic cell death, e.g., disorders characterized by dysregulation of apoptosis, including hyperproliferative diseases such as cancer. In certain embodiments, the compounds can be used to treat, ameliorate, or prevent cancer that is characterized by resistance to cancer therapies (e.g., those cancer cells which are chemoresistant, radiation resistant, hormone resistant, and the like). In certain embodiments, the cancer is selected from one or more of leukemia, colon cancer, CNS cancer, Non-Small lung cancer, melanoma, ovarian cancer, renal cancer, breast cancer, prostate cancer, esophageal cancer, cervical cancer and colorectal cancer.

The invention also provides pharmaceutical compositions comprising the compounds of the invention in a pharmaceutically acceptable carrier.

The invention also provides kits comprising a compound of the invention and instructions for administering the compound to an animal. The kits may optionally contain other therapeutic agents (e.g., anticancer agents or apoptosis-modulating agents) (e.g., therapeutic agents useful in treating any type of cancer) (e.g., therapeutic agents useful in treating any type of inflammatory disorder).

### BRIEF DESCRIPTION OF THE DRAWINGS

FIG. 1: In vivo efficacy studies. Efficacy study was performed on mice with subcutaneous CT-26 implantation. Mice were implanted with 1,000,000 cells into the right flank and randomized into three groups (n=5) eleven days later. Mice were dosed 5 times weekly with no dosing on weekends. Control tumors grew well, with the majority of mice reaching euthanasia criteria at Day 16. JR5-26B and JR4-187 showed efficacy.

### DEFINITIONS

The term “anticancer agent” as used herein, refer to any therapeutic agents (e.g., chemotherapeutic compounds and/or molecular therapeutic compounds), antisense therapies, radiation therapies, or surgical interventions, used in the treatment of hyperproliferative diseases such as cancer (e.g., in mammals, e.g., in humans).

The term “prodrug” as used herein, refers to a pharmacologically inactive derivative of a parent “drug” molecule that requires biotransformation (e.g., either spontaneous or enzymatic) within the target physiological system to release, or to convert (e.g., enzymatically, physiologically, mechanically, electromagnetically) the prodrug into the active drug. Prodrugs are designed to overcome problems associated with stability, water solubility, toxicity, lack of specificity, or limited bioavailability. Exemplary prodrugs comprise an active drug molecule itself and a chemical masking group (e.g., a group that reversibly suppresses the activity of the drug). Some prodrugs are variations or derivatives of compounds that have groups cleavable under metabolic conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987.

Exemplary prodrugs become pharmaceutically active in vivo or in vitro when they undergo solvolysis under physiological conditions or undergo enzymatic degradation or other biochemical transformation (e.g., phosphorylation, hydrogenation, dehydrogenation, glycosylation). Prodrugs often offer advantages of water solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common prodrugs include acid derivatives such as esters prepared by reaction of parent acids with a suitable alcohol (e.g., a lower alkanol) or esters prepared by reaction of parent alcohol with a suitable carboxylic acid, (e.g., an amino acid), amides prepared by reaction of the parent acid compound with an amine, basic groups reacted to form an acylated base derivative (e.g., a lower alkylamide), or phosphorus-containing derivatives, e.g., phosphate, phosphonate, and phosphoramidate esters, including cyclic phosphate, phosphonate, and phosphoramidate (see, e.g., US Patent Application Publication No. US 2007/0249564 A1; herein incorporated by reference in its entirety).

The term “pharmaceutically acceptable salt” as used herein, refers to any salt (e.g., obtained by reaction with an acid or a base) of a compound of the present invention that is physiologically tolerated in the target animal (e.g., a mammal). Salts of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxy ethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “solvate” as used herein, refers to the physical association of a compound of the invention with one or more solvent molecules, whether organic or inorganic. This physical association often includes hydrogen bonding. In certain instances, the solvate is capable of isolation, for example, when one or more solvate molecules are incorporated in the crystal lattice of the crystalline solid. “Solvate” encompasses both solution-phase and isolable solvates. Exemplary solvates include hydrates, ethanolates, and methanolates.

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, in one embodiment, a therapeutically effective amount will refer to the amount of a therapeutic agent that decreases the rate of tumor growth, decreases tumor mass, decreases the number of metastases, increases time to tumor progression, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “sensitize” and “sensitizing,” as used herein, refer to making, through the administration of a first agent (e.g., a compound of the invention having a quinolinyl-pyrazine-carboxamide (or similar) structure), an animal or a cell within an animal more susceptible, or more responsive, to the biological effects (e.g., promotion or retardation of an aspect of cellular function including, but not limited to, cell division, cell growth, proliferation, invasion, angiogenesis, necrosis, or apoptosis) of a second agent. The sensitizing effect of a first agent on a target cell can be measured as the difference in the intended biological effect (e.g., promotion or retardation of an aspect of cellular function including, but not limited to, cell growth, proliferation, invasion, angiogenesis, or apoptosis) observed upon the administration of a second agent with and without administration of the first agent. The response of the sensitized cell can be increased by at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least 300%, at least about 350%, at least about 400%, at least about 450%, or at least about 500% over the response in the absence of the first agent.

The term “dysregulation of apoptosis,” as used herein, refers to any aberration in the ability of (e.g., predisposition) a cell to undergo cell death via apoptosis. Dysregulation of apoptosis is associated with or induced by a variety of conditions, non-limiting examples of which include, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), and other conditions such as osteoarthritis and atherosclerosis.

The term “hyperproliferative disease,” as used herein, refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “normal cell,” as used herein, refers to a cell that is not undergoing abnormal growth or division. Normal cells are non-cancerous and are not part of any hyperproliferative disease or disorder.

The term “anti-neoplastic agent,” as used herein, refers to any compound that retards the proliferation, growth, or spread of a targeted (e.g., malignant) neoplasm.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, Pa., 19th ed. 1995.

### DETAILED DESCRIPTION OF THE INVENTION

RNA-Seq (RNA sequencing), can be used to analyze transcriptome (see, Costa-Silva, J.; et al., PLoS One. 2017, 1-18; McDermaid, A.; et al., Brief Bioinform. 2018). In RNA sequencing mRNA extracted from a sample is converted to cDNA using reverse transcription, fragments within lengths of a certain range are selected and adapters are ligated to each end of the cDNA. In the amplification step sequencing can be done in either unidirectional (single-end sequencing) or bidirectional (paired-end sequencing) and then associated to a reference genome database or assembled to obtain de novo transcripts, providing a genome-wide expression profile (see, Wang, Z.; et al., Nat. Rev. Genet., 2009, 10, 57-63). In directional sequencing, cDNA synthesis and adapter ligation can be done in a strand-specific manner. Advantage of directional sequencing is, apart from providing an insight into antisense transcripts and their potential role in regulation and strand information of non-coding RNAs, it aids in accurately quantifying overlapping transcripts. Strand specificity is not maintained in non-directional RNA-Seq protocols. However, the specifics of the sequencing protocols vary from one technology to the other. The length of produced reads depends on the technology applied, with newer high-throughput technologies producing longer reads (see, Seesi, S. A.; et al., Genomic Medicine. 2016, 237-250).

RNA sequencing is rapidly replacing gene expression microarrays as RNA-Seq can detect novel transcripts, allele-specific expression and splice junctions. RNA-Seq is advantageous as it is independent of the genome annotation for prior probe selection and avoids the related biases introduced during hybridization of microarrays (see, Zhao, S.; et al., PLoS One. 2014, 9, 78644).

Bru-Seq maps nascent RNA transcripts using bromouridine tagging (see, Paulsen M. T.; et al., PNAS 2013, 110, 2240-2245). The advantages of this method are it maps sequences of nascent RNA transcripts and determines relative transcription rate, detects long non-coding RNAs (IncRNAs) and detects transcription anywhere on the genome but due to the requirement for incubation in the presence of labeled nucleotides, it is limited to cell cultures and other artificial systems. Bru-seq results provide a comprehensive profile of nascent transcription during the immediate serum response and distinguishes nascent RNA from previously synthesized RNA thereby providing a genome-wide picture of RNA synthesis.

The interpretation of gene expression data is based on the function of individual genes as well as their role in biological pathways. However, for some genes, a small expression change may be not significant at a single gene level, but combination of minor changes of several genes may be relevant for a biological pathway (see, Han, Y.; et al., Bioinform Biol Insights. 2015, 9, 29-46; Rahmatallah, Y.; et al., BMC Bioinformatics. 2014, 15, 397). Bioinformatics analysis of these datasets and their comparisons with published RNA-seq and microarray data reveals similarity of new scaffolds with reported drug candidates.

Proteomics provide a comprehensive understanding of mechanisms that are responsible for the cytotoxicity of anticancer drugs, and based on the expression of protein helps in identification of drug targets (see, Wang, Y.; et al., Met Based Drugs. 2008, 1-9). One of the disadvantages of proteomics is membrane-bound proteins due to their poor solubility and low abundance, are disproportionally represented in proteome profiles (see, Smith, C. Nature, 2004, 428, 225-231). Identification of target proteins utilizing proteomics technique coupled with mass spectrometry is an evolving technology platform that has the potential to identify novel proteins involved in key biological processes in cells. There are two mass spectrometry-based methods currently used for protein profiling. The use of high-resolution two-dimensional electrophoresis to separate proteins from different samples, followed by selection and staining of differentially expressed proteins to be identified by mass spectrometry is the more established method. In the second approach isotope coded affinity tag (ICAT) reagents are utilized to differentially label proteins from two different complex mixtures, that are then digested to yield labeled peptides. The labeled mixtures are then combined, the peptides separated by multidimensional liquid chromatography and analyzed by tandem mass spectrometry. In this method, complexity of the mixtures omitting the non-cysteine residues is reduced as here the cysteine residues of proteins get covalently attached to the ICAT reagent. Proteomics coupled with bioinformatics process the raw mass spectral data into protein data (see, Yu, L. R.; Essentials of Genomic and Personalized Medicine 2010, 89-96). The most critical software programs take peptide mapping and/or tandem MS results and determine the protein or peptide sequence that are most closely related to the experimental data. One of the more intriguing MS-based proteomic techniques is to identify the proteins which are overexpressed thus aids in identification of the target protein of the given drug. There are several examples reported in literature about successful application of Bru-seq and proteomics for identification of mechanism of action of drugs.

Experiments conducted during the course of developing embodiments for the present invention utilized Bru-Seq and proteomics technologies followed by bioinformatics analysis to better elucidate the mechanism of action of several representative compounds disclosed herein. The expression of top 25 genes, proteins, and gene sets that were up- and down-regulated in response to treatment are fully detailed. Overexpression of genes such as INSIG1, DHCR7, MVK and MSMO1 suggests cholesterol biosynthesis pathway. INSIG1 (see, Janowski, B. A. PNAS 2002, 99, 12675-12680) plays an important role in the SREBP-mediated regulation of cholesterol biosynthesis. This gene encodes an endoplasmic reticulum membrane protein that regulates cholesterol metabolism, lipogenesis, and glucose homeostasis. DHCR7 (see, Prabhu, A. V.; et al., Prog. Lipid Res. 2016, 64, 138-151) encodes an enzyme that removes the C(7-8) double bond in the B ring of sterols and catalyzes the conversion of 7-dehydrocholesterol to cholesterol. The MVK gene provides instructions for making the mevalonate kinase enzyme. This enzyme converts mevalonic acid into mevalonate-5-phosphate which is a crucial intermediate for production of cholesterol. MVK gene is related to regulation of cholesterol biosynthesis by SREBP and terpenoid backbone biosynthesis pathway. MSMO1 (Methylsterol Monooxygenase 1) is a protein coding gene related to cholesterol biosynthesis III (via desmosterol) and terpenoid backbone biosynthesis pathway. MSMO1 is a sterol-C₄-methyl oxidase-like protein which was isolated based on its similarity to the yeast ERG25 protein. It contains a set of putative metal binding motifs with similarity to that seen in a family of membrane desaturases-hydroxylases. As revealed by Bru-seq, synthesis of INSIG1, DHCR7, MVK and MSMO1 RNAs were upregulated by treatment with J4. Upregulation was also observed in expression of DNA-damage-inducible transcript 4 (DDIT4) protein which acts as a negative regulator of mTOR, a serine/threonine kinase that regulates a variety of cellular functions such as growth, proliferation and autophagy. DDIT4 expression has been shown to be activated by upregulation of HIF-1 in response to hypoxia, DNA damage and energy stress. GPR-135 shows downregulation in Bru-seq analysis. GPR135 (G Protein-Coupled Receptor 135) is a protein coding gene which shows a reciprocal regulatory interaction with the melatonin receptor MTNR1B most likely through receptor heteromerization.

As revealed by Bru-seq analysis of J28, also show similar profile like J4. Overexpression of genes like INSIG1, DHCR7, MVK and FASN suggests cholesterol biosynthesis pathway as the mode of action of this series of compounds. The enzyme encoded by FASN catalyzes the synthesis of palmitate from acetyl-CoA and malonyl-CoA, in the presence of NADPH, into long-chain saturated fatty acids.

Cholesterol is a precursor for the synthesis of the steroid hormones, the bile acids, and vitamin D. The process of cholesterol synthesis involves five major steps starting from acetyl-CoA. The initial part of cholesterol biosynthesis is also called mevalonate pathway, where mevalonate is converted to the isoprene-based molecule, isopentenyl pyrophosphate (IPP). In the second part, IPP molecules are converted to squalene, which finally culminates into cholesterol.

MVK gene provides instructions for making the mevalonate kinase enzyme that converts mevalonic acid into mevalonate-5-phosphate, which is a key intermediate in “cholesterol biosynthesis pathway”. DHCR7 gene is responsible for generation of enzyme 7-dehydrocholesterol reductase that converts 7-dehydrocholesterol to cholesterol in the final step of “cholesterol biosynthesis pathway”. INSIG1 binds to the sterol-sensing domain of SCAP (SREBP cleavage activating protein) resulting in SCAP/SREBP complex stay longer in the ER, ultimately blocks SREBP from acting as a transcription factor for the SRE in the promoter region of the HMG-CoA-reductase gene and results in a decreased expression of HMG-CoA-reductase. Thus, INSIG1 plays an important role in the SREBP-mediated regulation of cholesterol biosynthesis. Thus, INSIG1, DHCR7 and MVK genes play a crucial role in “cholesterol biosynthesis pathway”.

Bru-seq analysis showed >1.5-fold change in expression INSIG1, DHCR7 and MVK upon treatment of MIA PaCa-2 cells with J4 or J28. This implies that the two compounds have similar mechanisms of action. Three highly upregulated genes INSIG1, DHCR7 and MVK related to cholesterol biosynthesis pathway could serve as potential drivers for anti-cancer activity of J4 and J28.

The Bru-seq results also show upregulation of AVPI1, CCNG2, TUBA1A, H2AFX, or HIST1H3C indicating the mechanism of action to be a pathway related to cell cycle regulation. Arginine vasopressin-induced protein 1 (AVPI1) may be involved in MAP kinase activation, epithelial sodium channel (ENaC) down-regulation and cell cycling. CCNG2 (Cyclin G2) is a protein coding gene related to Mitotic G1-G1/S phases and FoxO signaling pathway. TUBA1A is a structural gene that encodes for Tubulin, Alpha 1A product that participates in the formation of microtubules—structural proteins that participate in cytoskeletal structure. H2AFX (H2A Histone Family Member X) and HIST1H3C (Histone Cluster 1 H3 Family Member C) both are protein coding genes related to activated PKN1 stimulates transcription of AR (androgen receptor) regulated genes KLK2 and KLK3 and Cell Cycle, Mitotic pathway. Thus, another significant pathway upregulated following 4 h of J4/J28 treatment is Cell Cycle, Mitotic pathway.

Proteomics study of J4 (JR-1-235) revealed upregulation of Hydroxymethylglutaryl-CoA synthase, an enzyme which catalyzes the reaction in which Acetyl-CoA condenses with acetoacetyl-CoA to form 3-hydroxy-3-methylglutaryl-CoA (HMG-CoA). It is the second step in the mevalonate-dependent isoprenoid biosynthesis pathway. HMG-CoA is an intermediate in both cholesterol synthesis and ketogenesis. Thus, here the proteomics results are in accordance with the Bru-seq results, implying “cholesterol biosynthesis pathway” as key pathway activated by these series of compounds.

The Developmental Therapeutics Program (DTP) of NCI has evaluated more than 100,000 pure compounds and more than 34,000 crude extracts against the panel of human tumor cell lines. The resultant data are analyzed using a program called COMPARE, that rank the entire database of tested compounds in the order of the similarity of the responses. The results obtained with the COMPARE algorithm indicate that compounds high in this ranking may possess a mechanism of action like a known compound in NCI database (see, Holbeck, S. L.; et al, Mol. Cancer Ther. 2010, 9, 1451-1460). Thus, the NCI60 databases is highly useful to the cancer research community. Further experiments were conducted that tested some of the active compounds in a panel of NCI60 cell line to relate our compounds with some known inhibitors to have an idea of possible mechanism of action.

There are many documentations of cholesterol biosynthesis also affecting the immune system (see, Getz, G. S.; et al., Clin. Lipidol. 2014, 9, 657-671). The disruptions of cellular or organismal cholesterol homeostasis leading to cholesterol accumulation results in the amplification of inflammatory responses via enhanced TLR signaling or inflammasome activation. Cholesterol accumulation adversely affects diseases that are associated with chronic metabolic inflammation, including atherosclerosis and obesity. Therapeutic interventions such as increased production of APOA1-containing HDL, potentially benefits patients with atherosclerosis, obesity, insulin resistance and autoimmune diseases (see, Tall, A. R.; et al., Nat. Rev. Immunol. 2015, 15, 104-116). Regulation of mevalonate metabolism affects immune responses, low activity impairs cellular function and survival, whereas hyperactivity can lead to malignant transformation (see, Gruenbacher, G.; et al, Oncoimmunology 2017, 6, e1342917). Both restricted flux (below basal flux) as well as enhanced flux through the mevalonate pathway leads to distinct immune response. Thus, such results indicate that cholesterol pathway and mevalonate pathway influence the immune system thereby has crucial role in treatment of inflammatory diseases (see, Azzam, K. M.; et al., Trends Endocrinol Metab. 2012, 23, 169-178).

As such, the present invention provides a new class of small-molecules having a quinolinyl-pyrazine-carboxamide (or similar) structure which function as activators of the cholesterol biosynthesis pathway within cancer cells and/or immune cells (e.g., activating gene expression within one or more of the genes listed in Tables III-XIX) (e.g., activating gene expression of one or more of INSIG1, DHCR7, MVK and MSMO1) (e.g., de-activating gene expression of one or more of GPR135, SPDYA, ABCA1 and HRH4), which function as activators of the cell cycle regulation pathway within cancer cells and/or immune cells (e.g., activating gene expression one or more of AVPI1, CCNG2, TUBA1A, H2AFX, and HIST1H3C), which function as up-regulators of HMGCS1 protein expression within cancer cells and/or immune cells, and which function as effective therapeutic agents for treating, ameliorating, and preventing various forms of cancer and other inflammatory diseases.

In a particular embodiment, quinolinyl-pyrazine-carboxamide (or similar) compounds encompassed within Formula IA or IB are provided:

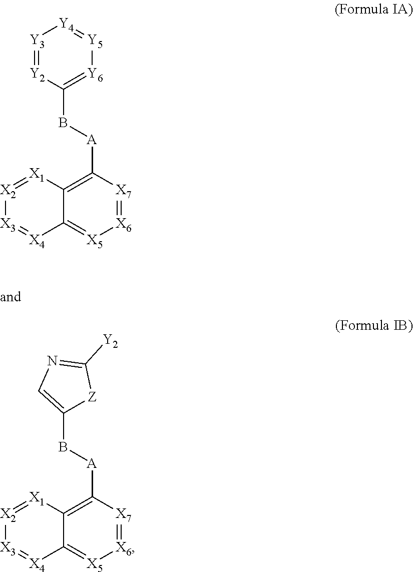

including pharmaceutically acceptable salts (e.g., 2,2,2-trifluoroacetate (TFA) salts and other salts) (e.g., physiologically tolerated acid addition salts), solvates, and/or prodrugs thereof.

Formulas IA and IB are not limited to a particular chemical moiety for A, B, X1, X2, X3, X4, X5, X6, X7, Y2, Y3, Y4, Y5, Y6 and Z. In some embodiments, the particular chemical moiety for A, B, X1, X2, X3, X4, X5, X6, X7, Y2, Y3, Y4, Y5, Y6 and Z independently include any chemical moiety that permits the resulting compound capable of activating the cholesterol biosynthesis pathway within cancer cells and/or immune cells (e.g., activating gene expression within one or more of the genes listed in Tables III-XIX) (e.g., activating gene expression of one or more of INSIG1, DHCR7, MVK and MSMO1) (e.g., de-activating gene expression of one or more of GPR135, SPDYA, ABCA1 and HRH4). In some embodiments, the particular chemical moiety for A, B, X1, X2, X3, X4, X5, X6, X7, Y2, Y3, Y4, Y5, and Y6 independently include any chemical moiety that permits the resulting compound capable of activating the cell cycle regulation pathway within cancer cells and/or immune cells (e.g., activating gene expression one or more of AVPI1, CCNG2, TUBA1A, H2AFX, and HIST1H3C). In some embodiments, the particular chemical moiety for A, B, X1, X2, X3, X4, X5, X6, X7, Y2, Y3, Y4, Y5, and Y6 independently include any chemical moiety that permits the resulting compound capable of up-regulating HMGCS1 protein expression within cancer cells and/or immune cells. In some embodiments, the particular chemical moiety for A, B, X1, X2, X3, X4, X5, X6, X7, Y2, Y3, Y4, Y5, and Y6 independently include any chemical moiety that permits the resulting compound capable of serving as an effective therapeutic agent for treating, ameliorating, and preventing various forms of cancer and other inflammatory diseases.

In some embodiments, X¹ is either CH or N.

In some embodiments, X², X³, X⁴, X⁵, X⁶ and X⁷ are independently selected from CR¹ or N, with the proviso that at least three of them must be CR¹.

In some embodiments, Y², Y³, Y⁴, Y⁵, Y⁶ are independently CH, CR² or N.

In some embodiments, Y⁶ is a bond, in which case one of Y³, Y⁴, or Y⁵ is NR³, O, or S, while the other two may be CR² or N.

In some embodiments, A and B are independently selected from a group consisting of NH, C═O, C═S, CH₂, C(R³)₂, CF₂, C—NMe₂, C═N—OR⁴, C═N—N(R⁵)₂, C═N—SO₂R⁶, or C═N—CN.

In some embodiments, Z is either O, S or NH.

In some embodiments, R¹ is independently H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆thioalkoxy, C₁₋₆thioalkoxy-C₃₋₇ cycloalkyl, C₁₋₆thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR¹⁰, SO₂R¹⁰, NO₂, COR⁷, C₁₋₆ alkyl-COR⁷, N(R¹⁰)C₂₋₆ alkyl-NR¹⁰R¹⁰, —N(R¹⁰)C₂₋₆ alkyl-R⁷, N(C₂₋₆ alkyl)₂-NR¹⁰, —O(CH₂)ₚR⁷, —S(CH₂)ₚR⁷, or —N(R¹⁰)C(═O)(CH₂)ₚR⁷, with a proviso that not more than three R¹ can be other than H.

In some embodiments, R² is independently H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆ thioalkoxy, C₁₋₆ thioalkoxy, C₁₋₆thioalkoxy-C₃₋₇ cycloalkyl, C₁₋₆thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR¹⁰, SO₂R¹⁰, NO₂, COR⁷, C₁₋₆ alkyl-COR⁷, N(R¹⁰)C₂₋₆alkyl-NR¹⁰R¹⁰, N(C₂₋₆alkyl)₂-NR¹⁰,

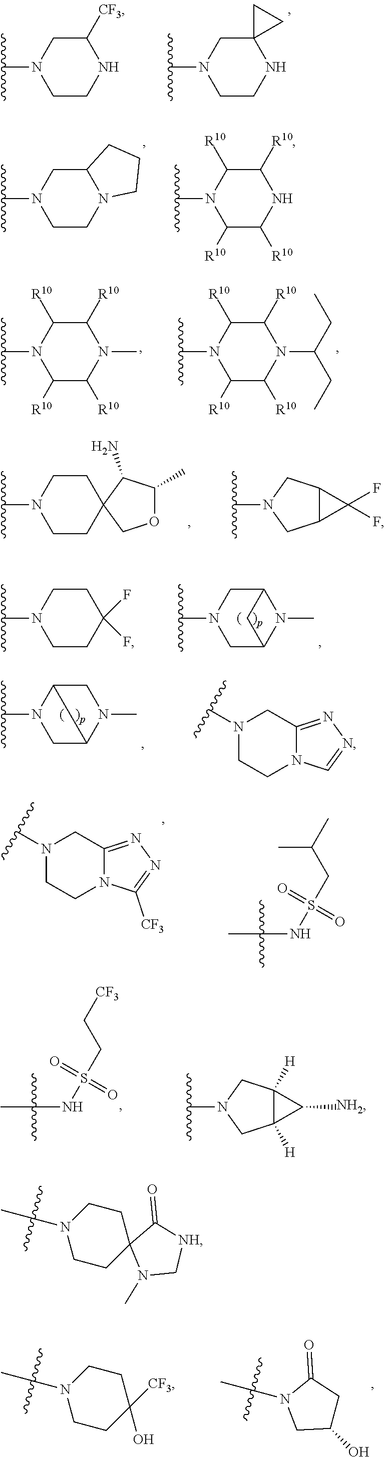

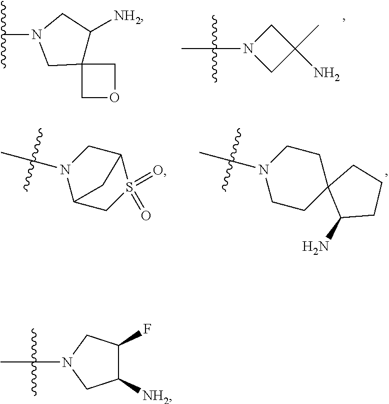

O(CH₂)ₚR⁷, —S(CH₂)ₚR⁷, or —N(R¹⁰)C(═O)(CH₂)ₚR⁷, with a proviso that not more than two R² can be other than H.

In some embodiments, R³ is hydrogen, C₁₋₆ alkyl, C₃₋₆ alkenyl, C₃₋₆alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, or C₁₋₆ alkyl-C₄₋₇ heterocycloalkyl.

In some embodiments, R⁴ is H or C₁₋₆ alkyl.

In some embodiments, each R⁵ is independently H or C₁₋₆ alkyl, or the two R⁵, taken together with the N atom to which they are both attached, form a heterocycloalkyl ring of 4-7 members, containing up to one other heteroatom selected from O, S, or NR³; R⁶ is C₁₋₆ alkyl or CF₃.

In some embodiments, R⁷ is OH, NR⁸R⁹, Q(CH₂)_{q}NR⁸R⁹, C₁₋₆ alkoxy, C₁₋₆ alkoxy-C₁₋₆ alkoxy, C₂₋₆ hydroxyalkoxy, cyclopropyl,

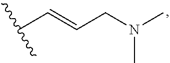 [SMILES: */C=C/CN(C)C]

oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, diazepanylamino, all of which may be optionally substituted with OH, OR¹⁰, oxo, halogen, R¹⁰, CH₂OR¹⁰, CH₂NR⁸R⁹ or CH₂CH₂CONR⁸R⁹.

In some embodiments, R⁸ and R⁹ are each independently H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, Cn C₆ acyl, 4-12 membered monocyclic or bicyclic heterocyclyl, 4-12 membered monocyclic or bicyclic heterocyclyl-C₁-C₆ alkyl-, C₆₋C₁₂ aryl, 5-11 membered heteroaryl; wherein R⁸ and R⁹ may be further independently substituted with up to three substituents chosen from hydroxyl, C₁₋₆ alkoxy, C₁₋₆ hydroxyalkyl, C₁₋₆ alkoxy-C₁₋₆ alkyl, C₁₋₆ alkoxy-C₁₋₆ alkoxy, C₂₋₆ hydroxyalkoxy, oxo, thiono, cyano or halo; or alternatively, R⁸ and R⁹, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³.

In some embodiments, each R¹⁰ is independently H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂— alkyl, NH—C₂₋₆ alkyl-NR⁸R⁹, C₁₋₆ alkoxy-C₁₋₆ alkyl or C₂₋₆ alkyl-NR⁸R⁹; alternatively, two R¹⁰ taken together with the same N atom to which they are both attached, form a heterocyclic ring of 4-7 members, containing up to one other heteroatom selected from O, S, or NR³.

In some embodiments, p=0, 1, 2, 3, or 4.

In some embodiments, x=0, 1, or 2.

In some embodiments, X1 is N, and A is NH thereby rendering a compound encompassed within Formula II

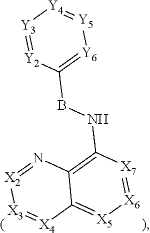

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵ Y⁶ are independently selected from CH, CR² or N; or Y⁶ is a bond, in which case one of Y³, Y⁴, or Y⁵ is NR³, O, or S, while the other two may be CR² or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;

wherein R¹, R², R³, R⁴, R⁵, R⁶, (R⁷-R¹⁰ embedded in R¹ and R²) are as described within Formula I.

In some embodiments, X1 is N, and A is C═O thereby rendering a compound encompassed within Formula III

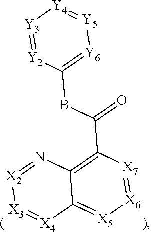

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵ Y⁶ are independently selected from CH, CR² or N;

wherein B is selected from a group consisting of NH, CH₂, C(R³)₂, C—NMe₂, C═N—OR⁴, C═N—N(R⁵)₂;

wherein R¹, R², R³, R⁴, R⁵, (R⁷-R¹⁰ embedded in R¹ and R²) are as described within Formula I.

In some embodiments, X1 is N, and A is CH₂ thereby rendering a compound encompassed within Formula IV

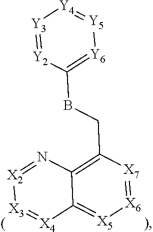

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵, Y⁶ are independently selected from CH, CR² or N; or Y⁶ is a bond, in which case one of Y³, Y⁴, or Y⁵ is NR³, O, or S, while the other two may be CR² or N;

wherein B selected from a group consisting of NH, C═O, C═S, CH₂, C(R³)₂, CF₂, C—NMe₂, C═N—OR⁴, C═N—N(R⁵)₂;

wherein R¹, R², R³, R⁴, R⁵, (R⁷-R¹⁰ embedded in R¹ and R²) are as described in Formula I.

In some embodiments, X1 is N, A is NH, and Y4 is C—R2 thereby rendering a compound encompassed within Formula V

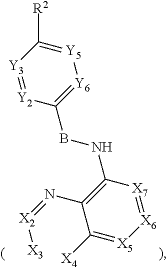

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁵, Y⁶ are independently selected from CH or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;

wherein R¹, R², R³, R⁴, R⁵, R⁶, (R⁷-R¹⁰ embedded in R¹ and R²) are as described within Formula I.

In some embodiments, X1 is N, A is NH, and X2 is C—O—CH₃ thereby rendering a compound encompassed within Formula VI

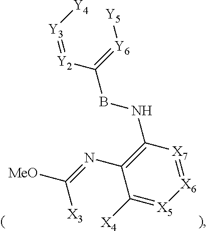

including pharmaceutically acceptable salts (e.g., 2,2,2-trifluoroacetate (TFA) salts and other salts) (e.g., physiologically tolerated acid addition salts), solvates, and/or prodrugs thereof,

wherein X³, X⁴, X⁵, X⁶ and X⁷ are independently selected from CR¹ or N;

wherein Y², Y³, Y⁴, Y⁵, Y⁶ are independently selected from CH, CR² or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;

wherein R¹, R², R³, R⁴, R⁵, R⁶, (R⁷-R¹⁰ embedded in R¹ and R²) are as described within Formula I.

In some embodiments, X1 is N, A is NH, and X6 is C—F thereby rendering a compound encompassed within Formula VII

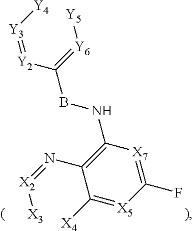

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵ and X⁷ are independently selected from CR¹ or N;

wherein Y², Y³, Y⁴, Y⁵, Y⁶ are independently selected from CH, CR² or N;

wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within Formula I.

In some embodiments, X1 is N, A is NH, B is CH, and X6 is C—CH₃ thereby rendering a compound encompassed within Formula VIII

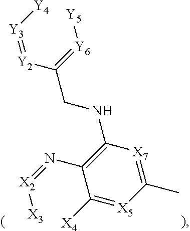

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵ and X⁷ are independently selected from CR¹ or N, with the proviso that at least two of them must be CR¹;

wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;

wherein R¹, R², R³, (R⁷-R¹⁰ embedded in R¹ and R²) are as described within Formula I.

In some embodiments, X1 is N, and A is NH, thereby rendering a compound encompassed within Formula IX

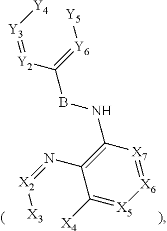

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, X⁶ and X⁷ are independently selected from CR¹ or N, with the proviso that at least three of them must be CR¹;

wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;

wherein B is selected from a group consisting of NH, C═O, C═S, CH₂, C(R³)₂, CF₂, C—NMe₂, C═N—OR⁴, C═N—N(R⁵)₂, C═N—SO₂R⁶, C═N—CN;

wherein R¹ is N(C₂₋₆ alkyl)₂—NH;

wherein R² is selected from H or Me;

wherein R³, R⁴, R⁵, R⁶ are as described within Formula I.

In some embodiments, X1 is N, X2 is CH, X3 is CH, X4 is CH, X5 is CH, X6 is C—R1, X7 is CH, and Y4 is

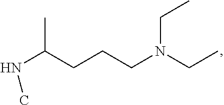

thereby rendering a compound encompassed within Formula X

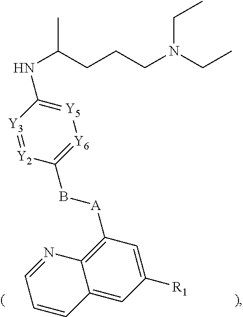

including pharmaceutically acceptable salts (e.g., 2,2,2-trifluoroacetate (TFA) salts and other salts) (e.g., physiologically tolerated acid addition salts), solvates, and/or prodrugs thereof,

wherein Y², Y³, Y⁵ Y⁶ are independently CH or N;

wherein A and B selected from a group consisting of NH, C═O, C═S, CH₂, C(R³)₂, CF₂, C—NMe₂, C═N—OR⁴, C═N—N(R⁵)₂, C═N—SO₂R⁶, C═N—CN;

wherein R¹, R³, R⁴, R⁵, R⁶, (R⁷-R¹⁰ embedded in R¹) are as described within Formula I.

In some embodiments, A is NH, B is C═O, X1 is N, X2 is CH, X3 is CH, X4 is CH, X5 is CH, X6 is C—R1, X7 is CH, and Y4 is

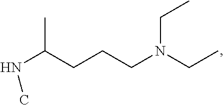

thereby rendering a compound encompassed within Formula XI

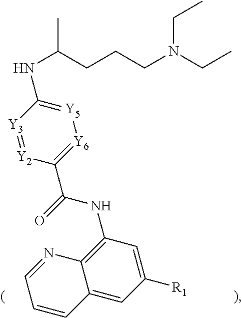

including pharmaceutically acceptable salts (e.g., 2,2,2-trifluoroacetate (TFA) salts and other salts) (e.g., physiologically tolerated acid addition salts), solvates, and/or prodrugs thereof, wherein Y², Y³, Y⁵, Y⁶ are independently CH or N;

wherein R¹, (R⁷-R¹⁰ embedded in R¹) are as described within Formula I.

In some embodiments, A is NH, B is C═O, X1 is N, and X2 is C—R1, thereby rendering a compound encompassed within Formula XII

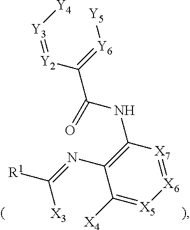

including pharmaceutically acceptable salts (e.g., 2,2,2-trifluoroacetate (TFA) salts and other salts) (e.g., physiologically tolerated acid addition salts), solvates, and/or prodrugs thereof,

wherein X³, X⁴, X⁵, X⁶ and X⁷ are independently selected from CH or N;

wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;

wherein R¹, R², R³, (R⁷-R¹⁰ embedded in R¹ and R²) are as described within Formula I.

In some embodiments, A is C═O, B is NH, X1 is N, X6 is C—R1, and Y4 is C—R2, thereby rendering a compound encompassed within Formula XIII

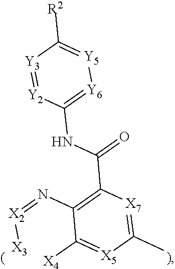

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, and X⁷ are independently selected from CR¹ or N, with the proviso that at least two of them must be CR¹;
wherein Y², Y³, Y⁵, Y⁶ are independently CH or N;
wherein R¹, R², (R⁷-R¹⁰ embedded in R¹ and R²) are as described within Formula I.

In some embodiments, A is NH, B is C═O, X1 is N, X2 is CH, X3 is CH, X4 is CH, X5 is CH, X6 is C—R1, X7 is CH, Y2 is N, Y3 is CH, Y4 is C—R2, Y5 is N, and Y6 is CH, thereby rendering a compound encompassed within Formula XIV

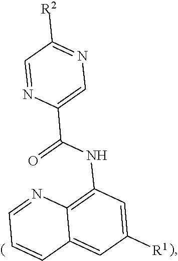

including pharmaceutically acceptable salts (e.g., 2,2,2-trifluoroacetate (TFA) salts and other salts) (e.g., physiologically tolerated acid addition salts), solvates, and/or prodrugs thereof,

wherein R¹ is independently H, Me and halogen;

wherein R², (R⁷-R¹⁰ embedded in R²) are as described within Formula I.

In some embodiments the compounds are encompassed within Formula XV:

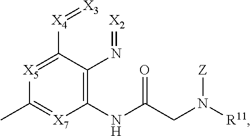

including pharmaceutically acceptable salts (e.g., 2,2,2-trifluoroacetate (TFA) salts and other salts) (e.g., physiologically tolerated acid addition salts), solvates, and/or prodrugs thereof,

wherein X², X³, X⁴, X⁵, and X⁷ are independently selected from CR¹ or N, with the proviso that at least two of them must be CR¹;

wherein Z is independently C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-membered mono- or bicyclo-heteroaryl), C₁₋₆thioalkoxy, C₁₋₆thioalkoxy, C₁₋₆thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, C₂₋₆ alkyl-NR¹⁰R¹⁰, —C₂₋₆alkyl-R⁷;

wherein R¹¹ is H or Me;

wherein R⁷ and R¹⁰, (R⁸-R⁹ embedded in R⁷ and R¹⁰) are as described within Formula I.

In some embodiments, compounds shown in Table I are contemplated for Formula I.

An important aspect of the present invention is that compounds of the invention induce cell cycle arrest and/or apoptosis and also potentiate the induction of cell cycle arrest and/or apoptosis either alone or in response to additional apoptosis induction signals. Therefore, it is contemplated that these compounds sensitize cells to induction of cell cycle arrest and/or apoptosis, including cells that are resistant to such inducing stimuli.

In some embodiments, the compositions and methods of the present invention are used to treat diseased cells, tissues, organs, or pathological conditions and/or disease states in an animal (e.g., a mammalian patient including, but not limited to, humans and veterinary animals). In this regard, various diseases and pathologies are amenable to treatment or prophylaxis using the present methods and compositions. A non-limiting exemplary list of these diseases and conditions includes, but is not limited to, any type of cancer including but not limited to pancreatic cancer, breast cancer, prostate cancer, lymphoma, skin cancer, colon cancer, melanoma, malignant melanoma, ovarian cancer, brain cancer, primary brain carcinoma, head and neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma, and the like, T and B cell mediated autoimmune diseases; inflammatory diseases; infections; hyperproliferative diseases; AIDS; degenerative conditions, vascular diseases, and the like. In some embodiments, the cancer cells being treated are metastatic. In other embodiments, the cancer cells being treated are resistant to anticancer agents.

Some embodiments of the present invention provide methods for administering an effective amount of a compound of the invention and at least one additional therapeutic agent (including, but not limited to, chemotherapeutic antineoplastics, apoptosis-modulating agents, antimicrobials, antivirals, antifungals, and anti-inflammatory agents) and/or therapeutic technique (e.g., surgical intervention, and/or radiotherapies).

In a particular embodiment, the additional therapeutic agent(s) is an anticancer agent. A number of suitable anticancer agents are contemplated for use in the methods of the present invention. Indeed, the present invention contemplates, but is not limited to, administration of numerous anticancer agents such as: agents that induce apoptosis; polynucleotides (e.g., anti-sense, ribozymes, siRNA); polypeptides (e.g., enzymes and antibodies); biological mimetics; alkaloids; alkylating agents; antitumor antibiotics; antimetabolites; hormones; platinum compounds; monoclonal or polyclonal antibodies (e.g., antibodies conjugated with anticancer drugs, toxins, defensins), toxins; radionuclides; biological response modifiers (e.g., interferons (e.g., IFN-α) and interleukins (e.g., IL-2)); adoptive immunotherapy agents; hematopoietic growth factors; agents that induce tumor cell differentiation (e.g., all-trans-retinoic acid); gene therapy reagents (e.g., antisense therapy reagents and nucleotides); tumor vaccines; angiogenesis inhibitors; proteosome inhibitors: NF-KB modulators; anti-CDK compounds; HDAC inhibitors; and the like. Numerous other examples of chemotherapeutic compounds and anticancer therapies suitable for co-administration with the disclosed compounds are known to those skilled in the art.

In certain embodiments, anticancer agents comprise agents that induce or stimulate apoptosis. Agents that induce apoptosis include, but are not limited to, radiation (e.g., X-rays, gamma rays, UV); tumor necrosis factor (TNF)-related factors (e.g., TNF family receptor proteins, TNF family ligands, TRAIL, antibodies to TRAIL-R1 or TRAIL-R2); kinase inhibitors (e.g., epidermal growth factor receptor (EGFR) kinase inhibitor, vascular growth factor receptor (VGFR) kinase inhibitor, fibroblast growth factor receptor (FGFR) kinase inhibitor, platelet-derived growth factor receptor (PDGFR) kinase inhibitor, and Bcr-Abl kinase inhibitors (such as GLEEVEC)); antisense molecules; antibodies (e.g., HERCEPTIN, RITUXAN, ZEVALIN, and AVASTIN); anti-estrogens (e.g., raloxifene and tamoxifen); anti-androgens (e.g., flutamide, bicalutamide, finasteride, aminoglutethamide, ketoconazole, and corticosteroids); cyclooxygenase 2 (COX-2) inhibitors (e.g., celecoxib, meloxicam, NS-398, and non-steroidal anti-inflammatory drugs (NSAIDs)); anti-inflammatory drugs (e.g., butazolidin, DECADRON, DELTASONE, dexamethasone, dexamethasone intensol, DEXONE, HEXADROL, hydroxychloroquine, METICORTEN, ORADEXON, ORASONE, oxyphenbutazone, PEDIAPRED, phenylbutazone, PLAQUENIL, prednisolone, prednisone, PRELONE, and TANDEARIL); and cancer chemotherapeutic drugs (e.g., irinotecan (CAMPTOSAR), CPT-11, fludarabine (FLUDARA), dacarbazine (DTIC), dexamethasone, mitoxantrone, MYLOTARG, VP-16, cisplatin, carboplatin, oxaliplatin, 5-FU, doxorubicin, gemcitabine, bortezomib, gefitinib, bevacizumab, TAXOTERE or TAXOL); cellular signaling molecules; ceramides and cytokines; staurosporine, and the like.

In still other embodiments, the compositions and methods of the present invention provide a compound of the invention and at least one anti-hyperproliferative or antineoplastic agent selected from alkylating agents, antimetabolites, and natural products (e.g., herbs and other plant and/or animal derived compounds).

Alkylating agents suitable for use in the present compositions and methods include, but are not limited to: 1) nitrogen mustards (e.g., mechlorethamine, cyclophosphamide, ifosfamide, melphalan (L-sarcolysin); and chlorambucil); 2) ethylenimines and methylmelamines (e.g., hexamethylmelamine and thiotepa); 3) alkyl sulfonates (e.g., busulfan); 4) nitrosoureas (e.g., carmustine (BCNU); lomustine (CCNU); semustine (methyl-CCNU); and streptozocin (streptozotocin)); and 5) triazenes (e.g., dacarbazine (DTIC; dimethyltriazenoimid-azolecarboxamide).

In some embodiments, antimetabolites suitable for use in the present compositions and methods include, but are not limited to: 1) folic acid analogs (e.g., methotrexate (amethopterin)); 2) pyrimidine analogs (e.g., fluorouracil (5-fluorouracil; 5-FU), floxuridine (fluorode-oxyuridine; FudR), and cytarabine (cytosine arabinoside)); and 3) purine analogs (e.g., mercaptopurine (6-mercaptopurine; 6-MP), thioguanine (6-thioguanine; TG), and pentostatin (2′-deoxycoformycin)).

In still further embodiments, chemotherapeutic agents suitable for use in the compositions and methods of the present invention include, but are not limited to: 1) vinca alkaloids (e.g., vinblastine (VLB), vincristine); 2) epipodophyllotoxins (e.g., etoposide and teniposide); 3) antibiotics (e.g., dactinomycin (actinomycin D), daunorubicin (daunomycin; rubidomycin), doxorubicin, bleomycin, plicamycin (mithramycin), and mitomycin (mitomycin C)); 4) enzymes (e.g., L-asparaginase); 5) biological response modifiers (e.g., interferon-alfa); 6) platinum coordinating complexes (e.g., cisplatin (cis-DDP) and carboplatin); 7) anthracenediones (e.g., mitoxantrone); 8) substituted ureas (e.g., hydroxyurea); 9) methylhydrazine derivatives (e.g., procarbazine (N-methylhydrazine; MIH)); 10) adrenocortical suppressants (e.g., mitotane (o,p′-DDD) and aminoglutethimide); 11) adrenocorticosteroids (e.g., prednisone); 12) progestins (e.g., hydroxyprogesterone caproate, medroxyprogesterone acetate, and megestrol acetate); 13) estrogens (e.g., diethylstilbestrol and ethinyl estradiol); 14) antiestrogens (e.g., tamoxifen); 15) androgens (e.g., testosterone propionate and fluoxymesterone); 16) antiandrogens (e.g., flutamide): and 17) gonadotropin-releasing hormone analogs (e.g., leuprolide).

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table II provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE II

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and ILEX |
| (IgG1κ anti CD52 antibody) | | Partners, LP, |
| | | Cambridge, MA |
| Alitretinoin | Panretin | Ligand Pharmaceuticals, |
| (9-cis-retinoic acid) | | Inc., San Diego CA |
| Allopurinol | Zyloprim | GlaxoSmithKline, |
| (1,5-dihydro-4 H-pyrazolo[3,4-d]pyrimidin-4- | | Research Triangle Park, |
| one monosodium salt) | | NC |
| Altretamine | Hexalen | US Bioscience, West |
| (N,N,N′,N′,N″,N″,-hexamethyl-1,3,5-triazine- | | Conshohocken, PA |
| 2,4,6-triamine) | | |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a, a, a′, a′- | | Pharmaceuticals, LP, |
| tetramethyl-5-(1H-1,2,4-triazol-1-ylmethyl)) | | Wilmington, DE |
| Arsenic trioxide | Trisenox | Cell Therapeutic, Inc., |
| | | Seattle, WA |
| Asparaginase | Elspar | Merck & Co., Inc., |
| (L-asparagine amidohydrolase, type EC-2) | | Whitehouse Station, NJ |
| BCG Live | TICE BCG | Organon Teknika, Corp., |
| (lyophilized preparation of an attenuated strain | | Durham, NC |
| of Mycobacterium bovis (Bacillus Calmette- | | |
| Gukin [BCG], substrain Montreal) | | |
| bexarotene capsules | Targretin | Ligand Pharmaceuticals |
| (4-[1-(5,6,7,8-tetrahydro-3,5,5,8,8- | | |
| pentamethyl-2-napthalenyl) ethenyl] benzoic | | |
| acid) | | |
| bexarotene gel | Targretin | Ligand Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-[(pentyloxy)carbonyl]- | | |
| cytidine) | | |
| Carboplatin | Paraplatin | Bristol-Myers Squibb |
| (platinum, diammine [1,1- | | |
| cyclobutanedicarboxylato(2-)-0,0′]-, (SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle Pharmaceuticals, |
| (as 4-[5-(4-methylphenyl)-3-(trifluoromethyl)- | | England |
| 1H-pyrazol-1-yl] | | |
| benzenesulfonamide) | | |
| Chlorambucil | Leukeran | GlaxoSmithKline |
| (4-[bis(2chlorethyl)amino]benzenebutanoic | | |
| acid) | | |
| Cisplatin | Platinol | Bristol-Myers Squibb |
| (PtCl₂H₆N₂) | | |
| Cladribine | Leustatin, 2- | R.W. Johnson |
| (2-chloro-2′-deoxy-b-D-adenosine) | CdA | Pharmaceutical |
| | | Research Institute, |
| | | Raritan, NJ |
| Cyclophosphamide | Cytoxan, Neosar | Bristol-Myers Squibb |
| (2-[bis(2-chloroethyl)amino] tetrahydro-2H- | | |
| 13,2-oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye Pharmaceuticals, |
| | | Inc., San Diego, CA |
| Dacarbazine | DTIC-Dome | Bayer AG, Leverkusen, |
| (5-(3,3-dimethyl-1-triazeno)-imidazole-4- | | Germany |
| carboxamide (DTIC)) | | |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., Thousand |
| (recombinant peptide) | | Oaks, CA |
| daunorubicin liposomal | DanuoXome | Nexstar |
| ((8S-cis)-8-acety1-10-[(3-amino-2,3,6-trideoxy- | | Pharmaceuticals, Inc., |
| ECOMMANDa-L-lyxo-hexopyranosyl)oxy]-7,8,9,10- | | Boulder, CO |
| tetrahydro-6,8,11-trihydroxy-1-methoxy-5,12- | | |
| naphthacenedione hydrochloride) | | |
| Daunorubicin HCl, daunomycin | Cerubidine | Wyeth Ayerst, Madison, |
| ((1S,3S)-3-Acetyl-1,2,3,4,6,11-hexahydro- | | NJ |
| 3,5,12-trihydroxy-10-methoxy-6,11-dioxo-1- | | |
| naphthacenyl 3-amino-2,3,6-trideoxy-(alpha)- | | |
| L-lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | Company | |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one | | |
| 4-acetate 2-benzoate, trihydrate) | | |
| Doxorubicin HCl | Adriamycin, | Pharmacia & Upjohn |
| (8S,10S)-10-[(3-amino-2,3,6-trideoxy-a-L- | Rubex | Company |
| lyxo-hexopyranosyl)oxy]-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia & Upjohn |
| | Intravenous | Company |
| | injection | |
| doxorubicin liposomal | Doxil | Sequus Pharmaceuticals, |
| | | Inc., Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott's B Solution | Elliott's B | Orphan Medical, Inc |
| | Solution | |
| Epirubicin | Ellence | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| [bis(2-chloroethyl)carbamate]17-(dihydrogen | | |
| phosphate), disodium salt, monohydrate, or | | |
| estradiol 3-[bis(2-chloroethyl)carbamate] 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-[4,6-O-(R)- | | |
| ethylidene-(beta)-D-glucopyranoside], 4′- | | |
| (dihydrogen phosphate)) | | |
| etoposide, VP-16 | Vepesid | Bristol-Myers Squibb |
| (4′-demethylepipodophyllotoxin 9-[4,6-0-(R)- | | |
| ethylidene-(beta)-D-glucopyranoside]) | | |
| Exemestane | Aromasin | Pharmacia & Upjohn |
| (6-methylenandrosta-1,4-diene-3, 17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, NJ |
| agent vidarabine, 9-b-D- | | |
| arabinofuranosyladenine (ara-A)) | | |
| Fluorouracil, 5-FU | Adrucil | ICN Pharmaceuticals, |
| (5-fluoro-2,4(1H, 3H)-pyrimidinedione) | | Inc., Humacao, Puerto |
| Rico | | |
| Fulvestrant | Faslodex | IPR Pharmaceuticals, |
| (7-alpha-[9-(4,4,5,5,5-penta | | Guayama, Puerto Rico |
| fluoropentylsulphinyl) nonyl]estra-1,3,5-(10)- | | |
| triene-3,17-beta-diol) | | |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| | | Pharmaceuticals |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal | | |
| antibody Ibritumomab and the linker-chelator | | |
| tiuxetan [N-[2-bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & Upjohn |
| (5, 12-Naphthacenedione, 9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | Gleevec | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-[(4-piperi- | | Company |
| dinopiperidino)carbonyloxy]-1H-pyrano[3′, 4′: | | |
| 6,7] indolizino[1,2-b] quinoline-3,14(4H, 12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., Seattle, |
| (L-Glutamic acid, N[4[[(2amino-5-formyl- | Leucovorin | WA |
| 1,4,5,6,7,8 hexahydro4oxo6- | | |
| pteridinyl)methyl]amino]benzoyl], calcium salt | | |
| (1:1)) | | |
| Levamisole HCl | Ergamisol | Janssen Research |
| ((−)-(S)-2,3,5,6-tetrahydro-6-phenylimidazo | | Foundation, Titusville, |
| [2,1-b] thiazole monohydrochloride | | NJ |
| C₁₁H₁₂N₂S•HCl) | | |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-[bis(2-chloroethyl) amino]-L-phenylalanine) | | |
| Mercaptopurine, 6-MP | Purinethol | GlaxoSmithKline |
| (1,7-dihydro-6 H-purine-6-thione | | |
| monohydrate) | | |
| Mesna | Mesnex | Asta Medica |
| (sodium 2-mercaptoethane sulfonate) | | |
| Methotrexate | Methotrexate | Lederle Laboratories |
| (N-[4-[[(2,4-diamino-6- | | |
| pteridinyl)methyl]methylamino]benzoyl]-L- | | |
| glutamic acid) | | |
| Methoxsalen | Uvadex | Therakos, Inc., Way |
| (9-methoxy-7H-furo[3,2-g][1]-benzopyran-7- | | Exton, Pa |
| one) | | |
| Mitomycin C | Mutamycin | Bristol-Myers Squibb |
| mitomycin C | Mitozytrex | SuperGen, Inc., Dublin, |
| | | CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex Corporation |
| (1,4-dihydroxy-5,8-bis[[2-[(2- | | |
| hydroxyethyl)amino]ethyl]amino]-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, Germany |
| Oprelvekin | Neumega | Genetics Institute, Inc., |
| (IL-11) | | Alexandria, VA |
| Oxaliplatin | Eloxatin | Sanofi Synthelabo, Inc., |
| (cis-[(1R,2R)-1,2-cyclohexanediamine-N,N′] | | NY, NY |
| [oxalato(2-)-O,40] platinum) | | |
| Paclitaxel | TAXOL | Bristol-Myers Squibb |
| (5β, 20-Epoxy-1,2a, 4,7β, 10β, 13a- | | |
| hexahydroxytax-11-en-9-one 4,10-diacetate 2- | | |
| benzoate 13-ester with (2R,3S)-N-benzoyl-3- | | |
| phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon Pharmaceuticals, |
| ((monomethoxypolyethylene glycol | (Pegademase | Inc., Bridgewater, NJ |
| succinimidyl) 11-17-adenosine deaminase) | Bovine) | |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, Inc., |
| (recombinant peptide) | | |
| Rituximab | Rituxan | Genentech, Inc., South |
| (recombinant anti-CD20 antibody) | | San Francisco, CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| [[(methylnitrosoamino)carbonyl]amino]-a(and | | |
| b)-D-glucopyranose and 220 mg citric acid | | |
| anhydrous) | | |
| Talc | Sclerosol | Bryan, Corp., Woburn, |
| (Mg₃Si₄O₁₀ (OH)2) | | MA |
| Tamoxifen | Nolvadex | AstraZeneca |
| ((Z)2-[4-(1,2-diphenyl-1-butenyl) phenoxy]-N, | | Pharmaceuticals |
| N-dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo[5,1-d]-as- | | |
| tetrazine-8-carboxamide) | | |
| teniposide, VM-26 | Vumon | Bristol-Myers Squibb |
| (4′-demethylepipodophyllotoxin 9-[4,6-0-(R)- | | |
| 2-thenylidene-(beta)-D-glucopyranoside]) | | |
| Testolactone | Teslac | Bristol-Myers Squibb |
| (13-hydroxy-3-oxo-13,17-secoandrosta-1,4- | | |
| dien-17-oic acid [dgr]-lactone) | | |
| Thioguanine, 6-TG | Thioguanine | GlaxoSmithKline |
| (2-amino-1,7-dihydro-6 H-purine-6-thione) | | |
| Thiotepa | Thioplex | Immunex Corporation |
| (Aziridine, 1,1′,1″-phosphinothioylidynetris-, or | | |
| Tris (1-aziridinyl) phosphine sulfide) | | |
| Topotecan HCl | Hycamtin | GlaxoSmithKline |
| ((S)-10-[(dimethylamino) methyl]-4-ethyl-4,9- | | |
| dihydroxy-1H-pyrano[3′, 4′: 6,7] indolizino | | |
| [1,2-b] quinoline-3,14-(4H, 12H)-dione | | |
| monohydrochloride) | | |
| Toremifene | Fareston | Roberts Pharmaceutical |
| (2-(p-[(Z)-4-chloro-1,2-diphenyl-1-butenyl]- | | Corp., Eatontown, NJ |
| phenoxy)-N,N-dimethylethylamine citrate | | |
| (1:1)) | | |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody | | |
| (I 131 is a radioimmunotherapeutic antibody)) | | |
| Trastuzumab | Herceptin | Genentech, Inc |
| (recombinant monoclonal IgGi kappa anti- | | |
| HER2 antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2- [1,2,3,4,6,11-hexahydro-2,5,12- | | |
| trihydroxy-7 methoxy-6,11-dioxo-[[4 2,3,6- | | |
| trideoxy-3-[(trifluoroacetyl)-amino-α-L-lyxo- | | |
| hexopyranosyl]oxyl]-2-naphthacenyl]-2- | | |
| oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl-phosphonoethyl) | | |
| phosphonic acid monohydrate) | | |

Anticancer agents further include compounds which have been identified to have anticancer activity. Examples include, but are not limited to, 3-AP, 12-O-tetradecanoylphorbol-13-acetate, 17AAG, 852A, ABI-007, ABR-217620, ABT-751, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflomithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hul4.18-interleukin-2 fusion protein, HuMax-CD4, iloprost, imiquimod, infliximab, interleukin-12, IPI-504, irofulven, ixabepilone, lapatinib, lenalidomide, lestaurtinib, leuprolide, LMB-9 immunotoxin, lonafamib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9, 06-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpimase, rebeccamycin analogue, rhuAngiostatin protein, rhuMab 2C₄, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifamib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” tenth edition, Eds. Hardman et al., 2002.

The present invention provides methods for administering a compound of the invention with radiation therapy. The invention is not limited by the types, amounts, or delivery and administration systems used to deliver the therapeutic dose of radiation to an animal. For example, the animal may receive photon radiotherapy, particle beam radiation therapy, other types of radiotherapies, and combinations thereof. In some embodiments, the radiation is delivered to the animal using a linear accelerator. In still other embodiments, the radiation is delivered using a gamma knife.

The source of radiation can be external or internal to the animal. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by animals. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

The animal may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to an animal, so long as the dose of radiation is tolerated by the animal without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. In one embodiment, the dose of radiation is fractionated for maximal target cell exposure and reduced toxicity.

In one embodiment, the total dose of radiation administered to an animal is about 0.01 Gray (Gy) to about 100 Gy. In another embodiment, about 10 Gy to about 65 Gy (e.g., about Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), or 1-2 Gy (e.g., 1.5-2 Gy). The daily dose of radiation should be sufficient to induce destruction of the targeted cells. If stretched over a period, in one embodiment, radiation is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. In one embodiment, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

Antimicrobial therapeutic agents may also be used as therapeutic agents in the present invention. Any agent that can kill, inhibit, or otherwise attenuate the function of microbial organisms may be used, as well as any agent contemplated to have such activities. Antimicrobial agents include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In some embodiments of the present invention, a compound of the invention and one or more therapeutic agents or anticancer agents are administered to an animal under one or more of the following conditions: at different periodicities, at different durations, at different concentrations, by different administration routes, etc. In some embodiments, the compound is administered prior to the therapeutic or anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the therapeutic or anticancer agent. In some embodiments, the compound is administered after the therapeutic or anticancer agent, e.g., 0.5, 1, 2, 3, 4, 5, 10, 12, or 18 hours, 1, 2, 3, 4, 5, or 6 days, or 1, 2, 3, or 4 weeks after the administration of the anti cancer agent. In some embodiments, the compound and the therapeutic or anticancer agent are administered concurrently but on different schedules, e.g., the compound is administered daily while the therapeutic or anticancer agent is administered once a week, once every two weeks, once every three weeks, or once every four weeks. In other embodiments, the compound is administered once a week while the therapeutic or anti cancer agent is administered daily, once a week, once every two weeks, once every three weeks, or once every four weeks.

Compositions within the scope of this invention include all compositions wherein the compounds of the present invention are contained in an amount which is effective to achieve its intended purpose. While individual needs vary, determination of optimal ranges of effective amounts of each component is within the skill of the art. Typically, the compounds may be administered to mammals, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the invention may be administered to any patient which may experience the beneficial effects of the compounds of the invention. Foremost among such patients are mammals, e.g., humans, although the invention is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

Bromouridine labeled RNA sequencing (Bru-seq) technique was used to better characterize transcriptional effects of the compounds of the present invention. Bru-seq captures nascent RNA and provides information on ongoing transcription genome-wide without interference by preexisting RNA.

INSIG1 mediates feedback control of cholesterol synthesis by controlling SCAP (SREBF Chaperone) and HMGCR 3-Hydroxy-3-Methylglutaryl-CoA Reductase). It functions by blocking the processing of sterol regulatory element-binding proteins (SREBPs) and initiates the sterol-mediated ubiquitin-mediated endoplasmic reticulum-associated degradation (ERAD) of HMGCR via recruitment of the reductase to the ubiquitin ligase, AMFR/gp78. The enzyme, 7-dehydrocholesterol reductase, encoded by the 7-dehydrocholesterol reductase (DHCR7) gene catalyzes the last step in cholesterol biosynthesis (see, Selma Feldman Witchel M D, Peter A. Lee M D, PhD, in Pediatric Endocrinology (Fourth Edition), 2014). Mevalonate kinase is an enzyme (specifically a kinase) that in humans is encoded by the MVK gene. Mevalonate kinase is the first enzyme to follow 3-hydroxy-3-methyl-glutaryl-CoA reductase (HMG-CoA reductase) in the mevalonate pathway and converts mevalonic acid to 5-phosphomevalonic acid. The mevalonate pathway produces cholesterol, a structural component of cellular membranes and precursor for bile acids and steroid hormones. In addition, the mevalonate pathway produces nonsterol isoprene compounds (see, Karyl S. Barron, Daniel L. Kastner, in Textbook of Pediatric Rheumatology (Seventh Edition), 2016). As revealed by Bru-seq, synthesis of INSIG1, DHCR7, MVK and MSMO1 RNAs was upregulated by treatment with either J4 (JR-1-235) (Table III). This implies cholesterol biosynthesis is the pathway involved in the mechanism of JR-1-235. Table (IV) lists the top 25 upregulated noncoding genes from Bru-seq data analysis of compound JR-1-235 when treated for 4 h in MIA PaCa-2 cells. A significant decrease in expression of GPR135, SPDYA and ABCA1 was also observed (Table V). Tables 3, 4, 5, 6, 7, 8, and 9 lists the top 25 upregulated genes from Bru-Seq analysis of compound JR-1-235 (H).

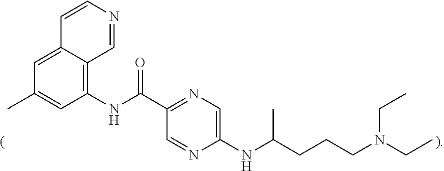 [SMILES: CCN(CC)CCCC(C)Nc1cnc(C(=O)Nc2cc(C)cc3ccncc23)cn1]

TABLE III
List of the top 25 upregulated protein-coding genes from Bru-seq data analysis
of compound JR-1-235 treated for 4 h in MIA PaCa-2 cells.

| No | Ensembl gene ID | Gene name | log2FC | Gene function |
|---|---|---|---|---|
| 1 | ENSG00000204386 | NEU1 | 2.68 | Neuraminidase 1 |
| | | | | (Glycoproteins and glycolipids biosynthesis |
| 2 | ENSG00000186480 | INSIG1 | 2.57 | Insulin Induced Gene 1 |
| | | | | Cholesterol metabolism, lipogenesis, and glucosc homeosta- |
| | | | | sis regulation |
| 3 | ENSG00000168209 | DDIT4 | 2.33 | DNA Damage Inducible Transcipt 4 |
| | | | | Cell growth, proliferation and survival regulation |
| 4 | ENSG00000172893 | DHCR7 | 2.13 | 7-Dehydrocholesterol Reductase |
| | | | | Cholesterol biosynthesis |
| 5 | ENSG00000099194 | SCD | 1.95 | Stearoyl-CoA Desaturase |
| | | | | Fatty acid biosynthesis |
| 6 | ENSG00000101670 | LIPG | 1.81 | Lipase G, Endothelial Type |
| | | | | Lipoprotein metabolism and vascular biology |
| 7 | ENSG00000160326 | SLC2A6 | 1.74 | Solute Carrier Family 2 Member 6/ GLUT9 |
| | | | | Facilitative glucose transporter |
| 8 | ENSG00000185813 | PCYT2 | 1.70 | Phosphate Cytidylytransferase 2, Ethanolamine |
| | | | | Phopholipid biosynthesis |
| 9 | ENSG00000110921 | MVK | 1.68 | Mevalonate Kinase |
| | | | | Cholesterol biosynthesis |
| 10 | ENSG00000100445 | SDR39U1 | 1.67 | Short Chain Dehydrogenase/Reductase Family 39U Member |
| | | | | 1 Oxidoreductase |
| 11 | ENSG00000052802 | MSMO1 | 1.65 | Methylsterol Monooxygenase 1 |
| | | | | Cholesterol biosynthesis |
| 12 | ENSG00000079459 | FDFT1 | 1.65 | Farnesyl-Diphosphate Farnesyltransferase 1 |
| | | | | Cholesterol biosynthesis |
| 13 | ENSG00000138764 | CCNG2 | 1.62 | Cyclin G2 |
| | | | | Growth and cell cycle progression regulation |
| 14 | ENSG00000104946 | TBC1D17 | 1.62 | TBC1 Domain Family Member 17 |
| | | | | Autophagy regulation |
| 15 | ENSG00000067064 | IDI1 | 1.49 | Isopentenyl-Diphosphate Delta Isomerase 1 |
| | | | | Cholesterol biosynthesis |
| 16 | ENSG00000119986 | AVPI1 | 1.37 | Arginine Vasopressin Induced 1 |
| | | | | Cell cyclc regulation |
| 17 | ENSG00000167508 | MVD | 1.36 | Mevalonate Diphosphate Decarboxylase |
| | | | | Cholesterol biosynthesis |
| 18 | ENSG00000112972 | HMGCS1 | 1.35 | 3-Hydroxy·3-Methylglutaryl-CoA Synthase 1 |
| | | | | Cholesterol biosynthesis |
| 19 | ENSG00000175197 | DDIT3 | 1.32 | DNA Damage Inducible Transcript 3 |
| | | | | ER stress response |
| 20 | ENSG00000134107 | BHLHE40 | 1.32 | Basic Helix-Loop-Helix Family Member E40 |
| | | | | Circadian rhythm and cell differentiation |
| 21 | ENSG00000189410 | SH2D5 | 1.29 | SH2 Domain Containing 5 |
| | | | | Synaptic plasticity regulation |
| 22 | ENSG00000274267 | HIST1H3B | 1.26 | Histone Cluster 1 H3 Family Member B |
| | | | | Nucleosome assembly, chromatin organization and silencing |
| 23 | ENSG00000160285 | LSS | 1.25 | Lanosterol Synthase |
| | | | | Cholesterol biosynthesis |
| 24 | ENSG00000168298 | HIST1H1E | 1.24 | Histone Cluster 1 H1 Family Member E |
| | | | | Nucleosome assembly, gene transcription regulation |
| 25 | ENSG00000167552 | TUBA1A | 1.24 | Tubulin Alpha 1a |
| | | | | Cell cycle, mitosis |

TABLE IV
List of the top 25 upregulated noncoding genes from Bru-seq data analysis of
compound JR-1-235 treated for 4 h in MIA PaCa-2 cells.

| No | Ensembl gene ID | Gene name | log2FC | Description |
|---|---|---|---|---|
| 1 | ENSG00000272993 | AC239868.4 | 1.12 | lincRNA |
| 2 | ENSG00000234264 | DEPDC1-AS1 | 1.05 | antisense_RNA |
| 3 | ENSG00000227533 | SLC2A1-AS1 | 1.02 | lincRNA |
| 4 | ENSG00000260000 | AL133338.1 | 1.02 | antisense_RNA |
| 5 | ENSG00000237232 | ZNF295-AS1 | 0.94 | lincRNA |
| 6 | ENSG00000183250 | LINC01547 | 0.93 | lincRNA |
| 7 | ENSG00000228436 | AL139260.1 | 0.88 | antisense_RNA |
| 8 | ENSG00000259827 | AC026461.1 | 0.86 | processed_transcript |
| 9 | ENSG00000255458 | AC108471.2 | 0.85 | lincRNA |
| 10 | ENSG00000265683 | SYPL1P2 | 0.84 | processed_pseudogene |
| 11 | ENSG00000255468 | AP001107.9 | 0.81 | antisense_RNA |
| 12 | ENSG00000249850 | KRT18P31 | 0.81 | processed_pseudogene |
| 13 | ENSG00000270019 | AC110769.2 | 0.80 | lincRNA |
| 14 | ENSG00000275560 | AC008115.3 | 0.78 | sense_intronic |
| 15 | ENSG00000274828 | AC068473.5 | 0.77 | lincRNA |
| 16 | ENSG00000215154 | AC141586.1 | 0.77 | transcribed_unprocessed_pseudogene |
| 17 | ENSG00000099251 | HSD17B7P2 | 0.76 | transcribed_unprocessed_pseudogene |
| 18 | ENSG00000255517 | AP002748.3 | 0.76 | antisense_RNA |
| 19 | ENSG00000247400 | DNAJC3-AS1 | 0.75 | lincRNA |
| 20 | ENSG00000255337 | AP001830.1 | 0.75 | antisense_RNA |
| 21 | ENSG00000250069 | AC011379.1 | 0.74 | sense_intronic |
| 22 | ENSG00000246339 | EXTL3-AS1 | 0.73 | antisense_RNA |
| 23 | ENSG00000214796 | AC098934.1 | 0.72 | transcribed_unprocessed_pseudogene |
| 24 | ENSG00000279048 | AC080080.1 | 0.72 | TEC, IncRNA |
| 25 | ENSG00000254682 | AP002387.1 | 0.72 | antisense_RNA |

In Tables V and XII the top upregulated and downregulated curated gene sets are summarized. The gene set REACTOME_CHOLESTEROL_BIOSYNTHESIS summarizes the genes involved in cholesterol biosynthesis pathway. The gene set PODAR_RESPONSE_TO_ADAPHOSTIN_UP shows collectively the genes up-regulated in MM1.S cells (multiple myeloma) treated with adaphostin, a tyrosine kinase inhibitor with anti cancer properties.

TABLE V
List of the top 25 upregulated gene sets from Bru-seq data analysis of
compound JR-1-235 - C2 treated for 4 h in MIA PaCa-2 cells.

| | | | | FDR q- | |
|---|---|---|---|---|---|
| No | Gene set | ES | NES | val | Size |
| 1 | PODAR_RESPONSE_TO_ADAPHOSTIN_UP | 0.678 | 2.520 | 0 | 100 |
| 2 | LEONARD_HYPDXIA | 0.811 | 2.480 | 0 | 33 |
| 3 | REACTOME_CHOLESTEROL_BIOSYNTHESIS | 0.913 | 2.477 | 0 | 19 |
| 4 | SCHMIDT_POR_TARGETS_IN_LIMB_BUD_UP | 0.921 | 2.469 | 0 | 19 |
| 5 | ENK_UV_RESPONSE_KERATINOCYTE_UP | 0.590 | 2.464 | 0 | 318 |
| 6 | HORTON_SREBF_TARGETS | 0.911 | 2.446 | 0 | 19 |
| 7 | DIRMEIER_LMP1_RESPONSE_EARLY | 0.764 | 2.410 | 0 | 37 |
| 8 | HELLER_SILENCED_BY_METHYLATION_DN | 0.714 | 2.396 | 0 | 50 |
| 9 | CUI_GLUCOSE_DEPRIVATION | 0.760 | 2.383 | 0 | 39 |
| 10 | WILCOX_RESPONSE_TO_PROGESTERONE_UP | 0.642 | 2.380 | 0 | 104 |
| 11 | REACTOME_RNA_POL_I_PROMOTER_OPENING | 0.820 | 2.377 | 0 | 26 |
| 12 | CHANG_CORE_SERUM_RESPONSE_DN | 0.624 | 2.370 | 0 | 134 |
| 13 | ZHANG_TLX_TARGETS_UP | 0.710 | 2.353 | 0 | 49 |
| 14 | ZHANG_TLX_TARGETS_36HR_UP | 0.615 | 2.339 | 0 | 125 |
| 15 | REACTOME_AMYLO1DS | 0.753 | 2.309 | 0 | 33 |
| 16 | ADDYA_ERYTHROID_DIFFERENTIATION_BY_HEMIN | 0.708 | 2.307 | 0 | 45 |
| 17 | DACOSTA_UV_RESPONSE_VIA_ERCC3_UP | 0.569 | 2.301 | 0 | 222 |
| 18 | DAZARD_RESPONSE_TO_UV_NHEK_UP | 0.602 | 2.291 | 0 | 135 |
| 19 | LE_EGR2_TARGETS_DN | 0.689 | 2.287 | 0 | 49 |
| 20 | KAN_RESPONSE_TO_ARSENIC_TRIOXIDE | 0.655 | 2.270 | 0 | 63 |
| 21 | QI_HYPDXIA | 0.624 | 2.258 | 0 | 84 |
| 22 | BURTON_ADIPOGENESIS_10 | 0.790 | 2.254 | 0 | 24 |
| 23 | REACTOME_UNFOLDED_PROTEIN_RESPONSE | 0.651 | 2.246 | 0 | 66 |
| 24 | GEORGANTAS_HSC_MARKERS | 0.736 | 2.234 | 0 | 32 |
| 25 | AMIT_SERUM_RESPONSE_60_MCF10A | 0.728 | 2.228 | 0 | 34 |

In Tables VI and XIII the top upregulated and downregulated Hallmark gene sets are summarized which represent specific well-defined biological states or processes and display coherent expression. These gene sets were generated by a computational methodology based on identifying overlaps between gene sets in other MSigDB collections and retaining genes that display coordinate expression. HALLMARK_CHOLESTEROL_HOMEOSTASIS summarizes all the genes involved in cholesterol homeostasis. HALLMARK_FATTY_ACID_METABOLISM describes genes encoding proteins involved in metabolism of fatty acids.

TABLE VI
List of the top 25 upregulated gene sets from Bru-seq data analysis of
compound JR-1-235 - (Hallmark) treated for 4 h in MIA PaCa-2 cells.

| No | Gene set | ES | NES | FDR q-val | Size |
|---|---|---|---|---|---|
| 1 | HALLMARK_CHOLESTEROL_HOMEOSTASIS | 0.759 | 2.533 | 0 | 51 |
| 2 | HALLMARK_MTORC1_SIGNALING | 0.626 | 2.450 | 0 | 169 |
| 3 | HALLMARK_HYPOXIA | 0.591 | 2.235 | 0 | 113 |
| 4 | HALLMARK_UNFOLDED_PROTEIN_RESPONSE | 0.582 | 2.173 | 0 | 99 |
| 5 | HALLMARK_P53_PATHWAY | 0.565 | 2.171 | 0 | 132 |
| 6 | HALLMARK_FATTY_ACID_METABOLISM | 0.551 | 2.054 | 0 | 110 |
| 7 | HALLMARK_TNFA_SIGNALING_VIA_NFKB | 0.536 | 2.043 | 1.76E−04 | 120 |
| 8 | HALLMARK_PI3K_AKT_MTOR_SIGNALING | 0.566 | 2.033 | 1.54E−04 | 78 |
| 9 | HALLMARK_MYOGENESIS | 0.541 | 1.885 | 7.58E−04 | 66 |
| 10 | HALLMARK_ANDROGEN_RESPONSE | 0.529 | 1.875 | 6.82E−04 | 75 |
| 11 | HALLMARK_APOPTOSIS | 0.485 | 1.804 | 0.00212473 | 100 |
| 12 | HALLMARK_ADIPOGENESIS | 0.465 | 1.784 | 0.00271651 | 140 |
| 13 | HALLMARK_UV_RESPONSE_UP | 0.478 | 1.769 | 0.00325392 | 98 |
| 14 | HALLMARK_ESTROGEN_RESPONSE_EARLY | 0.458 | 1.719 | 0.0049734 | 106 |
| 15 | HALLMARK_ESTROGEN_RESPONSE_LATE | 0.443 | 1.631 | 0.01332483 | 99 |
| 16 | HALLMARK_COAGULATION | 0.516 | 1.624 | 0.01386542 | 39 |
| 17 | HALLMARK_PEROXISOME | 0.457 | 1.612 | 0.01468971 | 72 |
| 18 | HALLMARK_GLYCOLYSIS | 0.417 | 1.603 | 0.01543783 | 136 |
| 19 | HALLMARK_OXIDATIVE_PHOSPHORYLATION | 0.403 | 1.601 | 0.01481233 | 183 |
| 20 | HALLMARK_IL2_STAT5_SIGNALING | 0.424 | 1.597 | 0.01492409 | 102 |
| 21 | HALLMARK_XENOBIOTIC_METABOLISM | 0.409 | 1.503 | 0.03254462 | 102 |
| 22 | HALLMARK_HEME_METABOLISM | 0.389 | 1.480 | 0.03905911 | 128 |
| 23 | HALLMARK_EPITHELIAL_MESENCHYMAL_TRANSITION | 0.407 | 1.436 | 0.05409918 | 72 |
| 24 | HALLMARK_DNA_REPAIR | 0.377 | 1.422 | 0.05914322 | 120 |
| 25 | HALLMARK_IL6_JAK_STAT3_SIGNALING | 0.443 | 1.405 | 0.06653281 | 39 |

Tables VII and XIV lists the top upregulated and downregulated gene sets from KEGG pathway. Kyoto Encyclopedia of Genes and Genomes (KEGG) is a database resource for understanding high-level functions and utilities of the biological system, such as the cell, from large-scale molecular datasets generated by genome sequencing and other high-throughput experimental technologies. KEGG LYSOSOME and KEGG_SYSTEMIC_LUPUS_ERYTHEMATOSUS two gene sets showed upregulation in Bru-seq analysis. This suggests autophagy can be the mechanism of action.

TABLE VII
List of the top 25 upregulated gene sets from Bru-seq data analysis of
compound JR-1-235 - (KEGG) treated for 4 h in MIA PaCa-2 cells.

| No | Gene set | ES | NES | FDR q-val | Size |
|---|---|---|---|---|---|
| 1 | KEGG_LYSOSOME | 0.611 | 2.224 | 0 | 88 |
| 2 | KEGG_SYSTEMIC_LUPUS_ERYTHEMATOSUS | 0.689 | 2.185 | 0 | 40 |
| 3 | KEGG_VIBRIO_CHOLERAE_INFECTION | 0.576 | 1.841 | 0.02962044 | 39 |
| 4 | KEGG_ADIPOCYTOKINE_SIGNALING_PATHWAY | 0.547 | 1.770 | 0.05885027 | 43 |
| 5 | KEGG_GLYCOLYSIS_GLUCONEOGENESIS | 0.578 | 1.735 | 0.07119057 | 30 |
| 6 | KEGG_PPAR_SIGNALING_PATHWAY | 0.583 | 1.733 | 0.06085466 | 30 |
| 7 | KEGG_MELANOGENESIS | 0.526 | 1.713 | 0.0670009 | 42 |
| 8 | KEGG_OXIDATIVE_PHOSPHORYLATION | 0.464 | 1.705 | 0.06385187 | 89 |
| 9 | KEGG_ACUTE_MYELOID_LEUKEMIA | 0.524 | 1.701 | 0.05843915 | 43 |
| 10 | KEGG_CITRATE_CYCLE_TCA_CYCLE | 0.561 | 1.683 | 0.06465316 | 28 |
| 11 | KEGG_INSULIN_SIGNALING_PATHWAY | 0.455 | 1.663 | 0.07323049 | 93 |
| 12 | KEGG_PORPHYRIN_AND_CHLOROPHYLL_METABOLISM | 0.613 | 1.657 | 0.07333874 | 20 |
| 13 | KEGG_SNARE_INTERACTIONS_IN_VESICULAR_TRANSPORT | 0.535 | 1.642 | 0.07890987 | 32 |
| 14 | KEGG_GLYCEROLIPID_METABOLISM | 0.573 | 1.640 | 0.07427081 | 23 |
| 15 | KEGG_MTOR_SIGNALING_PATHWAY | 0.530 | 1.631 | 0.07556786 | 34 |
| 16 | KEGG_PROSTATE_CANCER | 0.445 | 1.564 | 0.13153353 | 64 |
| 17 | KEGG_ENDOMETRIAL_CANCER | 0.482 | 1.543 | 0.1486858 | 39 |
| 18 | KEGG_VALINE_LEUCINE_AND_ISOLEUCINE_DEGRADATION | 0.491 | 1.536 | 0.14811713 | 36 |
| 19 | KEGG_PARKINSONS_DISEASE | 0.424 | 1.536 | 0.14076799 | 83 |
| 20 | KEGG_FRUCTOSE_AND_MANNOSE_METABOLISM | 0.547 | 1.529 | 0.14081863 | 21 |
| 21 | KEGG_PYRUVATE_METABOLISM | 0.525 | 1.529 | 0.13512571 | 25 |
| 22 | KEGG_ANTIGEN_PROCESSING_AND_PRESENTATION | 0.536 | 1.506 | 0.15364948 | 24 |
| 23 | KEGG_FATTY_ACID_METABOLISM | 0.507 | 1.505 | 0.14860834 | 28 |
| 24 | KEGG_MAPK_SIGNALING_PATHWAY | 0.393 | 1.504 | 0.14335358 | 129 |
| 25 | KEGG_ALDOSTERONE_REGULATED_SODIUM_REABSORPTION | 0.556 | 1.480 | 0.16494685 | 18 |

In Tables VIII and XV the top upregulated and downregulated GO gene sets are summarized. Gene sets in this collection are derived from Gene Ontology (GO) annotations. As evident from the upregulated gene sets, they are related to the chemical reactions and pathways resulting in the formation of sterols, steroids with one or more hydroxyl groups and a hydrocarbon side-chain in the molecule.

TABLE VIII
List of the top 25 upregulated gene sets from Bru-seq data analysis of
compound JR-1-235 - GO treated for 4 h in MIA PaCa-2 cells.

| No | Gene set | ES | NES | FDR q-val | Size |
|---|---|---|---|---|---|
| 1 | GO_STEROL_BIOSYNTHETIC_PROCESS | 0.878 | 2.656 | 0 | 30 |
| 2 | GO_STEROID_BIOSYNTHETIC_PROCESS | 0.784 | 2.632 | 0 | 50 |
| 3 | GO_STEROL_METABOLIC_PROCESS | 0.748 | 2.604 | 0 | 64 |
| 4 | GO_ALCOHOL_BIOSYNTHETIC_PROCESS | 0.728 | 2.531 | 0 | 63 |
| 5 | GO_STEROID_METABOLIC_PROCESS | 0.667 | 2.432 | 0 | 92 |
| 6 | GO_DNA_PACKAGING_COMPLEX | 0.755 | 2.422 | 0 | 47 |
| 7 | GO_ORGANIC_HYDROXY_COMPOUND_BIOSYNTHETIC_PROCESS | 0.651 | 2.372 | 0 | 80 |
| 8 | GO_RESPONSE_TO_TOPOLOGICALLY_INCORRECT_PROTEIN | 0.616 | 2.337 | 0 | 117 |
| 9 | GO_CELLULAR_RESPONSE_TO_TOPOLOGICALLY_INCORRECT_ | 0.635 | 2.333 | 0 | 92 |
| | PROTEIN | | | | |
| 10 | GO_IRE1_MEDIATED_UNFOLDED_PROTEIN_RESPONSE | 0.690 | 2.300 | 0 | 49 |
| 11 | GO_PROTEIN_DNA_COMPLEX | 0.619 | 2.288 | 0 | 97 |
| 12 | GO_SMALL_MOLECULE_BIOSYNTHETIC_PROCESS | 0.555 | 2.227 | 0 | 220 |
| 13 | GO_NUCLEAR_NUCLEOSOME | 0.832 | 2.218 | 8.42E−05 | 17 |
| 14 | GO_CHROMATIN_ASSEMBLY_OR_DISASSEMBLY | 0.576 | 2.165 | 3.14E−04 | 111 |
| 15 | GO_DNA_PACKAGING | 0.570 | 2.161 | 2.93E−04 | 120 |
| 16 | GO_ER_NUCLEUS_SIGNALING_PATHWAY | 0.739 | 2.156 | 4.13E−04 | 28 |
| 17 | GO_REGULATION_OF_ENDOPLASMIC_RETICULUM_STRESS_ | 0.774 | 2.132 | 6.48E−04 | 21 |
| | INDUCED_INTRINSIC_APOPTOTIC_SIGNALING_PATHWAY | | | | |
| 18 | GO_ALCOHOL_METABOLIC_PROCESS | 0.543 | 2.127 | 6.73E−04 | 172 |
| 19 | GO_ISOPRENOID_BIOSYNTHETIC_PROCESS | 0.796 | 2.093 | 0.00116171 | 17 |
| 20 | GO_CELLULAR_LIPID_CATABOLIC_PROCESS | 0.577 | 2.080 | 0.00143318 | 81 |
| 21 | GO_INTRINSIC_COMPONENT_OF_ENDOPLASMIC_RETICULUM_ | 0.577 | 2.075 | 0.00162813 | 77 |
| | MEMBRANE | | | | |
| 22 | GO_RESPONSE_TO_ENDOPLASMIC_RETICULUM_STRESS | 0.526 | 2.067 | 0.00170424 | 171 |
| 23 | GO_LYSOSOMAL_LUMEN | 0.637 | 2.060 | 0.00191643 | 43 |
| 24 | GO_FATTY_ACID_BIOSYNTHETIC_PROCESS | 0.626 | 2.043 | 0.00252606 | 45 |
| 25 | GO_PROTEIN_DNA_COMPLEX_SUBUNIT_ORGANIZATION | 0.521 | 2.041 | 0.00255718 | 157 |

In Tables IX and XVI the top upregulated and downregulated TFBT gene sets are summarized.

TABLE XI
List of the top 25 upregulated gene sets from
Bru-seq data analysis of compound JR-1-235-TFBT
treated for 4 h in MIA PaCa-2 cells.

| | | | | FDR | |
|---|---|---|---|---|---|
| No | Gene set | ES | NES | q-val | Size |
| 1 | KRCTCNNNNMANAGC_UNKNOWN | 0.799 | 2.360 | 0 | 27 |
| 2 | TCANNTGAY_SREBP1_01 | 0.506 | 2.091 | 5.80E−04 | 284 |
| 3 | TTTNNANAGCYR_UNKNOWN | 0.603 | 2.068 | 3.87E−04 | 59 |
| 4 | CCAATNNSNNNGCG_UNKNOWN | 0.609 | 1.988 | 0.00141612 | 41 |
| 5 | OCT1_Q5_01 | 0.524 | 1.931 | 0.00293242 | 100 |
| 6 | SREBP1_01 | 0.497 | 1.885 | 0.00650902 | 119 |
| 7 | BACH1_01 | 0.480 | 1.824 | 0.01768541 | 124 |
| 8 | OCT_Q6 | 0.490 | 1.809 | 0.02033098 | 99 |
| 9 | NFY_C | 0.462 | 1.802 | 0.01942871 | 147 |
| 10 | NFY_Q6_01 | 0.458 | 1.787 | 0.02158424 | 169 |
| 11 | TTGCWCAAY_CEBPB_02 | 0.582 | 1.756 | 0.02964076 | 32 |
| 12 | GCCNNNWTAAR_UNKNOWN | 0.497 | 1.742 | 0.03310944 | 66 |
| 13 | OCT1_B | 0.473 | 1.724 | 0.04026922 | 92 |
| 14 | SRF_01 | 0.582 | 1.711 | 0.04405247 | 26 |
| 15 | LFA1_Q6 | 0.461 | 1.706 | 0.04358217 | 102 |
| 16 | CHOP_01 | 0.451 | 1.704 | 0.04231808 | 116 |
| 17 | USF_02 | 0.426 | 1.679 | 0.05249307 | 171 |
| 18 | ELF1_Q6 | 0.435 | 1.678 | 0.05063425 | 126 |
| 19 | ARNT_02 | 0.424 | 1.678 | 0.04802818 | 174 |
| 20 | NRF2_01 | 0.412 | 1.672 | 0.04881492 | 220 |
| 21 | GGGYGTGNY_UNKNOWN | 0.397 | 1.651 | 0.06093134 | 372 |
| 22 | CREB_01 | 0.426 | 1.647 | 0.06064291 | 155 |
| 23 | TGANNYRGCA_TCF11MAFG_01 | 0.421 | 1.636 | 0.06533076 | 148 |
| 24 | GGAANCGGAANY_UNKNOWN | 0.444 | 1.631 | 0.0673534 | 93 |
| 25 | CREBP1_Q2 | 0.419 | 1.629 | 0.06608945 | 139 |

Table X through XVI lists the top 25 downregulated genes from Bru-Seq analysis of compound JR-1-235.

TABLE X
List of the top 25 downregulated genes from Bru-seq data analysis of
compound JR-1-235 treated for 4 h in MIA PaCa-2 cells.

| No | Ensembl gene ID | Gene name | log2FC | Gene function |
|---|---|---|---|---|
| 1 | ENSG00000181619 | GPR135 | −1.30 | G Protein-Coupled Receptor 135 |
| | | | | GPCR activity, B-arrestin recruitment |
| 2 | ENSG00000163806 | SPDYA | −1.18 | Speedy/RINGO Cell Cycle Regulator Family Member A |
| | | | | Cell cycle regulation |
| 3 | ENSG00000165029 | ABCA1 | −1.14 | ATP Binding Cassette Subfamily A Member 1 |
| | | | | Cholesterol transport (efflux) |
| 4 | ENSG00000134489 | HRH4 | −1.09 | Histamine Receptor H4 |
| | | | | Allergy response |
| 5 | ENSG00000159899 | NPR2 | −1.08 | Natriuretic Peptide Receptor 2 |
| | | | | Skeletal growth regulation (guanyl cyclase activity) |
| 6 | ENSG00000131746 | TNS4 | −1.05 | Tensin 4 |
| | | | | Cell migration and signaling by GPCR |
| 7 | ENSG00000161860 | SYCE2 | −0.98 | Synaptonetnal Complex Central Element Protein 2 |
| | | | | Cell cycle regulation |
| 8 | ENSG00000183691 | NOG | −0.93 | Noggin |
| | | | | Inactivates transforming growth factor-beta |
| 9 | ENSG00000101311 | FERMT1 | −0.91 | Fermitin Family Member 1 |
| | | | | Cytoskeletal signaling and adhesion |
| 10 | ENSG00000270898 | GPR75- | −0.89 | GPR75-ASB3 Protein |
| | | ASB3 | | Paralog of ASB3(class I MHC-mediated antigen processing & |
| | | | | presentation) |
| 11 | ENSG00000101188 | NTSR1 | −0.89 | Neurotensin Receptor 1 |
| | | | | GPCP activity (downstream MAP kinase activation: |
| | | | | antiapoptotic) |
| 12 | ENSG00000188818 | ZDHHC11 | −0.86 | Zinc Finger DHHC-Type Containing 11 |
| | | | | Protein palmitoylation (lipid modification) |
| 13 | ENSG00000204149 | AGAP6 | −0.85 | ArfGAP With GTPase Domain, Ankyrin Repeat And PH |
| | | | | Domain |
| | | | | GTPase-activating protein |
| 14 | ENSG00000255837 | TAS2R20 | −0.81 | Taste 2 Receptor Member 20 |
| | | | | GPCR signaling |
| 15 | ENSG00000183688 | RFLNB | −0.79 | Refilin B |
| | | | | Cartilaginous skeletal elements formation |
| 16 | ENSG00000183486 | MX2 | −0.78 | MX Dynamin Like GTPase 2 |
| | | | | GTPase activity (cell-cycle progression regulation) |
| 17 | ENSG00000179841 | AKAP5 | −0.77 | A-Kinase Anchoring Protein 5 |
| | | | | Glutamate binding, GPCR signaling |
| 18 | ENSG00000162614 | NEXN | −0.76 | Nexilin F-Actin Binding Protein |
| | | | | Cell adhesion and migration |
| 19 | ENSG00000142408 | CACNG8 | −0.75 | Calcium Voltage-Gated Channel Auxiliary Subunit Gamma 8 |
| | | | | Glutamate binding, activation of AMPA receptors & synaptic |
| | | | | plasticity |
| 20 | ENSG00000165555 | NOXRED1 | −0.73 | NADP Dependent Oxidoreductase Domain Containing 1 |
| | | | | Reductase activity |
| 21 | ENSG00000117425 | PTCH2 | −0.72 | Patched 2 |
| | | | | Tumor suppressor, Hedgehog receptor activity, ERK signaling |
| 22 | ENSG00000283374 | TXNRD3NB | −0.72 | Thioredoxin Reductase 3 Neighbor |
| 23 | ENSG00000214063 | TSPAN4 | −0.68 | Tetraspanin 4 |
| | | | | Integtrin and antigen binding (cell development, growth and |
| | | | | motility) |
| 24 | ENSG00000254206 | NPIPB11 | −0.67 | Nuclear Pore Complex Interacting Protein Family Member B11 |
| 25 | ENSG00000110092 | CCND1 | −0.67 | Cyclin D1 |
| | | | | Cell cycle regulation, ERK signaling |

TABLE XI
List of the top 25 downregulated protein-coding genes from Bru-seq data
analysis of compound JR-1-235 treated for 4 h in MIA PaCa-2 cells.

| No | Ensembl gene ID | Gene name | log2FC | Description |
|---|---|---|---|---|
| 1 | ENSG00000213976 | AC010615.1 | −1.64 | unprocessed_pseudogene |
| 2 | ENSG00000201558 | RNVU1-6 | −1.50 | snRNA |
| 3 | ENSG00000278996 | FP671120.1 | −1.32 | lincRNA |
| 4 | ENSG00000272807 | AC007038.2 | −1.27 | antisense_RNA |
| 5 | ENSG00000251221 | LINC01337 | −1.19 | lincRNA |
| 6 | ENSG00000260452 | TPRKBP2 | −1.15 | processed_pseudogene |
| 7 | ENSG00000267160 | AC091152.2 | −1.06 | antisense_RNA |
| 8 | ENSG00000272033 | AL136984.1 | −1.05 | sense_intronic |
| 9 | ENSG00000269998 | LINC01930 | −1.00 | lincRNA |
| 10 | ENSG00000248734 | AC008906.1 | −0.98 | antisense_RNA |
| 11 | ENSG00000269749 | AC005614.2 | −0.94 | processe_transcript |
| 12 | ENSG00000242539 | AC007620.2 | −0.89 | antisense_RNA |
| 13 | ENSG00000253929 | CASC21 | −0.87 | lincRNA |
| 14 | ENSG00000240695 | AC117382.1 | −0.85 | processed_pseudogene |
| 15 | ENSG00000276449 | AC004076.2 | −0.85 | antisense_RNA |
| 16 | ENSG00000229563 | LINC01204 | −0.84 | lincRNA |
| 17 | ENSG00000255670 | AC007619.1 | −0.84 | lincRNA |
| 18 | ENSG00000232498 | AL136987.1 | −0.83 | antisense_RNA |
| 19 | ENSG00000266993 | AL050343.1 | −0.82 | antisense_RNA |
| 20 | ENSG00000283415 | AC087280.2 | −0.80 | lincRNA |
| 21 | ENSG00000273489 | AC008264.2 | −0.80 | antisense_RNA |
| 22 | ENSG00000250072 | AC091940.1 | −0.79 | lincRNA |
| 23 | ENSG00000264895 | AC006141.1 | −0.78 | sense_intronic |
| 24 | ENSG00000256019 | AC018630.1 | −0.77 | processed_pseudogene |
| 25 | ENSG00000224903 | AC005534.1 | −0.75 | antisense_RNA |

TABLE XII
List of the top 25 downregulated gene sets from Bru-seq data analysis of
compound JR-1-235 - C2 treated for 4 h in MIA PaCa-2 cells.

| | | | | FDR q- | |
|---|---|---|---|---|---|
| No | Gene set | ES | NES | val | Size |
| 1 | DACOSTA_UV_RESPONSE_VIA_ERCC3_COMMON_DN | −0.439 | −2.348 | 0.001 | 418 |
| 2 | ZHANG_TLX_TARGETS_36HR_DN | −0.470 | −2.309 | 0.001 | 173 |
| 3 | HUTTMANN_B_CLL_POOR_SURVIVAL_DN | −0.606 | −2.076 | 0.026 | 30 |
| 4 | HAMAI_APOPTOSIS_VIA_TRAIL_UP | −0.368 | −1.942 | 0.096 | 438 |
| 5 | ZHANG_TLX_TARGETS_DN | −0.446 | −1.942 | 0.077 | 83 |
| 6 | KEGG_NEUROACTIVE_LIGAND_RECEPTOR_INTERACTION | −0.624 | −1.898 | 0.109 | 20 |
| 7 | ZHENG_FOXP3_TARGETS_IN_T_LYMPHOCYTE_DN | −0.611 | −1.893 | 0.097 | 19 |
| 8 | DACOSTA_UV_RESPONSE_VIA_ERCC3_TTD_DN | −0.469 | −1.878 | 0.100 | 57 |
| 9 | DAZARD_UV_RESPONSE_CLUSTER_G6 | −0.408 | −1.855 | 0.111 | 118 |
| 10 | BILD_CTNNBl_ONCOGENIC_SIGNATURE | −0.449 | −1.841 | 0.114 | 61 |
| 11 | SENGUPTA_NASOPHARYNGEAL_CARCINOMA_WITH_LMP1_UP | −0.365 | −1.837 | 0.108 | 215 |
| 12 | DOANE_BREAST_CANCER_CLASSES_DN | −0.578 | −1.830 | 0.105 | 23 |
| 13 | REACTOME_ASSOCIATION_OF_TRIC_CCT_WITH_TARGET_ | −0.551 | −1.804 | 0.125 | 23 |
| | PROTEINS_DURING_BIOSYNTHESIS | | | | |
| 14 | SENGUPTA_NASOPHARYNGEAL_CARCINOMA_UP | −0.360 | −1.794 | 0.127 | 201 |
| 15 | DACOSTA_UV_RESPONSE_VIA_ERCC3_XPCS_DN | −0.428 | −1.784 | 0.130 | 66 |
| 16 | TCGA_GLIOBLASTOMA_COPY_NUMBER_DN | −0.533 | −1.738 | 0.181 | 23 |
| 17 | MARTINEZ_RESPONSE_TO_TRABECTEDIN | −0.482 | −1.727 | 0.186 | 35 |
| 18 | MILI_PSEUDOPODIA_HAPTOTAXIS_UP | −0.322 | −1.718 | 0.190 | 440 |
| 19 | THUM_SYSTOLIC_HEART_FAILURE_DN | −0.352 | −1.673 | 0.260 | 150 |
| 20 | HAN_JNK_SINGALING_DN | −0.579 | −1.665 | 0.265 | 15 |
| 21 | LEE_TARGETS_OF_PTCH1_AND_SUFU_UP | −0.515 | −1.661 | 0.260 | 22 |
| 22 | GUENTHER_GROWTH_SPHERICAL_VS_ADHERENT_DN | −0.531 | −1.656 | 0.258 | 20 |
| 23 | GABRIELY_MIR21_TARGETS | −0.328 | −1.642 | 0.274 | 230 |
| 24 | RAMALHO_STEMNESS_UP | −0.331 | −1.615 | 0.324 | 180 |
| 25 | TURASHVILI_BREAST_LOBULAR_CARCINOMA_VS_DUCTAL_ | −0.476 | −1.603 | 0.337 | 27 |
| | NORMAL_DN | | | | |

TABLE XIII
List of the top 5 downregulated gene sets from Bru-seq data analysis of
compound JR-1-235 - HALLMARK treated for 4 h in MIA PaCa-2 cells.

| No | Gene set | ES | NES | FDR q-val | Size |
|---|---|---|---|---|---|
| 1 | HALLMARK_TGF_BETA_SIGNALING | −0.324 | −1.256 | 0.4975793 | 45 |
| 2 | HALLMARK_KRAS_SIGNALING_DN | −0.282 | −0.992 | 1 | 32 |
| 3 | HALLMARK_KRAS_SIGNALING_UP | −0.230 | −0.965 | 0.8892967 | 67 |
| 4 | HALLMARK_MYC_TARGETS_V1 | −0.172 | −0.831 | 1 | 192 |
| 5 | HALLMARK_MYC_TARGETS_V2 | −0.178 | −0.691 | 0.9630892 | 53 |

TABLE XIV
List of the top 23 downregulated gene sets from Bru-seq data analysis of
compound JR-1-235 - KEGG treated for 4 h in MIA PaCa-2 cells.

| No | Gene set | ES | NES | FDR q-val | Size |
|---|---|---|---|---|---|
| 1 | KEGG_NEUROACTIVE_LIGAND_RECEPTOR_INTERACTION | −0.624 | −1.955 | 0.01455422 | 20 |
| 2 | KEGG_TGF_BETA_SIGNALING_PATHWAY | −0.336 | −1.284 | 1 | 47 |
| 3 | KEGG_PURINE_METABOLISM | −0.285 | −1.277 | 0.94780767 | 93 |
| 4 | KEGG_CALCIUM_SIGNALING_PATHWAY | −0.324 | −1.264 | 0.7562894 | 51 |
| 5 | KEGG_ABC_TRANSPORTERS | −0.412 | −1.220 | 0.76165736 | 18 |
| 6 | KEGG_MISMATCH_REPAIR | −0.375 | −1.179 | 0.7785864 | 21 |
| 7 | KEGG_PYRIMIDINE_METABOLISM | −0.256 | −1.109 | 0.9242662 | 75 |
| 8 | KEGG_STARCH_AND_SUCROSE_METABOLISM | −0.342 | −1.006 | 1 | 17 |
| 9 | KEGG_WNT_SIGNALING_PATHWAY | −0.231 | −0.992 | 1 | 82 |
| 10 | KEGG_HEDGEHOG_SIGNALING_PATHWAY | −0.329 | −0.982 | 1 | 18 |
| 11 | KEGG_DNA_REPLICATION | −0.254 | −0.912 | 1 | 34 |
| 12 | KEGG_NUCLEOTIDE_EXCISION_REPAIR | −0.241 | −0.899 | 1 | 41 |
| 13 | KEGG_AMYOTROPHIC_LATERAL_SCLEROSIS_ALS | −0.247 | −0.838 | 1 | 29 |
| 14 | KEGG_AXON_GUIDANCE | −0.201 | −0.829 | 1 | 61 |
| 15 | KEGG_HOMOLOGOUS_RECOMBINATION | −0.260 | −0.820 | 1 | 23 |
| 16 | KEGG_CYTOSOLIC_DNA_SENSING_PATHWAY | −0.247 | −0.784 | 1 | 23 |
| 17 | KEGG_PATHOGENIC_ESCHERICHIA_COLI_INFECTION | −0.220 | −0.782 | 1 | 36 |
| 18 | KEGG_CELL_ADHESION_MOLECULES_CAMS | −0.231 | −0.738 | 1 | 20 |
| 19 | KEGG_ALANINE_ASPARTATE_AND_GLUTAMATE_METABOLISM | −0.250 | −0.732 | 1 | 15 |
| 20 | KEGG_RIG_I_LIKE_RECEPTOR_SIGNALING_PATHWAY | −0.197 | −0.724 | 1 | 36 |
| 21 | KEGG_RNA_POLYMERASE | −0.202 | −0.669 | 1 | 23 |
| 22 | KEGG_ECM_RECEPTOR_INTERACTION | −0.201 | −0.655 | 0.9980664 | 24 |
| 23 | KEGG_ADHERENS_JUNCTION | −0.158 | −0.628 | 0.9676537 | 49 |

TABLE XV
List of the top 25 downregulated gene sets from Bru-seq data analysis of
compound JR-1-235 - GO treated for 4 h in MIA PaCa-2 cells.

| No | Gene set | ES | NES | FDR q-val | Size |
|---|---|---|---|---|---|
| 1 | GO_G_PROTEIN_COUPLED_RECEPTOR_ACTIVITY | −0.476 | −1.804 | 1 | 47 |
| 2 | GO_NEGATIVE_REGULATION_OF_EPITHELIAL_CELL_ | −0.597 | −1.799 | 0.94753146 | 17 |
| | DIFFERENTIATION | | | | |
| 3 | GO_DENDRITIC_SHAFT | −0.568 | −1.746 | 1 | 20 |
| 4 | GO_DNA_DEPENDENT_DNA_REPLICATION_MAINTENANCE_ | −0.569 | −1.697 | 1 | 18 |
| | OF_FIDELITY | | | | |
| 5 | GO_POTASSIUM_CHANNEL_COMPLEX | −0.536 | −1.693 | 0.9618062 | 22 |
| 6 | GO_PHOSPHOLIPID_TRANSPORTER_ACTIVITY | −0.549 | −1.692 | 0.8079703 | 19 |
| 7 | GO_HOMOPHILIC_CELL_ADHESION_VIA_PLASMA_ | −0.581 | −1.691 | 0.702026 | 15 |
| | MEMBRANE_ADHESION_MOLECULES | | | | |
| 8 | GO_CELL_SURFACE_RECEPTOR_SIGNALING_PATHWAY_ | −0.570 | −1.653 | 0.832176 | 15 |
| | INVOLVED_IN_CELL_CELL_SIGNALING | | | | |
| 9 | GO_RESPONSE_TO_X_RAY | −0.501 | −1.636 | 0.84332436 | 24 |
| 10 | GO_BASAL_PART_OF_CELL | −0.526 | −1.629 | 0.79593754 | 21 |
| 11 | GO_GLUTAMINE_METABOLIC_PROCESS | −0.569 | −1.616 | 0.8008931 | 15 |
| 12 | GO_DNA_DOUBLE_STRAND_BREAK_PROCESSING | −0.551 | −1.613 | 0.74987507 | 16 |
| 13 | GO_SOMITE_DEVELOPMENT | −0.451 | −1.594 | 0.7962615 | 33 |
| 14 | GO_DNA_REPAIR_COMPLEX | −0.429 | −1.586 | 0.7809938 | 36 |
| 15 | GO_REGULATION_OF_ACUTE_INFLAMMATORY_RESPONSE | −0.521 | −1.555 | 0.900144 | 17 |
| 16 | GO_RECOMBINATIONAL_REPAIR | −0.382 | −1.548 | 0.8886256 | 62 |
| 17 | GO_MITOTIC_RECOMBINATION | −0.420 | −1.547 | 0.8417501 | 39 |
| 18 | GO_DNA_DEPENDENT_ATPASE_ACTIVITY | −0.369 | −1.545 | 0.80398434 | 72 |
| 19 | GO_DNA_HELICASE_ACTIVITY | −0.389 | −1.504 | 1 | 48 |
| 20 | GO_CILIUM_ORGANIZATION | −0.331 | −1.502 | 0.9635023 | 110 |
| 21 | GO_REGULATION_OF_MEMBRANE_LIPID_DISTRIBUTION | −0.521 | −1.498 | 0.9369633 | 16 |
| 22 | GO_STRAND_DISPLACEMENT | −0.471 | −1.493 | 0.9236681 | 22 |
| 23 | GO_RECIPROCAL_DNA_RECOMBINATION | −0.450 | −1.485 | 0.9346575 | 24 |
| 24 | GO_SOMATIC_RECOMBINATION_OF_IMMUNOGLOBULIN_ | −0.498 | −1.483 | 0.9062042 | 18 |
| | GENE_SEGMENTS | | | | |
| 25 | GO_CYTOKINE_RECEPTOR_ACTIVITY | −0.488 | −1.467 | 0.96156234 | 19 |

TABLE XVI
List of the top 25 downregulated gene sets from
Bru-seq data analysis of compound JR-1-235-TFBT
treated for 4 h in MIA PaCa-2 cells.

| | | | | FDR | |
|---|---|---|---|---|---|
| No | Gene set | ES | NES | q-val | Size |
| 1 | GGCKCATGS_UNKNOWN | −0.441 | −1.621 | 0.2535936 | 40 |
| 2 | IRF2_01 | −0.324 | −1.322 | 1 | 61 |
| 3 | PAX8_01 | −0.439 | −1.304 | 1 | 19 |
| 4 | TAAYNRNNTCC_UNKNOWN | −0.314 | −1.303 | 0.8453206 | 64 |
| 5 | MEIS1BHOXA9_01 | −0.320 | −1.269 | 0.87676543 | 52 |
| 6 | NKX3A_01 | −0.267 | −1.213 | 1 | 95 |
| 7 | PIT1_Q6 | −0.263 | −1.156 | 1 | 91 |
| 8 | FOXJ2_02 | −0.261 | −1.154 | 1 | 93 |
| 9 | HNF1_Q6 | −0.266 | −1.145 | 1 | 80 |
| 10 | HOX13_01 | −0.366 | −1.144 | 1 | 21 |
| 11 | PBX1_02 | −0.282 | −1.130 | 1 | 54 |
| 12 | RNTCANNRNNYNATTW_ | −0.370 | −1.127 | 1 | 18 |
| | UNKNOWN | | | | |
| 13 | TAAWWATAG_RSRFC4_Q2 | −0.271 | −1.112 | 1 | 65 |
| 14 | BRN2_01 | −0.247 | −1.086 | 1 | 95 |
| 15 | IK1_01 | −0.227 | −1.074 | 1 | 135 |
| 16 | EN1_01 | −0.276 | −1.062 | 1 | 42 |
| 17 | CDP_01 | −0.301 | −1.041 | 1 | 30 |
| 18 | YKACATTT_UNKNOWN | −0.221 | −1.039 | 1 | 145 |
| 19 | HMEF2_Q6 | −0.266 | −1.037 | 1 | 46 |
| 20 | POU1F1_Q6 | −0.236 | −1.032 | 1 | 99 |
| 21 | CDPCR1_01 | −0.259 | −1.030 | 1 | 53 |
| 22 | TAANNYSGCG_UNKNOWN | −0.253 | −1.012 | 1 | 61 |
| 23 | MEF2_Q6_01 | −0.220 | −0.985 | 1 | 96 |
| 24 | YNTTTNNNANGCARM_ | −0.345 | −0.971 | 1 | 15 |
| | UNKNOWN | | | | |
| 25 | MEF2_02 | −0.225 | −0.965 | 1 | 70 |

The up and downregulated gene lists were used to query the CMAP database for overall transcription profiles of reported pertubagens. The top 25 pertubagens (compounds) correlating with JR-1-235 transcription profile is reported in Table XVII and XVIII. Mostly HDAC inhibitor, EGFR inhibitor and kinase inhibitors were identified to have similar transcription profiles suggesting correlation in the mechanism of action. Compounds identified by CMAP do not show significant structural similarity with JR-1-235. However, correlation of these compounds hints potential mechanisms of JR-1-235 activity, and application of these compounds as tools for comparison might be a plausible approach to further characterize JR-1-235 in different biological systems.

TABLE XVII
List of the top CMAP hits positively correlated with JR-1-235 treated for 4 h in MIA PaCa-2 cells.

| | Median | | | |
|---|---|---|---|---|
| No | score | Name | Structure | Description |
| 1 | 99.51 | Trichostatin-a | 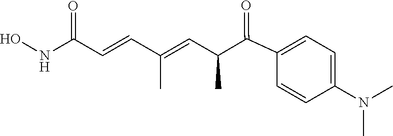 [SMILES: CC(/C=C/C(=O)NO)=C\[C@H](C)C(=O)c1ccc(N(C)C)cc1] | HDAC inhibitor, CDK expression enhancer, ID1 expression inhibitor |
| 2 | 99.31 | Vorinostat | 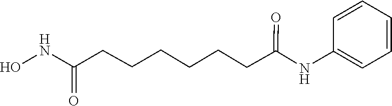 [SMILES: O=C(CCCCCCC(=O)Nc1ccccc1)NO] | HDAC inhibitor, cell cycle inhibitor |
| 3 | 99.24 | ISOX | 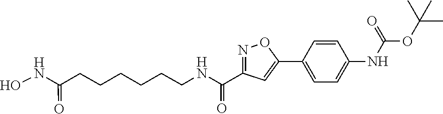 [SMILES: CC(C)(C)OC(=O)Nc1ccc(-c2cc(C(=O)NCCCCCCC(=O)NO)no2)cc1] | HDAC inhibitor |
| 4 | 99.12 | BIBX-1382 | 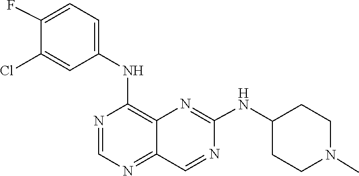 [SMILES: CN1CCC(Nc2ncc3ncnc(Nc4ccc(F)c(Cl)c4)c3n2)CC1] | EGFR inhibitor, tyrosine kinase inhibitor |
| 5 | 99.03 | BI-2536 | 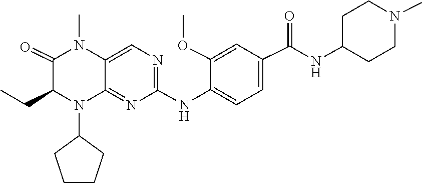 [SMILES: CC[C@H]1C(=O)N(C)c2cnc(Nc3ccc(C(=O)NC4CCN(C)CC4)cc3OC)nc2N1C1CCCC1] | PLK inhibitor, apoptosis stimulant, cell cycle inhibitor, protein kinase inhibitor |
| 6 | 98.99 | Bisindolylmal eimide | 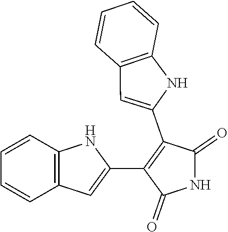 [SMILES: O=C1NC(=O)C(c2cc3ccccc3[nH]2)=C1c1cc2ccccc2[nH]1] | CDK inhibitor, PKC inhibitor, leucine rich repeat kinase inhibitor |
| 7 | 98.98 | BIBU-1361 | 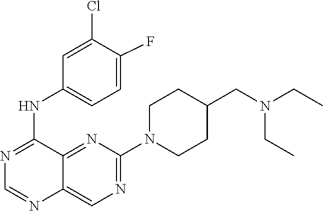 [SMILES: CCN(CC)CC1CCN(c2ncc3ncnc(Nc4ccc(F)c(Cl)c4)c3n2)CC1] | EGFR inhibitor |
| 8 | 98.95 | TG-101348 | 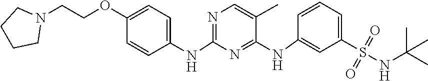 [SMILES: Cc1cnc(Nc2ccc(OCCN3CCCC3)cc2)nc1Nc1cccc(S(=O)(=O)NC(C)(C)C)c1] | JAK inhibitor, FLT3 inhibitor, RET tyrosine kinase inhibitor |
| 9 | 98.95 | Trichostatin-a | 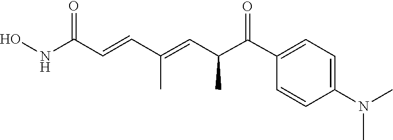 [SMILES: CC(/C=C/C(=O)NO)=C\[C@H](C)C(=O)c1ccc(N(C)C)cc1] | HDAC inhibitor, CDK expression enhancer, ID1 expression inhibitor |
| 10 | 98.91 | WT-171 | 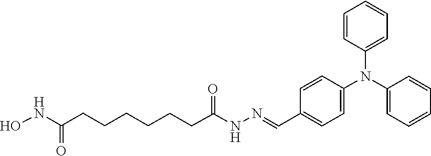 [SMILES: O=C(CCCCCCC(=O)N/N=C/c1ccc(N(c2ccccc2)c2ccccc2)cc1)NO] | HDAC inhibitor |
| 11 | 98.78 | NTNCB | 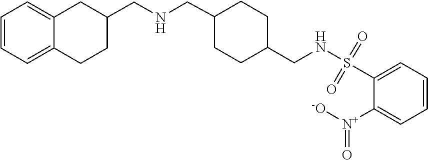 | neuropeptide receptor antagonist |
| 12 | 98.78 | Dacinostat | 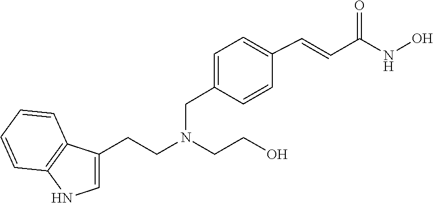 [SMILES: O=C(/C=C/c1ccc(CN(CCO)CCc2c[nH]c3ccccc23)cc1)NO] | HDAC inhibitor |
| 13 | 98.68 | THM-I-94 | 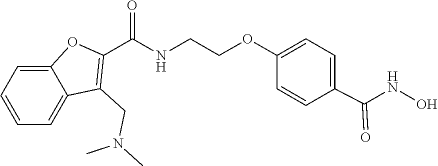 [SMILES: CN(C)Cc1c(C(=O)NCCOc2ccc(C(=O)NO)cc2)oc2ccccc12] | HDAC inhibitor, apoptosis stimulant, cell cycle inhibitor |
| 14 | 98.63 | HC-toxin | 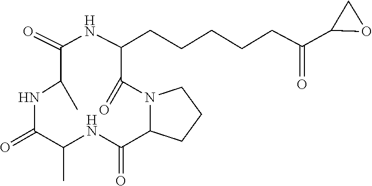 [SMILES: CC1NC(=O)C(C)NC(=O)C2CCCN2C(=O)C(CCCCCC(=O)C2CO2)NC1=O] | HDAC inhibitor |
| 15 | 98.60 | XMD-1150 | 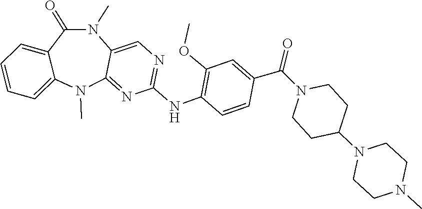 [SMILES: COc1cc(C(=O)N2CCC(N3CCN(C)CC3)CC2)ccc1Nc1ncc2c(n1)N(C)c1ccccc1C(=O)N2C] | leucine rich repeat kinase inhibitor |
| 16 | 98.57 | JWE-035 | 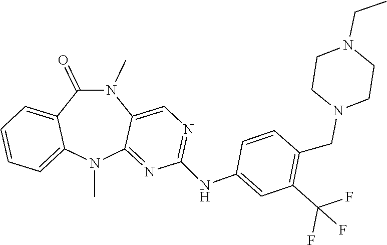 [SMILES: CCN1CCN(Cc2ccc(Nc3ncc4c(n3)N(C)c3ccccc3C(=O)N4C)cc2C(F)(F)F)CC1] | Aurora kinase inhibitor |
| 17 | 98.54 | Lasalocid | 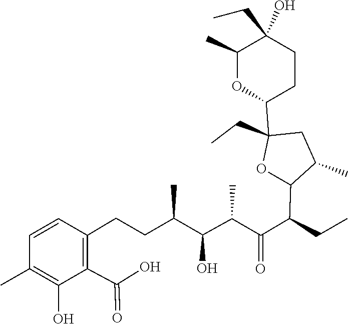 [SMILES: CC[C@@H](C(=O)[C@@H](C)[C@@H](O)[C@H](C)CCc1ccc(C)c(O)c1C(=O)O)C1O[C@](CC)([C@H]2CC[C@](O)(CC)[C@H](C)O2)C[C@@H]1C] | ionophore antibiotic |
| 18 | 98.49 | XMD-885 | 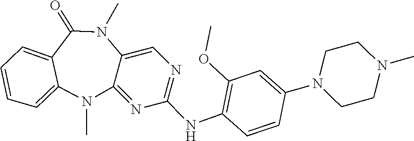 [SMILES: COc1cc(N2CCN(C)CC2)ccc1Nc1ncc2c(n1)N(C)c1ccccc1C(=O)N2C] | leucine rich repeat kinase inhibitor, MAP kinase inhibitor |
| 19 | 98.32 | Panobinostat | 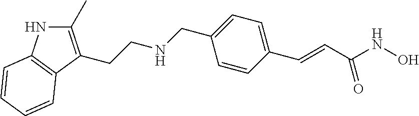 [SMILES: Cc1[nH]c2ccccc2c1CCNCc1ccc(/C=C/C(=O)NO)cc1] | HDAC inhibitor, apoptosis stimulant, cell cycle inhibitor |
| 20 | 98.31 | Reserpine | 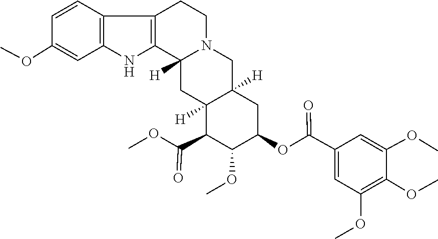 | vesicular monoamine transporter inhibitor |
| 21 | 98.22 | Trimipramine | 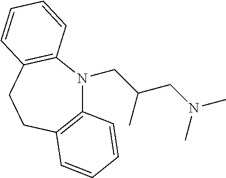 [SMILES: CC(CN(C)C)CN1c2ccccc2CCc2ccccc21] | adrenergic transmitter uptake inhibitor, dopamine receptor, norepinephrine transporter inhibitor, serotonin transporter (SERT) inhibitor, serotonin uptake inhibitor, tricyclic antidepressant, tricyclic antidepressant (TCA) |
| 22 | 98.10 | KIN001-220 | 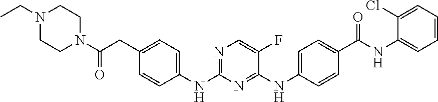 [SMILES: CCN1CCN(C(=O)Cc2ccc(Nc3ncc(F)c(Nc4ccc(C(=O)Nc5ccccc5Cl)cc4)n3)cc2)CC1] | Aurora kinase inhibitor |
| 23 | 98.05 | FIT | 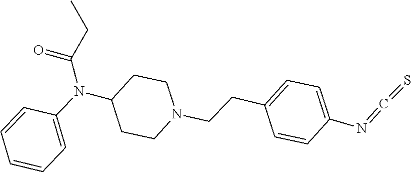 [SMILES: CCC(=O)N(c1ccccc1)C1CCN(CCc2ccc(N=C=S)cc2)CC1] | opioid receptor agonist |
| 24 | 97.96 | Alimemazine | 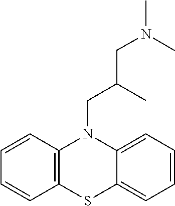 [SMILES: CC(CN(C)C)CN1c2ccccc2Sc2ccccc21] | histamine receptor ligand |
| 25 | 97.56 | Scriptaid | 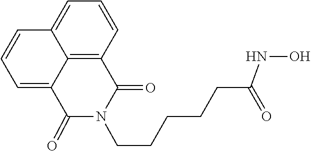 [SMILES: O=C(CCCCCN1C(=O)c2cccc3cccc(c23)C1=O)NO] | HDAC inhibitor |

TABLE XVIII
List of the top CMAP hits negatively correlated with JR-1-235 treated for 4 h in MIA PaCa-2 cells.

| | median | | | |
|---|---|---|---|---|
| No | score | name | Structure | description |
| 1 | −95.63 | PP-30 | 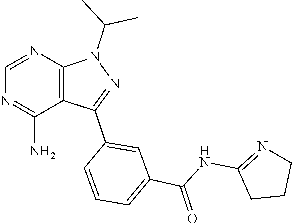 [SMILES: CC(C)n1nc(-c2cccc(C(=O)NC3=NCCC3)c2)c2c(N)ncnc21] | RAF inhibitor |
| 2 | −95.35 | calyculin | 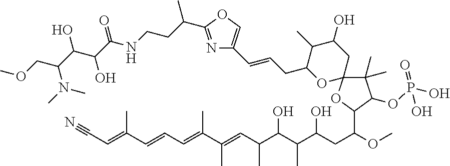 [SMILES: COCC(C(O)C(O)C(=O)NCCC(C)c1nc(/C=C/CC2OC3(CC(O)C2C)OC(C(CC(O)C(C)C(O)C(C)/C=C(C)/C(C)=C/C=C/C(C)=C/C#N)OC)C(OP(=O)(O)O)C3(C)C)co1)N(C)C] | protein phosphatase inhibitor |
| 3 | −91.32 | PHA-793887 | 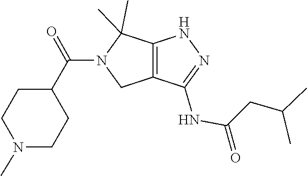 [SMILES: CC(C)CC(=O)Nc1n[nH]c2c1CN(C(=O)C1CCN(C)CC1)C2(C)C] | CDK inhibitor |
| 4 | −90.87 | ZG-10 | NA | JNK inhibitor |
| 5 | −89.24 | ochratoxin-a | 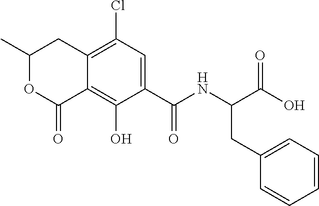 [SMILES: CC1Cc2c(Cl)cc(C(=O)NC(Cc3ccccc3)C(=O)O)c(O)c2C(=O)O1] | phenylalanyl tRNA synthetase inhibitor |
| 6 | −89.17 | SB-225002 | 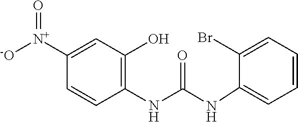 | CC chemokine receptor antagonist, Chemokine CXCR2 (IL-8 beta Receptor) Antagonists |
| 7 | −86.65 | parbendazole | 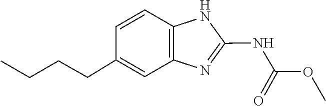 [SMILES: CCCCc1ccc2[nH]c(NC(=O)OC)nc2c1] | tubulin inhibitor |
| 8 | −85.04 | PIK-75 | 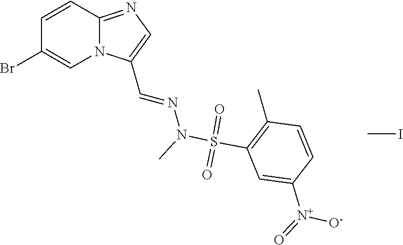 | DNA protein kinase inhibitor, P110 inhibitor, Phosphatidylinositol 3-kinase (PI3K) inhibitor, PI3K inhibitor |
| 9 | −85.01 | oxibendazole | 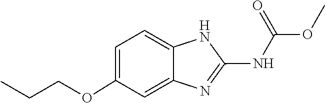 [SMILES: CCCOc1ccc2[nH]c(NC(=O)OC)nc2c1] | DNA polymerase inhibitor, tubulin inhibitor |
| 10 | −83.68 | JNK-9L | 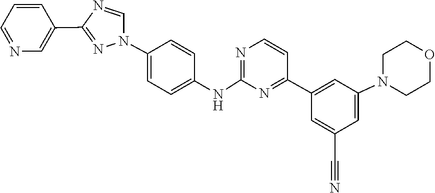 [SMILES: N#Cc1cc(-c2ccnc(Nc3ccc(-n4cnc(-c5cccnc5)n4)cc3)n2)cc(N2CCOCC2)c1] | JNK inhibitor |
| 11 | −83.59 | bisindolylmal eimide-ix | 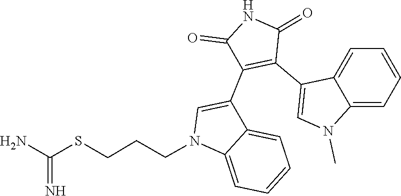 [SMILES: Cn1cc(C2=C(c3cn(CCCSC(=N)N)c4ccccc34)C(=O)NC2=O)c2ccccc21] | PKC inhibitor, glycogen synthase kinase inhibitor, leucine rich repeat kinase inhibitor, SIRT inhibitor |
| 12 | −83.48 | PF-562271 | 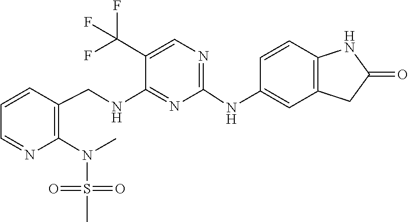 [SMILES: CN(c1ncccc1CNc1nc(Nc2ccc3c(c2)CC(=O)N3)ncc1C(F)(F)F)S(C)(=O)=O] | focal adhesion kinase inhibitor, angiogenesis inhibitor, apoptosis stimulant |
| 13 | −83.09 | estradiol- cypionate | 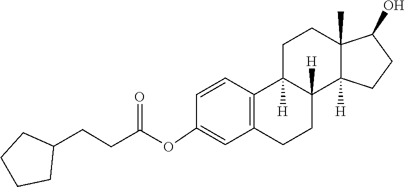 | estrogen receptor agonist |
| 14 | −80.95 | cosmosiin | 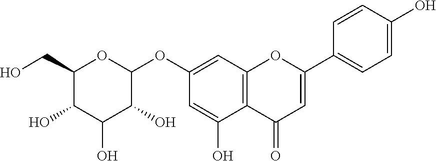 [SMILES: O=c1cc(-c2ccc(O)cc2)oc2cc(OC3O[C@H](CO)[C@@H](O)C(O)[C@H]3O)cc(O)c12] | cytochrome P450 inhibitor |
| 15 | −80.91 | chelidonine | 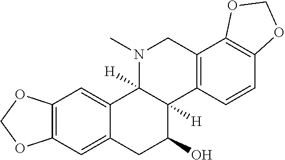 [SMILES: CN1Cc2c(ccc3c2OCO3)[C@@H]2[C@H]1c1cc3c(cc1C[C@@H]2O)OCO3] | tubulin polymerization inhibitor |
| 16 | −79.88 | cycloheximide | 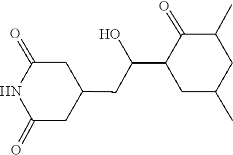 [SMILES: CC1CC(C)C(=O)C(C(O)CC2CC(=O)NC(=O)C2)C1] | glycogen synthase kinase inhibitor, protein synthesis inhibitor |
| 17 | −78.67 | triptolide | 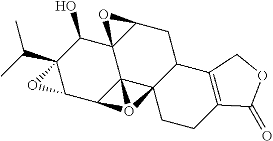 | RNA polymerase inhibitor |
| 18 | −77.88 | BRD- A12633378 | 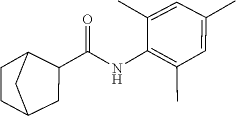 [SMILES: Cc1cc(C)c(NC(=O)C2CC3CCC2C3)c(C)c1] | voltage-gated potassium channel activator |
| 19 | −77.63 | aminogenistein | 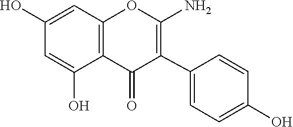 [SMILES: Nc1oc2cc(O)cc(O)c2c(=O)c1-c1ccc(O)cc1] | PKC inhibitor, src inhibitor |
| 20 | −77.43 | staurosporine | 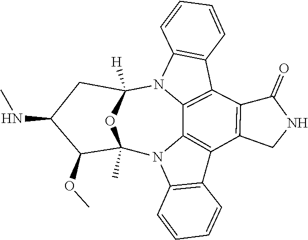 [SMILES: CN[C@H]1C[C@H]2O[C@@](C)([C@H]1OC)n1c3ccccc3c3c4c(c5c6ccccc6n2c5c31)C(=O)NC4] | PKC inhibitor, AKT inhibitor, BMX inhibitor, CDK inhibitor, CHK inhibitor, G protein coupled receptor agonist, glycogen synthase kinase inhibitor, leucine rich repeat kinase inhibitor, ribosomal protein inhibitor, sodium/hydrogen exchanger inhibitor |
| 21 | −77.25 | dactinomycin | 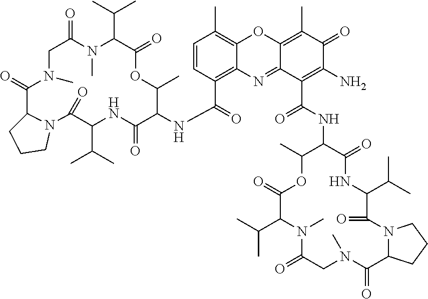 [SMILES: Cc1c2oc3c(C)ccc(C(=O)NC4C(=O)NC(C(C)C)C(=O)N5CCCC5C(=O)N(C)CC(=O)N(C)C(C(C)C)C(=O)OC4C)c3nc-2c(C(=O)NC2C(=O)NC(C(C)C)C(=O)N3CCCC3C(=O)N(C)CC(=O)N(C)C(C(C)C)C(=O)OC2C)c(N)c1=O] | DNA directed RNA polymerase inhibitor, nucleic acid synthesis inhibitor, protein synthesis inhibitor |
| 22 | −76.73 | NVP-BEZ235 | 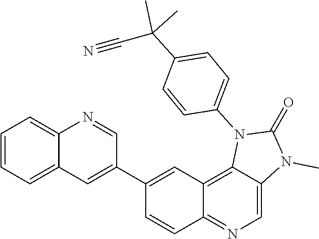 [SMILES: Cn1c(=O)n(-c2ccc(C(C)(C)C#N)cc2)c2c3cc(-c4cnc5ccccc5c4)ccc3ncc21] | mTOR inhibitor, PI3K inhibitor, protein kinase inhibitor |
| 23 | −76.59 | BRD- K85853281 | 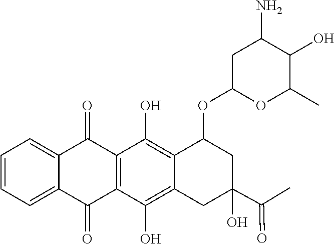 [SMILES: CC(=O)C1(O)Cc2c(O)c3c(c(O)c2C(OC2CC(N)C(O)C(C)O2)C1)C(=O)c1ccccc1C3=O] | radical formation stimulant, RNA synthesis inhibitor, topoisomerase inhibitor |
| 24 | −76.44 | A-443644 | 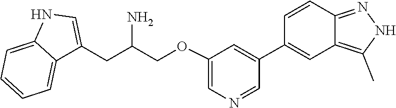 [SMILES: Cc1[nH]nc2ccc(-c3cncc(OCC(N)Cc4c[nH]c5ccccc45)c3)cc12] | AKT inhibitor |
| 25 | −76.11 | nifurtimox | 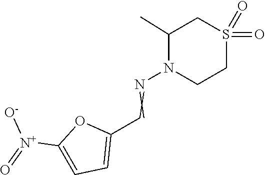 [SMILES: CC1CS(=O)(=O)CCN1N=Cc1ccc([N+](=O)[O-])o1] | DNA inhibitor |

Table XIX through XXV lists the top 25 upregulated genes from Bru-Seq analysis of compound J28 (JR-1-272). Bru-seq analysis of J28 was like J4. Overexpression of genes like INSIG1, DHCR7, MVK and FASN suggested cholesterol biosynthesis pathway as the mechanism of action of J28. Bru-seq also revealed synthesis of PCYT2, DOLK and HIST1H3B RNAs was upregulated in a dose-dependent manner by treatment with JR-1-272 (Table XIX). PCYT2 (Phosphate Cytidylyltransferase 2, Ethanolamine), encodes an enzyme that catalyzes the formation of CDP-ethanolamine from CTP and phosphoethanolamine in the Kennedy pathway of phospholipid synthesis. The protein encoded by DOLK gene catalyzes the CTP-mediated phosphorylation of dolichol and is involved in the synthesis of Dol-P-Man, which is an essential glycosyl carrier lipid for C- and O-mannosylation, N- and O-linked glycosylation of proteins, and for the biosynthesis of glycosyl phosphatidylinositol anchors in endoplasmic reticulum. HIST1H3B gene is intronless and encodes a replication-dependent histone that is a member of the histone H3 family. Transcripts from this gene lack polyA tails; instead, they contain a palindromic termination element. Table XX lists the top 25 upregulated noncoding genes from Bru-seq data analysis of compound JR-1-272 when treated for 4 h in MIA PaCa-2 cells. A significant decrease in expression of ZNF816, IFT80, CACNG8 and GPR135 was also observed (Table XVI).

TABLE XIX
List of the top 25 upregulated protein-coding genes from Bru-seq data
analysis of compound JR-1-272 treated for 4 h in MIA PaCa-2 cells.

| No | Ensembl gene ID | Gene name | log2FC | Gene function |
|---|---|---|---|---|
| 1 | ENSG00000185813 | PCYT2 | 2.03 | Phosphate Cytidylyltransferase 2, Ethanolamine |
| | | | | Phospholipid synthesis |
| 2 | ENSG00000105516 | DBP | 1.90 | D-Box Binding PAR BZIP Transcription Factor |
| | | | | Circadian rhythm regulation, DNA binding transcription factor |
| | | | | activity |
| 3 | ENSG00000274267 | HIST1H3B | 1.82 | Histone Cluster 1 H3 Family Member B |
| | | | | Transcription regulation, DNA repair, DNA replication and |
| | | | | chromosomal stability |
| 4 | ENSG00000173456 | RNF26 | 1.71 | Ring Finger Protein 26 |
| | | | | Protein ubiquitination, endosome organization |
| 5 | ENSG00000175283 | DOLK | 1.70 | Dolichol Kinase |
| | | | | Oligosaccharide synthesis |
| 6 | ENSG00000099194 | SCD | 1.67 | Stearoyl-CoA Desaturase |
| | | | | Fatty acid biosynthesis |
| 7 | ENSG00000100445 | SDR39U1 | 1.67 | Short Chain Dehydrogenase/Reductase Family 39U Member 1 |
| 8 | ENSG00000186480 | INSIG1 | 1.65 | Insulin Induced Gene 1 |
| | | | | Cholesterol mateabolism, lipogenesis, and glucose homeostasis |
| | | | | regulation |
| 9 | ENSG00000277224 | HIST1H2BF | 1.63 | Histone Cluster 1 H2B Family Member F |
| | | | | Replication-dependent, cell cycle regulation |
| 10 | ENSG00000182810 | DDX28 | 1.46 | DEAD-Box Heliease 28 |
| | | | | Translation initiation, nuclear and mitochondrial splicing, |
| | | | | ribosome assembly |
| 11 | ENSG00000278272 | HIST1H3C | 1.45 | Histone Cluster 1 H3 Family Member C |
| | | | | Cell cycle regulation |
| 12 | ENSG00000172893 | DHCR7 | 1.44 | 7-Dehydrocholesterol Reductase |
| | | | | Cholesterol biosynthesis |
| 13 | ENSG00000172057 | ORMDL3 | 1.39 | ORMDL Sphingolipid Biosynthesis Regulator 3 |
| | | | | Sphingolipid synthesis (negative regulator) |
| 14 | ENSG00000198931 | APRT | 1.39 | Adenine Phosphoribosyltransferase |
| | | | | Pyrimidine metabolism |
| 15 | ENSG00000110921 | MVK | 1.37 | Mevalonate Kinase |
| | | | | Cholesterol biosynthesis |
| 16 | ENSG00000124575 | HIST1H1D | 1.36 | Histone Cluster 1 H1 Family Member D |
| | | | | Apoptotic cleavage of cellular proteins |
| 17 | ENSG00000188486 | H2AFX | 1.36 | H2A Histone Family Member X |
| | | | | Cell cycle regulation |
| 18 | ENSG00000267261 | AC099811.2 | 1.35 | GTP binding and histone acetyltransferase activity |
| 19 | ENSG00000170619 | COMMD5 | 1.31 | COMM Domain Containing 5 |
| | | | | Cell proliferation (negative regulation) |
| 20 | ENSG00000172336 | POP7 | 1.30 | POP7 Homolog, Ribonuclease P/MRP Subunit |
| | | | | RNA transport, tRNA processing |
| 21 | ENSG00000275379 | HIST1H3I | 1.28 | Histone Cluster 1 H3 Family Member 1 |
| | | | | Cell cycle regulation |
| 22 | ENSG00000114779 | ABHD14B | 1.28 | Abhydrolase Domain Containing 14B |
| | | | | Metabolism, CYP450 |
| 23 | ENSG00000169710 | FASN | 1.28 | Fatty Acid Synthase |
| | | | | Cholesterol biosynthesis, AMPK signaling |
| 24 | ENSG00000163874 | ZC3H12A | 1.27 | Zinc Finger CCCH-Type Containing 12A |
| | | | | Transcriptional activator, cardiomyocytes cell death |
| 25 | ENSG00000197355 | UAP1L1 | 1.26 | UDP-N-Acetylglucosamine Pyrophosphorylase 1 Like 1 |
| | | | | Amino sugar metabolism |

TABLE XX
List of the top 25 upregulated noncoding genes from Bru-seq data analysis of
compound JR-1-272 treated for 4 h in MIA PaCa-2 cells.

| No | Ensembl gene ID | Gene name | log2FC | Description |
|---|---|---|---|---|
| 1 | ENSG00000252213 | SNORA74D | 1.76 | snoRNA |
| 2 | ENSG00000201302 | SNORA65 | 1.28 | snoRNA |
| 3 | ENSG00000227533 | SLC2A1-AS1 | 1.22 | lincRNA |
| 4 | ENSG00000246350 | AL049543.1 | 1.21 | antisense_RNA |
| 5 | ENSG00000268798 | AC027307.3 | 1.20 | lincRNA |
| 6 | ENSG00000243005 | RN7SL16P | 1.19 | misc_RNA |
| 7 | ENSG00000212232 | SNORD17 | 1.17 | snoRNA |
| 8 | ENSG00000260899 | AC106886.2 | 1.10 | processed_transcript |
| 9 | ENSG00000234912 | SNHG20 | 1.06 | processed_transcript |
| 10 | ENSG00000215154 | AC141586.1 | 1.01 | transcribed_unprocessed_pseudogene |
| 11 | ENSG00000217527 | RPS16P5 | 0.97 | transcribed_processed_pseudogene |
| 12 | ENSG00000227199 | ST7-AS1 | 0.95 | antisense_RNA |
| 13 | ENSG00000228709 | AP001065.1 | 0.95 | lincRNA |
| 14 | ENSG00000272993 | AC239868.4 | 0.91 | lincRNA |
| 15 | ENSG00000200087 | SNORA73B | 0.91 | snoRNA |
| 16 | ENSG00000207445 | SNORD15B | 0.90 | snoRNA |
| 17 | ENSG00000203288 | TDRKH-AS1 | 0.90 | antisense_RNA |
| 18 | ENSG00000261821 | AC090826.2 | 0.90 | antisense_RNA |
| 19 | ENSG00000249850 | KRT18P31 | 0.89 | processed_pseudogene |
| 20 | ENSG00000212607 | SNORA3B | 0.88 | snoRNA |
| 21 | ENSG00000206941 | SNORD15A | 0.86 | snoRNA |
| 22 | ENSG00000265683 | SYPL1P2 | 0.85 | processed_pseudogene |
| 23 | ENSG00000235701 | PCBP2P1 | 0.84 | processed_pseudogene |
| 24 | ENSG00000268362 | AC092279.1 | 0.84 | lincRNA |
| 25 | ENSG00000274828 | AC068473.5 | 0.84 | lincRNA |

In Tables XXI and XXVIII the top upregulated and downregulated curated gene sets are summarized. KIM_ALL_DISORDERS_DURATION_CORR_DN include all genes whose expression in brain significantly and negatively correlated with the duration of all psychiatric disorders studied. For compound JR-1-272 also an enrichment is observed in the cholesterol biosynthesis pathway.

TABLE XXI
List of the top 25 upregulated gene sets from Bru-seq data analysis of
compound JR-1-272 - C2 treated for 4 h in MIA PaCa-2 cells.

| | | | | FDR q- | |
|---|---|---|---|---|---|
| No | Gene set | ES | NES | val | Size |
| 1 | KIM_ALL_DISORDERS_DURATION_CORR_DN | 0.615 | 2.457 | 0 | 96 |
| 2 | REACTOME_RNA_POL_I_PROMOTER_OPENING | 0.769 | 2.411 | 0 | 26 |
| 3 | DAIRKEE_TERT_TARGETS_UP | 0.528 | 2.400 | 0 | 254 |
| 4 | REACTOME_CHOLESTEROL_BIOSYNTHESIS | 0.821 | 2.387 | 0 | 19 |
| 5 | REACTOME_AMYLOIDS | 0.710 | 2.374 | 0 | 33 |
| 6 | NIKOLSKY_BREAST_CANCER_16Q24_AMPLICON | 0.706 | 2.371 | 0 | 35 |
| 7 | RICKMAN_METASTASIS_DN | 0.556 | 2.370 | 0 | 138 |
| 8 | LEONARD_HYPDXIA | 0.716 | 2.369 | 0 | 32 |
| 9 | HORTON_SREBF_TARGETS | 0.807 | 2.353 | 0 | 19 |
| 10 | ENK_UV_RESPONSE_KERATINOCYTE_UP | 0.501 | 2.338 | 0 | 314 |
| 11 | BANDRES_RESPONSE_TO_CARMUSTIN_MGMT_48HR_DN | 0.612 | 2.308 | 0 | 64 |
| 12 | SCHMIDT_POR_TARGETS_IN_LIMB_BUD_UP | 0.788 | 2.303 | 0 | 19 |
| 13 | KEGG_SYSTEMIC_LUPUS_ERYTHEMATOSUS | 0.661 | 2.294 | 0 | 39 |
| 14 | AMUNDSON_GAMMA_RADIATION_RESPONSE | 0.673 | 2.280 | 0 | 37 |
| 15 | DACOSTA_UV_RESPONSE_VIA_ERCC3_UP | 0.506 | 2.272 | 0 | 216 |
| 16 | REACTOME_RNA_POL_I_TRANSCRIPTION | 0.622 | 2.250 | 0 | 50 |
| 17 | MARTENS_TRETINOIN_RESPONSE_UP | 0.552 | 2.244 | 6.31E−05 | 96 |
| 18 | DIRMEIER_LMPl_RESPONSE_EARLY | 0.670 | 2.238 | 5.96E−05 | 36 |
| 19 | CAMPS_COLON_CANCER_COPY_NUMBER_UP | 0.653 | 2.238 | 5.64E−05 | 41 |
| 20 | NIKOLSKY_BREAST_CANCER_16P13_AMPLICON | 0.699 | 2.231 | 5.36E−05 | 28 |
| 21 | QI_HYPOXIA | 0.559 | 2.230 | 5.11E−05 | 81 |
| 22 | FERREIRA_EWINGS_SARCOMA_UNSTABLE_VS_STABLE_DN | 0.623 | 2.223 | 4.87E−05 | 49 |
| 23 | REACTOME_PEPTIDE_CHAIN_ELONGATION | 0.564 | 2.220 | 4.66E−05 | 81 |
| 24 | DEBIASI_APOPTOSIS_BY_REOVIRUS_INFECTION_DN | 0.488 | 2.211 | 8.91E−05 | 212 |
| 25 | GINESTIER_BREAST_CANCER_ZNF217_AMPLIFIED_DN | 0.481 | 2.203 | 1.28E−04 | 251 |

In Tables XXII and XXIX the top upregulated and downregulated Hallmark gene sets are summarized which represent specific well-defined biological states or processes and display coherent expression. These gene sets were generated by a computational methodology based on identifying overlaps between gene sets in other MSigDB collections and retaining genes that display coordinate expression. Here too upregulation is observed in cholesterol homeostasis pathway.

TABLE XXII
List of the top 25 upregulated gene sets from Bru-seq data analysis of
compound JR-1-272 - HALLMARK treated for 4 h in MIA PaCa-2 cells.

| No | Gene set | ES | NES | FDR q-val | Size |
|---|---|---|---|---|---|
| 1 | HALLMARK_CHOLESTEROL_HOMEOSTASIS | 0.656 | 2.374 | 0 | 50 |
| 2 | HALLMARK_MYOGENESIS | 0.560 | 2.098 | 0 | 67 |
| 3 | HALLMARK_P53_PATHWAY | 0.471 | 1.986 | 0 | 129 |
| 4 | HALLMARK_HYPOXIA | 0.479 | 1.984 | 0 | 113 |
| 5 | HALLMARK_MTORC1_SIGNALING | 0.436 | 1.917 | 7.27E−04 | 168 |
| 6 | HALLMARK_UV_RESPONSE_UP | 0.436 | 1.773 | 0.00617189 | 96 |
| 7 | HALLMARK_UNFOLDED_PROTEIN_RESPONSE | 0.433 | 1.739 | 0.00879828 | 96 |
| 8 | HALLMARK_ADIPOGENESIS | 0.383 | 1.653 | 0.0231409 | 141 |
| 9 | HALLMARK_PI3K_AKT_MTOR_SIGNALING | 0.426 | 1.634 | 0.02460267 | 78 |
| 10 | HALLMARK_COAGULATION | 0.474 | 1.628 | 0.0244482 | 40 |
| 11 | HALLMARK_APOPTOSIS | 0.390 | 1.615 | 0.02488416 | 101 |
| 12 | HALLMARK_FATTY_ACID_METABOLISM | 0.392 | 1.605 | 0.02502945 | 109 |
| 13 | HALLMARK_OXIDATIVE_PHOSPHORYLATION | 0.351 | 1.548 | 0.03756334 | 180 |
| 14 | HALLMARK_HEME_METABOLISM | 0.367 | 1.548 | 0.03497548 | 126 |
| 15 | HALLMARK_WNT_BETA_CATENIN_SIGNALING | 0.481 | 1.483 | 0.05686833 | 24 |
| 16 | HALLMARK_PEROXISOME | 0.379 | 1.483 | 0.05366645 | 72 |
| 17 | HALLMARK_NOTCH_SIGNALING | 0.494 | 1.473 | 0.0552529 | 22 |
| 18 | HALLMARK_ESTROGEN_RESPONSE_EARLY | 0.354 | 1.458 | 0.06052842 | 106 |
| 19 | HALLMARK_APICAL_JUNCTION | 0.359 | 1.422 | 0.07641187 | 90 |
| 20 | HALLMARK_XENOBIOTIC_METABOLISM | 0.348 | 1.415 | 0.07732665 | 101 |
| 21 | HALLMARK_DNA_REPAIR | 0.336 | 1.407 | 0.0789391 | 120 |
| 22 | HALLMARK_ANDROGEN_RESPONSE | 0.356 | 1.381 | 0.09299158 | 75 |
| 23 | HALLMARK_GLYCOLYSIS | 0.321 | 1.363 | 0.10334915 | 136 |
| 24 | HALLMARK_ESTROGEN_RESPONSE_LATE | 0.335 | 1.358 | 0.1023042 | 97 |
| 25 | HALLMARK_BILE_ACID_METABOLISM | 0.361 | 1.330 | 0.12133909 | 54 |

Tables XXIII and XXX lists the top upregulated and downregulated gene sets from KEGG pathway. Compound JR-1-272 shows upregulation in KEGG gene sets SYSTEMIC_LUPUS_ERYTHEMATOSUS and LYSOSOME similar to compound JR-1-235. Systemic lupus erythematosus (SLE) is characterized by circulating IgG autoantibodies that are specific for self-antigens, such as DNA, nuclear proteins and certain cytoplasmic components. Immune complexes comprising autoantibody and self-antigen is deposited particularly in the renal glomeruli and mediate a systemic inflammatory response by activating complement or via Fc-gamma-R-mediated neutrophil and macrophage activation. Activation of complement leads to injury both through formation of the membrane attack complex (C5b-9) or by generation of the anaphylatoxin and cell activator C5a. Neutrophils and macrophages cause tissue injury by the release of oxidants and proteases.

TABLE XXIII
List of the top 25 upregulated gene sets from Bru-seq data analysis of
compound JR-1-272 - KEGG treated for 4 h in MIA PaCa-2 cells.

| No | Gene set | ES | NES | FDR q-val | Size |
|---|---|---|---|---|---|
| 1 | KEGG_SYSTEMIC_LUPUS_ERYTHEMATOSUS | 0.661 | 2.271 | 0 | 39 |
| 2 | KEGG_RIBOSOME | 0.534 | 2.148 | 5.93E−04 | 83 |
| 3 | KEGG_LYSOSOME | 0.494 | 1.958 | 0.00807093 | 87 |
| 4 | KEGG_ACUTE_MYELOID_LEUKEMIA | 0.524 | 1.872 | 0.02179753 | 43 |
| 5 | KEGG_VEGF_SIGNALING_PATHWAY | 0.501 | 1.705 | 0.11394524 | 37 |
| 6 | KEGG_SNARE_INTERACTIONS_IN_VESICULAR_TRANSPORT | 0.503 | 1.647 | 0.16105925 | 32 |
| 7 | KEGG_PEROXISOME | 0.443 | 1.643 | 0.14510117 | 57 |
| 8 | KEGG_VIBRIO_CHOLERAE_INFECTION | 0.474 | 1.626 | 0.14944817 | 39 |
| 9 | KEGG_NON_SMALL_CELL_LUNG_CANCER | 0.477 | 1.610 | 0.15560424 | 39 |
| 10 | KEGG_PROGESTERONE_MEDIATED_OOCYTE_MATURATION | 0.434 | 1.602 | 0.14908655 | 57 |
| 11 | KEGG_PPAR_SIGNALING_PATHWAY | 0.485 | 1.573 | 0.17668156 | 30 |
| 12 | KEGG_GLYCOLYSIS_GLUCONEOGENESIS | 0.478 | 1.563 | 0.17352776 | 31 |
| 13 | KEGG_OXIDATIVE_PHOSPHORYLATION | 0.387 | 1.544 | 0.18872698 | 90 |
| 14 | KEGG_BIOSYNTHESIS_OF_UNSATURATED_FATTY_ACIDS | 0.555 | 1.531 | 0.19661675 | 15 |
| 15 | KEGG_PROSTATE_CANCER | 0.402 | 1.527 | 0.18765812 | 65 |
| 16 | KEGG_ADIPOCYTOKINE_SIGNALING_PATHWAY | 0.429 | 1.493 | 0.22871217 | 41 |
| 17 | KEGG_ENDOMETRIAL_CANCER | 0.437 | 1.492 | 0.21617189 | 39 |
| 18 | KEGG_LONG_TERM_DEPRESSION | 0.461 | 1.491 | 0.2053485 | 29 |
| 19 | KEGG_MTOR_SIGNALING_PATHWAY | 0.447 | 1.469 | 0.22934978 | 34 |
| 20 | KEGG_FC_EPSILON_RI_SIGNALING_PATHWAY | 0.442 | 1.464 | 0.22614434 | 32 |
| 21 | KEGG_PORPHYRIN_AND_CHLOROPHYLL_METABOLISM | 0.496 | 1.448 | 0.24046895 | 20 |
| 22 | KEGG_PARKINSONS_DISEASE | 0.358 | 1.404 | 0.31236464 | 82 |
| 23 | KEGG_INSULIN_SIGNALING_PATHWAY | 0.349 | 1.403 | 0.30053458 | 93 |
| 24 | KEGG_RENAL_CELL_CARCINOMA | 0.393 | 1.392 | 0.3072466 | 47 |
| 25 | KEGG_CARDIAC_MUSCLE_CONTRACTION | 0.424 | 1.386 | 0.3072667 | 30 |

In Tables XXIV and XXXI the top upregulated and downregulated GO gene sets are summarized. Gene sets in this collection are derived from Gene Ontology (GO) annotations. As evident from the upregulated gene sets they are related to the chemical reactions and pathways resulting in the formation of sterols, steroids with one or more hydroxyl groups and a hydrocarbon side-chain in the molecule.

TABLE XXIV
List of the top 25 upregulated gene sets from Bru-seq data analysis of
compound JR-1-272 - GO treated for 4 h in MIA PaCa-2 cells.

| No | Gene set | ES | NES | FDR q-val | Size |
|---|---|---|---|---|---|
| 1 | GO_DNA_PACKAGING_COMPLEX | 0.727 | 2.600 | 0 | 46 |
| 2 | GO_STEROL_METABOLIC_PROCESS | 0.651 | 2.434 | 0 | 64 |
| 3 | GO_STEROL_BIOSYNTHETIC_PROCESS | 0.757 | 2.428 | 0 | 30 |
| 4 | GO_PROTEIN_DNA_COMPLEX | 0.554 | 2.251 | 0.00115446 | 95 |
| 5 | GO_STEROID_BIOSYNTHETIC_PROCESS | 0.605 | 2.176 | 0.00234233 | 49 |
| 6 | GO_NEGATIVE_REGULATION_OF_RESPONSE_TO_OXI- | 0.743 | 2.162 | 0.0029242 | 18 |
| | DATIVE_STRESS | | | | |
| 7 | GO_ALCOHOL_BIOSYNTHETIC_PROCESS | 0.573 | 2.162 | 0.00250645 | 63 |
| 8 | GO_STEROID_METABOLIC_PROCESS | 0.523 | 2.100 | 0.00686799 | 91 |
| 9 | GO_REGULATION_OF_STEROID_BIOSYNTHETIC_PROCESS | 0.710 | 2.097 | 0.0064944 | 20 |
| 10 | GO_NUCLEAR_NUCLEOSOME | 0.745 | 2.088 | 0.00619548 | 17 |
| 11 | GO_CYTOSOLIC_RIBOSOME | 0.516 | 2.082 | 0.00584473 | 99 |
| 12 | GO_CHROMATIN_SILENCING_AT_RDNA | 0.713 | 2.078 | 0.00564925 | 19 |
| 13 | GO_REGULATION_OF_STEROID_METABOLIC_PROCESS | 0.615 | 2.031 | 0.01310679 | 31 |
| 14 | GO_REGULATION_OF_ENDOPLASMIC_RETICULUM_STRESS_IN- | 0.678 | 2.024 | 0.01366348 | 21 |
| | DUCED_INTRINSIC_APOPTOTIC_SIGNALING_PATHWAY | | | | |
| 15 | GO_ALCOHOL_METABOLIC_PROCESS | 0.457 | 2.022 | 0.01290765 | 173 |
| 16 | GO_CELLULAR_RESPONSE_TO_TOPOLOGICALLY_IN- | 0.504 | 2.013 | 0.01465238 | 91 |
| | CORRECT_PROTEIN | | | | |
| 17 | GO_IRE1_MEDIATED_UNFOLDED_PROTEIN_RESPONSE | 0.556 | 2.001 | 0.01736414 | 48 |
| 18 | GO_INTRINSIC_COMPONENT_OF_ENDOPLASMIC_RETICU- | 0.508 | 2.000 | 0.01672084 | 77 |
| | LUM_MEMBRANE | | | | |
| 19 | GO_HEXOSE_METABOLIC_PROCESS | 0.500 | 1.992 | 0.01767695 | 91 |
| 20 | GO_STRUCTURAL_CONSTITUENT_OF_RIBOSOME | 0.452 | 1.990 | 0.01737348 | 172 |
| 21 | GO_CYTOSOLIC_LARGE_RIBOSOMAL_SUBUNIT | 0.536 | 1.982 | 0.01909871 | 56 |
| 22 | GO_DNA_PACKAGING | 0.468 | 1.976 | 0.01949975 | 119 |
| 23 | GO_RIBOSOMAL_SUBUNIT | 0.469 | 1.976 | 0.01865194 | 140 |
| 24 | GO_ORGANIC_HYDROXY_COMPOUND_BIOSYNTHETIC_PROCESS | 0.503 | 1.959 | 0.02204559 | 81 |
| 25 | GO_CHROMATIN_ASSEMBLY_OR_DISASSEMBLY | 0.470 | 1.952 | 0.02406231 | 109 |

In Tables XXV and XXXII the top upregulated and downregulated TFBT gene sets are summarized.

TABLE XXV
List of the top 25 upregulated gene sets from Bru-seq data
analysis of compound JR-1-272-TFBT treated for 4 h
in MIA PaCa-2 cells.

| No | Gene set | ES | NES | FDR q-val | Size |
|---|---|---|---|---|---|
| 1 | KRCTCNNNNMANAGC_UNKNOWN | 0.784 | 2.441 | 0 | 27 |
| 2 | TTTNNANAGCYR_UNKNOWN | 0.502 | 1.859 | 0.04500562 | 58 |
| 3 | TCANNTGAY_SREBP1_01 | 0.399 | 1.843 | 0.03823674 | 282 |
| 4 | BACH1_01 | 0.419 | 1.762 | 0.08577862 | 121 |
| 5 | SREBP1_01 | 0.417 | 1.757 | 0.07247698 | 120 |
| 6 | GGGYGTGNY_UNKNOWN | 0.361 | 1.708 | 0.10885043 | 373 |
| 7 | SRF_01 | 0.541 | 1.708 | 0.09360562 | 26 |
| 8 | GCCNNNWTAAR_UNKNOWN | 0.442 | 1.702 | 0.08781068 | 68 |
| 9 | GATGKMRGCG_UNKNOWN | 0.462 | 1.697 | 0.08292831 | 53 |
| 10 | GGAANCGGAANY_UNKNOWN | 0.421 | 1.695 | 0.07603078 | 91 |
| 11 | HEB_Q6 | 0.415 | 1.688 | 0.07496275 | 97 |
| 12 | BACH2_01 | 0.390 | 1.654 | 0.10251804 | 125 |
| 13 | PEA3_Q6 | 0.389 | 1.645 | 0.10454984 | 134 |
| 14 | ACTWSNACTNY_UNKNOWN | 0.432 | 1.640 | 0.10251217 | 65 |
| 15 | NFE2_01 | 0.387 | 1.630 | 0.10764118 | 126 |
| 16 | NFY_Q6_01 | 0.370 | 1.617 | 0.1173277 | 166 |
| 17 | SREBP1_Q6 | 0.386 | 1.611 | 0.1174248 | 117 |
| 18 | MAZR_01 | 0.380 | 1.598 | 0.12497794 | 120 |
| 19 | NRF2_01 | 0.356 | 1.597 | 0.12082852 | 223 |
| 20 | EVI1_05 | 0.421 | 1.596 | 0.11569235 | 71 |
| 21 | GGCNRNWCTTYS_UNKNOWN | 0.424 | 1.583 | 0.12609391 | 58 |
| 22 | OCT1Q5_01 | 0.383 | 1.568 | 0.14160684 | 98 |
| 23 | TGANNYRGCA_TCF11MAFG_01 | 0.359 | 1.552 | 0.16056855 | 145 |
| 24 | AP1_Q4_01 | 0.371 | 1.550 | 0.15625009 | 119 |
| 25 | CCCNNNNNNAAGWT_UNKNOWN | 0.409 | 1.550 | 0.15094358 | 62 |

Tables XXVI through XXXII lists the top 25 downregulated genes from Bru-Seq analysis of compound JR-1-272.

TABLE XXVI
List of the top 25 downregulated protein-coding genes from Bru-seq data
analysis of compound JR-1-272 treated for 4 h in MIA PaCa-2 cells.

| No | Ensembl gene ID | Gene name | log2FC | Gene function |
|---|---|---|---|---|
| 1 | ENSG00000180257 | ZNF816 | −1.58 | Zinc Finger Protein 816 |
| | | | | Transcriptional regulation |
| 2 | ENSG00000068885 | IFT80 | −1.12 | Intraflagellar Transport 80 |
| | | | | Organelle biogenesis, intraflagellar transport |
| 3 | ENSG00000142408 | CACNG8 | −1.10 | Calcium Voltage-Gated Channel Auxilliary Subunit Gamma 8 |
| | | | | Regulation of AMPA receptors channel gating, translation |
| | | | | initiatoin |
| 4 | ENSG00000181619 | GPR135 | −1.08 | G Protein-Coupled Receptor 135 |
| | | | | GPCR activity, regulatory interaction with metatoninR |
| 5 | ENSG00000163661 | PTX3 | −1.04 | Pentraxin 3 |
| | | | | Innate immune system |
| 6 | ENSG00000258472 | AC005726.2 | −0.99 | RNA gene |
| 7 | ENSG00000111700 | SLCO1B3 | −0.96 | Solute Carrier Organic Anion Transporter Family |
| | | | | Taxane pathway, metabolism, bilirubin transport |
| 8 | ENSG00000161509 | GRIN2C | −0.95 | Glutamate Ionotropic Receptor NMDA Type Subunit 2C |
| | | | | Memory, synaptic development |
| 9 | ENSG00000133863 | TEX15 | −0.95 | Testis Expressed 15, Meiosis And Synapsis Associated |
| | | | | Cell cycle |
| 10 | ENSG00000203668 | CHML | −0.94 | CHM Like, Rab Escort Protein 2 |
| | | | | Protein metabolism, GTPase activator activity |
| 11 | ENSG00000247595 | SPTY2D1-AS1 | −0.93 | SPTY2D1 Opposite Strand |
| 12 | ENSG00000075407 | ZNF37A | −0.93 | Zinc Finger Protein 37A |
| | | | | DNA binding transcription factor activity |
| 13 | ENSG00000144824 | PHLDB2 | −0.93 | Pleckstrin Homology Like Domain Family B Member 2 |
| | | | | Acetyl-choline receptor aggregation |
| 14 | ENSG00000185860 | CCDC190 | −0.91 | Coiled-Coil Domain Containing 190 |
| 15 | ENSG00000120217 | CD274 | −0.90 | CD274 Molecule |
| | | | | Innate immune system |
| 16 | ENSG00000179841 | AKAP5 | −0.90 | A-Kinase Anchoring Protein 5 |
| | | | | Activation of AMPA receptor, GPCR pathway |
| 17 | ENSG00000147174 | GCNA | −0.89 | Germ Cell Nuclear Acidic Peptidase |
| 18 | ENSG00000155090 | KLF10 | −0.88 | Kruppel Like Factor 10 |
| | | | | Transcriptional reprsessor (TGFB signaling) |
| 19 | ENSG00000101311 | FERMT1 | −0.88 | Fermitin Family Member 1 |
| | | | | Cytoskeletal signaling and adhesion |
| 20 | ENSG00000257062 | AC022335.1 | −0.86 | Transporter activity |
| 21 | ENSG00000134489 | HRH4 | −0.86 | Histamine Receptor H4 |
| | | | | GPCR, AKt signaling |
| 22 | ENSG00000283374 | TXNRD3NB | −0.86 | Thioredoxin Reductase 3 Neighbor |
| 23 | ENSG00000023909 | GCLM | −0.85 | Glutamate-Cysteine Ligase Modifier Subunit |
| | | | | Glutathione sunthesis, ferroptosis and metabolism |
| 24 | ENSG00000069493 | CLEC2D | −0.84 | C-Type Lectin Domain Family 2 Member D |
| | | | | Innate immune system |
| 25 | ENSG00000260729 | AC009690.1 | −0.83 | Hydrolase activity |

TABLE XXVII
List of the top 25 downregulated noncoding genes from Bru-seq data
analysis of compound JR-1-272 treated for 4 h in MIA PaCa-2 cells.

| No | Ensembl gene ID | Gene name | log2FC | Description |
|---|---|---|---|---|
| 1 | ENSG00000225761 | AL596247.1 | −2.37 | sense_intronic |
| 2 | ENSG00000278996 | FP671120.1 | −2.35 | lincRNA |
| 3 | ENSG00000272054 | AC007390.2 | −1.54 | sense_intronic |
| 4 | ENSG00000241170 | AP001992.1 | −1.36 | transcribed_processed_pseudogene |
| 5 | ENSG00000255670 | AC007619.1 | −1.30 | lincRNA |
| 6 | ENSG00000201558 | RNVU1-6 | −1.29 | snRNA |
| 7 | ENSG00000275119 | AC244131.2 | −1.22 | lincRNA |
| 8 | ENSG00000242615 | AC022415.1 | −1.20 | transcribed_processed_pseudogene |
| 9 | ENSG00000251562 | MALAT1 | −1.19 | lincRNA |
| 10 | ENSG00000264895 | AC006141.1 | −1.18 | sense_intronic |
| 11 | ENSG00000267160 | AC091152.2 | −1.18 | antisense_RNA |
| 12 | ENSG00000230732 | AC016949.1 | −1.18 | sense_intronic |
| 13 | ENSG00000223509 | AC135983.2 | −1.16 | transcribed_processed_pseudogene |
| 14 | ENSG00000256538 | AC046130.2 | −1.16 | lincRNA |
| 15 | ENSG00000241985 | WWTR1-IT1 | −1.14 | sense_intronic |
| 16 | ENSG00000272807 | AC007038.2 | −1.08 | antisense_RNA |
| 17 | ENSG00000269998 | LINC01930 | −1.08 | lincRNA |
| 18 | ENSG00000259456 | ADNP-AS1 | −1.07 | antisense_RNA |
| 19 | ENSG00000251221 | LINC01337 | −1.06 | lincRNA |
| 20 | ENSG00000231172 | AC007099.1 | −1.04 | antisense_RNA |
| 21 | ENSG00000267308 | LINC01764 | −1.03 | lincRNA |
| 22 | ENSG00000269749 | AC005614.2 | −1.01 | processed_transcript |
| 23 | ENSG00000225630 | MTND2P28 | −0.97 | unprocessed_pseudogene |
| 24 | ENSG00000242195 | SRRM1P2 | −0.97 | processed_pseudogene |
| 25 | ENSG00000253347 | AC040934.1 | −0.97 | antisense_RNA |

TABLE XXVIII
List of the top 25 downregulated gene sets from Bru-seq data analysis of
compound JR-1-272 - C2 treated for 4 h in MIA PaCa-2 cells.

| No | Gene set | ES | NES | FDR q-val | Size |
|---|---|---|---|---|---|
| 1 | HAMAI_APOPTOSIS_VIA_TRAIL_UP | −0.508 | −2.848 | 0.000 | 437 |
| 2 | ZHANG_TLX_TARGETS_36HR_DN | −0.518 | −2.618 | 0.000 | 173 |
| 3 | SENGUPTA_NASOPHARYNGEAL_CARCINOMA_WITH_LMP1_UP | −0.503 | −2.612 | 0.000 | 213 |
| 4 | DACOSTA_UV_RESPONSE_VIA_ERCC3_COMMON_DN | −0.448 | −2.492 | 0.000 | 418 |
| 5 | GABRIELY_MIR21_TARGETS | −0.470 | −2.457 | 0.000 | 229 |
| 6 | SENGUPTA_NASOPHARYNGEAL_CARCINOMA_UP | −0.477 | −2.454 | 0.000 | 200 |
| 7 | MILI_PSEUDOPODIA_HAPTOTAXIS_UP | −0.420 | −2.372 | 0.000 | 441 |
| 8 | DACOSTA_UV_RESPONSE_VIA_ERCC3_XPCS_DN | −0.520 | −2.258 | 0.001 | 66 |
| 9 | TAKEDA_TARGETS_OF_NUP98_HOXA9_FUSION_6HR_UP | −0.667 | −2.252 | 0.001 | 24 |
| 10 | SHEN_SMARCA2_TARGETS_UP | −0.402 | −2.248 | 0.001 | 400 |
| 11 | HUTTMANN_B_CLL_POOR_SURVIVAL_DN | −0.605 | −2.158 | 0.003 | 30 |
| 12 | NIKOLSKY_BREAST_CANCER_8Q12_Q22_AMPLICON | −0.499 | −2.099 | 0.007 | 64 |
| 13 | DE_YY1_TARGETS_DN | −0.471 | −2.072 | 0.010 | 74 |
| 14 | DAZARD_UV_RESPONSE_CLUSTER_G6 | −0.434 | −2.065 | 0.010 | 118 |
| 15 | BEGUM_TARGETS_OF_PAX3_FOXO1_FUSION_UP | −0.621 | −2.034 | 0.014 | 22 |
| 16 | ZHANG_TLX_TARGETS_DN | −0.457 | −2.032 | 0.014 | 83 |
| 17 | PYEON_CANCER_HEAD_AND_NECK_VS_CERVICAL_UP | −0.405 | −1.998 | 0.018 | 143 |
| 18 | RIGGINS_TAMOXIFEN_RESISTANCE_DN | −0.420 | −1.996 | 0.017 | 125 |
| 19 | TAKEDA_TARGETS_OF_NUP98_HOXA9_FUSION_3D_UP | −0.469 | −1.940 | 0.031 | 55 |
| 20 | ZHENG_BOUND_BY_FOXP3 | −0.362 | −1.929 | 0.034 | 285 |
| 21 | YANG_BREAST_CANCER_ESR1_LASER_DN | −0.521 | −1.924 | 0.034 | 34 |
| 22 | ZHANG_BREAST_CANCER_PROGENITORS_UP | −0.342 | −1.914 | 0.036 | 343 |
| 23 | SCHLOSSER_MYC_TARGETS_AND_SERUM_RESPONSE_DN | −0.485 | −1.909 | 0.037 | 43 |
| 24 | DOANE_BREAST_CANCER_CLASSES_DN | −0.562 | −1.907 | 0.036 | 23 |
| 25 | ACEVEDO_LIVER_CANCER_WITH_H3K27ME3_UP | −0.444 | −1.897 | 0.038 | 71 |

TABLE XXIX
List of the top 4 downregulated gene sets from Bru-seq data analysis of
compound JR-1-272 - HALLMARK treated for 4 h in MIA PaCa-2 cells.

| | | | | FDR | |
|---|---|---|---|---|---|
| No | Gene set | ES | NES | q-val | Size |
| 1 | HALLMARK_KRAS_SIGNAL- | −0.323 | −1.382 | | 67 |
| | ING_UP | | | | |
| 2 | HALLMARK_TGF_BETA_SIGNAL- | −0.268 | −1.054 | | 44 |
| | ING | | | | |
| 3 | HALLMARK_PROTEIN_SECRE- | −0.222 | −1.015 | | 90 |
| | TION | | | | |
| 4 | HALLMARK_MYC_TARGETS_V1 | −0.198 | −1.007 | | 193 |

TABLE XXX
List of the top 25 downregulated gene sets from Bru-seq data analysis of
compound JR-1-272 - KEGG treated for 4 h in MIA PaCa-2 cells.

| No | Gene set | ES | NES | FDR q-val | Size |
|---|---|---|---|---|---|
| 1 | KEGG_GLYCOSYLPHOSPHATIDYLINOSITOL_GPI_ANCHOR_BIOSYNTHESIS | −0.486 | −1.588 | 0.43818864 | 22 |
| 2 | KEGG_MISMATCH_REPAIR | −0.403 | −1.309 | 1 | 21 |
| 3 | KEGG_PROTEIN_EXPORT | −0.375 | −1.214 | 1 | 22 |
| 4 | KEGG_PURINE_METABOLISM | −0.238 | −1.090 | 1 | 93 |
| 5 | KEGG_NEUROACTIVE_LIGAND_RECEPTOR_INTERACTION | −0.331 | −1.080 | 1 | 21 |
| 6 | KEGG_ALANINE_ASPARTATE_AND_GLUTAMATE_METABOLISM | −0.362 | −1.074 | 1 | 15 |
| 7 | KEGG_ABC_TRANSPORTERS | −0.355 | −1.070 | 1 | 16 |
| 8 | KEGG_JAK_STAT_SIGNALING_PATHWAY | −0.257 | −1.052 | 1 | 55 |
| 9 | KEGG_NUCLEOTIDE_EXCISION_REPAIR | −0.273 | −1.048 | 1 | 41 |
| 10 | KEGG_TGF_BETA_SIGNALING_PATHWAY | −0.250 | −0.991 | 1 | 46 |
| 11 | KEGG_CALCIUM_SIGNALING_PATHWAY | −0.240 | −0.978 | 1 | 50 |
| 12 | KEGG_CELL_CYCLE | −0.190 | −0.882 | 1 | 108 |
| 13 | KEGG_PYRIMIDINE_METABOLISM | −0.198 | −0.876 | 1 | 75 |
| 14 | KEGG_CELL_ADHESION_MOLECULES_CAMS | −0.271 | −0.866 | 1 | 20 |
| 15 | KEGG_VIRAL_MYOCARDITIS | −0.259 | −0.861 | 1 | 22 |
| 16 | KEGG_DNA_REPLICATION | −0.223 | −0.814 | 1 | 34 |
| 17 | KEGG_STARCH_AND_SUCROSE_METABOLISM | −0.260 | −0.785 | 1 | 17 |
| 18 | KEGG_DILATED_CARDIOMYOPATHY | −0.222 | −0.781 | 1 | 29 |
| 19 | KEGG_WNT_SIGNALING_PATHWAY | −0.162 | −0.724 | 1 | 82 |
| 20 | KEGG_PATHOGENIC_ESCHERICHIA_COLI_INFECTION | −0.189 | −0.698 | 1 | 35 |
| 21 | KEGG_AMYOTROPHIC_LATERAL_SCLEROSIS_ALS | −0.197 | −0.684 | 1 | 29 |
| 22 | KEGG_PROPANOATE_METABOLISM | −0.198 | −0.669 | 1 | 25 |
| 23 | KEGG_CYTOSOLIC_DNA_SENSING_PATHWAY | −0.184 | −0.597 | 1 | 23 |
| 24 | KEGG_HEDGEHOG_SIGNALING_PATHWAY | −0.188 | −0.590 | 1 | 19 |
| 25 | KEGG_RNA_POLYMERASE | −0.129 | −0.431 | 0.9992127 | 23 |

TABLE XXXI
List of the top 25 downregulated gene sets from Bru-seq data analysis of
compound JR-1-272 - GO treated for 4 h in MIA PaCa-2 cells.

| No | Gene set | ES | NES | FDR q-val | Size |
|---|---|---|---|---|---|
| 1 | GO_KIDNEY_MORPHOGENESIS | −0.528 | −1.820 | 1 | 26 |
| 2 | GO_NEGATIVE_REGULATION_OF_EPITHELIAL_CELL_PROLIFERATION | −0.415 | −1.746 | 1 | 57 |
| 3 | GO_GLUTAMINE_METABOLIC_PROCESS | −0.590 | −1.707 | 1 | 15 |
| 4 | GO_RESPONSE_TO_X_RAY | −0.487 | −1.645 | 1 | 24 |
| 5 | GO_BASAL_PART_OF_CELL | −0.512 | −1.641 | 1 | 21 |
| 6 | GO_DNA_REPLICATION_INITIATION | −0.474 | −1.636 | 1 | 26 |
| 7 | GO_MITOTIC_RECOMBINATION | −0.432 | −1.634 | 1 | 39 |
| 8 | GO_RECIPROCAL_DNA_RECOMBINATION | −0.486 | −1.628 | 1 | 24 |
| 9 | GO_CILIUM_ORGANIZATION | −0.343 | −1.627 | 1 | 111 |
| 10 | GO_HOMEOSTASIS_OF_NUMBER_OF_CELLS_WITHIN_A_TISSUE | −0.526 | −1.618 | 0.9768239 | 18 |
| 11 | GO_PROTEIN_TRANSPORT_ALONG_MICROTUBULE | −0.495 | −1.610 | 0.9405433 | 22 |
| 12 | GO_STRAND_DISPLACEMENT | −0.488 | −1.598 | 0.9354117 | 22 |
| 13 | GO_NUCLEOCYTOPLASMIC_TRANSPORTER_ACTIVITY | −0.492 | −1.593 | 0.896948 | 22 |
| 14 | GO_GPI_ANCHOR_METABOLIC_PROCESS | −0.462 | −1.593 | 0.8341676 | 28 |
| 15 | GO_DNA_DEPENDENT_DNA_REPLICATION_MAINTENANCE_OF_FIDELITY | −0.517 | −1.584 | 0.8284021 | 18 |
| 16 | GO_DNA_DOUBLE_STRAND_BREAK_PROCESSING | −0.531 | −1.582 | 0.7875705 | 16 |
| 17 | GO_ANCHORED_COMPONENT_OF_MEMBRANE | −0.548 | −1.574 | 0.78832847 | 15 |
| 18 | GO_GLUTAMINE_FAMILY_AMINO_ACID_METABOLIC_PROCESS | −0.458 | −1.568 | 0.7757148 | 28 |
| 19 | GO_DNA_DEPENDENT_ATPASE_ACTIVITY | −0.358 | −1.562 | 0.7706629 | 72 |
| 20 | GO_MESONEPHRIC_TUBULE_MORPHOGENESIS | −0.501 | −1.555 | 0.7660868 | 18 |
| 21 | GO_MALE_MEIOSIS | −0.523 | −1.550 | 0.7601431 | 15 |
| 22 | GO_REGULATION_OF_RESPONSE_TO_INTERFERON_GAMMA | −0.521 | −1.545 | 0.7491881 | 17 |
| 23 | GO_DNA_REPAIR_COMPLEX | −0.410 | −1.544 | 0.7229545 | 36 |
| 24 | GO_DNA_DEPENDENT_DNA_REPLICATION | −0.345 | −1.540 | 0.7095914 | 83 |
| 25 | GO_LYSOPHOSPHOLIPID_ACYLTRANSFERASE_ACTIVITY | −0.526 | −1.539 | 0.68688023 | 15 |

TABLE XXXII
List of the top 25 downregulated gene sets from Bru-seq data
analysis of compound JR-1-272-TFBT
treated for 4 h in MIA PaCa-2 cells.

| No | Gene set | ES | NES | FDR q-val | Size |
|---|---|---|---|---|---|
| 1 | POU3F2_01 | -0.443 | -1.657 | 0.39121717 | 37 |
| 2 | HOX13_01 | -0.477 | -1.570 | 0.4447494 | 21 |
| 3 | PAX8_01 | -0.503 | -1.563 | 0.3131025 | 19 |
| 4 | CDP_01 | -0.427 | -1.488 | 0.44670406 | 29 |
| 5 | PIT1_Q6 | -0.325 | -1.466 | 0.43412176 | 91 |
| 6 | PBX1_01 | -0.297 | -1.344 | 0.9584583 | 95 |
| 7 | EN1_01 | -0.350 | -1.341 | 0.84534574 | 42 |
| 8 | YKACATTT_UNKNOWN | -0.273 | -1.323 | 0.8542393 | 145 |
| 9 | HMEF2_Q6 | -0.330 | -1.322 | 0.76531255 | 45 |
| 10 | FOXJ2_02 | -0.288 | -1.314 | 0.73387516 | 92 |
| 11 | GGCKCATGS_UNKNOWN | -0.339 | -1.305 | 0.71688175 | 41 |
| 12 | GCGSCMNTTT_UNKNOWN | -0.310 | -1.293 | 0.7142225 | 58 |
| 13 | YTATTTTNR_MEF2_02 | -0.236 | -1.290 | 0.67760056 | 285 |
| 14 | PAX2_02 | -0.269 | -1.268 | 0.74662375 | 112 |
| 15 | MEIS1BHOXA9_01 | -0.311 | -1.264 | 0.7142713 | 51 |
| 16 | NKX3A_01 | -0.280 | -1.259 | 0.6958817 | 93 |
| 17 | HNF1_Q6 | -0.283 | -1.255 | 0.67633516 | 80 |
| 18 | CEBPA_01 | -0.265 | -1.252 | 0.6525275 | 113 |
| 19 | TGATTTRY_GFI1_01 | -0.264 | -1.215 | 0.80217177 | 99 |
| 20 | MEF2_01 | -0.299 | -1.207 | 0.8096997 | 50 |
| 21 | STAT5A_04 | -0.265 | -1.205 | 0.78265405 | 94 |
| 22 | WTGAAAT_UNKNOWN | -0.226 | -1.204 | 0.7518113 | 260 |
| 23 | TAANNYSGCG_UNKNOWN | -0.289 | -1.196 | 0.75633866 | 60 |
| 24 | IRF2_01 | -0.285 | -1.195 | 0.73193073 | 62 |
| 25 | HNF1_01 | -0.277 | -1.195 | 0.7040818 | 66 |

The up and downregulated gene lists were used to query the CMAP database for overall transcription profiles of reported pertubagens. The top 25 pertubagens (compounds) correlating with JR-1-272 transcription profile is reported in Tables XXXIII and XXXIV. Mostly HD AC inhibitor, EGFR inhibitor and kinase inhibitors were identified to have similar transcription profiles suggesting correlation in the mechanism of action. Compounds identified by CMAP do not show significant structural similarity with JR-1-272. However, correlation of these compounds hints on potential mechanisms of JR-1-272 activity, and application of these compounds as tools for comparison might be a plausible approach to further characterize JR-1-272 in different biological systems.

TABLE XXXIII
List of the top CMAP hits positively correlated with JR-1-272 treated for 4 h in MIA PaCa-2 cells.

| No | Median score | Name | Structure | Description |
|---|---|---|---|---|
| 1 | 99.42 | Bisindolyl- maleimide | 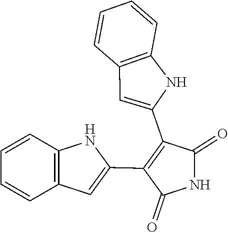 [SMILES: O=C1NC(=O)C(c2cc3ccccc3[nH]2)=C1c1cc2ccccc2[nH]1] | CDK inhibitor, PKC inhibitor, leucine rich repeat kinase inhibitor |
| 2 | 99.13 | Trichostatin-a | 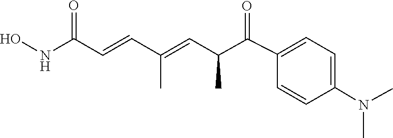 [SMILES: CC(/C=C/C(=O)NO)=C\[C@H](C)C(=O)c1ccc(N(C)C)cc1] | HDAC inhibitor, CDK expression enhancer, ID1 expression inhibitor |
| 3 | 99.12 | Trichostatin-a | 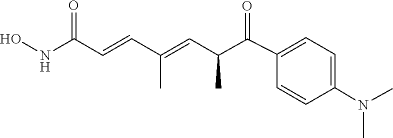 [SMILES: CC(/C=C/C(=O)NO)=C\[C@H](C)C(=O)c1ccc(N(C)C)cc1] | HDAC inhibitor, CDK expression enhancer, ID1 expression inhibitor |
| 4 | 99.09 | Vorinostat | 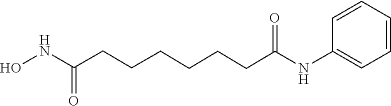 [SMILES: O=C(CCCCCCC(=O)Nc1ccccc1)NO] | HDAC inhibitor, cell cycle inhibitor |
| 5 | 98.97 | ISOX | 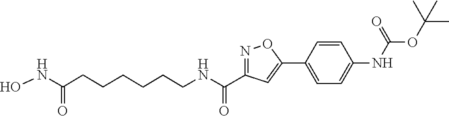 [SMILES: CC(C)(C)OC(=O)Nc1ccc(-c2cc(C(=O)NCCCCCCC(=O)NO)no2)cc1] | HDAC inhibitor |
| 6 | 98.79 | BIBX-1382 | 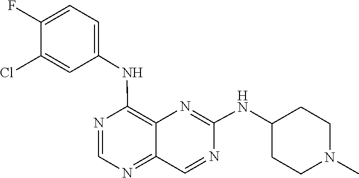 [SMILES: CN1CCC(Nc2ncc3ncnc(Nc4ccc(F)c(Cl)c4)c3n2)CC1] | EGFR inhibitor, tyrosine kinase inhibitor |
| 7 | 98.76 | Panobinostat | 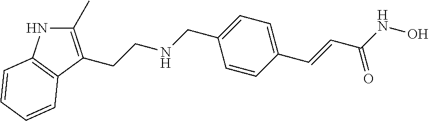 [SMILES: Cc1[nH]c2ccccc2c1CCNCc1ccc(/C=C/C(=O)NO)cc1] | HDAC inhibitor, apoptosis stimulant, cell cycle inhibitor |
| 8 | 98.73 | WT-171 | 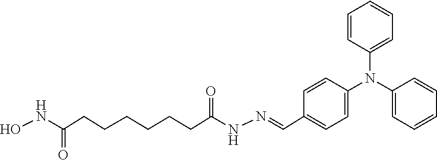 [SMILES: O=C(CCCCCCC(=O)N/N=C/c1ccc(N(c2ccccc2)c2ccccc2)cc1)NO] | HDAC inhibitor |
| 9 | 98.49 | BIBU-1361 | 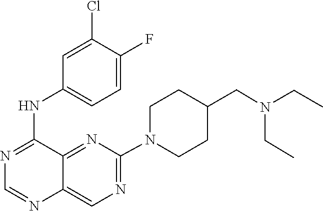 [SMILES: CCN(CC)CC1CCN(c2ncc3ncnc(Nc4ccc(F)c(Cl)c4)c3n2)CC1] | EGFR inhibitor |
| 10 | 97.99 | BRD- K06956503 | 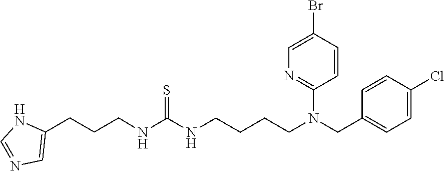 [SMILES: S=C(NCCCCN(Cc1ccc(Cl)cc1)c1ccc(Br)cn1)NCCCc1cnc[nH]1] | glucosylceramidase inhibitor |
| 11 | 97.96 | HC-toxin | 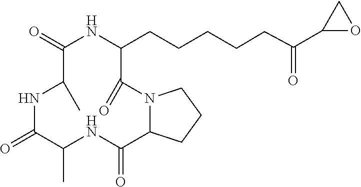 [SMILES: CC1NC(=O)C(C)NC(=O)C2CCCN2C(=O)C(CCCCCC(=O)C2CO2)NC1=O] | HDAC inhibitor |
| 12 | 97.09 | Importazole | 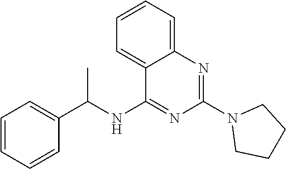 [SMILES: CC(Nc1nc(N2CCCC2)nc2ccccc12)c1ccccc1] | importin-ÃfÅ½Ã,Â² transport receptor inhibitor |
| 13 | 96.67 | Rimcazole | 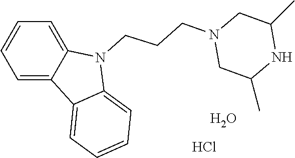 | sigma receptor antagonist, dopamine reuptake inhibitor |
| 14 | 96.43 | Maprotiline | 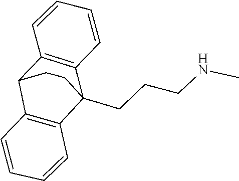 [SMILES: CNCCCC12CCC(c3ccccc31)c1ccccc12] | norepinephrine reputake inhibitor, tricyclic antidepressant (TCA) |
| 15 | 96.38 | NTNCB | 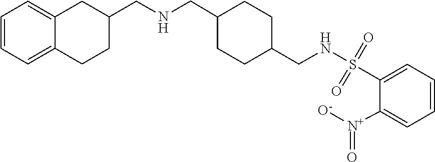 [SMILES: O=[N+]([O-])c1ccccc1S(=O)(=O)NCC1CCC(CNCC2CCc3ccccc3C2)CC1] | neuropeptide receptor antagonist |
| 16 | 96.33 | IKK-16 | 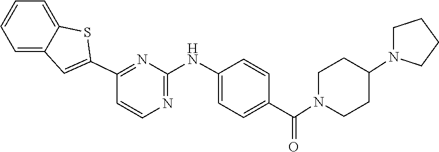 [SMILES: O=C(c1ccc(Nc2nccc(-c3cc4ccccc4s3)n2)cc1)N1CCC(N2CCCC2)CC1] | IKK inhibitor |
| 17 | 96.17 | BI-2536 | 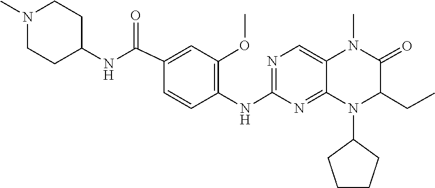 [SMILES: CCC1C(=O)N(C)c2cnc(Nc3ccc(C(=O)NC4CCN(C)CC4)cc3OC)nc2N1C1CCCC1] | PLK inhibitor, apoptosis stimulant, cell cycle inhibitor, protein kinase inhibitor |
| 18 | 95.69 | Alimemazine | 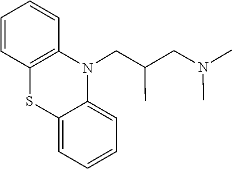 [SMILES: CC(CN(C)C)CN1c2ccccc2Sc2ccccc21] | histamine receptor ligand |
| 19 | 95.42 | Amlodipine | 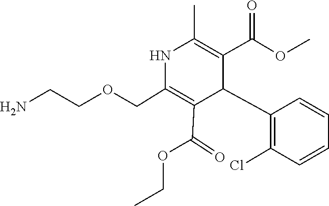 [SMILES: CCOC(=O)C1=C(COCCN)NC(C)=C(C(=O)OC)C1c1ccccc1Cl] | breast cancer resistance protein inhibitor, calcium channel blocker, calcium channel inhibitor, L-type calcium channel blocker |
| 20 | 95.38 | SU-11274 | 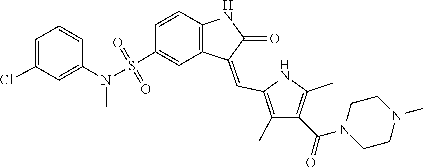 [SMILES: Cc1[nH]c(/C=C2\C(=O)Nc3ccc(S(=O)(=O)N(C)c4cccc(Cl)c4)cc32)c(C)c1C(=O)N1CCN(C)CC1] | hepatocyte growth factor receptor inhibitor, tyrosine kinase inhibitor |
| 21 | 94.92 | TG-101348 | 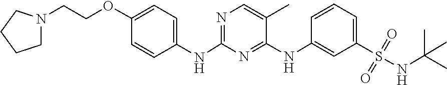 [SMILES: Cc1cnc(Nc2ccc(OCCN3CCCC3)cc2)nc1Nc1cccc(S(=O)(=O)NC(C)(C)C)c1] | JAK inhibitor, FLT3 inhibitor, RET tyrosine kinase inhibitor |
| 22 | 94.85 | NNC-05-2090 | 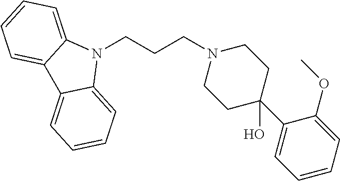 [SMILES: COc1ccccc1C1(O)CCN(CCCn2c3ccccc3c3ccccc32)CC1] | GABA uptake inhibitor, GAT inhibitor |
| 23 | 94.75 | Perospirone | 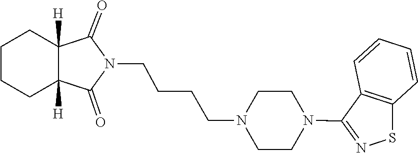 [SMILES: O=C1[C@H]2CCCC[C@H]2C(=O)N1CCCCN1CCN(c2nsc3ccccc23)CC1] | dopamine receptor antagonist, serotonin receptor antagonist |
| 24 | 94.12 | THM-I-94 | 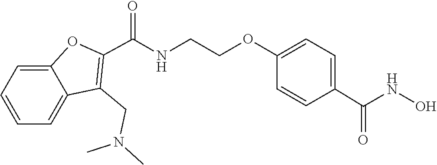 [SMILES: CN(C)Cc1c(C(=O)NCCOc2ccc(C(=O)NO)cc2)oc2ccccc12] | HDAC inhibitor, apoptosis stimulant, cell cycle inhibitor |
| 25 | 93.92 | SN-38 | 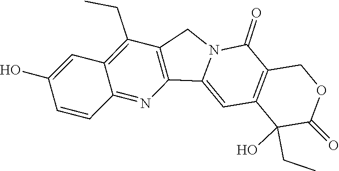 [SMILES: CCc1c2c(nc3ccc(O)cc13)-c1cc3c(c(=O)n1C2)COC(=O)C3(O)CC] | topoisomerase inhibitor |

TABLE XXXIV
List of the top CMAP hits negatively correlated with JR-1-272 treated for 4 h in MIA PaCa-2 cells.

| No | Median score | name | Structure | Description |
|---|---|---|---|---|
| 1 | −99.23 | calyculin | 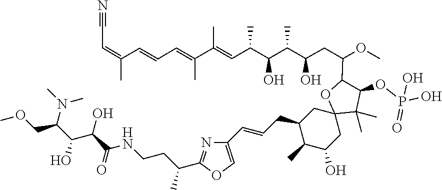 [SMILES: COC[C@H]([C@@H](O)[C@@H](O)C(=O)NCC[C@@H](C)c1nc(/C=C/C[C@H]2CC3(C[C@H](O)[C@H]2C)OC(C(C[C@@H](O)[C@@H](C)[C@@H](O)[C@@H](C)/C=C(C)/C(C)=C/C=C/C(C)=C\C#N)OC)[C@@H](OP(=O)(O)O)C3(C)C)co1)N(C)C] | protein phosphatase inhibitor |
| 2 | −94.07 | cycloheximide | 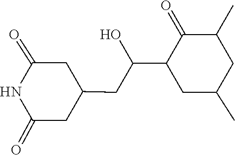 [SMILES: CC1CC(C)C(=O)C(C(O)CC2CC(=O)NC(=O)C2)C1] | glycogen synthase kinase inhibitor, protein synthesis inhibitor |
| 3 | −91.99 | homo- harringtonine | 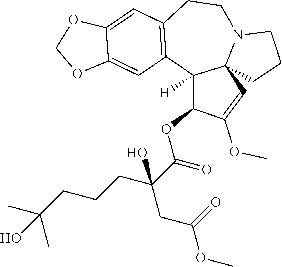 [SMILES: COC(=O)C[C@](O)(CCCC(C)(C)O)C(=O)O[C@@H]1C(OC)=C[C@]23CCCN2CCc2cc4c(cc2[C@H]13)OCO4] | apoptosis stimulant, protein synthesis inhibitor |
| 4 | −91.42 | emetine | 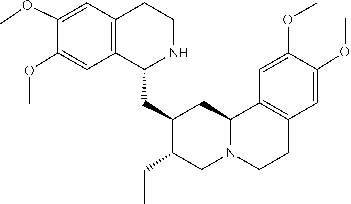 | protein synthesis inhibitor |
| 5 | −90.18 | narciclasine | 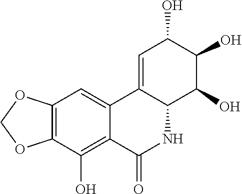 [SMILES: O=C1N[C@@H]2C(=C[C@H](O)[C@@H](O)[C@H]2O)c2cc3c(c(O)c21)OCO3] | coflilin signaling pathway activator, LIM kinase activator, ROCK activator |
| 6 | −88.01 | emetine | 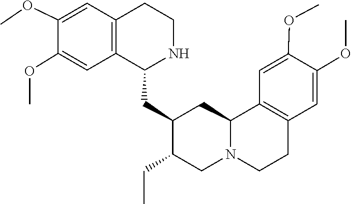 | protein synthesis inhibitor |
| 7 | −87.64 | cycloheximide | 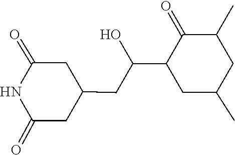 [SMILES: CC1CC(C)C(=O)C(C(O)CC2CC(=O)NC(=O)C2)C1] | glycogen synthase kinase inhibitor, protein synthesis inhibitor |
| 8 | −87.46 | cobalt(II)- chloride | Co²⁺ Cl⁻ Cl⁻ | HSP agonist |
| 9 | −87.39 | helveticoside | 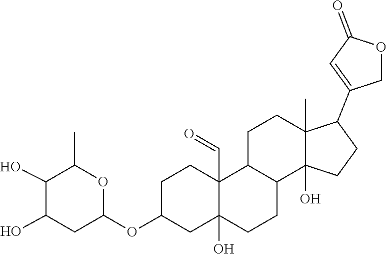 [SMILES: CC1OC(OC2CCC3(C=O)C4CCC5(C)C(C6=CC(=O)OC6)CCC5(O)C4CCC3(O)C2)CC(O)C1O] | ATPase inhibitor |
| 10 | −86.90 | ochratoxin-a | 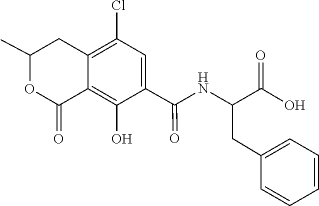 [SMILES: CC1Cc2c(Cl)cc(C(=O)NC(Cc3ccccc3)C(=O)O)c(O)c2C(=O)O1] | phenylalanyl tRNA synthetase inhibitor |
| 11 | −85.66 | anisomycin | 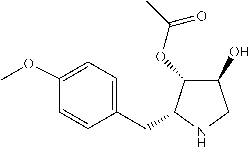 [SMILES: COc1ccc(C[C@H]2NC[C@H](O)[C@H]2OC(C)=O)cc1] | DNA synthesis inhibitor |
| 12 | −84.98 | homo- harringtonine | 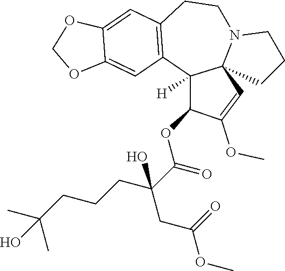 [SMILES: COC(=O)C[C@](O)(CCCC(C)(C)O)C(=O)O[C@@H]1C(OC)=C[C@]23CCCN2CCc2cc4c(cc2[C@H]13)OCO4] | apoptosis stimulant, protein synthesis inhibitor |
| 13 | −84.18 | vecuronium | 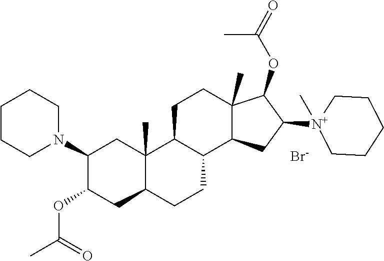 [SMILES: CC(=O)O[C@H]1C[C@@H]2CC[C@@H]3[C@H](CC[C@@]4(C)[C@H]3C[C@H]([N+]3(C)CCCCC3)[C@@H]4OC(C)=O)[C@@]2(C)C[C@@H]1N1CCCCC1 ; [Br-]] | acetylcholine receptor antagonist |
| 14 | −83.35 | puromycin | 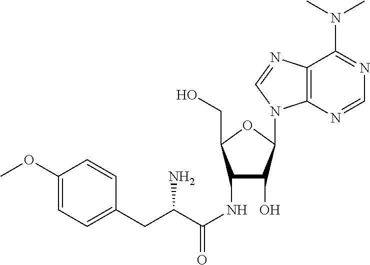 | adenosine receptor agonist, protein synthesis inhibitor |
| 15 | −82.60 | sarmentogenin | 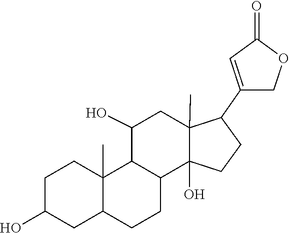 [SMILES: CC12CCC(O)CC1CCC1C2C(O)CC2(C)C(C3=CC(=O)OC3)CCC12O] | ATPase inhibitor |
| 16 | −81.62 | GR-32191 | 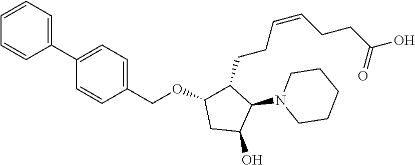 [SMILES: O=C(O)CC/C=C\CC[C@@H]1[C@@H](N2CCCCC2)[C@@H](O)C[C@@H]1OCc1ccc(-c2ccccc2)cc1] | thromboxane receptor antagonist, prostanoid receptor antagonist |
| 17 | −81.52 | hydroxychole sterol | 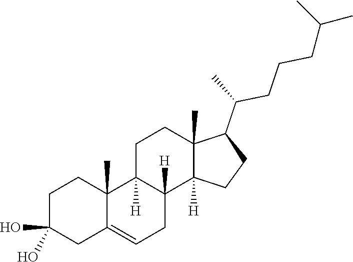 | LXR agonist, ABC transporter expression enhancer, alpha secretase activator, beta secretase inhibitor, glutamate receptor modulator |
| 18 | −81.51 | equol | 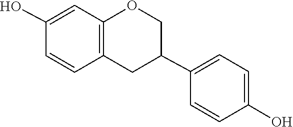 [SMILES: Oc1ccc(C2COc3cc(O)ccc3C2)cc1] | estrogen receptor agonist |
| 19 | −81.22 | retinol | 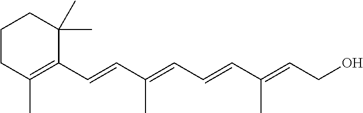 [SMILES: CC1=C(/C=C/C(C)=C/C=C/C(C)=C/CO)C(C)(C)CCC1] | RAR receptor binder |
| 20 | −81.03 | bicuculline | 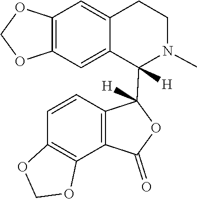 [SMILES: CN1CCc2cc3c(cc2[C@H]1[C@@H]1OC(=O)c2c1ccc1c2OCO1)OCO3] | GABA receptor antagonist |
| 21 | −80.74 | piperacillin | 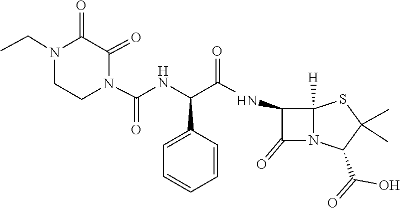 [SMILES: CCN1CCN(C(=O)N[C@@H](C(=O)N[C@@H]2C(=O)N3[C@@H]2SC(C)(C)[C@@H]3C(=O)O)c2ccccc2)C(=O)C1=O] | cell wall synthesis inhibitor |
| 22 | −80.31 | genipin | 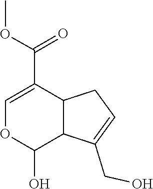 [SMILES: COC(=O)C1=COC(O)C2C(CO)=CCC12] | aglycone, nitric oxide production inhibitor |
| 23 | −80.30 | TUL-XXI039 | 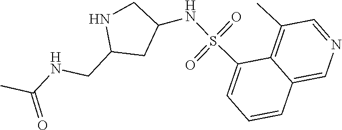 [SMILES: CC(=O)NCC1CC(NS(=O)(=O)c2cccc3cncc(C)c23)CN1] | serine/threonine kinase inhibitor |
| 24 | −79.22 | amonafide | 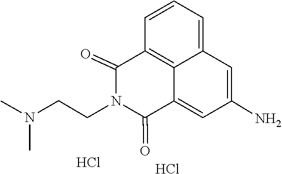 [SMILES: CN(C)CCN1C(=O)c2cccc3cc(N)cc(c23)C1=O ; Cl ; Cl] | topoisomerase inhibitor, DNA intercalating drug |
| 25 | −78.91 | PLX-4720 | 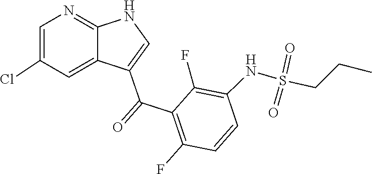 [SMILES: CCCS(=O)(=O)Nc1ccc(F)c(C(=O)c2c[nH]c3ncc(Cl)cc23)c1F] | RAF inhibitor |

Proteomics study of JR-1-235 revealed Hydroxymethylglutaryl-CoA synthase, cytoplasmic (HMGCS1) which is involved in the subpathway that synthesizes (R)-mevalonate from acetyl-CoA is upregulated (Table XXXVIII). Among its related pathways are regulation of cholesterol biosynthesis by SREBP (SREBF) and terpenoid backbone biosynthesis. Hence the proteomics data also supports the cholesterol biosynthesis pathway involved in the mechanism of action of this set of compounds.

TABLE XXXV
JR-1-235 downregulated proteins

| | | | Abundance ratio | | Protein FDR |
|---|---|---|---|---|---|
| No | Protein name | Abundance ratio | (log2) | Coverage | confidence |
| 1 | MBT domain-containing | 0.233 | −2.1 | 1.27 | MEDIUM |
| | protein 1 | | | | |
| 2 | Elongation factor G, | 0.245 | −2.03 | 32.22 | HIGH |
| | mitochondrial | | | | |
| 3 | Aldehyde dehydrogenase X | 0.278 | −1.85 | 16.25 | HIGH |
| 4 | LanC-like protein 2 | 0.327 | −1.61 | 6.89 | HIGH |
| 5 | 28S ribosomal protein S18b, | 0.337 | −1.57 | 10.47 | HIGH |
| | mitochondrial | | | | |
| 6 | Transcription elongation factor | 0.364 | −1.46 | 21.69 | HIGH |
| | 1 homolog | | | | |
| 7 | DNA topoisomerase 2-alpha | 0.381 | −1.39 | 44.55 | HIGH |
| 8 | FAST kinase domain- | 0.382 | −1.39 | 3.27 | HIGH |
| | containing protein 5, | | | | |
| | mitochondrial | | | | |
| 9 | Scaffold attachment factor B1 | 0.401 | −1.32 | 23.17 | HIGH |
| 10 | 28S ribosomal protein S25, | 0.402 | −1.32 | 13.87 | HIGH |
| | mitochondrial | | | | |
| 11 | 28S ribosomal protein S16, | 0.403 | −1.31 | 14.60 | HIGH |
| | mitochondrial | | | | |
| 12 | Scaffold attachment factor B2 | 0.405 | −1.3 | 14.06 | HIGH |
| 13 | Constitutive coactivator of | 0.419 | −1.25 | 8.13 | HIGH |
| | peroxisome proliferator- | | | | |
| | activated receptor gamma | | | | |
| 14 | 28S ribosomal protein S22, | 0.432 | −1.21 | 22.50 | HIGH |
| | mitochondrial | | | | |
| 15 | Keratin, type I cytoskeletal 20 | 0.439 | −1.19 | 5.90 | HIGH |
| 16 | RNA-binding motif protein, X | 0.441 | −1.18 | 52.94 | HIGH |
| | chromosome | | | | |
| 17 | 28S ribosomal protein S17, | 0.452 | −1.15 | 30.77 | HIGH |
| | mitochondrial | | | | |
| 18 | Protein FAM83H | 0.46 | −1.12 | 2.29 | HIGH |
| 19 | Interleukin-36 beta | 0.462 | −1.12 | 6.10 | HIGH |
| 20 | Histone H1.2 | 0.462 | −1.11 | 59.15 | HIGH |
| 21 | NADH dehydrogenase | 0.468 | −1.1 | 12.57 | HIGH |
| | [ubiquinone] iron-sulfur | | | | |
| | protein 4, mitochondrial | | | | |
| 22 | NADH dehydrogenase | 0.473 | −1.08 | 10.39 | HIGH |
| | [ubiquinone] 1 alpha | | | | |
| | subcomplex subunit 6 | | | | |
| 23 | Proliferation marker protein | 0.474 | −1.08 | 38.11 | HIGH |
| | Ki-67 | | | | |
| 24 | Nucleolar transcription factor 1 | 0.484 | −1.05 | 21.99 | HIGH |
| 25 | D-dopachrome decarboxylase | 0.487 | −1.04 | 34.75 | HIGH |

TABLE XXXVI
JR-1-235 upregulated proteins

| | | | Abundance ratio | | Protein FDR |
|---|---|---|---|---|---|
| No | Protein name | Abundance ratio | (log2) | Coverage | confidence |
| 1 | Serine/threonine-protein | 3.158 | 1.66 | 1.24 | LOW |
| | kinase PLK4 | | | | |
| 2 | Protein FAM177A1 | 3.085 | 1.63 | 4.69 | MEDIUM |
| 3 | Dermcidin | 2.454 | 1.3 | 20.00 | HIGH |
| 4 | Apolipoprotein B-100 | 2.151 | 1.1 | 1.95 | HIGH |
| 5 | Acyl-CoA desaturase | 2.148 | 1.1 | 5.85 | HIGH |
| 6 | Iron-responsive element- | 2.136 | 1.09 | 1.66 | HIGH |
| | binding protein 2 | | | | |
| 7 | Apolipoprotein C-III | 2.072 | 1.05 | 16.16 | HIGH |
| 8 | Filamin-C | 2.026 | 1.02 | 5.50 | HIGH |
| 9 | Phosphatidylserine synthase 1 | 1.946 | 0.96 | 1.69 | MEDIUM |
| 10 | Hydroxymethylglutaryl- | 1.944 | 0.96 | 17.88 | HIGH |
| | CoA synthase, cytoplasmic | | | | |
| 11 | E3 ubiquitin-protein ligase | 1.906 | 0.93 | 2.20 | LOW |
| | TRIM69 | | | | |
| 12 | Apolipoprotein A-I | 1.891 | 0.92 | 5.99 | HIGH |
| 13 | Kinesin-like protein KIF27 | 1.89 | 0.92 | 1.64 | HIGH |
| 14 | Mixed lineage kinase | 1.861 | 0.9 | 6.16 | HIGH |
| | domain-like protein | | | | |
| 15 | Bleomycin hydrolase | 1.845 | 0.88 | 1.54 | MEDIUM |
| 16 | Muellerian-inhibiting factor | 1.824 | 0.87 | 1.79 | LOW |
| 17 | 60S ribosomal protein L37 | 1.808 | 0.85 | 39.18 | HIGH |
| 18 | Copper-transporting | 1.807 | 0.85 | 0.48 | MEDIUM |
| | ATPase 2 | | | | |
| 19 | Putative ankyrin repeat | 1.786 | 0.84 | 0.43 | LOW |
| | domain-containing protein | | | | |
| | 31 | | | | |
| 20 | 40S ribosomal protein S8 | 1.777 | 0.83 | 55.29 | HIGH |
| 21 | 60S ribosomal protein L19 | 1.761 | 0.82 | 39.29 | HIGH |
| 22 | GH3 domain-containing | 1.742 | 0.8 | 1.70 | MEDIUM |
| | protein | | | | |
| 23 | 40S ribosomal protein S26 | 1.717 | 0.78 | 55.65 | HIGH |
| 24 | 60S ribosomal protein L27a | 1.713 | 0.78 | 34.46 | HIGH |
| 25 | Protein PIMREG | 1.711 | 0.77 | 3.63 | LOW |

TABLE XXXVII
JR-1-235 downregulated proteins

| | | | Abundance ratio | | Protein FDR |
|---|---|---|---|---|---|
| No | Protein name | Abundance ratio | (log2) | Coverage | confidence |
| 1 | Elongation factor G, | 0.245 | −2.03 | 32.22 | HIGH |
| | mitochondrial | | | | |
| 2 | Aldehyde dehydrogenase X | 0.278 | −1.85 | 16.25 | HIGH |
| 3 | LanC-like protein 2 | 0.327 | −1.61 | 6.89 | HIGH |
| 4 | 28S ribosomal protein S18b, | 0.337 | −1.57 | 10.47 | HIGH |
| | mitochondrial | | | | |
| 5 | Transcription elongation factor | 0.364 | −1.46 | 21.69 | HIGH |
| | 1 homolog | | | | |
| 6 | DNA topoisomerase 2-alpha | 0.381 | −1.39 | 44.55 | HIGH |
| 7 | FAST kinase domain- | 0.382 | −1.39 | 3.27 | HIGH |
| | containing protein 5, | | | | |
| | mitochondrial | | | | |
| 8 | Scaffold attachment factor B1 | 0.401 | −1.32 | 23.17 | HIGH |
| 9 | 28S ribosomal protein S25, | 0.402 | −1.32 | 13.87 | HIGH |
| | mitochondrial | | | | |
| 10 | 28S ribosomal protein S16, | 0.403 | −1.31 | 14.60 | HIGH |
| | mitochondrial | | | | |
| 11 | Scaffold attachment factor B2 | 0.405 | −1.3 | 14.06 | HIGH |
| 12 | Constitutive coactivator of | 0.419 | −1.25 | 8.13 | HIGH |
| | peroxisome proliferator- | | | | |
| | activated receptor gamma | | | | |
| 13 | 28S ribosomal protein S22, | 0.432 | −1.21 | 22.50 | HIGH |
| | mitochondrial | | | | |
| 14 | Keratin, type I cytoskeletal 20 | 0.439 | −1.19 | 5.90 | HIGH |
| 15 | RNA-binding motif protein, X | 0.441 | −1.18 | 52.94 | HIGH |
| | chromosome | | | | |
| 16 | 28S ribosomal protein S17, | 0.452 | −1.15 | 30.77 | HIGH |
| | mitochondrial | | | | |
| 17 | Protein FAM83H | 0.46 | −1.12 | 2.29 | HIGH |
| 18 | Histone H1.2 | 0.462 | −1.11 | 59.15 | HIGH |
| 19 | NADH dehydrogenase | 0.468 | −1.1 | 12.57 | HIGH |
| | [ubiquinone] iron-sulfur | | | | |
| | protein 4, mitochondrial | | | | |
| 20 | NADH dehydrogenase | 0.473 | −1.08 | 10.39 | HIGH |
| | [ubiquinone] 1 alpha | | | | |
| | subcomplex subunit 6 | | | | |
| 21 | Proliferation marker protein | 0.474 | −1.08 | 38.11 | HIGH |
| | Ki-67 | | | | |
| 22 | Nucleolar transcription factor 1 | 0.484 | −1.05 | 21.99 | HIGH |
| 23 | D-dopachrome decarboxylase | 0.487 | −1.04 | 34.75 | HIGH |
| 24 | NADH-ubiquinone | 0.488 | −1.03 | 30.40 | HIGH |
| | oxidoreductase 75 kDa | | | | |
| | subunit, mitochondrial | | | | |
| 25 | Aconitate hydratase, | 0.489 | −1.03 | 40.90 | HIGH |
| | mitochondrial | | | | |

TABLE XXXVIII
JR-1-235 upregulated proteins

| | | | Abundance ratio | | Protein FDR |
|---|---|---|---|---|---|
| No | Protein name | Abundance ratio | (log2) | Coverage | confidence |
| 1 | Dermcidin | 2.454 | 1.3 | 20.00 | HIGH |
| 2 | Apolipoprotein B-100 | 2.151 | 1.1 | 1.95 | HIGH |
| 3 | Acyl-CoA desaturase | 2.148 | 1.1 | 5.85 | HIGH |
| 4 | Iron-responsive element- | 2.136 | 1.09 | 1.66 | HIGH |
| | binding protein 2 | | | | |
| 5 | Apolipoprotein C-III | 2.072 | 1.05 | 16.16 | HIGH |
| 6 | Filamin-C | 2.026 | 1.02 | 5.50 | HIGH |
| 7 | Hydroxymethylglutaryl- | 1.944 | 0.96 | 17.88 | HIGH |
| | CoA synthase, cytoplasmic | | | | |
| 8 | Apolipoprotein A-I | 1.891 | 0.92 | 5.99 | HIGH |
| 9 | Kinesin-like protein KIF27 | 1.89 | 0.92 | 1.64 | HIGH |
| 10 | Mixed lineage kinase | 1.861 | 0.9 | 6.16 | HIGH |
| | domain-like protein | | | | |
| 11 | 60S ribosomal protein L37 | 1.808 | 0.85 | 39.18 | HIGH |
| 12 | 40S ribosomal protein S8 | 1.777 | 0.83 | 55.29 | HIGH |
| 13 | 60S ribosomal protein L19 | 1.761 | 0.82 | 39.29 | HIGH |
| 14 | 40S ribosomal protein S26 | 1.717 | 0.78 | 55.65 | HIGH |
| 15 | 60S ribosomal protein L27a | 1.713 | 0.78 | 34.46 | HIGH |
| 16 | Sequestosome-1 | 1.69 | 0.76 | 58.18 | HIGH |
| 17 | Protein cornichon homolog 4 | 1.656 | 0.73 | 14.39 | HIGH |
| 18 | 40S ribosomal protein S30 | 1.649 | 0.72 | 33.90 | HIGH |
| 19 | Deoxyribonuclease-2-alpha | 1.649 | 0.72 | 4.72 | HIGH |
| 20 | Diphthine methyltransferase | 1.633 | 0.71 | 5.97 | HIGH |
| 21 | 60S ribosomal protein L36a- | 1.623 | 0.7 | 39.62 | HIGH |
| | like | | | | |
| 22 | Beta-2-glycoprotein 1 | 1.622 | 0.7 | 5.22 | HIGH |
| 23 | Alanine aminotransferase 2 | 1.616 | 0.69 | 6.88 | HIGH |
| 24 | Lumican | 1.612 | 0.69 | 5.92 | HIGH |
| 25 | Squalene synthase | 1.597 | 0.68 | 21.34 | HIGH |

In order to understand the selectivity of these compounds in different cancer types, some of them were tested against a panel of 60 cell lines (Table XXXIX).

TABLE XXXIX
Results of JR-1-235, JR-1-157, JR-1-242, JR-3-6 and JR-2-298 in a panel of NCI60 cell lines.
As evident from the results JR-1-157 and JR-1-242 are most active in melanoma cell line
LOX IMVI apart from pancreatic cell line MIA PaCa-2.

| | | GIPRCNT | GIPRCNT | GIPRCNT | GIPRCNT | GIPRCNT |
|---|---|---|---|---|---|---|
| PANEL NAME | CELLNAME | of JR-1-235 | of JR-1-157 | of JR-1-242 | of JR-3-6 | of JR-2-298 |
| Leukemia | CCRF-CEM | 54.24127 | 5.287604 | −7.64925 | 26.30561 | 19.66437 |
| Leukemia | HL-60(TB) | 76.80054 | −63.3005 | 72.3913 | 77.06738 | 85.54454 |
| Leukemia | K-562 | 25.87631 | −45.7589 | −57.0747 | 19.84321 | 3.945656 |
| Leukemia | MOLT-4 | 46.46107 | 2.900801 | 53.42747 | 24.47506 | 21.9615 |
| Leukemia | RPMI-8226 | 77.97016 | 2.195726 | 23.80071 | 93.13265 | 75.09198 |
| Leukemia | SR | 81.88484 | 1.416206 | −5.71043 | 29.46357 | 2.838637 |
| Non-Small Cell | A549/ATCC | 72.79696 | 23.42792 | 81.17586 | 88.09551 | 58.01082 |
| Lung Cancer | | | | | | |
| Non-Small Cell | EKVX | 77.76789 | 66.07156 | 104.0141 | 82.71214 | 73.30195 |
| Lung Cancer | | | | | | |
| Non-Small Cell | HOP-62 | 91.15376 | 43.04074 | 94.18225 | 85.23725 | 64.07519 |
| Lung Cancer | | | | | | |
| Non-Small Cell | HOP-92 | 51.40301 | 38.77949 | 49.4978 | 49.08774 | 37.51522 |
| Lung Cancer | | | | | | |
| Non-Small Cell | NCI-H226 | 60.38681 | 39.12922 | 61.44056 | 100.3613 | 85.09095 |
| Lung Cancer | | | | | | |
| Non-Small Cell | NCI-H23 | 96.30444 | 80.42987 | 96.09779 | 83.43447 | 81.36732 |
| Lung Cancer | | | | | | |
| Non-Small Cell | NCI-H322M | 84.17614 | 35.29949 | 75.46682 | 69.28567 | 63.79946 |
| Lung Cancer | | | | | | |
| Non-Small Cell | NCI-H460 | 79.50406 | 3.469368 | 1.048484 | 80.61737 | 69.54597 |
| Lung Cancer | | | | | | |
| Non-Small Cell | NCI-H522 | 96.8365 | 4.414693 | 5.069086 | 54.48122 | 52.68604 |
| Lung Cancer | | | | | | |
| Colon Cancer | COLO 205 | 37.0068 | −66.6925 | 71.36528 | 13.90185 | 3.107909 |
| Colon Cancer | HCC-2998 | 89.79627 | 32.58744 | 90.92323 | 85.58186 | 66.68672 |
| Colon Cancer | HCT-116 | 18.04625 | −52.4351 | −36.0085 | 11.00216 | 9.520187 |
| Colon Cancer | HCT-15 | 59.89069 | 3.732284 | −25.625 | 40.51756 | 24.49644 |
| Colon Cancer | HT29 | 37.31238 | −49.3651 | −47.1759 | 36.78303 | 11.79949 |
| Colon Cancer | KM12 | 97.89775 | 30.30254 | 36.32424 | 81.23639 | 75.33702 |
| Colon Cancer | SW-620 | 75.25877 | 0.61885 | 2.118123 | 40.94409 | 24.30046 |
| CNS Cancer | SF-268 | 66.12481 | 35.84677 | 63.02817 | 77.44564 | 51.92831 |
| CNS Cancer | SF-295 | 98.94038 | 63.03419 | 100.8191 | 89.29602 | 71.23389 |
| CNS Cancer | SF-539 | 86.86979 | −73.563 | 0.456658 | 85.49552 | 83.11985 |
| CNS Cancer | SNB-19 | 89.23704 | 49.64959 | 94.85367 | 87.74213 | 83.08791 |
| CNS Cancer | SNB-75 | 78.26339 | 41.69555 | 75.89036 | 82.71391 | 65.6595 |
| CNS Cancer | U251 | 78.38799 | −35.3044 | 56.1223 | 63.59794 | 50.2764 |
| Melanoma | LOX IMVI | 86.99522 | −83.8894 | −89.8952 | 65.75543 | 33.26765 |
| Melanoma | MALME-3M | 107.0521 | −71.7698 | −69.7804 | 59.5595 | −8.34344 |
| Melanoma | M14 | 98.61199 | −73.1499 | −75.6475 | 86.06322 | 21.90496 |
| Melanoma | MDA-MB-435 | 83.67863 | −57.8225 | −58.2282 | 77.28257 | 25.54192 |
| Melanoma | SK-MEL-2 | 104.3875 | 57.84086 | 70.47304 | 104.1313 | 96.72341 |
| Melanoma | SK-MEL-5 | 83.12211 | −3.69857 | 5.402367 | 97.38072 | 69.97823 |
| Melanoma | UACC-257 | 71.74632 | −32.9601 | −39.0086 | 91.12031 | 76.76646 |
| Melanoma | UACC-62 | 98.61959 | −52.4398 | −57.758 | 88.004 | 0.997215 |
| Ovarian Cancer | IGROV1 | 68.67848 | 44.22333 | 78.33071 | 100.6006 | 41.73694 |
| Ovarian Cancer | OVCAR-3 | 20.45842 | −23.663 | −30.7372 | 43.52243 | 5.413319 |
| Ovarian Cancer | OVCAR-4 | 58.84914 | 51.31999 | 98.13095 | 6.040094 | 12.23741 |
| Ovarian Cancer | OVCAR-5 | 97.57641 | 53.28481 | 91.29471 | 11.15693 | 77.12087 |
| Ovarian Cancer | OVCAR-8 | 86.9118 | 27.33993 | 68.23993 | 83.3637 | 60.52276 |
| Ovarian Cancer | NCI/ADR-RES | 92.66711 | 52.18646 | 76.11521 | 72.41172 | 66.76373 |
| Ovarian Cancer | SK-OV-3 | 103.7568 | 79.51289 | 86.95434 | 87.6273 | 98.32399 |
| Renal Cancer | 786-0 | 88.22138 | −44.1831 | 73.41071 | 100.1067 | 68.3555 |
| Renal Cancer | A498 | 70.32967 | 53.98858 | 46.17337 | 89.09644 | 62.96812 |
| Renal Cancer | ACHN | 77.87431 | −54.7701 | −86.4619 | 88.67147 | 36.59481 |
| Renal Cancer | CAKI-1 | 93.44504 | 23.13603 | 94.68733 | 28.3672 | 36.65925 |
| Renal Cancer | RXF 393 | 72.29089 | 18.74549 | 45.96231 | 52.44572 | 33.76924 |
| Renal Cancer | SN12C | 77.91783 | 31.6819 | 78.47419 | 54.07432 | 54.47064 |
| Renal Cancer | TK-10 | 86.85583 | 57.60967 | 90.98451 | 74.97901 | 63.68393 |
| Renal Cancer | UO-31 | 63.32941 | 23.82761 | 69.07386 | 66.56503 | 29.10216 |
| Prostate Cancer | PC-3 | 83.07756 | 39.37926 | 69.51624 | 29.0295 | 60.96073 |
| Prostate Cancer | DU-145 | 80.67995 | 53.10317 | 78.78831 | 63.01379 | 63.30395 |
| Breast Cancer | MCF7 | 57.27496 | −2.14482 | 20.05966 | 75.22164 | 2.310697 |
| Breast Cancer | MDA-MB- | 89.17445 | −29.6765 | 71.84716 | 10.16076 | 59.41615 |
| | 231/ATCC | | | | | |
| Breast Cancer | HS 578T | 79.89354 | 51.40995 | 69.51314 | 71.76825 | 69.28373 |
| Breast Cancer | BT-549 | 93.26317 | 80.08238 | 83.21918 | 82.21634 | 77.38403 |
| Breast Cancer | T-47D | 32.641 | −49.3289 | 83.88074 | 18.04511 | 14.34482 |
| Breast Cancer | MDA-MB-468 | 9.358453 | −28.0145 | 79.14835 | −5.12406 | 6.238015 |

GIPRCNT: % Growth inhibition
Tests performed at NCI.

Cell Culture. MIA PaCa-2 pancreatic cancer cell lines were obtained from the ATCC. MIA PaCa-2 cells were cultured as monolayer and maintained in RPMI1640 supplemented with 10% fetal bovine serum (FBS) in a humidified atmosphere with 5% CO₂ at 37° C.

Bru-seq Analysis for Nascent RNA Synthesis. Briefly, 4×106 MIA PaCa-2 cells were placed in 10 cm dishes on Day 1. On Day 2, cells were treated with DMSO, JR-1-235 or JR-1-272 for 4 h. Bromouridine was added into the media to label newly synthesized nascent RNA during the last 30 min of treatment to a final concentration of 2 mM. Cells were then collected in TRIZOL, and total RNA was isolated. Bromouridine-containing RNA was immunocaptured from total RNA, converted into cDNA libraries and deep sequenced at the University of Michigan Sequencing Core. Sequencing reads were mapped to the HG19 reference genome. Preranked gene lists were generated for each treatment through ranking genes by fold changes in RNA synthesis levels compared with control, and analyzed with GSEA (Broad Institute, MA).

General Methods. Reagents and anhydrous solvents were used without further purification and purchased from commercial sources. A Biotage Initiator+ was used to perform microwave catalyzed reactions in sealed vials. Reaction progress was monitored by UV absorbance using thin-layer chromatography (TLC) on aluminum-backed precoated silica plates from Silicycle (SiliaPlate, 200 μm thickness, F254). Purifications using flash chromatography were performed using Silicycle silica gel (SiliaFlash F60, 40-63 μm, 230-400 mesh, PN R10030B), and a small percentage of compounds were purified using a Biotage Isolera chromatography system equipped with 10 and 25 g Ultra-SNAP Cartridge columns (25 μM spherical silica). Glassware for reactions were oven-dried in preparation, and reactions were performed using nitrogen or argon atmosphere using standard inert conditions. ¹H NMR spectra were obtained using a Bruker (300 or 400 MHz) instrument. Spectral data are reported using the following abbreviations: s=singlet, d=doublet, t=triplet, q=quartet, m=multiplet, dd=doublet of doublets, and coupling constants are reported in Hz, followed by integration. A Shimadzu LCMS 20-20 system was utilized for generating HPLC traces, obtaining mass spectrometry data, and evaluating purity. The system is equipped with a PDA UV detector and Kinetex 2.6 μm, XB-C18 100 Å, 75 mm×4.6 mm column, which was used at room temperature. HPLC gradient method utilized a 1% to 90% MeCN in H₂O with 0.01% formic acid over 20 min with a 0.50 mL/min flow rate. Purity of final compounds (>95%) was assessed at 254 nm using the described column and method. Reverse-phase preparatory purifications were performed on a Shimadzu LC-20 modular HPLC system. This system utilized a PDA detector and a Kinetex 5 μm XB-C18 100 Å, 150 mm×21.2 mm column. Purification methods used a 27 min gradient from 10% to 90% MeCN in H₂O with 0.02% trifluoroacetic acid.

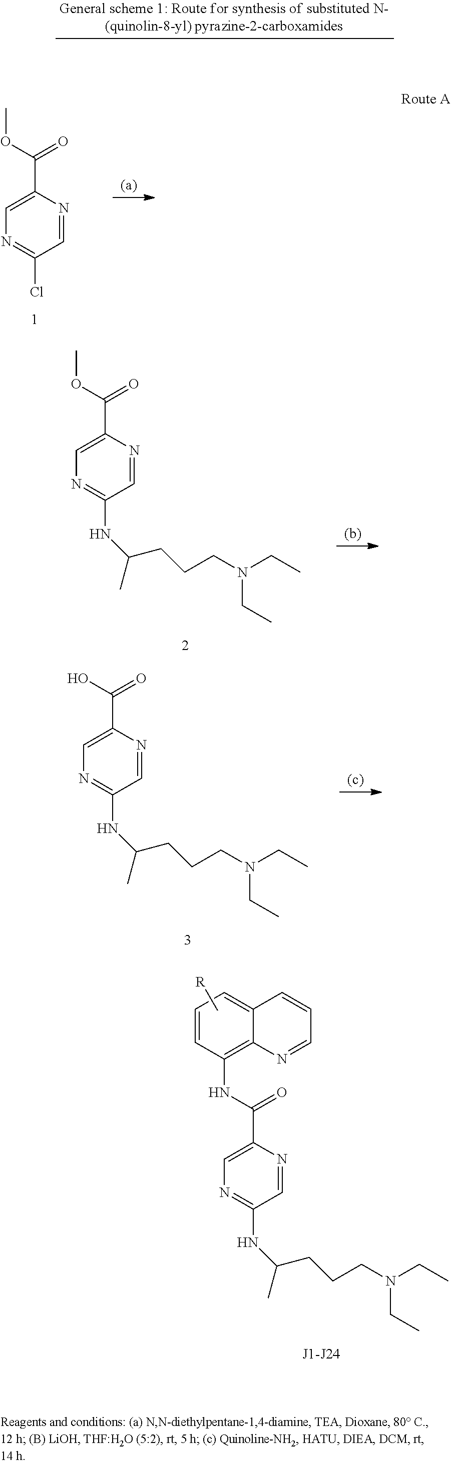

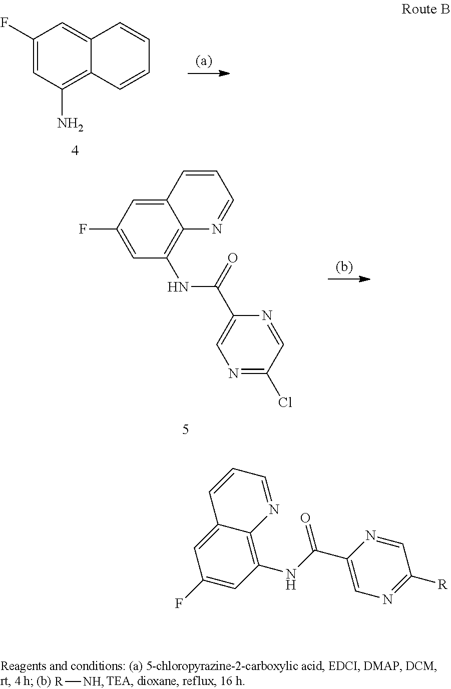

General Protocol A, Substitution of Chlorine with Amine:

To a stirred solution of 5-chloro-N-(6-fluoroquinolin-8-yl)pyrazine-2-carboxamide (5) (1 mmol) in dioxane (5 mL), appropriate amine (1.5 mmol) was added in the presence of triethylamine (2 equiv). The reaction mixture was heated to 80° C. for 12-14 h. Upon cooling, the solvent was removed under vacuum. The crude was purified by column chromatography using DCM/MeOH (95:5) or reverse phase HPLC (MeCN/water) to yield the corresponding compounds.

### General Protocol B, Basic Hydrolysis of Ester:

Ester derivatives (1 mmol) were dissolved in tetrahydrofuran/water (5:1, 10 ml), treated with lithium hydroxide (1.5 mmol) and stirred at room temperature for 5-8 h. Upon completion, solvent was concentrated; washed with DCM (2×). The aqueous layer was acidified with HCl until pH 2-3 was reached and then extracted with DCM (3×). The organic layer was dried with MgSO₄ and concentrated, the crude was used without further purification.

General Protocol C, Amidation (See, El-Faham, A.; et al., Chem. Rev., 2011, III, 6557-6602):

To a stirred solution of corresponding acid (1.0 mmol) and amine (1.0 mmol) in DCM or DMF, DIEPA (3 mmol) and HATU (1.5 or 2.0 mmol) were added at room temperature. The resulting mixture was stirred at that temperature for 12-16 h. On completion of the reaction as monitored by TLC or LCMS, it was diluted with DCM and washed with water two times. The solvent was concentrated. Purification of the crude was done either by column chromatography using DCM/MeOH (9:1) or reverse phase HPLC (MeCN/water).

### J1: 5-((5-(diethylamino)pentan-2-yl)amino)-N-(2-methylquinolin-8-yl)pyrazine-2-carboxamide

5-((5-(diethylamino)pentan-2-yl)amino)pyrazine-2-carboxylic acid (3) (44 mg, 0.16 mmol), 2-methylquinolin-8-amine (25 mg, 0.16 mmol), HATU (91 mg, 0.24 mmol), and DIEPA (0.08 mL, 0.48 mmol) were dissolved in 5 mL DMF. Following general protocol C, 5-((5-(diethylamino)pentan-2-yl)amino)-N-(2-methylquinolin-8-yl)pyrazine-2-carboxamide was recovered as a brown liquid (24 mg, 38%). ¹H NMR (300 MHz, CD₃OD-d₄) δ 8.83-8.67 (m, 2H), 8.28 (d, J=8.4 Hz, 1H), 8.03 (d, J=1.3 Hz, 1H), 7.65 (dd, J=8.4, 1.4 Hz, 1H), 7.55 (dd, J=16.4, 8.1 Hz, 2H), 4.26 (q, J=6.5 Hz, 1H), 3.23 (q, J=7.4 Hz, 6H), 2.82 (s, 3H), 1.94-1.58 (m, 4H), 1.31 (t, J=7.2 Hz, 9H). LCMS (ESI) 421.3 [M+H]⁺.

### J2: 5-((5-(diethylamino)pentan-2-yl)amino)-N-(3-methylquinolin-8-yl)pyrazine-2-carboxamide

5-((5-(diethylamino)pentan-2-yl)amino)pyrazine-2-carboxylic acid (3) (44 mg, 0.16 mmol), 3-methylquinolin-8-amine (25 mg, 0.16 mmol), HATU (91 mg, 0.24 mmol), and DIEPA (0.08 mL, 0.48 mmol) were dissolved in 5 mL DMF. Following general protocol C₁₋₅-((5-(diethylamino)pentan-2-yl)amino)-N-(3-methylquinolin-8-yl)pyrazine-2-carboxamide was recovered as a brown liquid (26 mg, 40%). ¹H NMR (300 MHz, CD₃OD-d₄) δ 8.89-8.64 (m, 3H), 8.10 (s, 1H), 8.02 (s, 1H), 7.66-7.47 (m, 2H), 4.27 (q, J=6.5 Hz, 1H), 3.23 (q, J=7.5 Hz, 6H), 2.57 (s, 3H), 1.92-1.57 (m, 4H), 1.31 (t, J=6.9 Hz, 9H). LCMS (ESI) 421.3 [M+H]⁺.

### J3: 5-((5-(diethylamino)pentan-2-yl)amino)-N-(5-methylquinolin-8-yl)pyrazine-2-carboxamide

5-((5-(diethylamino)pentan-2-yl)amino)pyrazine-2-carboxylic acid (3) (44 mg, 0.16 mmol), 5-methylquinolin-8-amine (25 mg, 0.16 mmol), HATU (91 mg, 0.24 mmol), and DIEPA (0.8 mL, 0.48 mmol) were dissolved in 5 mL DMF. Following general protocol C₁₋₅-((5-(diethylamino)pentan-2-yl)amino)-N-(5-methylquinolin-8-yl)pyrazine-2-carboxamide was recovered as a yellow liquid (23 mg, 38%). ¹H NMR (300 MHz, CD₃OD-d₄) δ 8.99-8.90 (m, 1H), 8.80-8.66 (m, 2H), 8.49 (dd, J=8.5, 1.6 Hz, 1H), 7.99 (d, J=1.3 Hz, 1H), 7.62 (dd, J=8.5, 4.2 Hz, 1H), 7.43 (d, J=7.9 Hz, 1H), 4.33-4.06 (m, 1H), 2.68 (s, 3H), 2.57 (q, J=7.2 Hz, 6H), 1.60 (s, 4H), 1.28 (d, J=6.5 Hz, 3H), 1.05 (t, J=7.2 Hz, 6H). LCMS (ESI) 421.3 [M+H]⁺.

### J4:5-((5-(diethylamino)pentan-2-yl)amino)-N-(6-methylquinolin-8-yl)pyrazine-2-carboxamide

5-((5-(diethylamino)pentan-2-yl)amino)pyrazine-2-carboxylic acid (3) (44 mg, 0.16 mmol), 6-methylquinolin-8-amine (25 mg, 0.16 mmol), HATU (91 mg, 0.24 mmol), and DIEPA (0.08 mL, 0.48 mmol) were dissolved in 5 mL DMF. Following general protocol C₁₋₅-((5-(diethylamino)pentan-2-yl)amino)-N-(6-methylquinolin-8-yl)pyrazine-2-carboxamide was recovered as a yellow liquid (32 mg, 47%). ¹H NMR (300 MHz, CD₃OD-d₄) δ 11.45 (s, 1H), 8.89 (dd, J=7.1, 1.9 Hz, 1H), 8.75 (d, J=1.3 Hz, 1H), 8.33 (d, J=8.6 Hz, 1H), 8.03 (d, J=1.3 Hz, 1H), 7.70-7.52 (m, 3H), 4.27 (q, J=6.5 Hz, 1H), 3.23 (q, J=8.7, 8.0 Hz, 6H), 2.58 (s, 3H), 1.77 (dt, J=30.5, 7.8 Hz, 4H), 1.31 (t, J=7.2 Hz, 9H). LCMS (ESI) 421.3 [M+H]⁺.

### J5: 5-((5-(diethylamino)pentan-2-yl)amino)-N-(7-methylquinolin-8-yl)pyrazine-2-carboxamide

5-((5-(diethylamino)pentan-2-yl)amino)pyrazine-2-carboxylic acid (3) (44 mg, 0.16 mmol), 7-methylquinolin-8-amine (25 mg, 0.16 mmol), HATU (91 mg, 0.24 mmol), and DIEPA (0.08 mL, 0.48 mmol) were dissolved in 5 mL DMF. Following general protocol C₁₋₅-((5-(diethylamino)pentan-2-yl)amino)-N-(7-methylquinolin-8-yl)pyrazine-2-carboxamide was recovered as a brown liquid (25 mg, 37%). ¹H NMR (300 MHz, CD₃OD-d₄) δ 9.07 (t, J=7.1 Hz, 2H), 8.74 (d, J=1.3 Hz, 1H), 8.21 (d, J=8.5 Hz, 1H), 8.04 (d, J=1.3 Hz, 1H), 8.02-7.83 (m, 2H), 4.28 (q, J=6.5 Hz, 1H), 3.24 (q, J=7.4 Hz, 6H), 2.61 (s, 3H), 2.00-1.56 (m, 4H), 1.48-1.06 (m, 9H). LCMS (ESI) 421.3 [M+H]⁺.

### J6: N-(2-chloroquinolin-8-yl)-5-((5-(diethylamino)pentan-2-yl)amino)pyrazine-2-carboxamide

5-((5-(diethylamino)pentan-2-yl)amino)pyrazine-2-carboxylic acid (3) (31 mg, 0.11 mmol), 2-chloroquinolin-8-amine (20 mg, 0.11 mmol), HATU (65 mg, 0.17 mmol), and DIEPA (0.06 mL, 0.33 mmol) were dissolved in 5 mL DMF. Following general protocol C, N-(2-chloroquinolin-8-yl)-5-((5-(diethylamino)pentan-2-yl)amino)pyrazine-2-carboxamide was recovered as a brown liquid (28 mg, 56%). ¹H NMR (300 MHz, CD₃OD-d₄) δ 11.45 (s, 1H), 8.89 (dd, J=7.1, 1.9 Hz, 1H), 8.75 (d, J=1.3 Hz, 1H), 8.33 (d, J=8.6 Hz, 1H), 8.03 (d, J=1.3 Hz, 1H), 7.70-7.52 (m, 3H), 4.27 (q, J=6.5 Hz, 1H), 3.23 (q, J=8.7, 8.0 Hz, 6H), 1.77 (dt, J=30.4, 7.8 Hz, 4H), 1.31 (t, J=7.2 Hz, 9H). LCMS (ESI) 441.2 [M+H]⁺.

### J7: N-(3-chloroquinolin-8-yl)-5-((5-(diethylamino)pentan-2-yl)amino)pyrazine-2-carboxamide

5-((5-(diethylamino)pentan-2-yl)amino)pyrazine-2-carboxylic acid (3) (31 mg, 0.11 mmol), 3-chloroquinolin-8-amine (20 mg, 0.11 mmol), HATU (65 mg, 0.17 mmol), and DIEPA (0.06 mL, 0.33 mmol) were dissolved in 5 mL DMF. Following general protocol C, N-(3-chloroquinolin-8-yl)-5-((5-(diethylamino)pentan-2-yl)amino)pyrazine-2-carboxamide was recovered as a brown liquid (24 mg, 48%). ¹H NMR (300 MHz, CD₃OD-d₄) δ 8.87 (td, J=3.3, 2.0 Hz, 2H), 8.77 (d, J=1.3 Hz, 1H), 8.42 (d, J=2.4 Hz, 1H), 8.01 (d, J=1.3 Hz, 1H), 7.76-7.53 (m, 2H), 4.28 (q, J=6.5 Hz, 1H), 3.22 (q, J=7.4 Hz, 6H), 1.90-1.53 (m, 4H), 1.31 (t, J=7.2 Hz, 9H). LCMS (ESI) 441.2 [M+H]⁺.

### J8: N-(4-chloroquinolin-8-yl)-5-((5-(diethylamino)pentan-2-yl)amino)pyrazine-2-carboxamide

5-((5-(diethylamino)pentan-2-yl)amino)pyrazine-2-carboxylic acid (3) (31 mg, 0.11 mmol), 4-chloroquinolin-8-amine (20 mg, 0.11 mmol), HATU (65 mg, 0.17 mmol), and DIEPA (0.06 mL, 0.33 mmol) were dissolved in 5 mL DMF. Following general protocol C, N-(4-chloroquinolin-8-yl)-5-((5-(diethylamino)pentan-2-yl)amino)pyrazine-2-carboxamide was recovered as a brownish yellow liquid (21 mg, 44%). ¹H NMR (300 MHz, CD₃OD-d₄) δ 8.89 (dd, J=7.8, 1.2 Hz, 1H), 8.78 (d, J=4.7 Hz, 1H), 8.71 (d, J=1.3 Hz, 1H), 7.97 (d, J=1.3 Hz, 1H), 7.91 (d, J=8.5 Hz, 1H), 7.75-7.61 (m, 2H), 4.25 (q, J=6.5 Hz, 1H), 3.22 (qt, J=7.1, 4.7 Hz, 6H), 1.75 (dq, J=29.6, 7.4 Hz, 4H), 1.35-1.22 (m, 9H). LCMS (ESI) 441.2 [M+H]⁺.

### J9: N-(5-chloroquinolin-8-yl)-5-((5-(diethylamino)pentan-2-yl)amino)pyrazine-2-carboxamide

5-((5-(diethylamino)pentan-2-yl)amino)pyrazine-2-carboxylic acid (3) (31 mg, 0.11 mmol), 5-chloroquinolin-8-amine (20 mg, 0.11 mmol), HATU (65 mg, 0.17 mmol), and DIEPA (0.06 mL, 0.33 mmol) were dissolved in 5 mL DMF. Following general protocol C, N-(5-chloroquinolin-8-yl)-5-((5-(diethylamino)pentan-2-yl)amino)pyrazine-2-carboxamide was recovered as a dark yellow semi solid (14 mg, 28%). ¹H NMR (300 MHz, CD₃OD-d₄) δ 9.16 (d, J=8.2 Hz, 1H), 9.03 (d, J=5.7 Hz, 1H), 8.74 (d, J=1.3 Hz, 1H), 8.40 (d, J=9.3 Hz, 1H), 8.02 (d, J=10.0 Hz, 2H), 7.93 (dd, J=8.2, 5.5 Hz, 1H), 4.27 (d, J=6.8 Hz, 1H), 3.29-3.13 (m, 6H), 1.77 (dd, J=30.4, 7.2 Hz, 4H), 1.43-1.18 (m, 9H). LCMS (ESI) 441.2 [M+H]⁺.

### J10: N-(6-chloroquinolin-8-yl)-5-((5-(diethylamino)pentan-2-yl)amino)pyrazine-2-carboxamide

5-((5-(diethylamino)pentan-2-yl)amino)pyrazine-2-carboxylic acid (3) (31 mg, 0.11 mmol), 6-chloroquinolin-8-amine (20 mg, 0.11 mmol), HATU (65 mg, 0.17 mmol), and DIEPA (0.6 mL, 0.33 mmol) were dissolved in 5 mL DMF. Following general protocol C, N-(6-chloroquinolin-8-yl)-5-((5-(diethylamino)pentan-2-yl)amino)pyrazine-2-carboxamide was recovered as brown liquid (10 mg, 21%). ¹H NMR (300 MHz, CD₃OD-d₄) δ 8.96-8.90 (m, 1H), 8.87 (d, J=2.2 Hz, 1H), 8.78 (d, J=1.3 Hz, 1H), 8.35-8.24 (m, 1H), 8.02 (d, J=1.3 Hz, 1H), 7.69 (d, J=2.3 Hz, 1H), 7.63 (dd, J=8.3, 4.3 Hz, 1H), 4.28 (q, J=6.6 Hz, 1H), 3.23 (q, J=7.3 Hz, 6H), 1.77 (dt, J=29.7, 7.6 Hz, 4H), 1.41-1.20 (m, 9H). LCMS (ESI) 441.2 [M+H]⁺.

### J11: 5-((5-(diethylamino)pentan-2-yl)amino)-N-(2-methoxyquinolin-8-yl)pyrazine-2-carboxamide

5-((5-(diethylamino)pentan-2-yl)amino)pyrazine-2-carboxylic acid (3) (31 mg, 0.11 mmol), 2-methoxyquinolin-8-amine (20 mg, 0.11 mmol), HATU (65 mg, 0.17 mmol), and DIEPA (0.06 mL, 0.33 mmol) were dissolved in 5 mL DMF. Following general protocol C, 5-((5-(diethylamino)pentan-2-yl)amino)-N-(2-methoxyquinolin-8-yl)pyrazine-2-carboxamide was recovered as brown liquid (23 mg, 47%). ¹H NMR (300 MHz, CD₃OD-d₄) δ 11.59 (s, 1H), 8.74-8.59 (m, 2H), 8.09 (d, J=8.9 Hz, 1H), 7.87 (d, J=1.3 Hz, 1H), 7.49 (dd, J=8.2, 1.3 Hz, 1H), 7.37 (t, J=7.9 Hz, 1H), 6.96 (d, J=8.8 Hz, 1H), 4.24 (q, J=6.5 Hz, 1H), 4.12 (s, 3H), 3.23 (q, J=7.3 Hz, 6H), 1.76 (dt, J=30.3, 8.5 Hz, 4H), 1.31 (t, J=12 Hz, 9H). LCMS (ESI) 437.3 [M+H]⁺.

### J12: 5-((5-(diethylamino)pentan-2-yl)amino)-N-(5-methoxyquinolin-8-yl)pyrazine-2-carboxamide

5-((5-(diethylamino)pentan-2-yl)amino)pyrazine-2-carboxylic acid (3) (31 mg, 0.11 mmol), 5-methoxyquinolin-8-amine (20 mg, 0.11 mmol), HATU (65 mg, 0.17 mmol), and DIEPA (0.06 mL, 0.33 mmol) were dissolved in 5 mL DMF. Following general protocol C, 5-((5-(diethylamino)pentan-2-yl)amino)-N-(5-methoxyquinolin-8-yl)pyrazine-2-carboxamide was recovered as yellow liquid (20 mg, 41%). ¹H NMR (300 MHz, CD₃OD-d₄) δ 8.92 (dd, J=4.3, 1.7 Hz, 1H), 8.75-8.60 (m, 3H), 7.99 (d, J=1.3 Hz, 1H), 7.57 (dd, J=8.5, 4.3 Hz, 1H), 7.01 (d, J=8.6 Hz, 1H), 4.24 (q, J=6.5 Hz, 1H), 4.04 (s, 3H), 3.29-3.10 (m, 6H), 1.74 (ddd, J=33.9, 14.6, 7.3 Hz, 4H), 1.41-1.19 (m, 9H). LCMS (ESI) 437.3 [M+H]⁺.

### J13: 5-((5-(diethylamino)pentan-2-yl)amino)-N-(6-methoxyquinolin-8-yl)pyrazine-2-carboxamide

5-((5-(diethylamino)pentan-2-yl)amino)pyrazine-2-carboxylic acid (3) (31 mg, 0.11 mmol), 6-methoxyquinolin-8-amine (20 mg, 0.11 mmol), HATU (65 mg, 0.17 mmol), and DIEPA (0.06 mL, 0.33 mmol) were dissolved in 5 mL DMF. Following general protocol C₁₋₅-((5-(diethylamino)pentan-2-yl)amino)-N-(6-methoxyquinolin-8-yl)pyrazine-2-carboxamide was recovered as yellow liquid (18 mg, 36%). ¹H NMR (300 MHz, CD₃OD-d₄) δ 8.73 (dd, J=6.7, 1.5 Hz, 2H), 8.51 (d, J=2.7 Hz, 1H), 8.20 (dd, J=8.3, 1.6 Hz, 1H), 8.00 (d, J=1.3 Hz, 1H), 7.50 (dd, J=8.3, 4.2 Hz, 1H), 7.01 (d, J=2.7 Hz, 1H), 4.26 (q, J=6.5 Hz, 1H), 3.96 (s, 3H), 3.23 (q, J=7.6 Hz, 6H), 1.76 (dt, J=31.1, 7.6 Hz, 4H), 1.49-1.13 (m, 9H). LCMS (ESI) 437.3 [M+H]⁺.

### J14: 5-((5-(diethylamino)pentan-2-yl)amino)-N-(7-methoxyquinolin-8-yl)pyrazine-2-carboxamide

5-((5-(diethylamino)pentan-2-yl)amino)pyrazine-2-carboxylic acid (3) (31 mg, 0.11 mmol), 7-methoxyquinolin-8-amine (20 mg, 0.11 mmol), HATU (65 mg, 0.17 mmol), and DIEPA (0.06 mL, 0.33 mmol) were dissolved in 5 mL DMF. Following general protocol C₁₋₅-((5-(diethylamino)pentan-2-yl)amino)-N-(7-methoxyquinolin-8-yl)pyrazine-2-carboxamide was recovered as yellow liquid (17 mg, 35%). ¹H NMR (300 MHz, CD₃OD-d₄) δ 9.16 (d, J=8.2 Hz, 1H), 9.03 (d, J=5.7 Hz, 1H), 8.74 (d, J=1.3 Hz, 1H), 8.40 (d, J=9.3 Hz, 1H), 8.02 (d, J=10.0 Hz, 2H), 7.93 (dd, J=8.2, 5.5 Hz, 1H), 4.27 (d, J=6.8 Hz, 1H), 4.18 (s, 3H), 3.29-3.12 (m, 6H), 1.77 (dd, J=30.5, 7.3 Hz, 4H), 1.40-1.24 (m, 9H). LCMS (ESI) 437.3 [M+H]⁺.

### J15: 5-((5-(diethylamino)pentan-2-yl)amino)-N-(2,5-dimethoxyquinolin-8-yl)pyrazine-2-carboxamide

5-((5-(diethylamino)pentan-2-yl)amino)pyrazine-2-carboxylic acid (3) (15 mg, 0.05 mmol), 5-dimethoxyquinolin-8-amine (10 mg, 0.05 mmol), HATU (29 mg, 0.08 mmol), and DIEPA (0.03 mL, 0.15 mmol) were dissolved in 5 mL DMF. Following general protocol C, 5-((5-(diethylamino)pentan-2-yl)amino)-N-(2,5-dimethoxyquinolin-8-yl)pyrazine-2-carboxamide was recovered as yellow liquid (8 mg, 34%). ¹H NMR (300 MHz, CD₃OD-d₄) δ 11.39 (s, 1H), 8.70 (s, 1H), 8.62 (d, J=8.6 Hz, 1H), 8.42 (d, J=9.0 Hz, 1H), 7.91 (s, 1H), 6.95 (d, J=9.0 Hz, 1H), 6.85 (d, J=8.6 Hz, 1H), 4.25 (q, J=6.5 Hz, 1H), 4.15 (s, 3H), 4.00 (s, 3H), 3.23 (q, J=7.6 Hz, 6H), 1.94-1.60 (m, 4H), 1.31 (t, J=6.9 Hz, 9H). LCMS (ESI) 467.7 [M+H]⁺.

### J16: 5-((5-(diethylamino)pentan-2-yl)amino)-N-(5-fluoroquinolin-8-yl)pyrazine-2-carboxamide

5-((5-(diethylamino)pentan-2-yl)amino)pyrazine-2-carboxylic acid (3) (17 mg, 0.06 mmol), 5-fluoroquinolin-8-amine (10 mg, 0.06 mmol), HATU (35 mg, 0.09 mmol), and DIEPA (0.4 mL, 0.18 mmol) were dissolved in 5 mL DMF. Following general protocol C₁₋₅-((5-(diethylamino)pentan-2-yl)amino)-N-(5-fluoroquinolin-8-yl)pyrazine-2-carboxamide was recovered as yellow liquid (8 mg, 31%). ¹H NMR (300 MHz, CD₃OD-d₄) δ 9.01 (dd, J=4.3, 1.6 Hz, 1H), 8.82 (dd, J=8.7, 5.4 Hz, 1H), 8.75 (d, J=1.3 Hz, 1H), 8.53 (dd, J=8.5, 1.6 Hz, 1H), 8.01 (d, J=1.3 Hz, 1H), 7.69 (dd, J=8.5, 4.3 Hz, 1H), 7.42-7.23 (m, 1H), 4.27 (q, J=6.7 Hz, 1H), 3.32-3.12 (m, 6H), 1.98-1.55 (m, 4H), 1.45-1.09 (m, 9H). LCMS (ESI) 425.4 [M+H]⁺.

### J17: 5-((5-(diethylamino)pentan-2-yl)amino)-N-(6-fluoroquinolin-8-yl)pyrazine-2-carboxamide

5-chloropyrazine-2-carboxylic acid (300 mg, 2 mmol) and 6-fluoroquinolin-8-amine (320 mg, 2 mmol) using EDCI (300 mg, 4 mmol) and DMAP (cat.) were dissolved in DCM following the general protocol for amidation (route B). The crude was purified by crystallization in DCM/MeOH and subjected to the next reaction. 5-chloro-N-(6-fluoroquinolin-8-yl)pyrazine-2-carboxamide (100 mg, 0.33 mmol) on treatment with N,N-diethylpentane-1,4-diamine (57 mg, 0.36 mmol) and triethyl amine (0.12 mL, 0.82 mmol) in 5 mL dioxane under refluxing condition afforded the title compound as yellow liquid. Yield 65%. ¹H NMR (300 MHz, CD₃OD-d₄) δ 8.74 (dd, J=4.3, 1.5 Hz, 1H), 8.64 (d, J=1.3 Hz, 1H), 8.52 (dd, J=11.3, 2.8 Hz, 1H), 8.17 (dd, J=8.3, 1.6 Hz, 1H), 7.90 (d, J=1.3 Hz, 1H), 7.49 (dd, J=8.3, 4.2 Hz, 1H), 7.17 (dd, J=9.0, 2.8 Hz, 1H), 4.21 (q, J=6.5 Hz, 1H), 3.21 (p, J=7.0 Hz, 6H), 1.76 (dt, J=35.8, 7.9 Hz, 4H), 1.39-1.20 (m, 9H). LCMS (ESI) 425.4 [M+H]⁺

### J18: 5-((5-(diethylamino)pentan-2-yl)amino)-N-(6-(trifluoromethyl)quinolin-8-yl)pyrazine-2-carboxamide

5-((5-(diethylamino)pentan-2-yl)amino)pyrazine-2-carboxylic acid (3) (15 mg, 0.05 mmol), 6-(trifluoromethyl)quinolin-8-amine (10 mg, 0.05 mmol), HATU (29 mg, 0.08 mmol), and DIEPA (0.03 mL, 0.15 mmol) were dissolved in 5 mL DMF. Following general protocol C, 5-((5-(diethylamino)pentan-2-yl)amino)-N-(6-(trifluoromethyl)quinolin-8-yl)pyrazine-2-carboxamide was recovered as yellow liquid (3 mg, 11%). ¹H NMR (300 MHz, CD₃OD-d₄) δ 9.19 (dd, J=4.3, 1.5 Hz, 1H), 8.91 (d, J=1.3 Hz, 1H), 8.55 (dd, J=8.4, 1.6 Hz, 1H), 8.25 (d, J=6.5 Hz, 1H), 8.10-7.98 (m, 2H), 7.81 (d, J=6.5 Hz, 1H), 4.32 (q, J=6.6 Hz, 1H), 3.24 (q, J=7.3 Hz, 6H), 1.78 (dq, J=21.0, 7.5 Hz, 4H), 1.32 (t, J=7.3 Hz, 9H). LCMS (ESI) 475.2 [M+H]⁺.

### J19: 5-((5-(diethylamino)pentan-2-yl)amino)-N-(6-hydroxyquinolin-8-yl)pyrazine-2-carboxamide

To a solution of J13 (20 mg, 0.11 mmol) in DCM at −78° C., BBr₃ (0.15 mL, 0.15 mmol) was added and stirred for 2 h. Then the temperature was raised to 25° C. and stirring continued for another 8 h. Reaction was quenched by addition of MeOH, followed by extraction with DCM (3×) and the organic layer was washed with water and concentrated. Column chromatography using DCM/MeOH (9:1) afforded 5-((5-(diethylamino)pentan-2-yl)amino)-N-(6-hydroxyquinolin-8-yl)pyrazine-2-carbonamide as yellow solid (12 mg, 19%). ¹H NMR (300 MHz, CD₃OD-d₄) δ 8.76 (d, J=1.3 Hz, 1H), 8.73-8.62 (m, 1H), 8.49 (d, J=2.6 Hz, 1H), 8.20-8.09 (m, 1H), 8.02 (d, J=1.3 Hz, 1H), 7.49 (dd, J=8.3, 4.3 Hz, 1H), 6.91 (d, J=2.6 Hz, 1H), 4.27 (q, J=6.6 Hz, 1H), 3.23 (q, J=7.4 Hz, 6H), 1.77 (dt, J=31.0, 7.8 Hz, 4H), 1.41-1.20 (m, 9H). LCMS (ESI) 423.2 [M+H]⁺.

### J20: 5-((5-(diethylamino)pentan-2-yl)amino)-N-(1,7-naphthyridin-8-yl)pyrazine-2-carboxamide

5-((5-(diethylamino)pentan-2-yl)amino)pyrazine-2-carboxylic acid (3) (15 mg, 0.05 mmol), 1,7-naphthyridin-8-amine (8 mg, 0.05 mmol), HATU (29 mg, 0.08 mmol), and DIEPA (0.03 mL, 0.15 mmol) were dissolved in 5 mL DMF. Following general protocol C₁₋₅-((5-(diethylamino)pentan-2-yl)amino)-N-(1,7-naphthyridin-8-yl)pyrazine-2-carboxamide was recovered as yellow liquid (4 mg, 21%). ¹H NMR (300 MHz, CD₃OD-d₄) δ 9.10 (dd, J=12.7, 3.0 Hz, 2H), 8.79 (d, J=1.3 Hz, 1H), 8.62-8.40 (m, 1H), 8.15-7.91 (m, 2H), 7.74 (dd, J=8.3, 4.3 Hz, 1H), 4.29 (q, J=6.4 Hz, 1H), 3.33-3.00 (m, 6H), 1.77 (dq, J=23.3, 7.9 Hz, 4H), 1.32 (t, J=7.0 Hz, 9H).

### J21: N-(6-cyanoquinoline-8-yl)-5-((5-(diethylamino)pentan-2-yl)amino)pyrazine-2-carboxamide

Treatment of A-(6-bromoquinolin-8-yl)-5-((5-(diethylamino)pentan-2-yl)amino)pyrazine-2-carboxamide (J22) (50 mg, 0.10 mmol) with Zn(CN)₂ (17.5 mg, 15 mmol) and Pd(PPh₃)₄ (10 mg, 0.009 mmol) in DMF (3 mL) at 100° C. for 12 h afforded the title compound. Crude was filtered through celite and concentrated. Purification was done by reverse phase HPLC to afford J21 as pale-yellow liquid (13 mg, 31%). ¹H NMR (300 MHz, CD₃OD-d₄) δ 9.05 (d, J=11.9 Hz, 2H), 8.79 (s, 1H), 8.47 (d, J=8.7 Hz, 1H), 8.16 (s, 1H), 8.01 (s, 1H), 7.74 (s, 1H), 4.30 (s, 1H), 3.26-3.15 (m, 6H), 1.77 (d, J=32.6 Hz, 4H), 1.32 (t, J=6.9 Hz, 9H). LCMS (ESI) 432.2 [M+H]⁺.

### J22: N-(6-bromoquinolin-8-yl)-5-((5-(diethylamino)pentan-2-yl)amino)pyrazine-2-carboxamide

Treatment of compound N-(6-bromoquinolin-8-yl)-5-chloropyrazine-2-carboxamide (5) (250 mg, 0.70 mmol) with N,N-diethylpentane-1,4-diamine (110 mg, 0.70 mmol) following the general protocol A afforded A-(6-bromoquinolin-8-yl)-5-((5-(diethylamino)pentan-2-yl)amino)pyrazine-2-carboxamide as brown liquid (104 mg, 31%). LCMS (ESI) 485.2 [M+H]⁺

### J23: N-(6-(4-chlorophenyl)quinolin-8-yl)-5-((5-(diethylamino)pentan-2-yl)amino)pyrazine-2-carboxamide

To a solution of A-(6-bromoquinolin-8-yl)-5-((5-(diethylamino)pentan-2-yl)amino)pyrazine-2-carboxamide (J22) (50 mg, 0.10 mmol) in DMF in sealed tube, 4-chlorophenylboronic acid pinacol ester (37 mg, 0.15 mmol), CsCO₃ (100 mg, 0.31 mmol) and Pd(dppf)Ch (5 mg, 0.03 mmol) were heated at 100° C. for 12 h. On completion the contents were filtered through celite pad and the filtrate was concentrated and purified by HPLC to afford dark brown powder (5 mg, 10%). ¹H NMR (300 MHz, CD₃OD-d₄) δ 9.17 (s, 1H), 8.91 (s, 1H), 8.77 (s, 1H), 8.38 (d, J=8.4 Hz, 1H), 8.02 (s, 1H), 7.87 (s, 1H), 7.81 (d, J=8.3 Hz, 2H), 7.60 (dd, J=8.4, 4.3 Hz, 1H), 7.53 (d, J=8.1 Hz, 2H), 4.27 (s, 1H), 3.23 (q, J=7.6 Hz, 6H), 1.78 (d, J=33.1 Hz, 4H), 1.32 (t, J=7.3 Hz, 9H). LCMS (ESI) 517.2 [M+H]⁺.

### J24: N-(5-chloro-6-methylquinolin-8-yl)-5-((5-(diethylamino)pentan-2-yl)amino)pyrazine-2-carboxamide

5-((5-(diethylamino)pentan-2-yl)amino)pyrazine-2-carboxylic acid (3) (28 mg, 0.10 mmol), 5-chloro-6-methylquinolin-8-amine (20 mg, 0.10 mmol), HATU (57 mg, 0.15 mmol), and DIEPA (0.06 mL, 0.30 mmol) were dissolved in 5 mL DMF. Following general protocol C, A-(5-chloro-6-methylquinolin-8-yl)-5-((5-(diethylamino)pentan-2-yl)amino)pyrazine-2-carboxamide was recovered as yellow liquid (7 mg, 19%). ¹H NMR (300 MHz, CD₃OD-d₄) δ 8.92 (d, J=4.2 Hz, 1H), 8.83 (s, 1H), 8.76 (d, J=1.2 Hz, 1H), 8.70-8.60 (m, 1H), 8.01 (d, J=1.3 Hz, 1H), 7.69 (dd, J=8.6, 4.2 Hz, 1H), 4.28 (q, J=6.5 Hz, 1H), 3.23 (q, J=7.4 Hz, 6H), 2.64 (s, 3H), 1.95-1.58 (m, 4H), 1.32 (t, J=7.0 Hz, 9H). LCMS (ESI) 455.6 [M+H]⁺.

### J25: N-(5-chloro-2-methoxyquinolin-8-yl)-5-((5-(diethylamino)pentan-2-yl)amino)pyrazine-2-carboxamide

5-((5-(diethylamino)pentan-2-yl)amino)pyrazine-2-carboxylic acid (3) (14 mg, 0.05 mmol), 5-chloro-2-methoxyquinolin-8-amine (10 mg, 0.05 mmol), HATU (38 mg, 0.10 mmol), and DIEPA (0.3 mL, 0.15 mmol) were dissolved in 5 mL DMF. Following general protocol C, N-(5-chloro-2-methoxyquinolin-8-yl)-5-((5-(diethylamino)pentan-2-yl)amino)pyrazine-2-carboxamide was recovered as brown liquid (1 mg, 5%). ¹H NMR (300 MHz, CD₃OD-d₄) δ 11.64 (s, 1H), 8.81-8.66 (m, 2H), 8.51 (d, J=9.1 Hz, 1H), 7.95 (s, 1H), 7.53 (d, J=8.4 Hz, 1H), 7.19 (d, J=9.1 Hz, 1H), 4.24 (m, 4H), 3.23 (q, J=7.3 Hz, 6H), 1.71 (s, 4H), 1.32 (t, J=6.8 Hz, 9H). LCMS (ESI) 471.4 [M+H]⁺.

### J26: N-(5-((2-(diethylamino)ethyl)amino)quinolin-8-yl)pyrazine-2-carboxamide

N⁵-(2-(diethylamino)ethyl)quinoline-5,8-diamine (80 mg, 0.31 mmol), pyrazine-2-carboxylic acid (38 mg, 0.31 mmol), HATU (235 mg, 0.62 mmol), and DIEPA (0.2 mL, 0.93 mmol) were dissolved in 5 mL DMF. Following general protocol C, N-(5-((2-(diethylamino)ethyl)amino)quinolin-8-yl)pyrazine-2-carboxamide was recovered as brown liquid (18 mg, 16%). ¹H NMR (300 MHz, CD₃OD-d₄) δ 9.40 (d, J=1.4 Hz, 1H), 8.97 (dd, J=4.4, 1.5 Hz, 1H), 8.87 (d, J=2.5 Hz, 1H), 8.83-8.80 (m, 1H), 8.76 (dd, J=8.6, 1.5 Hz, 1H), 8.66 (d, J=8.5 Hz, 1H), 7.66 (dd, J=8.6, 4.4 Hz, 1H), 6.89 (d, J=8.5 Hz, 1H), 3.79 (t, J=6.1 Hz, 2H), 3.56 (t, J=6.1 Hz, 2H), 3.43-3.35 (m, 4H), 1.37 (t, J=7.3 Hz, 6H).

### J27: N-(5-((2-(diethylamino)ethyl)amino)quinolin-8-yl)-5-methylpyrazine-2-carboxamide

N⁵-(2-(diethylamino)ethyl)quinoline-5,8-diamine (80 mg, 0.31 mmol), 5-methylpyrazine-2-carboxylic acid (42 mg, 0.31 mmol), HATU (235 mg, 0.62 mmol), and DIEPA (0.2 mL, 0.93 mmol) were dissolved in 5 mL DMF. Following general protocol C, N-(5-((2-(diethylamino)ethyl)amino)quinolin-8-yl)-5-methylpyrazine-2-carboxamide was recovered as brown liquid (23 mg, 20%). ¹H NMR (300 MHz, CD₃OD-d₄) δ 9.25 (d, J=1.4 Hz, 1H), 9.00-8.92 (m, 1H), 8.79-8.66 (m, 2H), 8.65 (dd, J=8.5, 1.5 Hz, 1H), 7.61 (dd, J=8.6, 4.3 Hz, 1H), 6.87 (d, J=8.5 Hz, 1H), 3.78 (t, J=6.1 Hz, 2H), 3.56 (t, J=6.0 Hz, 2H), 3.39 (td, J=7.1, 3.0 Hz, 4H), 2.71 (s, 3H), 1.37 (t, J=7.3 Hz, 6H).

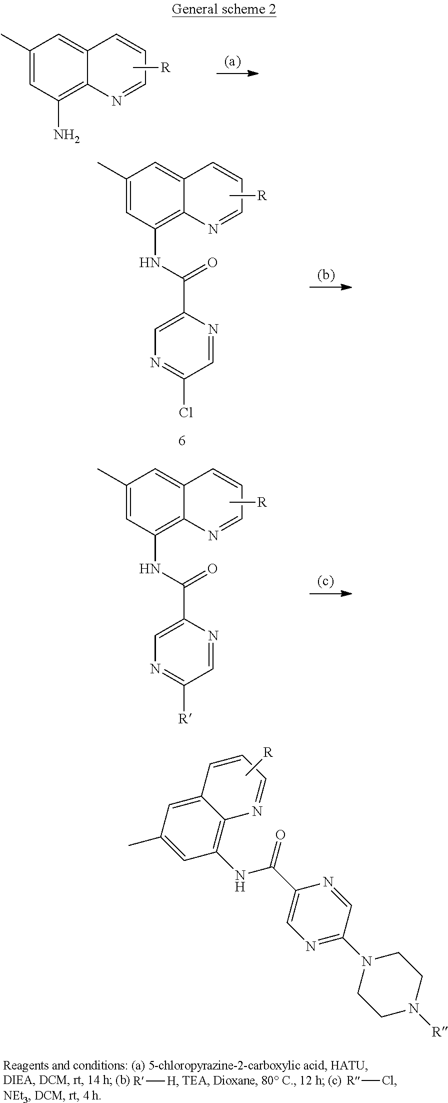

### J28: N-(6-methylquinolin-8-yl)-5-(piperazin-1-yl)pyrazine-2-carboxamide

Treatment of 5-chloro-N-(6-methylquinolin-8-yl)pyrazine-2-carboxamide (6, R═H) (100 mg, 0.34 mmol) with piperazine (37 mg, 0.38 mmol) following the general protocol A for substitution of aromatic chlorine with amine afforded N-(6-methylquinolin-8-yl)-5-(piperazin-1-yl)pyrazine-2-carboxamide as bright yellow solid (90 mg, 74%). ¹H NMR (300 MHz, DMSO-d₆) δ 11.68 (s, 1H), 9.04-8.82 (m, 3H), 8.75 (d, J=1.7 Hz, 1H), 8.62 (s, 1H), 8.38-8.29 (m, 1H), 7.64 (dd, J=8.3, 4.2 Hz, 1H), 7.51 (s, 1H), 4.00 (s, 4H), 3.48 (s, 3H), 3.28 (s, 4H). LCMS (ESI) 349.1 [M+H]⁺.

### J29: N-(6-methylquinolin-8-yl)-5-(4-(methylsulfonyl)piperazin-1-yl)pyrazine-2-carboxamide

To a stirred solution of (J28) (10 mg, 0.03 mmol) in dichloromethane on treatment with methylsulfonyl chloride (2 μL, 0.03 mmol) and triethyl amine (8 μL, 0.06 mmol) afforded J29. The crude was purified by reverse phase HPLC separation to obtain J29 as white solid (8 mg, 65%). ¹H NMR (300 MHz, CDCl₃-d) δ 11.75 (s, 1H), 9.05 (d, J=1.2 Hz, 1H), 8.87 (s, 2H), 8.27 (s, 1H), 8.11 (d, J=8.2 Hz, 1H), 7.46 (dd, J=8.3, 4.2 Hz, 1H), 7.35 (s, 1H), 3.94 (t, J=5.1 Hz, 4H), 3.51-3.30 (m, 4H), 2.86 (s, 3H), 2.60 (s, 3H). LCMS (ESI) 427.2 [M+H]⁺

### J30: 5-(4-(cyclopropanecarbonyl)piperazin-1-yl)-N-(6-methylquinolin-8-yl)pyrazine-2-carboxamide

To a stirred solution of (J28) (30 mg, 0.09 mmol) in dichloromethane on treatment with cyclopropane carbonyl chloride (10 μL, 0.09 mmol) and triethyl amine (14 μL, 0.1 mmol) afforded J30. The crude was purified by reverse phase HPLC separation to obtain J30 as white solid (15 mg, 41%). ¹H NMR (300 MHz, CDCl₃-d) δ 11.48 (s, 1H), 9.03 (d, J=1.3 Hz, 1H), 8.92 (dd, J=4.5, 1.7 Hz, 1H), 8.78 (d, J=1.8 Hz, 1H), 8.27-8.11 (m, 2H), 7.55 (dd, J=8.3, 4.4 Hz, 1H), 7.43 (s, 1H), 4.43 (s, 4H), 3.81 (s, 4H), 2.69-2.48 (m, 3H), 1.89-1.69 (m, 1H), 1.16-1.03 (m, 2H), 0.98-0.81 (m, 2H).

### J31: N-(6-methylquinolin-8-yl)-5-((2-(piperazin-1-yl)ethyl)amino)pyrazine-2-carboxamide

To a stirred solution of 6-methylquinolin-8-amine (30 mg, 0.19 mmol) in DMF, 5-((2-(4-(tert-butoxycarbonyl)piperazin-1-yl)ethyl)amino)pyrazine-2-carboxylic acid (87 mg, 0.25 mmol), HATU (144 mg, 0.38 mmol) and DIEA (105 μL, 0.57 mmol) were added and stirring continued for 16 h. On completion of the reaction as monitored by TLC, followed by extraction with DCM (3×) and the organic layer was washed with water and concentrated. The crude was purified by column chromatography with DCM/MeOH (95:5). The product obtained was treated with TFA (0.5 ml) in DCM (2 ml) and stirred at rt for 2 h. On completion the contents were concentrated and purified by reverse phase HPLC to obtain J31 as yellow solid (5 mg). ¹H NMR (400 MHz, DMSO-d₆) δ 11.56 (s, 1H), 8.91 (dd, J=4.2, 1.6 Hz, 1H), 8.82-8.68 (m, 2H), 8.34 (dd, J=8.3, 1.6 Hz, 1H), 8.13 (d, J=1.3 Hz, 1H), 8.04 (s, 1H), 7.64 (dd, J=8.3, 4.2 Hz, 1H), 7.49 (t, J=1.4 Hz, 1H), 3.63 (d, J=3.9 Hz, 4H), 3.23 (s, 4H), 2.97 (s, 4H), 2.54 (d, J=0.9 Hz, 3H). LCMS (ESI) 392.1 [M+H]⁺

### J32:5-(4-(2-aminoethyl)piperazin-1-yl)-N-(6-methylquinolin-8-yl)pyrazine-2-carboxamide

To a stirred solution of A-(6-methylquinolin-8-yl)-5-(piperazin-1-yl)pyrazine-2-carboxamide (200 mg, 0.56 mmol) in 5 mL DMF, 2-(2-bromoethyl)isoindoline-1,3-dione (220 mg, 0.86 mmol), K₂CO₃ (140 mg, 1.02 mmol) and NaI (cat.) were added and the contents were heated at 70° C. for 16 h. On completion of the reaction as shown by TLC, it was diluted with ethyl acetate and washed with water. The crude was concentrated to obtain 5-(4-(2-(1,3-dioxoisoindolin-2-yl)ethyl)piperazin-1-yl)-N-(6-methylquinolin-8-yl)pyrazine-2-carboxamide (150 mg, 0.28 mmol).

A solution of 5-(4-(2-(1,3-dioxoisoindolin-2-yl)ethyl)piperazin-1-yl)-N-(6-methylquinolin-8-yl)pyrazine-2-carboxamide (100 mg, 0.19 mmol) in ethanol (5 mL) was treated with hydrazine monohydrate (20 μL, 0.38 mmol) at room temperature and the resulting mixture was refluxed for 1 h. The reaction mixture was cooled down to room temperature. The precipitate was filtered off and the filtrate was concentrated. The residue was diluted with 10 mL EtOAc and the precipitate was filtered off. The filtrate was concentrated to dryness to give the desired product as yellow solid (51 mg). ¹H NMR (400 MHz, DMSO-d₆) δ 11.67 (s, 1H), 8.91 (dd, J=4.2, 1.6 Hz, 1H), 8.87 (d, J=1.3 Hz, 1H), 8.74 (d, J=1.8 Hz, 1H), 8.63 (d, J=1.4 Hz, 1H), 8.35 (dd, J=8.4, 1.6 Hz, 1H), 8.08 (s, 3H), 7.64 (dd, J=8.3, 4.3 Hz, 1H), 7.50 (dd, J=1.9, 1.1 Hz, 1H), 5.21 (s, 4H), 4.01 (s, 4H), 3.23 (s, 4H), 2.54 (d, J=1.0 Hz, 3H). LCMS (ESI) 392.1 [M+H]⁺

### J33: N-(6-methylquinolin-8-yl)-5-(piperidin-4-yloxy)pyrazine-2-carboxamide

To a stirred solution of 6-methylquinolin-8-amine (62 mg, 0.39 mmol) in DMF, 5-((1-(tert-butoxycarbonyl)piperidin-4-yl)oxy)pyrazine-2-carboxylic acid (150 mg, 0.46 mmol), HATU (297 mg, 0.78 mmol) and DIEA (204 μL, 1.17 mmol) were added and stirring continued for 16 h. On completion of the reaction as monitored by TLC, followed by extraction with DCM (3×) and the organic layer was washed with water and concentrated. The crude was purified by column chromatography using DCM/MeOH (9:1). The product obtained was treated with TFA (0.5 ml) in DCM (5 ml) and stirred at rt for 2 h. On completion the contents where concentrated and purified by reverse phase HPLC to obtain J33 as yellow solid (36 mg, 25% over two steps). ¹H NMR (400 MHz, DMSO-d₆) δ 11.70 (s, 1H), 8.98 (d, J=1.3 Hz, 1H), 8.93 (dd, J=4.2, 1.6 Hz, 1H), 8.77 (d, J=1.8 Hz, 1H), 8.67 (s, 1H), 8.55 (d, J=1.3 Hz, 1H), 8.36 (dd, J=8.3, 1.7 Hz, 1H), 7.65 (dd, J=8.3, 4.3 Hz, 1H), 7.56-7.49 (m, 1H), 5.40 (dt, J=8.1, 4.1 Hz, 1H), 3.33 (m, 2H), 3.19 (ddd, J=12.6, 8.7, 3.6 Hz, 2H), 2.55 (d, J=0.9 Hz, 3H), 2.27-2.16 (m, 2H), 1.98 (tt, J=9.2, 4.1 Hz, 2H). LCMS (ESI) 364.2 [M+H]⁺

### J34: 5-((l-(3-(tert-butylamino)-3-oxopropyl)piperidin-4-yl)oxy)-N-(6-methylquinolin-8-yl)pyrazine-2-carboxamide

To a stirred solution of J33 (15 mg, 0.04 mmol) in dioxane (2 ml), K₂CO₃ (17 mg, 0.12 mmol) and N-(tert-butyl)-3-chloropropanamide (8 mg, 0.05 mmol) were added and heated at reflux for 3 h. After completion of the reaction, the crude was filtered, and the filtrate was concentrated. Reverse phase HPLC purification afforded J34 as off-white solid (6 mg, 30%). ¹H NMR (300 MHz, CD₃OD-d₄) δ 8.92 (d, J=1.3 Hz, 1H), 8.82 (d, J=4.3 Hz, 1H), 8.72 (s, 1H), 8.30 (d, J=1.4 Hz, 1H), 8.20 (d, J=8.2 Hz, 1H), 7.52 (dd, J=8.3, 4.2 Hz, 1H), 7.43 (s, 1H), 5.26 (d, J=4.0 Hz, 1H), 3.78 (t, J=6.3 Hz, 1H), 2.91 (s, 2H), 2.75 (t, J=7.1 Hz, 2H), 2.57 (s, 3H), 2.52 (s, 1H), 2.37 (dt, J=12.7, 6.7 Hz, 2H), 2.15 (s, 2H), 2.01-1.84 (m, 2H), 1.35 (dd, J=5.8, 1.0 Hz, 9H).

### J35: 5-(N-(2-(dimethylamino)ethyl)sulfamoyl)-N-(6-methylquinolin-8-yl)pyrazine-2-carboxamide

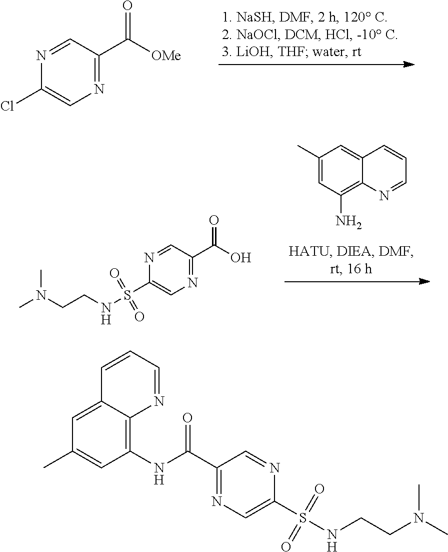

NaHS (312 mg, 6 mmol) was dissolved in 10 mL of DMF, to which methyl 5-chloropyrazine-2-carboxylate (800 mg, 4.2 mmol) were added. After refluxing for 2 h at 120° C., DMF was removed under vacuum. The mixture was diluted with EtOAc, extracted with EtOAc (2×) and washed with 2 N HCl, water and concentrated. The crude was subjected to the next reaction without further purification.

To 10 mL of a round-bottomed flask were added DCM (30 mL) and 1M aqueous HCl (3 mL, 5.0 equiv.) and the suspension was cooled to −5 to −10° C. (bath temp.) using ice-salt bath. To the well-stirred suspension was added methyl 5-mercaptopyrazine-2-carboxylate (100 mg, 0.59 mmol) and the resulting yellow mixture was stirred for 10 min, then NaOCl (6% solution, 2.5 mL, 3.3 equiv.) was added dropwise over 5 min (see, Wright, S. W.; et al., J. Org. Chem., 2006, 77, 1080-1084). After the addition, the mixture was stirred for 10 min at the same temperature and decanted to a separatory funnel. The separated organic layer was immediately added dropwise to a pre-cooled, amine solution in MeOH (80 mL) and DCM (5 mL) at 0° C. The resulting white suspension was warmed to room temperature and stirred for 2 h. The aqueous layer in the separatory funnel was washed twice with DCM and the organic layers were concentrated. The crude was dissolved in THF and subjected to ester hydrolysis following the protocol as mentioned above.

To a stirred solution of 6-methylquinolin-8-amine (52 mg, 0.33 mmol) in DMF, 5-(N-(2-(dimethylamino)ethyl)sulfamoyl)pyrazine-2-carboxylic acid (7) (100 mg, 0.36 mmol), HATU (250 mg, 0.66 mmol) and DIEA (159 μL, 0.99 mmol) were added and stirring continued for 16 h. On completion of the reaction as monitored by TLC, the mixture was diluted with EtOAc, extracted with EtOAc (2×) and washed with water and concentrated. The crude was purified by reverse phase HPLC to afford J35 as white solid (17 mg, 13%). ¹H NMR (300 MHz, CD₃OD-d₄) δ 9.55 (d, J=1.3 Hz, 1H), 9.34 (d, J=1.3 Hz, 1H), 8.86 (d, J=4.5 Hz, 1H), 8.79 (s, 1H), 8.26 (d, J=7.5 Hz, 1H), 7.57 (dd, J=8.3, 4.3 Hz, 1H), 7.52 (s, 1H), 3.61 (t, J=5.8 Hz, 2H), 3.38 (t, J=5.7 Hz, 2H), 3.01 (s, 6H), 2.60 (s, 3H). LCMS (ESI) 415.4 [M+H]⁺

### J36: 5-((5-(ethyl(2-hydroxyethyl)amino)pentan-2-yl)amino)-N-(6-methylquinolin-8-yl)pyrazine-2-carboxamide

5-((5-(ethyl(2-hydroxyethyl)amino)pentan-2-yl)amino)pyrazine-2-carboxylic acid (3) (44 mg, 0.16 mmol), 6-methylquinolin-8-amine (25 mg, 0.16 mmol), HATU (91 mg, 0.24 mmol), and DIEPA (0.08 mL, 0.48 mmol) were dissolved in 5 mL DMF. Following general protocol C₁₋₅-((5-(ethyl(2-hydroxyethyl)amino)pentan-2-yl)amino)-N-(6-methylquinolin-8-yl)pyrazine-2-carboxamide was obtained as a brown liquid (32 mg, 47%). ¹H NMR (300 MHz, CD₃OD-d₄) δ 8.85 (d, J=4.3 Hz, 1H), 8.76 (s, 1H), 8.69 (s, 1H), 8.27 (d, J=8.3 Hz, 1H), 8.02 (s, 1H), 7.56 (dd, J=8.3, 4.3 Hz, 1H), 7.47 (s, 1H), 4.41-4.04 (m, 1H), 3.92-3.79 (m, 2H), 3.27 (q, J=7.6, 6.3 Hz, 6H), 2.58 (s, 3H), 1.84 (d, J=9.6 Hz, 2H), 1.79-1.59 (m, 2H), 1.33 (t, J=6.7 Hz, 6H).

### J37: 5-((3H-[1,2,3]triazolo[4,5-b]pyridin-3-yl)oxy)-N-(2-methoxy-6-methylquinolin-8-yl)pyrazine-2-carboxamide

2-methoxy-6-methylquinolin-8-amine (73 mg, 0.39 mmol), 5-chloropyrazine-2-carboxylic acid (74 mg, 0.47 mmol), HATU (296 mg, 0.78 mmol) and DIEA (0.20 mL, 1.17 mmol) were dissolved in 5 mL DMF. Following general protocol C₁₋₅-((3H-[1.2.3]triazolo[4,5-b]pyridin-3-yl)oxy)-N-(2-methoxy-6-methylquinolin-8-yl)pyrazine-2-carboxamide was obtained as a white solid (98 mg, 59%). ¹H NMR (300 MHz, CDCl₃-d) δ 9.35 (s, 1H), 8.92 (d, J=1.1 Hz, 1H), 8.85 (d, J=1.1 Hz, 1H), 8.76 (d, J=4.7 Hz, 1H), 8.54 (d, J=8.5 Hz, 1H), 8.03 (d, J=9.1 Hz, 1H), 7.82 (d, J=8.6 Hz, 1H), 7.62-7.46 (m, 2H), 6.93 (d, J=9.1 Hz, 1H), 4.09 (s, 3H), 2.44 (s, 3H). LCMS (ESI) 429.1 [M+H]⁺.

### J38: N-(2-methoxy-6-methylquinolin-8-yl)-5-(piperazin-1-yl)pyrazine-2-carboxamide

2-methoxy-6-methylquinolin-8-amine (30 mg, 0.16 mmol), 5-(4-(tert-butoxycarbonyl)piperazin-1-yl)pyrazine-2-carboxylic acid (64 mg, 0.19 mmol), HATU (122 mg, 0.32 mmol) and DIEA (0.10 mL, 0.48 mmol) were dissolved in 5 mL DMF. Following general protocol C, tert-butyl 4-(5-((2-methoxy-6-methylquinolin-8-yl)carbamoyl)pyrazin-2-yl)piperazine-1-carboxylate was obtained as a brown solid. Subsequent Boc-deprotection using TFA (1 ml), followed by reverse phase HPLC purification afforded J38 as brown powder (22 mg, 38% yield over two steps). ¹H NMR (300 MHz, CD₃OD-d₄) δ 8.87 (d, J=1.3 Hz, 1H), 8.45 (s, 1H), 8.13 (d, J=9.1 Hz, 1H), 7.78 (d, J=8.6 Hz, 1H), 7.63 (d, J=8.6 Hz, 1H), 7.04-6.84 (m, 1H), 4.19-3.97 (m, 7H), 3.40 (t, J=5.3 Hz, 4H), 2.42 (s, 3H). LCMS (ESI) 379.1 [M+H]⁺.

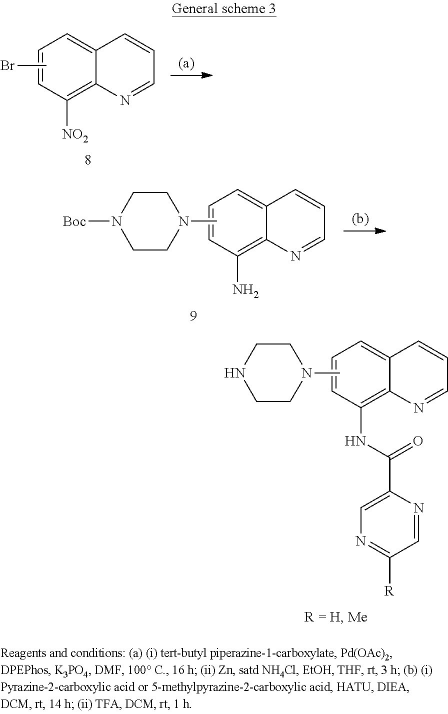

General Protocol D, Buchwald Hartwig Amination (See, Margolis, B. I; et al., J. Org. Chem., 2007, 72, 2232-2235):

An oven-dried sealed tube was charged with the 8-nitro-bromoquinoline (100 mg, 0.4 mmol), Pd(OAc)₂ (4 mg, 4 mol %), DPEphos (18 mg, 8 mol %), K₃PO₄ (212 mg, 1 mmol), and tert-butyl piperazine-1-carboxy late (90 mg, 0.5 mmol) in dioxane (4 mL). The resulting mixture was purged with argon or nitrogen for several minutes. The tube was quickly capped, then heated to 90° C. for 18 h and cooled. The mixture was filtered through celite, and the filtrate was concentrated. The crude was purified by column chromatography using EtOAc/Hexane (3:7).

### General Protocol E, Reduction of Nitro to Amine:

To a stirred solution of nitro quinoline (100 mg, 0.3 mmol) in THF: EtOH (2:1), satd NH₄Cl (2 mL) and Zn powder (520 mg, 8 mmol) were added at 0° C. The contents were warmed to room temperature and stirred for another 2 h. On consumption of starting material, the contents were filtered, and filtrate was concentrated and re-dissolved in DCM. The organic solution was then washed with water, and brine. The organic layer was dried over magnesium sulfate, filtered, concentrated, and purified by column chromatography using EtOAc/Hexane (1:1).

General Route for Amidation with Pyrazine Carboxylic Acid

To a stirred solution of 5-methylpyrazine-2-carboxylic acid/pyrazine-2-carboxylic acid (1.0 equiv.) and bromoquinolin-8-amine (1.0 equiv) in DCM, DIEPA (3 equiv) and HATU (1.5 or 2.0 equiv) were added at room temperature following general protocol C. The resulting mixture was stirred at that temperature for 12-14 h. On completion of the reaction, it was diluted with DCM and the organic layer was washed with water. The crude was concentrated and purified by column chromatography on silica gel using DCM/MeOH (95:5).

### J39: 5-methyl-N-(2-(piperazin-1-yl)quinolin-8-yl)pyrazine-2-carboxamide

5-methyl-N-(2-(piperazin-1-yl)quinolin-8-yl)pyrazine-2-carboxamide was synthesized following the general scheme 3 starting from 2-bromo-8-nitroquinoline (100 mg, 0.4 mmol). The title compound was obtained as brown solid (18 mg, 13%). ¹H NMR (300 MHz, DMSO-d₆) δ 11.91 (s, 1H), 9.24 (s, 1H), 9.06 (s, 1H), 8.79 (s, 1H), 8.63 (d, J=7.6 Hz, 1H), 8.22 (d, J=9.1 Hz, 1H), 7.55 (d, J=8.1 Hz, 1H), 7.45 (d, J=9.2 Hz, 1H), 7.35 (t, J=8.0 Hz, 1H), 4.06 (s, 4H), 3.37 (s, 4H), 2.66 (s, 3H). LCMS (ESI) 349.1 [M+H]⁺.

### J40: 5-methyl-N-(3-(piperazin-1-yl)quinolin-8-yl)pyrazine-2-carboxamide

5-methyl-N-(3-(piperazin-1-yl)quinolin-8-yl)pyrazine-2-carboxamide was synthesized following the general scheme 3 starting from 3-bromo-8-nitroquinoline (100 mg, 0.4 mmol). The title compound was obtained as brown solid (33 mg, 24%). ¹H NMR (400 MHz, DMSO-d₆) δ 11.81 (s, 1H), 9.28 (d, J=1.4 Hz, 1H), 9.04 (d, J=2.8 Hz, 1H), 8.85 (s, 1H), 8.80 (d, J=1.5 Hz, 1H), 8.66 (dd, J=5.7, 3.2 Hz, 1H), 7.78 (d, J=2.8 Hz, 1H), 7.61-7.57 (m, 2H), 3.61 (t, J=5.2 Hz, 4H), 3.35 (d, J=5.1 Hz, 4H), 2.68 (s, 3H). LCMS (ESI) 349.1 [M+H]⁺.

### J41: N-(5-(piperazin-1-yl)quinolin-8-yl)pyrazine-2-carboxamide

N-(5-(piperazin-1-yl)quinolin-8-yl)pyrazine-2-carboxamide was synthesized following the general scheme 3 starting from 5-bromo-8-nitroquinoline (100 mg, 0.4 mmol). The title compound was obtained as yellow solid (28 mg, 21%). ¹H NMR (300 MHz, CD₃OD-d₄) δ 9.43 (s, 1H), 8.98 (d, J=4.3 Hz, 1H), 8.93-8.80 (m, 3H), 8.69 (d, J=8.5 Hz, 1H), 7.67 (dd, J=8.6, 4.2 Hz, 1H), 7.38 (d, J=8.3 Hz, 1H), 3.56 (t, J=5.1 Hz, 4H), 3.42-3.30 (m, 4H). LCMS (ESI) 335.3 [M+H]⁺.

### J42: N-(6-(piperazin-1-yl)quinolin-8-yl)pyrazine-2-carboxamide

N-(6-(piperazin-1-yl)quinolin-8-yl)pyrazine-2-carboxamide was synthesized following the general scheme 3 starting from 6-bromo-8-nitroquinoline (100 mg, 0.4 mmol). The title compound was obtained as yellow solid (29 mg, 21%). ¹H NMR (300 MHz, DMSO-d₆) δ 11.88 (s, 1H), 9.41 (s, 1H), 9.03 (d, J=2.5 Hz, 1H), 8.95 (s, 1H), 8.85-8.74 (m, 2H), 8.29-8.23 (m, 1H), 7.61 (dd, J=8.4, 4.3 Hz, 1H), 7.15 (d, J=2.5 Hz, 1H), 3.52 (d, J=5.2 Hz, 4H), 3.35 (s, 4H). LCMS (ESI) 335.3 [M+H]⁺.

### J43: 5-methyl-N-(6-(piperazin-1-yl)quinolin-8-yl)pyrazine-2-carboxamide

5-methyl-N-(6-(piperazin-1-yl)quinolin-8-yl)pyrazine-2-carboxamide was synthesized following the general scheme 3 starting from 6-bromo-8-nitroquinoline (100 mg, 0.4 mmol). The title compound was obtained as yellow solid (27 mg, 19%). ¹H NMR (300 MHz, DMSO-d₆) δ 11.76 (s, 1H), 9.31-8.97 (m, 2H), 8.77 (dd, J=4.2, 2.1 Hz, 3H), 8.23 (dd, J=8.3, 1.5 Hz, 1H), 7.57 (dd, J=8.3, 4.2 Hz, 1H), 7.10 (d, J=2.5 Hz, 1H), 3.53 (t, J=5.1 Hz, 4H), 3.37 (d, J=5.1 Hz, 4H), 2.64 (s, 3H). LCMS (ESI) 349.1 [M+H]⁺.

### General Scheme for Synthesis of J44-J64

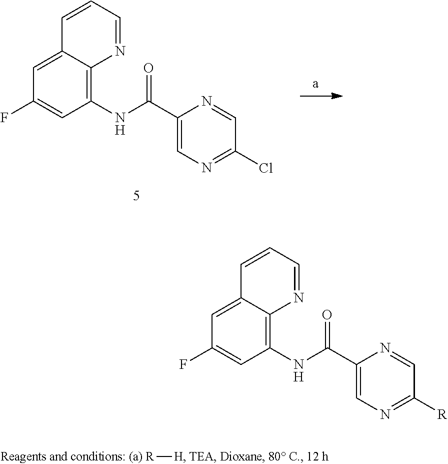

J44: N-(6-fluoroquinolin-8-yl)-5-(piperazin-1-yl)pyrazine-2-carboxamide

Treatment of 5-chloro-N-(6-fluoroquinolin-8-yl)pyrazine-2-carboxamide (5) (40 mg, 0.13 mmol) with piperazine (11 mg, 0.13 mmol) following the general protocol A, afforded the title compound as light brown solid (38 mg, 83%). ¹H NMR (300 MHz, DMSO-d₆) δ 11.77 (s, 1H), 9.03 (s, 1H), 8.96 (d, J=4.2 Hz, 1H), 8.88 (s, 1H), 8.68 (d, J=11.2 Hz, 1H), 8.61 (s, 1H), 8.45 (d, J=8.4 Hz, 1H), 7.73 (dd, J=8.2, 4.5 Hz, 1H), 7.54 (d, J=9.4 Hz, 1H), 4.01 (s, 4H), 3.28 (s, 4H). LCMS (ESI) 353.3 [M+H]⁺.

### J45: (R)—N-(6-fluoroquinolin-8-yl)-5-(2-methylpiperazin-1-yl)pyrazine-2-carboxamide

Treatment of 5-chloro-N-(6-fluoroquinolin-8-yl)pyrazine-2-carboxamide (5) (40 mg, 0.13 mmol) with tert-butyl (A)-3-methylpiperazine-1-carboxylate (27 mg, 0.13 mmol) following the general protocol A afforded a Boc protected intermediate. The Boc deprotection was achieved dissolving the intermediate in 1 ml of TFA:DCM (1:1), on completion of the reaction the crude was concentrated. White solid obtained as product after HPLC purification (15 mg, 31% over two steps). ¹H NMR (400 MHz, DMSO-d₆) δ 11.76 (s, 1H), 9.25 (s, 1H), 8.98-8.92 (m, 1H), 8.91-8.85 (m, 1H), 8.68 (dd, J=11.3, 2.8 Hz, 1H), 8.59-8.53 (m, 1H), 8.48-8.39 (m, 1H), 7.73 (dd, J=8.3, 4.3 Hz, 1H), 7.54 (dd, J=9.3, 2.8 Hz, 1H), 5.15-4.86 (m, 1H), 4.57 (d, J=14.8 Hz, 1H), 3.48-3.39 (m, 3H), 3.30 (d, J=12.4 Hz, 1H), 3.14 (d, J=12.1 Hz, 1H), 1.35 (d, J=7.0 Hz, 3H). LCMS (ESI) 367.2 [M+H]⁺.

### J46: (R)—N-(6-fluoroquinolin-8-yl)-5-(3-methylpiperazin-1-yl)pyrazine-2-carboxamide

Treatment of 5-chloro-N-(6-fluoroquinolin-8-yl)pyrazine-2-carboxamide (5) (40 mg, 0.13 mmol) with tert-butyl (R)-2-methylpiperazine-1-carboxylate (27 mg, 0.13 mmol) following the general protocol A afforded a Boc protected intermediate. The Boc deprotection was achieved dissolving intermediate in 1 ml of TFA:DCM (1:1), on completion of the reaction the crude was concentrated. White solid obtained as product after HPLC purification (12 mg, 25% over two steps). ¹H NMR (400 MHz, DMSO-d₆) δ 11.76 (s, 1H), 9.32 (s, 1H), 8.95 (dt, J=3.4, 1.7 Hz, 2H), 8.86 (dt, J=3.0, 1.3 Hz, 1H), 8.73-8.56 (m, 2H), 8.44 (dt, J=8.4, 1.8 Hz, 1H), 7.79-7.64 (m, 1H), 7.52 (dt, J=9.3, 2.5 Hz, 1H), 4.61 (dd, J=13.4, 5.6 Hz, 2H), 3.62-3.32 (m, 3H), 3.27-3.06 (m, 2H), 1.39-1.22 (m, 3H). LCMS (ESI) 367.2 [M+H]⁺.

### J47: 5-((3S,5R)-3,5-dimethylpiperazin-1-yl)-N-(6-fluoroquinolin-8-yl)pyrazine-2-carboxamide

Treatment of 5-chloro-N-(6-fluoroquinolin-8-yl)pyrazine-2-carboxamide (5) (40 mg, 0.13 mmol) with (2S,6R)-2,6-di methyl piperazine (15 mg, 0.13 mmol) following the general protocol A afforded 5-((3S,5R)-3,5-dimethylpiperazin-1-yl)-N-(6-fluoroquinolin-8-yl)pyrazine-2-carboxamide as white solid (21 mg, 42%). ¹H NMR (400 MHz, DMSO-d₆) δ 11.81 (s, 1H), 9.20 (s, 1H), 8.96 (dd, J=4.2, 1.6 Hz, 1H), 8.89 (t, J=1.3 Hz, 1H), 8.74-8.53 (m, 2H), 8.46 (dt, J=8.4, 1.6 Hz, 1H), 7.74 (dd, J=8.3, 4.2 Hz, 1H), 7.55 (dd, J=9.2, 2.7 Hz, 1H), 4.76 (d, J=14.1 Hz, 2H), 3.73 (m, 2H), 3.02 (dd, J=14.2, 11.5 Hz, 2H), 1.31 (d, J=6.5 Hz, 6H). LCMS (ESI) 381.1 [M+H]⁺.

### J48: 5-(3,3-dimethylpiperazin-1-yl)-N-(6-fluoroquinolin-8-yl)pyrazine-2-carboxamide

Treatment of 5-chloro-N-(6-fluoroquinolin-8-yl)pyrazine-2-carboxamide (5) (40 mg, 0.13 mmol) with 2,2-dimethylpiperazine (15 mg, 0.13 mmol) following the general protocol A afforded 5-(3,3-dimethylpiperazin-1-yl)-N-(6-fluoroquinolin-8-yl)pyrazine-2-carboxamide as white solid (32 mg, 64%). ¹H NMR (300 MHz, DMSO-d₆) δ 11.67 (s, 1H), 8.90 (dd, J=4.2, 1.6 Hz, 1H), 8.76 (d, J=1.2 Hz, 1H), 8.63-8.53 (m, 2H), 8.40 (dd, J=8.4, 1.6 Hz, 1H), 7.68 (dd, J=8.3, 4.2 Hz, 1H), 7.46 (dd, J=8.3 Hz, 1H), 4.02 (d, J=5.9 Hz, 2H), 3.88 (s, 2H), 3.24 (d, J=5.7 Hz, 2H), 1.37 (s, 6H). LCMS (ESI) 381.1 [M+H]⁺.

### J49: N-(6-fluoroquinolin-8-yl)-5-(4,7-diazaspiro[2.5]octan-7-yl)pyrazine-2-carboxamide

Treatment of 5-chloro-N-(6-fluoroquinolin-8-yl)pyrazine-2-carboxamide (5) (20 mg, 0.06 mmol) with 4,7-diazaspiro[2.5]octane hydrochloride (12 mg, 0.06 mmol) following the general protocol A afforded N-(6-fluoroquinolin-8-yl)-5-(4,7-diazaspiro[2.5]octan-7-yl)pyrazine-2-carboxamide as white solid (12 mg, 48%). ¹H NMR (300 MHz, DMSO-d₆) δ 11.79 (s, 1H), 9.32 (s, 1H), 8.95 (d, J=4.0 Hz, 1H), 8.86 (s, 1H), 8.66 (d, J=18.2 Hz, 2H), 8.51-8.35 (m, 1H), 7.74 (dd, J=8.4, 4.3 Hz, 1H), 7.55 (d, J=9.1 Hz, 1H), 4.09 (s, 2H), 3.97 (s, 2H), 3.41 (s, 2H), 1.12-0.84 (m, 4H). LCMS (ESI) 379.1 [M+H]⁺.

### J50: N-(6-fluoroquinolin-8-yl)-5-(3-(trifluoromethyl)piperazin-1-yl)pyrazine-2-carboxamide

Treatment of 5-chloro-N-(6-fluoroquinolin-8-yl)pyrazine-2-carboxamide (5) (40 mg, 0.13 mmol) with 2-(trifluoromethyl)piperazine (40 mg, 0.13 mmol) following the general protocol A afforded N-(6-fluoroquinolin-8-yl)-5-(3-(trifluoromethyl)piperazin-1-yl)pyrazine-2-carboxamide as white solid (31 mg, 64%). ¹H NMR (300 MHz, DMSO-d₆) δ 11.73 (s, 1H), 8.94 (dd, J=4.2, 1.6 Hz, 1H), 8.86 (d, J=1.2 Hz, 1H), 8.73-8.53 (m, 2H), 8.43 (dd, J=8.4, 1.6 Hz, 1H), 7.71 (dd, J=8.4, 4.2 Hz, 1H), 7.52 (dd, J=9.3, 2.8 Hz, 1H), 4.64 (d, J=13.6 Hz, 1H), 4.33 (d, J=13.8 Hz, 1H), 3.98 (s, 1H), 3.40 (dd, J=13.6, 10.0 Hz, 2H), 3.24 (d, J=12.9 Hz, 1H), 2.98 (t, J=11.2 Hz, 1H). LCMS (ESI) 421.1 [M+H]⁺.

### J51: N-(6-fluoroquinolin-8-yl)-5-(4-(methylsulfonyl)piperazin-1-yl)pyrazine-2-carboxamide

Treatment of 5-chloro-N-(6-fluoroquinolin-8-yl)pyrazine-2-carboxamide (5) (40 mg, 0.13 mmol) with 1-(methylsulfonyl)piperazine (21 mg, 0.13 mmol) following the general protocol A afforded A-(6-fluoroquinolin-8-yl)-5-(4-(methylsulfonyl)piperazin-1-yl)pyrazine-2-carboxamide as white solid (25 mg, 49%). ¹H NMR (400 MHz, DMSO-d₆) δ 11.75 (s, 1H), 9.05-8.92 (m, 1H), 8.87 (d, J=2.6 Hz, 1H), 8.69 (dd, J=11.4, 3.2 Hz, 1H), 8.60 (d, J=2.6 Hz, 1H), 8.52-8.35 (m, 1H), 7.73 (dd, J=8.3, 4.3 Hz, 1H), 7.54 (dd, J=9.5, 3.1 Hz, 1H), 3.94 (d, J=4.8 Hz, 4H), 3.36-3.26 (m, 4H), 2.94 (d, J=2.4 Hz, 3H). LCMS (ESI) 431.0 [M+H]⁺.

### J52: N-(6-fluoroquinolin-8-yl)-5-(4-methylpiperazin-1-yl)pyrazine-2-carboxamide

Treatment of compound 5-chloro-N-(6-fluoroquinolin-8-yl)pyrazine-2-carboxamide (5) (20 mg, 0.06 mmol) with 1-(methyl)piperazine (6 mg, 0.06 mmol) following the general protocol A afforded N-(6-fluoroquinolin-8-yl)-5-(4-methylpiperazin-1-yl)pyrazine-2-carboxamide as white solid (12 mg, 53%). ¹H NMR (300 MHz, CD₃OD-d₄) δ 8.97 (d, J=1.4 Hz, 1H), 8.88 (dd, J=4.2, 1.6 Hz, 1H), 8.72 (dd, J=11.2, 2.8 Hz, 1H), 8.51 (d, J=1.4 Hz, 1H), 8.33 (dd, J=8.4, 1.7 Hz, 1H), 7.63 (dd, J=8.4, 4.3 Hz, 1H), 7.35 (dd, J=9.0, 2.7 Hz, 1H), 3.33 (s, 8H), 3.02 (s, 3H). LCMS (ESI) 367.2 [M+H]⁺.

### J53: N-(6-fluoroquinolin-8-yl)-5-(3-hydroxypyrrolidin-1-yl)pyrazine-2-carboxamide

Treatment of compound 5-chloro-N-(6-fluoroquinolin-8-yl)pyrazine-2-carboxamide (5) (30 mg, 0.10 mmol) with pyrrolidin-3-ol (9 mg, 0.10 mmol) following the general protocol A afforded N-(6-fluoroquinolin-8-yl)-5-(3-hydroxypyrrolidin-1-yl)pyrazine-2-carboxamide as off-white solid (23 mg, 51%). ¹H NMR (300 MHz, DMSO-d₆) δ 11.69 (s, 1H), 8.94 (dd, J=4.2, 1.6 Hz, 1H), 8.79 (d, J=1.2 Hz, 1H), 8.66 (dd, J=11.4, 2.8 Hz, 1H), 8.42 (dd, J=8.4, 1.6 Hz, 1H), 8.13 (s, 1H), 7.70 (dd, J=8.4, 4.3 Hz, 1H), 7.49 (dd, J=9.3, 2.9 Hz, 1H), 5.15 (s, 1H), 4.46 (s, 1H), 3.64 (dd, J=16.8, 8.7 Hz, 3H), 2.03 (d, J=22.1 Hz, 2H). LCMS (ESI) 354.1 [M+H]⁺.

### J54: N-(6-fluoroquinolin-8-yl)-5-(hexahydropyrrolo[1,2-a]pyrazin-2(1H)-yl)pyrazine-2-carboxamide

Treatment of compound 5-chloro-N-(6-fluoroquinolin-8-yl)pyrazine-2-carboxamide (5) (30 mg, 0.10 mmol) with octahydropyrrolo[1,2-a]pyrazine (13 mg, 0.10 mmol) following the general protocol A afforded N-(6-fluoroquinolin-8-yl)-5-(hexahydropyrrolo[1,2-a]pyrazin-2(1H)-yl)pyrazine-2-carboxamide as off-light brown solid obtained (21 mg, 41%). ¹H NMR (300 MHz, DMSO-d₆) δ 11.71 (s, 1H), 8.96 (d, J=4.2 Hz, 1H), 8.81 (d, J=1.8 Hz, 1H), 8.68 (d, J=11.4 Hz, 1H), 8.54 (s, 1H), 8.47-8.38 (m, 1H), 7.72 (dd, J=8.5, 4.3 Hz, 1H), 7.57-7.43 (m, 1H), 4.69 (d, J=12.4 Hz, 1H), 4.52 (d, J=12.9 Hz, 1H), 3.21-2.97 (m, 3H), 2.76 (t, J=11.3 Hz, 1H), 2.26-1.79 (m, 4H), 1.72 (s, 2H), 1.50-1.28 (m, 1H). LCMS (ESI) 393.2 [M+H]⁺.

### J55: N-(6-fluoroquinolin-8-yl)-5-(4-(pyrrolidin-1-yl)piperidin-1-yl)pyrazine-2-carboxamide

Treatment of compound 5-chloro-N-(6-fluoroquinolin-8-yl)pyrazine-2-carboxamide (5) (20 mg, 0.06 mmol) with 4-(pyrrolidin-1-yl)piperidine (20 mg, 0.06 mmol) following the general protocol A afforded N-(6-fluoroquinolin-8-yl)-5-(4-(pyrrolidin-1-yl)piperidin-1-yl)pyrazine-2-carboxamide as white solid (9 mg, 36%). ¹H NMR (300 MHz, DMSO-d₆) δ 9.89 (s, 1H), 8.95 (dd, J=4.3, 1.6 Hz, 1H), 8.84 (d, J=1.3 Hz, 1H), 8.67 (dd, J=11.3, 2.9 Hz, 1H), 8.61 (d, J=1.4 Hz, 1H), 8.44 (dd, J=8.4, 1.6 Hz, 1H), 7.72 (dd, J=8.4, 4.2 Hz, 1H), 7.53 (dd, J=9.3, 2.8 Hz, 1H), 4.69 (d, J=13.7 Hz, 2H), 3.55 (s, 2H), 3.22-2.97 (m, 4H), 2.55 (s, 1H), 2.27-2.13 (m, 2H), 2.02 (s, 2H), 1.86 (t, J=6.5 Hz, 2H), 1.62 (q, J=11.2 Hz, 2H). LCMS (ESI) 421.1 [M+H]⁺.

### J56: 5-(3-fluoropiperidin-1-yl)-N-(6-fluoroquinolin-8-yl)pyrazine-2-carboxamide

Treatment of compound 5-chloro-N-(6-fluoroquinolin-8-yl)pyrazine-2-carboxamide (5) (40 mg, 0.13 mmol) with 3-fluoropiperidine (14 mg, 0.13 mmol) following the general protocol A afforded 5-(3-fluoropiperidin-1-yl)-N-(6-fluoroquinolin-8-yl)pyrazine-2-carboxamide as white solid (28 mg, 58%). ¹H NMR (300 MHz, DMSO-d₆) δ 11.67 (s, 1H), 8.94 (dd, J=4.2, 1.6 Hz, 1H), 8.79 (d, J=1.3 Hz, 1H), 8.66 (dd, J=11.4, 2.8 Hz, 1H), 8.55 (d, J=1.4 Hz, 1H), 8.42 (dd, J=8.4, 1.6 Hz, 1H), 7.70 (dd, J=8.3, 4.3 Hz, 1H), 7.49 (dd, J=9.3, 2.8 Hz, 1H), 4.89 (d, J=47.3 Hz, 1H), 4.39-4.25 (m, 1H), 4.14 (d, J=13.8 Hz, 1H), 3.83-3.62 (m, 1H), 3.46 (t, J=11.2 Hz, 1H), 1.97-1.86 (m, 2H), 1.80 (d, J=11.7 Hz, 1H), 1.63 (s, 1H). LCMS (ESI) 370.1 [M+H]⁺.

### J57: 5-(6,6-difluoro-3-azabicyclo[3.1.0]hexan-3-yl)-N-(6-fluoroquinolin-8-yl)pyrazine-2-carboxamide

Treatment of compound 5-chloro-N-(6-fluoroquinolin-8-yl)pyrazine-2-carboxamide (5) (40 mg, 0.13 mmol) with 6,6-difluoro-3-azabicyclo[3.1.0]hexane (20 mg, 0.13 mmol) following the general protocol A afforded 5-(6,6-difluoro-3-azabicyclo[3.1.0]hexan-3-yl)-N-(6-fluoroquinolin-8-yl)pyrazine-2-carboxamide as white solid (24 mg, 48%). ¹H NMR (300 MHz, CDCl₃-d) δ 11.80 (s, 1H), 9.02 (d, J=1.4 Hz, 1H), 8.87 (dd, J=4.2, 1.6 Hz, 1H), 8.82 (dd, J=11.2, 2.8 Hz, 1H), 8.12 (dd, J=8.4, 1.7 Hz, 1H), 7.93 (d, J=1.4 Hz, 1H), 7.49 (dd, J=8.3, 4.2 Hz, 1H), 7.15 (dd, J=8.7, 2.7 Hz, 1H), 4.02 (d, J=11.3 Hz, 2H), 3.96-3.82 (m, 2H), 2.62-2.46 (m, 2H). LCMS (ESI) 386.1 [M+H]⁺.

### J58: 5-((2-(1,1-dioxidothiomorpholino)ethyl)amino)-N-(6-fluoroquinolin-8-yl)pyrazine-2-carboxamide

Treatment of compound 5-chloro-N-(6-fluoroquinolin-8-yl)pyrazine-2-carboxamide (5) (30 mg, 0.10 mmol) with 4-(2-aminoethyl)thiomorpholine 1,1-dioxide hydrochloride (25 mg, 0.10 mmol) following the general protocol A afforded 5-((2-(1,1-dioxidothiomorpholino)ethyl)amino)-N-(6-fluoroquinolin-8-yl)pyrazine-2-carboxamide as off-white solid (22 mg, 38%). ¹H NMR (400 MHz, DMSO-d₆) δ 11.67 (s, 1H), 8.97 (dd, J=4.2, 1.6 Hz, 1H), 8.80 (d, J=1.3 Hz, 1H), 8.70 (dd, J=11.4, 2.8 Hz, 1H), 8.45 (dd, J=8.4, 1.6 Hz, 1H), 8.14 (d, J=1.4 Hz, 2H), 7.73 (dd, J=8.3, 4.2 Hz, 1H), 7.52 (dd, J=9.3, 2.8 Hz, 1H), 3.68 (d, J=6.0 Hz, 2H), 3.43 (d, J=20.0 Hz, 8H), 3.16 (s, 2H). LCMS (ESI) 445.1 [M+H]⁺.

### J59: N-(6-fluoroquinolin-8-yl)-5-((3-(hydroxyamino)-3-oxopropyl)amino)pyrazine-2-carboxamide

Treatment of compound 5-chloro-N-(6-fluoroquinolin-8-yl)pyrazine-2-carboxamide (5) (100 mg, 0.33 mmol) with methyl 3-aminopropanoate hydrochloride (34 mg, 0.33 mmol) following the general protocol A afforded ethyl 3-((5-((6-fluoroquinolin-8-yl)carbamoyl)pyrazin-2-yl)amino)propanoate. The latter (30 mg) on treatment with hydroxyl amine hydrochloride (55 mg, 0.8 mmol) and KOH (90 mg, 1.6 mmol) in MeOH at 0° C. and subsequent acidification and extraction with DCM afforded the title compound in 78% yield (see, Sparks, S. M.; et al., J. Org. Chem., 2004, 69, 3025-3035) as brown liquid. ¹H NMR (400 MHz, DMSO-d₆) δ 11.63 (s, 1H), 10.47 (s, 1H), 9.00-8.88 (m, 1H), 8.76 (t, J=6.8 Hz, 2H), 8.70-8.60 (m, 1H), 8.48-8.35 (m, 1H), 8.10 (d, J=22.3 Hz, 2H), 7.71 (dt, J=8.0, 3.6 Hz, 1H), 7.49 (dq, J=8.9, 2.8 Hz, 1H), 3.61 (q, J=6.5 Hz, 2H), 2.33 (t, J=6.8 Hz, 2H).

### J60: (E)-5-(4-(4-(dimethylamino)but-2-enoyl)piperazin-1-yl)-N-(6-fluoroquinolin-8-yl)pyrazine-2-carboxamide

To a stirred solution of J44 (30 mg, 0.08 mmol) in DMF, (Z)-4-(dimethylamino)but-2-enoic acid (14 mg, 0.08 mmol), HATU (45 mg, 0.12 mmol) and DIPEA (29 μl, 0.16 mmol) were added and stirring continued for 16 h. On completion of the reaction as monitored by tlc, the mixture was extracted with DCM and organic layer was washed with water. The crude was purified by reverse phase HPLC to afford the product as yellow solid (12 mg, 30%). ¹H NMR (400 MHz, DMSO-d₆) δ 11.52 (s, 1H), 8.81 (dd, J=4.2, 1.6 Hz, 1H), 8.66 (s, 1H), 8.50 (dd, J=11.3, 2.8 Hz, 1H), 8.39-8.22 (m, 2H), 7.60 (dd, J=8.3, 4.2 Hz, 1H), 7.35 (dd, J=9.2, 2.8 Hz, 1H), 6.95 (d, J=15.1 Hz, 1H), 6.64 (dt, J=14.7, 7.1 Hz, 1H), 3.86-3.59 (m, 10H), 2.80 (s, 6H). LCMS (ESI) 464.2 [M+H]⁺.

### J61: N-(6-fluoroquinolin-8-yl)-5-morpholinopyrazine-2-carboxamide

Treatment of compound 5-chloro-N-(6-fluoroquinolin-8-yl)pyrazine-2-carboxamide (5) (40 mg, 0.13 mmol) with morpholine (11 mg, 0.13 mmol) following the general protocol A afforded the title compound as white powder (33 mg, 71%). ¹H NMR (300 MHz, DMSO-d₆) 11.73 (s, 1H), 8.96 (d, J=3.4 Hz, 1H), 8.84 (d, J=1.3 Hz, 1H), 8.68 (dd, J=11.4, 2.8 Hz, 1H), 8.52 (d, J=1.4 Hz, 1H), 8.44 (dd, J=8.3, 1.7 Hz, 1H), 7.72 (dd, J=8.4, 4.2 Hz, 1H), 7.53 (dd, J=9.3, 2.9 Hz, 1H), 3.76 (s, 8H). LCMS (ESI) 354.2 [M+H]⁺.

### J62: 5-(4,4-difluoropiperidin-1-yl)-N-(6-fluoroquinolin-8-yl)pyrazine-2-carboxamide

Treatment of compound 5-chloro-N-(6-fluoroquinolin-8-yl)pyrazine-2-carboxamide (5) (40 mg, 0.13 mmol) with 4,4-difluoropiperidine (16 mg, 0.13 mmol) following the general protocol A afforded the title compound as brown powder (28 mg, 56%). ¹H NMR (300 MHz, DMSO-d₆) δ 11.71 (s, 1H), 8.94 (dd, J=4.2, 1.7 Hz, 1H), 8.82 (d, J=1.3 Hz, 1H), 8.69-8.56 (m, 2H), 8.42 (dd, J=8.4, 1.6 Hz, 1H), 7.71 (dd, J=8.3, 4.2 Hz, 1H), 7.50 (dd, J=9.3, 2.9 Hz, 1H), 3.92 (t, J=5.9 Hz, 4H), 2.22-1.93 (m, 4H). LCMS (ESI) 388.2 [M+H]⁺.

### J63: N-(6-fluoroquinolin-8-yl)-5-(8-methyl-3,8-diazabicyclo[3.2.1]octan-3-yl)pyrazine-2-carboxamide

Treatment of compound 5-chloro-N-(6-fluoroquinolin-8-yl)pyrazine-2-carboxamide (5) (40 mg, 0.13 mmol) with rac-(1R,5S)-8-methyl-3,8-diazabicyclo[3.2.1]octane dihydrochloride (17 mg, 0.13 mmol) following the general protocol A afforded A-(6-fluoroquinolin-8-yl)-5-(8-methyl-3,8-diazabicyclo[3.2.1]octan-3-yl)pyrazine-2-carboxamide as white solid (18 mg, 35%). ¹H NMR (300 MHz, DMSO-d₆) δ 11.78 (s, 1H), 8.97 (d, J=4.0 Hz, 1H), 8.89 (s, 1H), 8.69 (d, J=11.4 Hz, 1H), 8.57 (s, 1H), 8.46 (d, J=8.0 Hz, 1H), 7.80-7.66 (m, 1H), 7.55 (d, J=8.7 Hz, 1H), 4.50 (d, J=13.7 Hz, 2H), 4.15 (s, 2H), 3.49 (s, 2H), 2.80 (s, 3H), 2.23 (s, 2H), 1.89 (d, J=9.7 Hz, 2H). LCMS (ESI) 393.2 [M+H]⁺.

### J64: N-(6-fluoroquinolin-8-yl)-5-(5-methyl-2,5-diazabicyclo[2.2.1]heptan-2-yl)pyrazine-2-carboxamide

Treatment of compound 5-chloro-N-(6-fluoroquinolin-8-yl)pyrazine-2-carboxamide (5) (40 mg, 0.13 mmol) with 2-Methyl-2,5-diazabicyclo[2.2.1]heptane (15 mg, 0.13 mmol) following the general protocol A afforded A-(6-fluoroquinolin-8-yl)-5-(5-methyl-2,5-diazabicyclo[2.2.1]heptan-2-yl)pyrazine-2-carboxamide as white solid (10 mg, 19%). LCMS (ESI) 379.1 [M+H]⁺.

### J65: 2-((5-(diethylamino)pentan-2-yl)amino)-N-(6-methylquinolin-8-yl)acetamide

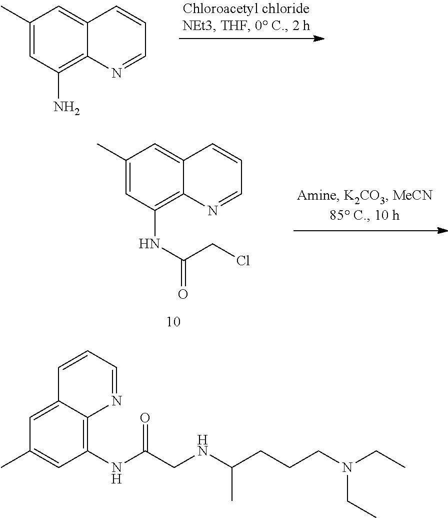 [SMILES: Cc1cc(N)c2ncccc2c1 ; Cc1cc(NC(=O)CCl)c2ncccc2c1 ; CCN(CC)CCCC(C)NCC(=O)Nc1cc(C)cc2cccnc12]

To a stirred solution of 6-methylquinolin-8-amine (160 mg, 1 mmol) in THF, chloroacetyl chloride (158 μL, 2 mmol) and triethyl amine (144 μL, 1 mmol) were added at 0° C. The temperature was slowly raised to rt. On completion the reaction was quenched with DCM, followed by extraction with DCM (3×) and the organic layer was washed with water, concentrated and without further purification was subjected to the next reaction. To a stirred solution of 2-chloro-N-(6-methylquinolin-8-yl)acetamide (80 mg, 0.34 mmol) in MeCN, N,N-diethylpentane-1,4-diamine (54 mg, 0.34 mmol) and K₂CO₃ (94 mg, 0.68 mmol) were added and stirred at 85° C. for 10 h. The crude was purified by column chromatography to afford dark brown liquid (82 mg, 23% over two steps). ¹H NMR (300 MHz, CD₃OD-d₄) δ 8.84 (dd, J=4.2, 1.7 Hz, 1H), 8.54 (d, J=1.8 Hz, 1H), 8.26 (dd, J=8.3, 1.7 Hz, 1H), 7.55 (dd, J=8.3, 4.2 Hz, 1H), 7.51 (d, J=1.8 Hz, 1H), 4.32 (s, 2H), 3.54-3.41 (m, 1H), 3.28 (d, J=7.3 Hz, 3H), 3.25-3.16 (m, 3H), 2.55 (d, J=0.9 Hz, 3H), 2.04-1.60 (m, 4H), 1.46 (d, J=6.6 Hz, 3H), 1.35 (t, J=7.3 Hz, 6H). LCMS (ESI) 357.1 [M+H]⁺.

### J66: 2-((2-(4-methylpiperazin-1-yl)ethyl)amino)-N-(6-methylquinolin-8-yl)acetamide

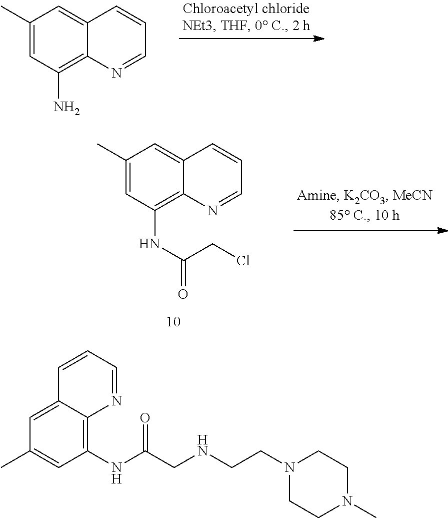

To a stirred solution of 6-methylquinolin-8-amine (160 mg, 1 mmol) in THF, chloroacetyl chloride (158 μL, 2 mmol) and triethyl amine (144 μL, 1 mmol) was added at 0° C. The temperature was slowly raised to rt. On completion the reaction was quenched with DCM, followed by extraction with DCM (3×) and the organic layer was washed with water, concentrated and without further purification was subjected to the next reaction. To a stirred solution of 2-chloro-N-(6-methylquinolin-8-yl)acetamide (80 mg, 0.34 mmol) in MeCN, 2-(4-methylpiperazin-1-yl)ethan-1-amine (49 mg, 0.34 mmol) and K₂CO₃ (94 mg, 0.68 mmol) were added and stirred at 85° C. for 10 h. The crude was purified by column chromatography to afford dark brown liquid in 61% yield over two steps. ¹H NMR (300 MHz, CD₃OD-d₄) δ 8.85 (dd, J=4.2, 1.6 Hz, 1H), 8.53 (s, 1H), 8.26 (dd, J=8.3, 1.7 Hz, 1H), 7.56 (dd, J=8.3, 4.2 Hz, 1H), 7.51 (s, 1H), 4.32 (s, 2H), 3.63-3.46 (m, 3H), 3.37 (d, J=5.6 Hz, 2H), 3.19 (s, 4H), 2.88 (d, J=12.7 Hz, 6H), 2.56 (d, J=1.0 Hz, 3H). LCMS (ESI) 342.1 [M+H]⁺

### J67: 6-methyl-N-((5-(piperazin-1-yl)pyrazin-2-yl)methyl)quinolin-8-amine

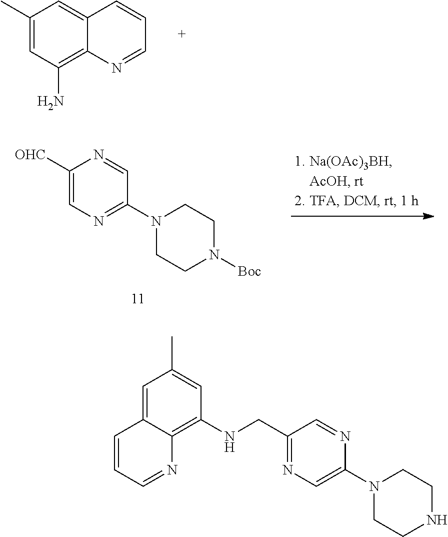 [SMILES: Cc1cc(N)c2ncccc2c1 ; CC(C)(C)OC(=O)N1CCN(c2cnc(C=O)cn2)CC1 ; Cc1cc(NCc2cnc(N3CCNCC3)cn2)c2ncccc2c1]

To a stirred solution of 6-methylquinolin-8-amine (22 mg, 0.14 mmol) in DCM, tert-butyl 4-(5-formylpyrazin-2-yl)piperazine-1-carboxylate (35 mg, 0.12 mmol), Sodium triacetoxyborohydride (60 mg, 0.28 mmol) and AcOH (16 μL, 0.14 mmol) were added and stirred at rt for 12 h. On completion the contents were filtered, filtrate was concentrated and followed by extraction with DCM (3×) and the organic layer was washed with water and concentrated. The crude was purified by column chromatography using EtOAc/Hexane (3:7). Subsequent Boc-deprotection using DCM:TFA (1:1, 1 ml) afforded the title compound as yellow powder in 39% yield over two steps. ¹H NMR (300 MHz, DMSO-d₆) δ 9.07 (s, 2H), 8.68 (dd, J=4.2, 1.7 Hz, 1H), 8.42 (s, 1H), 8.22 (d, J=1.3 Hz, 1H), 8.11 (dd, J=8.3, 1.6 Hz, 1H), 7.47 (dd, J=8.3, 4.1 Hz, 1H), 6.88 (s, 1H), 6.58 (d, J=1.7 Hz, 1H), 4.52 (s, 2H), 3.77 (t, J=5.2 Hz, 4H), 3.21 (s, 4H), 2.36 (s, 3H). LCMS (ESI) 335.1 [M+H]⁺

### J68:6-methyl-N-(6-(4-methylpiperazin-1-yl)pyridin-3-yl)quinoline-8-carboxamide

6-(4-methylpiperazin-1-yl)pyridin-3-amine (35 mg, 0.18 mmol), 6-methylquinoline-8-carboxylic acid (35 mg, 0.18 mmol), HATU (102 mg, 0.27 mmol) and DIEA (0.10 mL, 0.54 mmol) were dissolved in 5 mL DMF. Following general protocol C₁₋₆-methyl-N-(6-(4-methylpiperazin-1-yl)pyridin-3-yl)quinoline-8-carboxamide was obtained as a brown solid (12 mg, 19%). LCMS (ESI) 362.2 [M+H]⁺.

### J69: 6-methyl-N-(4-(4-(methylsulfonyl)piperazin-1-yl)phenyl)quinoline-8-carboxamide

4-(4-(methylsulfonyl)piperazin-1-yl)aniline (46 mg, 0.18 mmol), 6-methylquinoline-8-carboxylic acid (35 mg, 0.18 mmol), HATU (102 mg, 0.27 mmol) and DIEA (0.1 mL, 0.54 mmol) were dissolved in 5 mL DMF. Following general protocol C₁₋₆-methyl-N-(4-(4-(methylsulfonyl)piperazin-1-yl)phenyl)quinoline-8-carboxamide was obtained as a greenish yellow solid (15 mg, 18%). ¹H NMR (300 MHz, DMSO-d₆) δ 13.14 (s, 1H), 9.08 (s, 1H), 8.51 (d, J=12.8 Hz, 2H), 8.02 (s, 1H), 7.76 (d, J=8.6 Hz, 2H), 7.70 (dd, J=8.6, 4.3 Hz, 1H), 7.04 (d, J=8.7 Hz, 2H), 3.26 (s, 8H), 2.94 (s, 3H), 2.59 (s, 3H). LCMS (ESI) 425.1 [M+H]⁺.

### J70: 4-(4-methylpiperazin-1-yl)-N-(6-methylquinolin-8-yl)benzamide

4-(4-methylpiperazin-1-yl)benzoic acid (55 mg, 0.25 mmol), 6-methylquinolin-8-amine (40 mg, 0.25 mmol), HATU (142 mg, 0.38 mmol) and DIEA (0.15 mL, 0.75 mmol) were dissolved in 5 mL DMF. Following general protocol C, 4-(4-methylpiperazin-1-yl)-N-(6-methylquinolin-8-yl)benzamide was obtained as a brown solid (13 mg, 15%). LCMS (ESI) 361.2 [M+H]⁺.

### J71: N-(5-chloro-6-fluoroquinolin-8-yl)-5-((5-(diethylamino)pentan-2-yl)amino)pyrazine-2-carboxamide

Treatment of 5-chloro-N-(5-chloro-6-fluoroquinolin-8-yl)pyrazine-2-carboxamide (40 mg, 0.12 mmol) with N,N-diethylpentane-1,4-diamine (23 mg, 0.15 mmol) following the general protocol A, afforded the title compound as light brown liquid (30 mg, 55%). ¹H NMR (300 MHz, Methanol-d₄) δ 8.94 (s, 1H), 8.76 (s, 2H), 8.63 (s, 1H), 7.99 (s, 1H), 7.76 (s, 1H), 4.28 (d, J=7.2 Hz, 1H), 3.24 (q, J=7.4 Hz, 6H), 1.93-1.59 (m, 4H), 1.32 (t, J=7.2 Hz, 9H). LCMS (ESI) 459.2 [M+H]⁺.

### J72: N,N-diethyl-N-(5-(((6-methylquinolin-8-yl)amino)methyl)pyrazin-2-yl)pentane-1,4-diamine

To a stirred solution of 6-methylquinolin-8-amine (22 mg, 0.14 mmol) in DCM, 5-((5-(diethylamino)pentan-2-yl)amino)pyrazine-2-carbaldehyde (31 mg, 0.12 mmol), sodium triacetoxyborohydride (60 mg, 0.28 mmol) and AcOH (16 μL, 0.14 mmol) were added and stirred at rt for 12 h. On completion the contents were filtered, filtrate was concentrated and followed by extraction with DCM (3×) and the organic layer was washed with water and concentrated. The crude was purified by HPLC using MeCN/water solvent to afford title compound as yellow liquid (4 mg, 7%). LCMS (ESI) 407.3 [M+H]⁺.

### J73:5-(4-methylpiperazin-1-yl)-N-(naphthalen-1-yl)pyrazine-2-carboxamide

J73 was synthesized following the method as for J52, starting naphthalen-1-amine (100 mg, 0.70 mmol). White solid (26 mg, 75%). ¹H NMR (400 MHz, DMSO-d₆) δ 10.49 (s, 1H), 8.84 (s, 1H), 8.56 (s, 1H), 8.04-7.89 (m, 2H), 7.86 (t, J=8.0 Hz, 2H), 7.62-7.53 (m, 3H), 4.68 (s, 2H), 3.50 (m, 2H), 3.20 (s, 4H), 2.87 (s, 3H). LCMS (ESI) 348.2 [M+H]+.

### J74: N-(naphthalen-1-yl)-5-(piperazin-1-yl)pyrazine-2-carboxamide

J74 was synthesized following the method as for J44, starting naphthalen-1-amine (100 mg, 0.70 mmol). White solid (26 mg, 81%). ¹H NMR (400 MHz, DMSO-d₆) δ 10.48 (s, 1H), 9.02 (s, 1H), 8.83 (s, 1H), 8.54 (s, 1H), 8.06-7.90 (m, 2H), 7.86 (dd, J=15.2, 7.8 Hz, 2H), 7.58 (dd, J=7.9, 3.4 Hz, 3H), 4.00 (t, J=5.1 Hz, 4H), 3.29 (t, J=5.3 Hz, 4H). LCMS (ESI) 334.2 [M+H]+.

### J75: 5-(5,6-dihydro-[1,2,4]triazolo[4,3-a]pyrazin-7(8H)-yl)-N-(6-fluoroquinolin-8-yl)pyrazine-2-carboxamide

Treatment of compound 5-chloro-N-(6-fluoroquinolin-8-yl)pyrazine-2-carboxamide (5) (25 mg, 0.08 mmol) with 5,6,7,8-tetrahydro-[1,2,4]triazolo[4,3-a]pyrazine (10 mg, 0.08 mmol) following the general protocol A afforded the title compound as white powder (16 mg, 51%). ¹H NMR (300 MHz, DMSO-d₆) δ 11.77 (s, 1H), 9.11-8.82 (m, 2H), 8.77-8.55 (m, 3H), 8.45 (d, J=8.4 Hz, 1H), 7.74 (s, 1H), 7.55 (s, 1H), 5.18 (s, 2H), 4.28 (s, 4H). LCMS (ESI) 391.1 [M+H]⁺.

### J76: N-(6-fluoroquinolin-8-yl)-5-(3-(trifluoromethyl)-5,6-dihydro-[1,2,4]triazolo[4,3-a]pyrazin-7(8H)-yl)pyrazine-2-carboxamide

Treatment of compound 5-chloro-N-(6-fluoroquinolin-8-yl)pyrazine-2-carboxamide (5) (25 mg, 0.08 mmol) with 3-(trifluoromethyl)-5,6,7,8-tetrahydro-[1,2,4]triazolo[4,3-a]pyrazine (15 mg, 0.08 mmol) following the general protocol A afforded the title compound as white powder (13 mg, 35%). LCMS (ESI) 459.1 [M+H]⁺.

### J77: 5-(2,5-diazabicyclo[2.2.1]heptan-2-yl)-N-(6-fluoroquinolin-8-yl)pyrazine-2-carboxamide

Treatment of compound 5-chloro-N-(6-fluoroquinolin-8-yl)pyrazine-2-carboxamide (5) (25 mg, 0.08 mmol) with 2,5-diazabicyclo[2.2.1]heptane (8 mg, 0.08 mmol) following the general protocol A afforded the title compound as white powder (21 mg, 72%). ¹H NMR (300 MHz, DMSO-d₆) δ 11.76 (d, J=7.3 Hz, 1H), 9.06-8.80 (m, 2H), 8.68 (dd, J=11.3, 2.9 Hz, 1H), 8.51-8.28 (m, 2H), 7.95 (s, 1H), 7.73 (dd, J=8.5, 4.8 Hz, 1H), 7.54 (dd, J=9.2, 2.9 Hz, 1H), 5.14 (s, 1H), 4.60 (s, 1H), 3.75 (s, 2H), 2.89 (d, J=1.0 Hz, 2H), 2.73 (d, J=0.9 Hz, 2H). LCMS (ESI) 365.1 [M+H]⁺.

### J78: 5-(4-(diethylamino)piperidin-1-yl)-N-(6-fluoroquinolin-8-yl)pyrazine-2-carboxamide

Treatment of compound 5-chloro-N-(6-fluoroquinolin-8-yl)pyrazine-2-carboxamide (5) (50 mg, 0.16 mmol) with N,N-diethylpiperidin-4-amine (24 mg, 0.16 mmol) following the general protocol A afforded the title compound as white powder (39 mg, 58%). ¹H NMR (300 MHz, DMSO-d₆) δ 11.64 (d, J=4.4 Hz, 1H), 8.92 (p, J=1.8 Hz, 1H), 8.75 (d, J=3.7 Hz, 1H), 8.64 (dt, J=11.5, 3.1 Hz, 1H), 8.50-8.43 (m, 1H), 8.40 (dq, J=8.5, 1.6 Hz, 1H), 7.69 (dt, J=7.8, 3.1 Hz, 1H), 7.47 (dt, J=9.2, 3.2 Hz, 1H), 4.56 (d, J=13.2 Hz, 2H), 3.00 (t, J=12.6 Hz, 2H), 2.81 (t, J=11.3 Hz, 1H), 2.50-2.41 (m, 4H), 1.78 (d, J=12.5 Hz, 2H), 1.51-1.30 (m, 2H), 0.95 (t, J=7.0 Hz, 6H). LCMS (ESI) 423.2 [M+H]⁺.

### J79: 5-((3S,4S)-4-amino-3-methyl-2-oxa-8-azaspiro[4.5]decan-8-yl)-N-(6-fluoroquinolin-8-yl)pyrazine-2-carboxamide

Treatment of compound 5-chloro-N-(6-fluoroquinolin-8-yl)pyrazine-2-carboxamide (5) (40 mg, 0.13 mmol) with (3S,4S)-3-methyl-2-oxa-8-azaspiro[4.5]decan-4-amine (31 mg, 0.13 mmol) following the general protocol A afforded the title compound as white powder (28 mg, 49%). ¹H NMR (300 MHz, Methanol-d₄) δ 8.82 (dd, J=4.3, 1.6 Hz, 1H), 8.79 (d, J=1.3 Hz, 1H), 8.63 (dd, J=11.2, 2.8 Hz, 1H), 8.31 (d, J=1.4 Hz, 1H), 8.26 (dd, J=8.4, 1.6 Hz, 1H), 7.57 (dd, J=8.3, 4.2 Hz, 1H), 7.27 (dd, J=9.0, 2.8 Hz, 1H), 4.56-4.22 (m, 3H), 4.03 (d, J=9.3 Hz, 1H), 3.91 (d, J=9.2 Hz, 1H), 3.47 (d, J=4.2 Hz, 1H), 3.23 (ddd, J=18.0, 9.8, 4.0 Hz, 2H), 1.99-1.66 (m, 4H), 1.34 (d, J=6.5 Hz, 3H). LCMS (ESI) 437.2 [M+H]⁺.

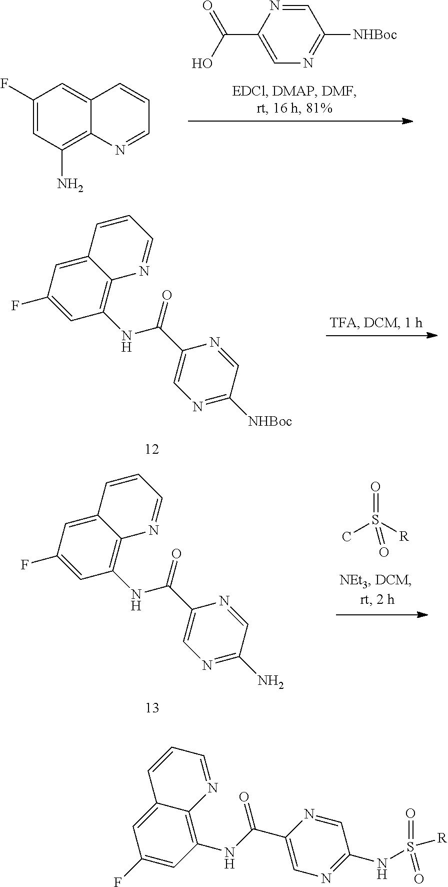

### General Protocol for Synthesis of Sulfonamide Derivatives

5-((tert-butoxycarbonyl)amino)pyrazine-2-carboxylic acid (146 mg, 0.61 mmol) and 5-fluoroquinolin-8-amine (100 mg, 0.61 mmol), EDCl (186 mg, 1.2 mmol) and DMAP (cat.) were dissolved in DCM following the general protocol for amidation (route B). After completion of the reaction as monitored by TLC, the crude was concentrated and purified by column chromatography. White solid (158 mg) obtained was dissolved in DCM (1 mL), TFA (1 mL) was added to it and stirred at room temperature for 1 h. The crude was concentrated and subjected to the next reaction without further purification.

To a stirred solution of 5-amino-N-(6-fluoroquinolin-8-yl)pyrazine-2-carboxamide (20 mg, 0.07 mmol) in DCM (2 mL), sulfonyl chloride (0.07 mmol) and triethyl amine (0.07 mmol) were added at 0° C. and stirred for 2 h at rt. On completion of the reaction as monitored by LCMS, work up was done using DCM/water. The crude was purified by HPLC analysis (MeCN: water).

J80: N-(6-fluoroquinolin-8-yl)-5-((2-methylpropyl)sulfonamido)pyrazine-2-carboxamide

White solid (17 mg, 0.61%). ¹H NMR (300 MHz, Acetone-d₆) δ 9.11 (d, J=1.4 Hz, 1H), 8.97 (ddd, J=4.2, 1.6, 0.6 Hz, 1H), 8.81 (dd, J=11.3, 2.8 Hz, 1H), 8.62 (d, J=1.4 Hz, 1H), 8.43 (dd, J=8.4, 1.6 Hz, 1H), 7.71 (ddd, J=8.3, 4.2, 0.8 Hz, 1H), 7.45 (dd, J=9.1, 2.8 Hz, 1H), 3.59 (d, J=6.5 Hz, 2H), 2.36 (dt, J=13.4, 6.7 Hz, 1H), 1.14 (d, J=6.7 Hz, 6H). LCMS (ESI) 404.0 [M+H]⁺.

### J81: N-(6-fluoroquinolin-8-yl)-5-((3,3,3-trifluoropropyl)sulfonamido)pyrazine-2-carboxamide

White solid (17 mg, 0.55%). ¹H NMR (300 MHz, Acetone-d₆) δ 9.14 (d, J=1.4 Hz, 1H), 9.03-8.92 (m, 1H), 8.81 (dd, J=11.3, 2.8 Hz, 1H), 8.62 (d, J=1.4 Hz, 1H), 8.43 (dd, J=8.4, 1.6 Hz, 1H), 7.72 (ddd, J=8.4, 4.2, 0.8 Hz, 1H), 7.45 (dd, J=9.1, 2.8 Hz, 1H), 4.03-3.93 (m, 2H), 3.00-2.87 (m, 2H). LCMS (ESI) 444.0 [M+H]⁺.

### J82: 5-((1R,5S,6s)-6-amino-3-azabicyclo[3.1.0]hexan-3-yl)-N-(6-fluoroquinolin-8-yl)pyrazine-2-carboxamide

Treatment of compound 5-chloro-N-(6-fluoroquinolin-8-yl)pyrazine-2-carboxamide (5) (30 mg, 0.10 mmol) with tert-butyl ((1R,5S,6s)-3-azabicyclo[3.1.0]hexan-6-yl)carbamate (36 mg, 0.18 mmol) following the general protocol A, followed by Boc-deprotection using TFA afforded the title compound as off-white solid (25 mg, 76% over two steps). ¹H NMR (300 MHz, DMSO-d₆) δ 11.75 (s, 1H), 8.95 (d, J=4.1 Hz, 1H), 8.84 (s, 1H), 8.68 (d, J=11.1 Hz, 1H), 8.45 (d, J=8.3 Hz, 1H), 8.23 (d, J=6.7 Hz, 3H), 7.78-7.67 (m, 1H), 7.53 (d, J=9.6 Hz, 1H), 3.91 (d, J=11.3 Hz, 2H), 3.69 (d, J=11.1 Hz, 2H), 2.19 (s, 2H). LCMS (ESI) 365.1 [M+H]⁺.

### J83: 5-((3S,4R)-3-amino-4-fluoropyrrolidin-1-yl)-N-(6-fluoroquinolin-8-yl)pyrazine-2-carboxamide

Treatment of compound 5-chloro-N-(6-fluoroquinolin-8-yl)pyrazine-2-carboxamide (5) (30 mg, 0.10 mmol) with tert-butyl ((3S,4R)-4-fluoropyrrolidin-3-yl)carbamate (36 mg, 0.18 mmol) following the general protocol A, followed by Boc-deprotection using TFA afforded the title compound as off-white solid (28 mg, 76% over two steps). ¹H NMR (300 MHz, DMSO-d₆) δ 11.79 (s, 1H), 8.96 (d, J=4.1 Hz, 1H), 8.91 (s, 1H), 8.75-8.54 (m, 3H), 8.46 (d, J=8.3 Hz, 1H), 8.32 (s, 1H), 7.74 (dd, J=8.5, 4.2 Hz, 1H), 7.55 (d, J=9.2 Hz, 1H), 4.22-3.78 (m, 6H). LCMS (ESI) 371.1 [M+H]⁺.

### J84: N-(6-fluoroquinolin-8-yl)-5-(1-methyl-4-oxo-1,3,8-triazaspiro[4.5]decan-8-yl)pyrazine-2-carboxamide

Treatment of compound 5-chloro-N-(6-fluoroquinolin-8-yl)pyrazine-2-carboxamide (5) (40 mg, 0.13 mmol) with 1-methyl-1,3,8-triazaspiro[4.5]decan-4-one (22 mg, 0.13 mmol) following the general protocol A afforded the title compound as white powder (29 mg, 51%). ¹H NMR (300 MHz, DMSO-d₆) δ 11.73 (s, 1H), 8.95 (d, J=4.2 Hz, 1H), 8.83 (s, 1H), 8.68 (dd, J=11.5, 2.8 Hz, 1H), 8.56 (s, 1H), 8.44 (d, J=8.4 Hz, 1H), 7.72 (dd, J=8.4, 4.3 Hz, 1H), 7.52 (dd, J=9.3, 2.8 Hz, 1H), 4.55-4.30 (m, 4H), 3.83-3.73 (m, 4H), 2.56 (s, 2H), 1.93 (s, 3H). LCMS (ESI) 436.2 [M+H]⁺.

### J85: N-(6-fluoroquinolin-8-yl)-5-(4-hydroxy-4-(trifluoromethyl)piperidin-1-yl)pyrazine-2-carboxamide

Treatment of compound 5-chloro-N-(6-fluoroquinolin-8-yl)pyrazine-2-carboxamide (5) (30 mg, 0.10 mmol) with 4-(trifluoromethyl)piperidin-4-ol (33 mg, 0.18 mmol) following the general protocol A afforded the title compound as white solid (18 mg, 41%). ¹H NMR (300 MHz, DMSO-d₆) δ 11.73 (s, 1H), 8.96 (d, J=4.2 Hz, 1H), 8.83 (s, 1H), 8.68 (dd, J=11.5, 2.7 Hz, 1H), 8.57 (s, 1H), 8.44 (d, J=8.3 Hz, 1H), 7.72 (dd, J=8.5, 4.3 Hz, 1H), 7.58-7.46 (m, 1H), 6.24 (s, 1H), 4.56 (d, J=13.5 Hz, 2H), 3.26 (d, J=11.7 Hz, 2H), 1.77 (d, J=13.4 Hz, 4H). LCMS (ESI) 436.1 [M+H]⁺.

### J86: (S)—N-(6-fluoroquinolin-8-yl)-5-(4-hydroxy-2-oxopyrrolidin-1-yl)pyrazine-2-carboxamide

Treatment of compound 5-chloro-N-(6-fluoroquinolin-8-yl)pyrazine-2-carboxamide (5) (30 mg, 0.10 mmol) with (S)-4-hydroxypyrrolidin-2-one (18 mg, 0.18 mmol) following the general protocol A afforded the title compound as white solid (11 mg, 29%). ¹H NMR (300 MHz, DMSO-d6) δ 11.88 (s, 1H), 9.70 (s, 1H), 9.18 (s, 1H), 8.99 (d, J=4.2 Hz, 1H), 8.68 (d, J=10.5 Hz, 1H), 8.46 (d, J=8.3 Hz, 1H), 7.74 (dd, J=8.5, 4.3 Hz, 1H), 7.58 (d, J=9.3 Hz, 1H), 5.49 (d, J=3.6 Hz, 1H), 4.49 (s, 1H), 4.11 (t, J=5.9 Hz, 1H), 3.98 (d, J=11.8 Hz, 1H), 3.05 (dd, J=17.4, 5.9 Hz, 1H). LCMS (ESI) 368.3 [M+H]⁺.

### J87: 5-(8-amino-2-oxa-6-azaspiro[3.4]octan-6-yl)-N-(6-fluoroquinolin-8-yl)pyrazine-2-carboxamide

Treatment of compound 5-chloro-N-(6-fluoroquinolin-8-yl)pyrazine-2-carboxamide (5) (30 mg, 0.10 mmol) with tert-butyl (2-oxa-6-azaspiro[3.4]octan-8-yl)carbamate (41 mg, 0.18 mmol) following the general protocol A, followed by Boc-deprotection using TFA afforded the title compound as off-white solid (19 mg, 48%). ¹H NMR (300 MHz, DMSO-d₆) δ 11.71 (s, 1H), 8.94 (d, J=3.9 Hz, 1H), 8.85 (s, 1H), 8.66 (dd, J=11.1, 2.9 Hz, 1H), 8.43 (d, J=14.8 Hz, 3H), 8.24 (d, J=4.4 Hz, 1H), 7.71 (dd, J=8.5, 4.1 Hz, 1H), 7.56-7.46 (m, 1H), 4.91 (d, J=7.3 Hz, 1H), 4.61 (dt, J=11.9, 6.6 Hz, 3H), 4.34 (s, 1H), 4.17 (d, J=11.6 Hz, 1H), 3.95 (d, J=11.8 Hz, 1H), 3.82 (d, J=10.2 Hz, 1H), 2.51 (s, 1H). LCMS (ESI) 395.2 [M+H]⁺.

### J88:5-(3-amino-3-methylazetidin-1-yl)-N-(6-fluoroquinolin-8-yl)pyrazine-2-carboxamide

Treatment of compound 5-chloro-N-(6-fluoroquinolin-8-yl)pyrazine-2-carboxamide (5) (30 mg, 0.10 mmol) with tert-butyl (3-methylazetidin-3-yl)carbamate (33 mg, 0.18 mmol) following the general protocol A, followed by Boc-deprotection using TFA afforded the title compound as off-white solid (22 mg). ¹H NMR (300 MHz, DMSO-d₆) δ 11.76 (s, 1H), 8.96 (d, J=4.2 Hz, 1H), 8.86 (s, 1H), 8.73-8.62 (m, 1H), 8.53 (s, 2H), 8.45 (d, J=8.3 Hz, 1H), 8.20 (s, 1H), 7.73 (dd, J=8.5, 4.2 Hz, 1H), 7.58-7.48 (m, 1H), 4.29 (d, J=10.0 Hz, 2H), 4.20 (d, J=10.0 Hz, 2H), 1.63 (s, 3H). LCMS (ESI) 353.2 [M+H]⁺.

### J89: 5-(2,2-dioxido-2-thia-5-azabicyclo[2.2H]heptan-5-yl)-N-(6-fluoroquinolin-8-yl)pyrazine-2-carboxamide

Treatment of compound 5-chloro-N-(6-fluoroquinolin-8-yl)pyrazine-2-carboxamide (5) (30 mg, 0.10 mmol) with 2-thia-5-azabicyclo[2.2.1]heptane 2,2-dioxide (26 mg, 0.18 mmol) following the general protocol A afforded the title compound as brown solid (9 mg, 21%). ¹H NMR (300 MHz, DMSO-d₆) δ 11.76 (s, 1H), 8.96 (d, J=4.2 Hz, 1H), 8.88 (s, 1H), 8.68 (dd, J=11.2, 2.8 Hz, 1H), 8.52-8.28 (m, 2H), 7.73 (dd, J=8.3, 4.2 Hz, 1H), 7.62-7.42 (m, 1H), 5.23 (s, 1H), 4.28-4.15 (m, 1H), 4.17-3.99 (m, 2H), 3.86 (d, J=11.7 Hz, 1H), 3.17 (d, J=5.0 Hz, 1H), 2.60 (s, 2H). LCMS (ESI) 414.2 [M+H]⁺.

### J90: (R)-5-(1-amino-8-azaspiro[4.5]decan-8-yl)-N-(6-fluoroquinolin-8-yl)pyrazine-2-carboxamide

Treatment of compound 5-chloro-N-(6-fluoroquinolin-8-yl)pyrazine-2-carboxamide (5) (30 mg, 0.10 mmol) with tert-butyl (R)-8-azaspiro[4.5]decan-1-amine (28 mg, 0.18 mmol) following the general protocol A afforded the title compound as off-white solid (18 mg, 42%). ¹H NMR (300 MHz, DMSO-d₆) δ 11.68 (s, 1H), 8.93 (d, J=4.2 Hz, 1H), 8.79 (s, 1H), 8.65 (dd, J=11.4, 2.7 Hz, 1H), 8.54 (s, 1H), 8.42 (d, J=8.3 Hz, 1H), 7.95-7.86 (m, 2H), 7.70 (dd, J=8.4, 4.2 Hz, 1H), 7.49 (dd, J=9.3, 2.7 Hz, 1H), 4.40 (t, J=16.5 Hz, 2H), 3.29-3.06 (m, 3H), 2.07 (d, J=8.3 Hz, 1H), 1.88-1.39 (m, 9H). LCMS (ESI) 421.2 [M+H]⁺.

### J91: 2-((5-(diethylamino)pentan-2-yl)amino)-N-(6-fluoroquinolin-8-yl)thiazole-4-carboxamide

An oven-dried sealed tube was charged with the 2-bromo-N-(6-fluoroquinolin-8-yl)thiazole-4-carboxamide (30 mg, 0.08 mmol), Pd(dba)₃ (4 mg, 5 mol %), BINAP (7 mg, 14 mol %), K₃PO₄ (102 mg, 0.48 mmol), and N,N-diethylpentane-1,4-diamine (64 mg, 0.4 mmol) in dioxane (4 mL). The resulting mixture was purged with argon or nitrogen for several minutes. The tube was quickly capped, then heated to 90° C. for 18 h and cooled. The mixture was filtered through celite, and the filtrate was concentrated. The crude was purified by column chromatography using EtOAc/Hexane (3:7). Brown liquid (10 mg, 30%) ¹H NMR (400 MHz, Methanol-d₄) δ 8.92-8.80 (m, 1H), 8.63 (dd, J=11.1, 2.8 Hz, 1H), 8.35-8.27 (m, 1H), 7.62 (dd, J=8.3, 4.2 Hz, 1H), 7.50 (s, 1H), 7.33 (dd, J=9.0, 2.8 Hz, 1H), 4.10 (q, J=7.1 Hz, 1H), 3.26-3.07 (m, 6H), 1.99-1.72 (m, 4H), 1.40 (d, J=6.5 Hz, 3H), 1.21 (dt, J=17.8, 7.3 Hz, 6H). LCMS (ESI) 430.3 [M+H]⁺.

### J92: 2-((3S,4R)-3-amino-4-fluoropyrrolidin-1-yl)-N-(6-fluoroquinolin-8-yl)thiazole-4-carboxamide

Treatment of compound 2-bromo-N-(6-fluoroquinolin-8-yl)thiazole-4-carboxamide (30 mg, 0.08 mmol) with tert-butyl ((3S,4R)-4-fluoropyrrolidin-3-yl)carbamate (0.4 mmol) following the method as used for J92, followed by Boc deprotection using TFA, afforded the title compound as off-white solid (8 mg, 26%). LCMS (ESI) 376.2 [M+H]⁺. J93: N-(6-fluoroquinolin-8-yl)-2-(trifluoromethyl)thiazole-4-carboxamide 2-(trifluoromethyl)thiazole-4-carboxylic acid (78 mg, 0.4 mmol), 5-fluoroquinolin-8-amine (50 mg, 0.3 mmol), EDCI (70 mg, 0.45 mmol) and DMAP (cat.) were dissolved in DCM following the general protocol for amidation. The crude was purified by trituration with MeOH to afford the title compound (81 mg, 79%). ¹H NMR (400 MHz, Chloroform-d) δ 11.56 (s, 1H), 8.93 (dd, J=4.3, 1.6 Hz, 1H), 8.79 (dd, J=10.9, 2.7 Hz, 1H), 8.60 (s, 1H), 8.22 (dd, J=8.3, 1.6 Hz, 1H), 7.58 (dd, J=8.3, 4.3 Hz, 1H), 7.26 (dd, J=8.5, 2.8 Hz, 1H). LCMS (ESI) 342.1 [M+H]⁺.

### J94: Ethyl 4-((6-fluoroquinolin-8-yl)carbamoyl)thiazole-2-carboxylate

2-(ethoxycarbonyl)thiazole-4-carboxylic acid (70 mg, 0.35 mmol), 5-fluoroquinolin-8-amine (50 mg, 0.3 mmol), EDCI (70 mg, 0.45 mmol) and DMAP (cat.) were dissolved in DCM following the general protocol for amidation. The crude was purified by trituration with MeOH to afford the title compound (68 mg, 65%). ¹H NMR (400 MHz, Chloroform-d) δ 11.59 (s, 1H), 8.95 (dd, J=4.3, 1.6 Hz, 1H), 8.78 (dd, J=10.9, 2.7 Hz, 1H), 8.60 (d, J=0.9 Hz, 1H), 8.22 (dd, J=8.3, 1.6 Hz, 1H), 7.57 (ddd, J=8.3, 4.3, 0.7 Hz, 1H), 7.25 (dd, J=8.5, 2.7 Hz, 1H), 4.58 (q, J=7.2 Hz, 2H), 1.54 (t, J=7.2 Hz, 3H). LCMS (ESI) 346.1 [M+H]⁺.

### J95: 4-((6-fluoroquinolin-8-yl)carbamoyl)thiazole-2-carboxylic Acid

Compound J95 (30 mg, 0.08 mmol) was hydrolized following the general protocol for ester hydrolysis as mentioned above to afford the title compound as white powder (14 mg, 0.05 mmol) in 55% yield. ¹H NMR (400 MHz, DMSO-d6) δ 11.42 (d, J=1.5 Hz, 1H), 9.00 (dd, J=4.2, 1.7 Hz, 1H), 8.90 (s, 1H), 8.69 (dd, J=11.2, 2.8 Hz, 1H), 8.47 (dd, J=8.4, 1.6 Hz, 1H), 7.75 (dd, J=8.3, 4.2 Hz, 1H), 7.59 (dd, J=9.3, 2.8 Hz, 1H). LCMS (ESI) 318.1 [M+H]⁺.

### Example II

This example the testing of representative compounds for in-vivo efficacy.

### In Vivo Efficacy Studies:

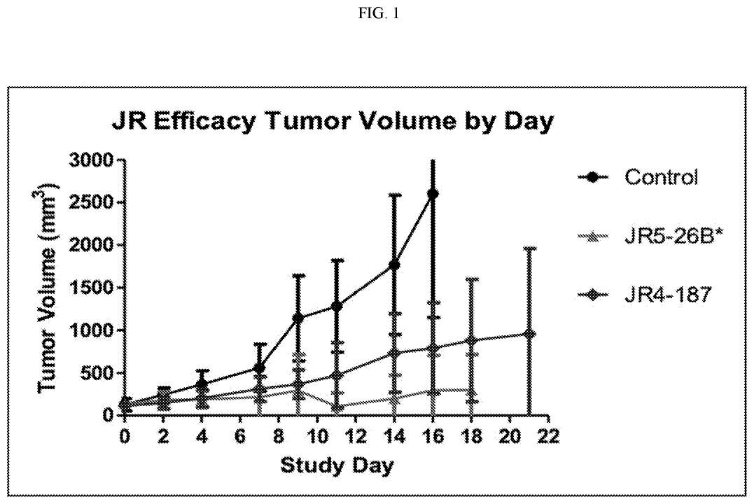

Efficacy study was performed on mice with subcutaneous CT-26 implantation. Mice were implanted with 1,000,000 cells into the right flank and randomized into three groups (n =5) eleven days later. Mice were dosed 5 times weekly with no dosing on weekends. Control tumors grew well, with the majority of mice reaching euthanasia criteria at Day 16. JR5-26B and JR4-187 showed efficacy (FIG. 1).

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound described by Formula IA: 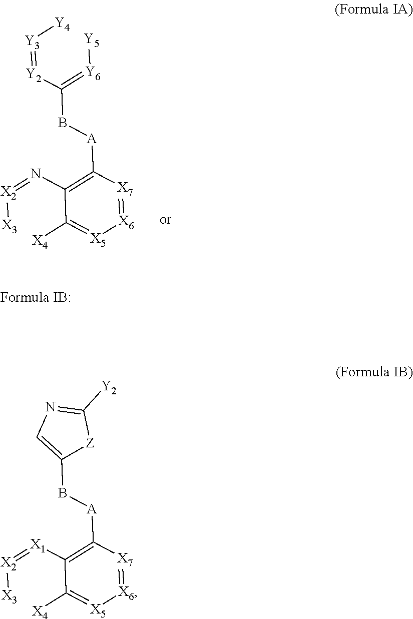
including pharmaceutically acceptable salts (e.g., 2,2,2-trifluoroacetate (TFA) salts and other salts) (e.g., physiologically tolerated acid addition salts), solvates, and/or prodrugs thereof;
wherein A, B, X1, X2, X3, X4, X5, X6, X7, Y2, Y3, Y4, Y5, Y6 and Z independently include any chemical moiety that renders the resulting compound capable of one or more of:
serving as an effective therapeutic agent for treating, ameliorating, and preventing various forms of cancer and other inflammatory diseases;
activating the cholesterol biosynthesis pathway within cancer cells and/or immune cells;
activating the cell cycle regulation pathway within cancer cells and/or immune cells; and
up-regulating HMGCS1 protein expression within cancer cells and/or immune cells.

2-4. (canceled)

5. The compound of claim 1,
wherein X¹ is either CH or N;
wherein X², X³, X⁴, X⁵, X⁶ and X⁷ are independently selected from CR¹ or N, with the proviso that at least three of them must be CR¹;
wherein Y², Y³, Y⁴, Y⁵ Y⁶ are independently CH, CR² or N;
wherein Y⁶ is a bond, in which case one of Y³, Y⁴, or Y⁵ is NR³, O, or S, while the other two may be CR² or N;
wherein A and B are independently selected from a group consisting of NH, C═O, C═S, CH₂, C(R³)₂, CF₂, C—NMe₂, C═N—OR⁴, C═N—N(R⁵)₂, C═N—SO₂R⁶, or C═N—CN;
wherein Z is either O, S or NH;
wherein R¹ is independently H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR¹⁰, SO₂R¹⁰, NO₂, COR⁷, C₁₋₆ alkyl-COR⁷, N(R¹⁰)C₂₋₆ alkyl-NR¹⁰R¹⁰, —N(R¹⁰)C₂₋₆ alkyl-R⁷, N(C₂₋₆ alkyl)₂-NR¹⁰, —O(CH₂)ₚR⁷, —S(CH₂)ₚR⁷, or —N(R¹⁰)C(═O)(CH₂)ₚR⁷, with a proviso that not more than three R¹ can be other than H;
wherein R² is independently H, halogen, C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆ alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆ alkoxy, C₁₋₆ alkoxy-C₃₋₇ cycloalkyl, C₁₋₆ alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆ acyloxy, C₁₋₆ acyloxy, C₁₋₆ acyloxy-C₃₋₇ cycloalkyl, C₁₋₆ acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆thioalkoxy, C₁₋₆ thioalkoxy, C₁₋₆ thioalkoxy-C₃₋₇ cycloalkyl, C₁₋₆ thioalkoxy-C₄₋₇ heterocycloalkyl, C₁₋₆ thioalkoxy-phenyl, C₁₋₆ thioalkoxy-naphthyl, C₁₋₆ thioalkoxy-(5-10 membered mono- or bicyclo-heteroaryl), amino, C₁₋₆ monoalkylamino, C₁₋₆ dialkylamino, C₁₋₆ acyl, C₁₋₆ acylamino, cyano, CF₃, OCF₃, SOR¹⁰, SO₂R¹⁰, NO₂, COR⁷, C₁₋₆ alkyl-COR⁷, N(R¹⁰)C₂₋₆ alkyl-NR¹⁰R¹⁰, N(C₂₋₆ alkyl)₂-NR¹⁰, 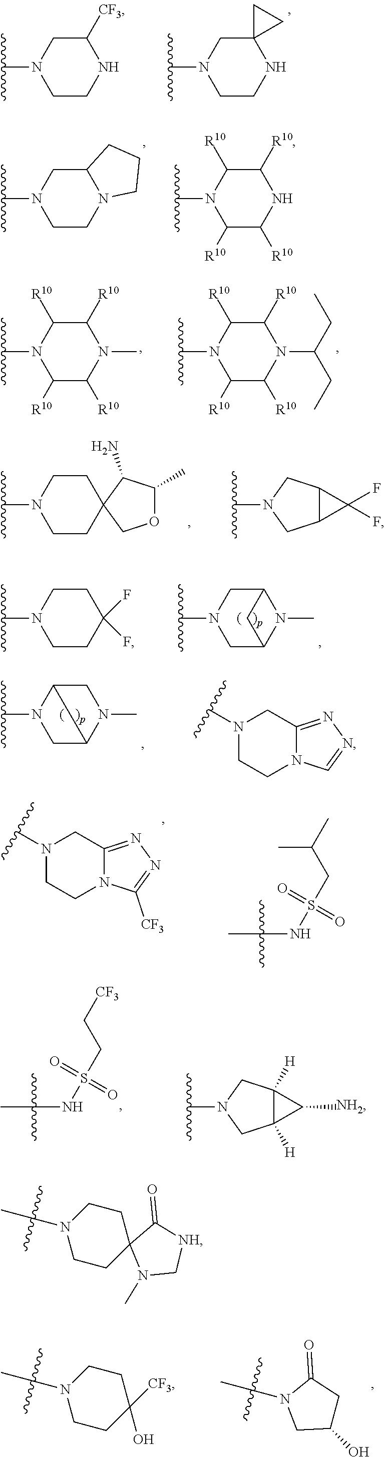 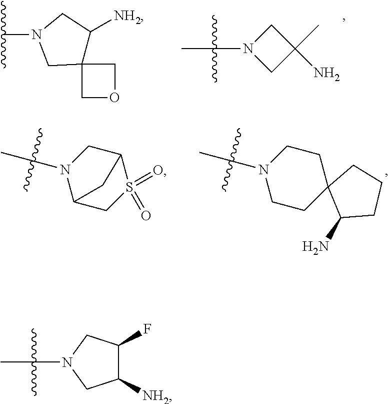
CF₃, CO₂Et, CO₂H, —N(R¹⁰)C₂₋₆ alkyl-R⁷, —O(CH₂)ₚR⁷, —S(CH₂)ₚR⁷, or —N(R¹⁰)C(═O)(CH₂)ₚR⁷, with a proviso that not more than two R² can be other than H;
wherein R³ is hydrogen, C₁₋₆ alkyl, C₃₋₆ alkenyl, C₃₋₆ alkynyl, C₃₋₇ cycloalkyl, C₄₋₇ heterocycloalkyl, phenyl, naphthyl, 5-10 membered mono- or bicyclic heteroaryl, C₁₋₆ alkyl-C₃₋₇ cycloalkyl, or C₁₋₆ alkyl-C₄₋₇ heterocycloalkyl;
wherein R⁴ is H or C₁₋₆ alkyl;
wherein each R⁵ is independently H or C₁₋₆ alkyl, or the two R⁵, taken together with the N atom to which they are both attached, form a heterocycloalkyl ring of 4-7 members, containing up to one other heteroatom selected from O, S, or NR³;
wherein R⁶ is C₁₋₆ alkyl or CF₃;
wherein R⁷ is OH, NR⁸R⁹, O(CH₂)_{q}NR⁸R⁹, C₁₋₆ alkoxy, C₁₋₆ alkoxy-C₁₋₆ alkoxy, C₂₋₆ hydroxyalkoxy, cyclopropyl, 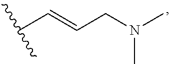 [SMILES: */C=C/CN(C)C]
oxetanyl, oxetanyloxy, oxetanylamino, oxolanyl, oxolanyloxy, oxolanylamino, oxanyl oxanyloxy, oxanylamino, oxepanyl, oxepanyloxy, oxepanylamino, azetidinyl, azetidinyloxy, azetidylamino, pyrrolidinyl, pyrolidinyloxy, pyrrolidinylamino, piperidinyl, piperidinyloxy, piperidinylamino, azepanyl, azepanyloxy, azepanylamino, dioxolanyl, dioxanyl, morpholino, thiomorpholino, thiomorpholino-S,S-dioxide, piperazino, dioxepanyl, dioxepanyloxy, dioxepanylamino, oxazepanyl, oxazepanyloxy, oxazepanylamino, diazepanyl, diazepanyloxy, diazepanylamino, all of which may be optionally substituted with OH, OR¹⁰, oxo, halogen, R¹⁰, CH₂OR¹⁰, CH₂NR⁸R⁹ or CH₂CH₂CONR⁸R⁹;
wherein R⁸ and R⁹ are each independently H, —CD₃, C₁₋₆ alkyl, C₃₋₆ alkenyl, C₃₋₆ alkynyl, C₃₋₈ cycloalkyl, —(C₁₋₃ alkyl)-(C₃₋₈ cycloalkyl), C₃₋₈ cycloalkenyl, C₁-C₆ acyl, 4-12 membered monocyclic or bicyclic heterocyclyl, 4-12 membered monocyclic or bicyclic heterocyclyl-C₁-C₆ alkyl-, C₆₋C₁₂ aryl, 5-11 membered heteroaryl; wherein R⁸ and R⁹ may be further independently substituted with up to three substituents chosen from hydroxyl, C₁₋₆alkoxy, C₁₋₆ hydroxy alkyl, C₁₋₆ alkoxy-C₁₋₆ alkyl, C₁₋₆alkoxy-C₁₋₆ alkoxy, C₂₋₆ hydroxyalkoxy, oxo, thiono, cyano or halo; or alternatively, R⁸ and R⁹, taken together with the N atom to which they are both attached, form a heterocycloalkyl ring of 4-7 members, containing up to one other heteroatom selected from O, S, or NR³, or a heterobicycloalkyl ring of 6-12 members which may be fused, bridged or spiro, and contain up to two other heteroatoms chosen from O, S(O)ₓ, or NR³;
wherein each R¹⁰ is independently H, —CD₃, C₁₋₆ alkyl, C₃₋₆ cycloalkyl, phenyl, naphthyl, 5-10 membered mono- or bicyclo-heteroaryl, C₂₋₆ hydroxyalkyl, —SO₂-alkyl, NH—C₂₋₆ alkyl-NR⁸R⁹, C₁₋₆ alkoxy-C₁₋₆ alkyl or C₂₋₆ alkyl-NR⁸R⁹; alternatively, two R¹⁰ taken together with the same N atom to which they are both attached, form a heterocyclic ring of 4-7 members, containing up to one other heteroatom selected from O, S, or NR³;
wherein p=0, 1, 2, 3, or 4;
wherein x=0, 1, or 2.

6. The compound of claim 5,
wherein the compound is encompassed within Formula II 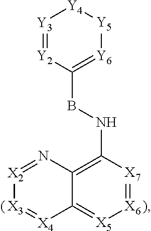
including pharmaceutically acceptable salts, solvates, and/or prodrugs thereof,
wherein X², X³, X⁴, X⁵, X⁶ and X⁷ are independently selected from CR¹ or N, with the proviso that at least three of them must be CR¹;
wherein Y², Y³, Y⁴, Y⁵ Y⁶ are independently selected from CH, CR² or N; or Y⁶ is a bond, in which case one of Y³, Y⁴, or Y⁵ is NR³, O, or S, while the other two may be CR² or N;
wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within claim 5; or
wherein the compound is encompassed within Formula III 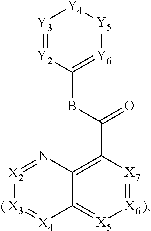
including pharmaceutically acceptable salts, solvates, and/or prodrugs thereof,
wherein X², X³, X⁴, X⁵, X⁶ and X⁷ are independently selected from CR¹ or N, with the proviso that at least three of them must be CR¹;
wherein Y², Y³, Y⁴, Y⁵ Y⁶ are independently selected from CH, CR² or N;
wherein B is selected from a group consisting of NH, CH₂, C(R³)₂, C—NMe₂, C═N—OR⁴, C═N—N(R⁵)₂;
wherein R¹, R², R³, R⁴, R⁵, (R⁷-R¹⁰ embedded in R¹ and R²) are as described within claim 5; or
wherein the compound is encompassed within Formula IV 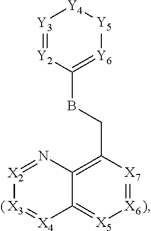
including pharmaceutically acceptable salts, solvates, and/or prodrugs thereof,
wherein X², X³, X⁴, X⁵, X⁶ and X⁷ are independently selected from CR¹ or N, with the proviso that at least three of them must be CR¹;
wherein Y², Y³, Y⁴, Y⁵, Y⁶ are independently selected from CH, CR² or N; or Y⁶ is a bond, in which case one of Y³, Y⁴, or Y⁵ is NR³, O, or S, while the other two may be CR² or N;
wherein B selected from a group consisting of NH, C═O, C═S, CH₂, C(R³)₂, CF₂, C—NMe₂, C═N—OR⁴, C═N—N(R⁵)₂;
wherein R¹, R², R³, R⁴, R⁵, (R⁷-R¹⁰ embedded in R¹ and R²) are as described in claim 5; or
wherein the compound is encompassed within Formula V 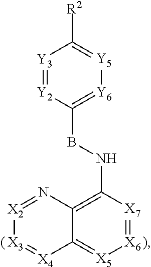
including pharmaceutically acceptable salts, solvates, and/or prodrugs thereof,
wherein X², X³, X⁴, X⁵, X⁶ and X⁷ are independently selected from CR¹ or N, with the proviso that at least three of them must be CR¹;
wherein Y², Y³, Y⁵, Y⁶ are independently selected from CH or N;
wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within claim 5; or
wherein the compound is encompassed within Formula VI 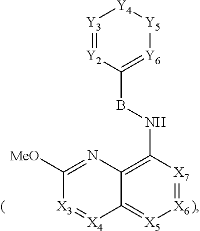
including pharmaceutically acceptable salts, solvates, and/or prodrugs thereof,
wherein X³, X⁴, X⁵, X⁶ and X⁷ are independently selected from CR¹ or N;
wherein Y², Y³, Y⁴, Y⁵, Y⁶ are independently selected from CH, CR² or N;
wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within claim 5; or
wherein the compound is encompassed within Formula VII 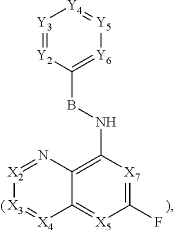
including pharmaceutically acceptable salts, solvates, and/or prodrugs thereof,
wherein X², X³, X⁴, X⁵ and X⁷ are independently selected from CR¹ or N;
wherein Y², Y³, Y⁴, Y⁵, Y⁶ are independently selected from CH, CR² or N;
wherein B is selected from a group consisting of C═O, C═S, CH₂, C(R³)₂, CF₂, C—NMe₂, C═N—OR⁴, C═N—N(R⁵)₂, C═N—SO₂R⁶, C═N—CN;
wherein R¹, R², R³, R⁴, R⁵, R⁶, (R⁷-R¹⁰ embedded in R¹ and R²) are as described within claim 5; or
wherein the compound is encompassed within Formula VIII, 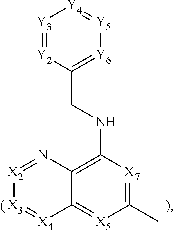
including pharmaceutically acceptable salts, solvates, and/or prodrugs thereof,
wherein X², X³, X⁴, X⁵ and X⁷ are independently selected from CR¹ or N, with the proviso that at least two of them must be CR¹;
wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;
wherein R¹, R², R³, (R⁷-R¹⁰ embedded in R¹ and R²) are as described within claim 5; or
wherein the compound is encompassed within Formula IX 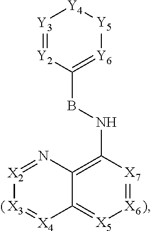
including pharmaceutically acceptable salts, solvates, and/or prodrugs thereof,
wherein X², X³, X⁴, X⁵, X⁶ and X⁷ are independently selected from CR¹ or N, with the proviso that at least three of them must be CR¹;
wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;
wherein B is selected from a group consisting of NH, C═O, C═S, CH₂, C(R³)₂, CF₂, C—NMe₂, C═N—OR⁴, C═N—N(R⁵)₂, C═N—SO₂R⁶, C═N—CN;
wherein R¹ is N(C₂₋₆ alkyl)₂—NH;
wherein R² is selected from H or Me;
wherein R³, R⁴, R⁵, R⁶ are as described within claim 5; or
wherein the compound is encompassed within Formula X 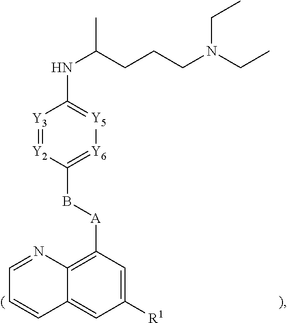
including pharmaceutically acceptable salts, solvates, and/or prodrugs thereof,
wherein Y², Y³, Y⁵ Y⁶ are independently CH or N;
wherein A and B selected from a group consisting of NH, C═O, C═S, CH₂, C(R³)₂, CF₂, C—NMe₂, C═N—OR⁴, C═N—N(R⁵)₂, C═N—SO₂R⁶, C═N—CN;
wherein R¹, R³, R⁴, R⁵, R⁶, (R⁷-R¹⁰ embedded in R¹) are as described within claim 5; or
wherein the compound is encompassed within Formula XI 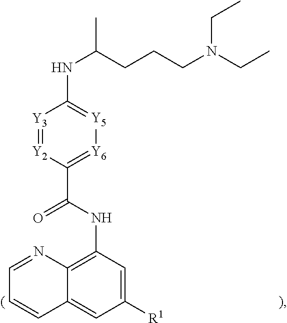
including pharmaceutically acceptable salts, solvates, and/or prodrugs thereof,
wherein Y², Y³, Y⁵, Y⁶ are independently CH or N;
wherein R¹, (R⁷-R¹⁰ embedded in R¹) are as described within claim 5; or
wherein the compound is encompassed within Formula XII 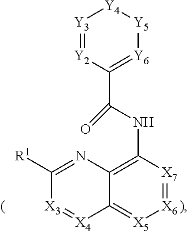
including pharmaceutically acceptable salts, solvates, and/or prodrugs thereof,
wherein X³, X⁴, X⁵, X⁶ and X⁷ are independently selected from CH or N;
wherein Y², Y³, Y⁴, Y⁵, Y⁶ are independently CH, CR² or N; or Y⁶ is a bond, in which case one of Y³, Y⁴, or Y⁵ is NR³, O, or S, while the other two may be CR² or N;
wherein R¹, R², R³, (R⁷-R¹⁰ embedded in R¹ and R²) are as described within claim 5; or
wherein the compound is encompassed within Formula XIII 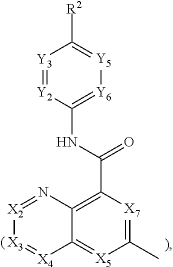
including pharmaceutically acceptable salts, solvates, and/or prodrugs thereof,
wherein X², X³, X⁴, X⁵, and X⁷ are independently selected from CR¹ or N, with the proviso that at least two of them must be CR¹;
wherein Y², Y³, Y⁵, Y⁶ are independently CH or N;
wherein R¹, R², (R⁷-R¹⁰ embedded in R¹ and R²) are as described within claim 5; or
wherein the compound is encompassed within Formula XIV 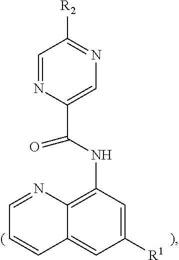
including pharmaceutically acceptable salts (e.g., 2,2,2-trifluoroacetate (TFA) salts and other salts) (e.g., physiologically tolerated acid addition salts), solvates, and/or prodrugs thereof,
wherein R¹ is independently H, Me and halogen;
wherein R², (R⁷-R¹⁰ embedded in R²) are as described within claim 5; or
wherein the compound is encompassed within Formula XV 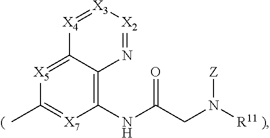
including pharmaceutically acceptable salts, solvates, and/or prodrugs thereof,
wherein X², X³, X⁴, X⁵, and X⁷ are independently selected from CR¹ or N, with the proviso that at least two of them must be CR¹;
wherein Z is independently C₁₋₆ alkyl, C₂₋₆ alkenyl, C₂₋₆ alkynyl, C₃₋₇ cycloalkyl, C₄₋₇ heterocycloalkyl, C₁₋₆ alkyl-C₃₋₇ cycloalkyl, C₁₋₆ alkyl-C₄₋₇ heterocycloalkyl, C₁₋₆ alkyl-phenyl, C₁₋₆ alkyl-naphthyl, C₁₋₆ alkyl-(5-10 membered mono- or bicyclo-heteroaryl), C₂₋₆ alkenyl-C₃₋₇ cycloalkyl, C₂₋₆ alkenyl-C₄₋₇ heterocycloalkyl, C₂₋₆ alkenyl-phenyl, C₂₋₆ alkenyl-naphthyl, C₂₋₆ alkenyl-(5-10 membered mono- or bicyclo-heteroaryl), C₂₋₆ alkynyl-C₃₋₇ cycloalkyl, C₂₋₆ alkynyl-C₄₋₇ heterocycloalkyl, C₂₋₆alkynyl-phenyl, C₂₋₆ alkynyl-naphthyl, C₂₋₆ alkynyl-(5-10 membered mono- or bicyclo-heteroaryl), phenyl, naphthyl, 5-10 membered mono- or bicyclo-heteroaryl, hydroxyl, C₁₋₆alkoxy, C₁₋₆ alkoxy-C₃₋₇ cycloalkyl, C₁₋₆alkoxy-C₄₋₇ heterocycloalkyl, C₁₋₆ alkoxy-phenyl, C₁₋₆ alkoxy-naphthyl, C₁₋₆ alkoxy-(5-10 membered mono- or bicyclo-heteroaryl), C₁₋₆acyloxy, C₁₋₆acyloxy, C₁₋₆acyloxy-C₃₋₇ cycloalkyl, C₁₋₆acyloxy-C₄₋₇ heterocycloalkyl, C₁₋₆ acyloxy-phenyl, C₁₋₆ acyloxy-naphthyl, C₁₋₆ acyloxy-(5-10 membered mono- or bicyclo-heteroaryl), C₁₋₆thioalkoxy, C₁₋₆thioalkoxy, C₁₋₆ thioalkoxy-C₃₋₇ cycloalkyl, C₁₋₆thioalkoxy-C₄₋₇heterocycloalkyl, C₁₋₆ thioalkoxy-phenyl, C₁₋₆ thioalkoxy-naphthyl, C₁₋₆thioalkoxy-(5-10 membered mono- or bicyclo-heteroaryl), amino, C₁₋₆monoalkylamino, C₁₋₆ dialkylamino, C₁₋₆ acyl, C₁₋₆ acylamino, C₂₋₆ alkyl-NR¹⁰R¹⁰, —C₂₋₆ alkyl-R⁷;
wherein R¹¹ is H or Me;
wherein R⁷ and R¹⁰, (R⁸-R⁹ embedded in R⁷ and R¹⁰) are as described within claim 5.

7-19. (canceled)

20. The compound of claim 1, wherein the compound is shown in Table I.

21. (canceled)

22. The compound of claim 1, wherein the compound is comprised within a pharmaceutical composition.

23. A method of treating, ameliorating, or preventing a hyperproliferative condition and/or inflammatory condition, comprising administering to a patient a therapeutically effective amount of the pharmaceutical composition of claim 22.

24. The method of claim 23, wherein the inflammatory condition is a chronic auto immune disorder and/or a viral infection.

25. The method of claim 23, wherein the hyperproliferative condition is diabetes and/or cancer.

26. The method of claim 25, wherein the cancer is one or more of leukemia, colon cancer, CNS cancer, Non-Small lung cancer, melanoma, ovarian cancer, renal cancer, breast cancer, prostate cancer, esophageal cancer, cervical cancer and colorectal cancer.

27. The method of claim 25, further comprising administering to said patient one or more anticancer agents, wherein said anticancer agent one or more of a chemotherapeutic agent, and radiation therapy.

28. The method of claim 23, wherein the patient is a human patient.

29. The method of claim 23, wherein administration of the compound results in activating the cholesterol biosynthesis pathway within cancer cells and/or immune cells.

30. The method of claim 23, wherein administration of the compound results in activating gene expression within one or more of the genes listed in Table III-XIX within cancer cells and/or immune cells.

31. The method of claim 23, wherein administration of the compound results in activating gene expression of one or more of INSIG1, DHCR7, MVK and MSMO1 within cancer cells and/or immune cells.

32. The method of claim 23, wherein administration of the compound results in de-activating gene expression of one or more of GPR135, SPDYA, ABCA1 and HRH4.

33. The method of claim 23, wherein administration of the compound results in activating the cell cycle regulation pathway within cancer cells and/or immune cells.

34. The method of claim 23, wherein administration of the compound results in activating gene expression of one or more of AVPI1, CCNG2, TUBA1A, H2AFX, and HIST1H3C within cancer cells and/or immune cells.

35. The method of claim 23, wherein administration of the compound results in up-regulating HMGCS1 protein expression within cancer cells and/or immune cells.

36-42. (canceled)

43. A kit comprising a compound of claim 1 and instructions for administering said compound to a patient having a hyperproliferative condition and/or inflammatory condition.
